(19) United States
(12) Patent Application Publication
(10) Pub. No.: US 2006/0116410 A1
(43) Pub. Date: Jun. 1, 2006

(54) 4-Aminomethyl benzamidine derivatives

(76) Inventors: David William Banner, Basel (CH); Peter Mohr, Basel (CH); Ulrike Obst, Reinach (CH); Christoph Martin Stahl, Freiburg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/217,860

(22) Filed: Sep. 1, 2005

(30) Foreign Application Priority Data
Sep. 6, 2004 (EP) 04104292.0

(51) Int. Cl. A61K 31/42; A61K 31/4172; A61K 31/415; A61K 31/165

(52) U.S. Cl. 514/378; 514/400; 514/406; 514/616; 548/334.5; 548/374.1; 564/147; 548/248

(57) ABSTRACT

The invention is concerned with novel 4-aminomethyl benzamidine derivatives of formula (I) 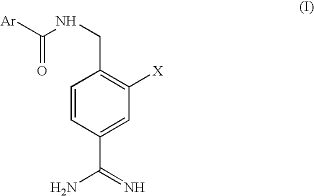 wherein Ar and X are as defined in the description and in the claims, as well as prodrugs and pharmaceutically acceptable salts thereof These compounds inhibit the formation of coagulation factors Xa, IXa and thrombin induced by factor VIIa and tissue factor and can be used as medicaments.

### FIELD OF INVENTION

This invention relates to novel 4-aminomethyl benzamidine derivatives which are factor VIIa inhibitors, pharmaceutical compositions containing them, their use as medicaments and methods for preparing them.

Inhibitors of factor VIIa had previously been suggested for the inhibition of the formation of thrombin and for the treatment of related diseases (WO 00/35858). However, there is still a need for novel factor VIIa inhibitors which exhibit improved pharmacological properties.

The present invention provides the novel compounds of Formula (I) which are factor VIIa inhibitors. The compounds of the present invention exhibit improved pharmacological properties compared to the known compounds.

### DETAILED DESCRIPTION OF THE INVENTION

This invention provides compounds of Formula (I) 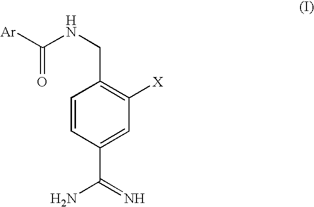
wherein
Ar is aryl or heteroaryl, which is optionally substituted by one, two or three substituents independently selected from the group consisting of halogen, C₁₋₆ alkyl, C₂₋₆ alkenyl, C₂₋₆ alkynyl, C₃₋₇ cycloalkyl, C₃₋₇ cycloalkyl C₁₋₆ alkyl, optionally substituted heterocyclyl, optionally substituted heteroaryl, optionally substituted aryl, optionally substituted aryl-C₁₋₆ alkyl, optionally substituted heteroaryl-C₁₋₆ alkyl, optionally substituted heterocyclyl-C₁₋₆ alkyl, optionally substituted aryloxy-C₁₋₆ alkyl, optionally substituted heteroaryloxy-C₁₋₆ alkyl, optionally substituted heterocyclyloxy-C₁₋₆ alkyl, optionally substituted aryl-C₁₋₆ alkoxy, optionally substituted heteroaryl-C₁₋₆ alkoxy, optionally substituted heterocyclyl-C₁₋₆ alkoxy, fluoro C₁₋₆ alkyl, hydroxy, C₁₋₆ alkoxycarbonyl, carboxy, nitro, cyano, hydroxy C₁₋₆ alkyl-aminocarbonyl, optionally substituted heterocyclyl-carbonyl, optionally substituted heteroaryl-carbonyl, optionally substituted aryl-carbonyl, C₁₋₆ alkoxy C₁₋₆ alkyl-aminocarbonyl, C₁₋₆ alkoxy C₁₋₆ alkoxy, C₁₋₆ alkoxy and amino, in which C₁₋₆ alkoxy may optionally be substituted by one or two substituents independently selected from the group consisting of hydroxy, optionally substituted heteroaryl, optionally substituted aryl, optionally substituted heterocyclyl, carbamoyl, mono- or di-C₁₋₆ alkyl substituted aminocarbonyl, carboxyl and C₁₋₆ alkoxycarbonyl, and amino may optionally be substituted by one or two substituents independently selected from the group consisting of optionally substituted aryl-sulfanyl, optionally substituted aryl-sulfinyl, optionally substituted aryl-sulfonyl, optionally substituted heteroaryl-sulfanyl, optionally substituted heteroaryl-sulfinyl, optionally substituted heteroaryl-sulfonyl, optionally substituted heterocyclyl-sulfanyl, optionally substituted heterocyclyl-sulfinyl, optionally substituted heterocyclyl-sulfonyl, optionally substituted aryl-C₁₋₆ alkylsulfanyl, optionally substituted aryl-C₁₋₆ alkylsulfinyl, optionally substituted aryl-C₁₋₆ alkylsulfonyl, optionally substituted heteroaryl-C₁₋₆ alkylsulfanyl, optionally substituted heteroaryl-C₁₋₆ alkylsulfinyl, optionally substituted heteroaryl-C₁₋₆ alkylsulfonyl, optionally substituted heterocyclyl-C₁₋₆ alkylsulfanyl, optionally substituted heterocyclyl-C₁₋₆ alkylsulfinyl, optionally substituted heterocyclyl-C₁₋₆ alkylsulfonyl, optionally substituted heteroaryl-C₁₋₆ alkyl, optionally substituted aryl-C₁₋₆ alkyl, optionally substituted heterocyclyl-C₁₋₆ alkyl, optionally substituted aryl-C₁₋₆ alkylcarbonyl, optionally substituted heteroaryl-C₁₋₆ alkylcarbonyl, optionally substituted heterocyclyl-C₁₋₆ alkylcarbonyl, mono- or di-C₁₋₆ alkyl substituted aminocarbonyl-C₁₋₆ alkyl, C₁₋₆ alkoxycarbonyl-C₁₋₆ alkyl, C₁₋₆ alkyl, carbamoyl C₁₋₆ alkyl, C₁₋₆ alkylcarbamoyl, C₁₋₆ alkylcarbonyl, C₁₋₆ alkylsulfanyl, C₁₋₆ alkylsulfinyl and C₁₋₆ alkylsulfonyl;
X is X-1: —O—(CH₂)ₙ—Y—R², X-2: —N(R¹)—(CH₂)ₙ—Y—R², X-3: —NO₂ or X-4: hydrogen;
Y is Y-1: —C(═O)—, Y-2: 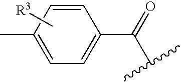
   or absent;
R¹ is hydrogen, C₁₋₆ alkyl, C₃₋₇ cycloalkyl, C₃₋₇ cycloalkyl C₁₋₆ alkyl, optionally substituted aryl-C₁₋₆ alkyl or hydroxy C₁₋₆ alkyl, C₁₋₆ alkoxy C₁₋₆ alkyl;
R² is hydrogen, C₁₋₆ alkyl, C₃₋₇ cycloalkyl, C₃₋₇ cycloalkyl C₁₋₆ alkyl, C₂₋₆ alkenyl, C₂₋₆ alkynl, C₁₋₆ alkoxy, hydroxy, optionally substituted aryl, optionally substituted heterocyclyl, optionally substituted aryl-C₁₋₆ alkyl, optionally substituted heteroaryl-C₁₋₆ alkyl, optionally substituted heterocyclyl-C₁₋₆ alkyl, nitro, cyano, heteroaryl optionally substituted by one or two substituents independently selected from the group consisting of C₁₋₆ alkyl, carboxy, carbamoyl and C₁₋₆ alkoxycarbonyl or amino optionally substituted by one or two substituents independently selected from the group consisting of C₁₋₆ alkyl, C₃₋₇ cycloalkyl, C₃₋₇ cycloalkyl C₁₋₆ alkyl, C₁₋₆ alkylcarbonyl, C₁₋₆ alkylsulfanyl, C₁₋₆ alkylsulfinyl, C₁₋₆ alkylsulfonyl, optionally substituted aryl-carbonyl, optionally substituted heteroaryl-carbonyl, optionally substituted heterocyclyl-carbonyl, optionally substituted aryl, optionally substituted aryl-C₁₋₆ alkyl, optionally substituted heterocyclyl-C₁₋₆ alkyl, C₁₋₆ alkoxy C₁₋₆ alkyl C₁₋₆ alkoxycarbonyl-C₁₋₆ alkyl, carboxyl-C₁₋₆ alkyl, optionally substituted heteroaryl, optionally substituted heteroaryl-C₁₋₆ alkyl, optionally substituted heterocyclyl and hydroxy C₁₋₆ alkyl;
R³ is hydrogen, halogen or C₁₋₆ alkyl;
n is an integer from 0 to 2;
provided that X is not C₁₋₆ alkoxy;
and prodrugs and pharmaceutically acceptable salts thereof.

This invention also provides a process for the manufacture of the above compounds, pharmaceutical preparations which contain such compounds as well as the use of these compounds for the production of pharmaceutical preparations.

This invention also provides pharmaceutical compositions comprising a compound as defined above and a pharmaceutically acceptable carrier and/or adjuvant. The compounds of the invention are useful for the treatment and/or prophylaxis of diseases which are associated with the formation of clotting factors Xa, IXa and thrombin induced by factor VIIa and tissue factor, particularly as therapeutically active substances for the treatment and/or prophylaxis of arterial and venous thrombosis, deep vein thrombosis, pulmonary embolism, unstable angina pectoris, cardiac infarction, stroke due to atrial fibrillation, inflammation, arteriosclerosis and/or tumour.

This invention also provides a method for the therapeutic and/or prophylactic treatment of diseases which are asscociated with the formation of clotting factors Xa, IXa and thrombin induced by factor VIIa and tissue factor, particularly for the therapeutic and/or prophylactic treatment of arterial and venous thrombosis, deep vein thrombosis, pulmonary embolism, unstable angina pectoris, cardiac infarction, stroke due to atrial fibrillation, inflammation, arteriosclerosis and/or tumour, which method comprises administering a compound as defined above to a human being or animal.

### DEFINITIONS

Unless otherwise indicated, the following definitions are set forth to illustrate and define the meaning and scope of the various terms used to describe the invention herein.

The term “halogen” means fluorine, chlorine, bromine and iodine, with fluorine and chlorine being preferred.

The term “C₁₋₆ alkyl”, alone or in combination with other groups, means a branched or straight-chain monovalent alkyl radical, having one to six carbon atoms. This term is further exemplified by such radicals as methyl, ethyl, n-propyl, isopropyl, n-butyl, s-butyl, t-butyl. C₁₋₄ alkyl is more preferred.

The term “fluoro C₁₋₆ alkyl” means C₁₋₆ alkyl groups which are mono- or multiply substituted with fluorine. Examples of fluoroalkyl groups are e.g. CFH₂, CF₂H, CF₃, CF₃CH₂, CF₃(CH₂)₂, (CF₃)₂CH and CF₂H—CF₂. Trifluoromethyl is preferred.

The term “C₃₋₇ cydoalkyl”, alone or in combination with other groups, means a saturated monovalent cyclic hydrocarbon radical of three to seven ring carbons, e.g., cyclopropyl, cyclobutyl, cyclohexyl.

The term “alkoxy”, alone or in combination with other groups, means the group R′—O—, wherein R′ is a C₁₋₆ alkyl.

The term “C₂₋₆ alkenyl”, alone or in combination with other groups, means a straight-chain or branched hydrocarbon residue comprising an olefinic bond, having two to six carbon atoms, such as e.g. ethenyl, 2-propenyl.

The term “C₂₋₆ alkynyl”, alone or in combination with other groups, means a straight-chain or branched hydrocarbon residue comprising a tripple bond, having two to six carbon atoms, such as e.g. ethynyl, 2-propinyl.

The term “aryl”, alone or in combination with other groups, means a phenyl or a naphthyl group, preferably a phenyl group. The term “optionally substituted aryl” means an aryl group described above, which is optionally substituted by one to five, preferably one to three substituents independently selected from the group consisting of halogen, hydroxy, trifluoromethyl, C₁₋₆ alkyl, halo C₁₋₆ alkyl, C₁₋₆ alkoxy, amino, nitro, aminocarbonyl, carboxyl, C₁₋₆ alkoxycarbonyl and C₁₋₆ alkylcarbonyl, preferably selected from the group consisting of halogen, hydroxy, trifluoromethyl, C₁₋₆ alkyl, halo C₁₋₆ alkyl, C₁₋₆ alkoxy, amino, nitro, aminocarbonyl and C₁₋₆ alkylcarbonyl, more preferably selected from the group consisting of halogen, C₁₋₆ alkyl and aminocarbonyl.

The term “heterocyclyl”, alone or combination with other groups, means non-aromatic monocyclic radicals of three to eight ring atoms in which one or two ring atoms are heteroatoms selected from NR^{x} {wherein R^{x} is hydrogen or C₁₋₆ alkyl}, O, or S(O)ₙ (where n is an integer from 0 to 2), the remaining ring atoms being C. The non-aromatic monocyclic ring may optionally be fused to a C₃₋₇ cycloalkyl, aryl or heteroaryl ring, preferably a phenyl ring, with the understanding that the attachment point of the heterocyclyl radical is on the non-aromatic monocyclic ring. One or two carbon atoms of the non-aromatic monocyclic ring may optionally be replaced with a carbonyl group. Examples of suitable heterocyclyl groups are pyrrolidinyl, pyrrolinyl, imidazolidinyl, imidazolinyl, pyrazolidinyl, pyrazolinyl, piperidyl, piperazinyl, morpholinyl, pyranyl, tetrahydropyranyl, 4,5-dihydro-oxazolyl, 4,5-dihydro-thiazolyl. Preferred heterocyclyl groups are piperidinyl, morpholinyl, pyrrolidinyl, tetrahydrofuranyl, 1,3-dioxo-isoindolinyl and tetrahydropyranyl, especially morpholinyl. The term “optionally substituted heterocyclyl” means a heterocyclyl group described above, which is optionally substituted independently with one, two, or three substituents, preferably one or two substituents selected from the group consisting of halogen, hydroxy, trifluoromethyl, C₁₋₆ alkyl, halo C₁₋₆ alkyl, C₁₋₆ alkoxy, amino, nitro, aminocarbonyl, carboxyl, C₁₋₆ alkoxycarbonyl and C₁₋₆ alkylcarbonyl, preferably selected from the group consisting of halogen, hydroxy, trifluoromethyl, C₁₋₆ alkyl, halo C₁₋₆ alkyl, C₁₋₆ alkoxy, amino, nitro, aminocarbonyl and C₁₋₆ alkylcarbonyl, more preferably selected from the group consisting of halogen, C₁₋₆ alkyl and aminocarbonyl.

The term “heteroaryl”, alone or combination with other groups, means a monocyclic or bicyclic radical of 5 to 12 ring atoms having at least one aromatic ring containing one, two, or three ring heteroatoms selected from N, O, and S, the remaining ring atoms being C, with the understanding that the attachment point of the heteroaryl radical will be on an aromatic ring. A preferred heteroaryl is a monocyclic radical of five or six ring atoms having one or two ring heteroatoms selected from N and O. Examples of suitable heteroaryls are furyl, pyridyl, pyridazinyl, pyrimidinyl, pyrazinyl, pyrazolyl, isoxazolyl, oxazolyl, oxadiazolyl, imidazolyl, pyrrolyl, tetrazolyl. Pyridyl, pyrazolyl, oxazolyl imidazolyl and isoxazolyl are more preferred.

The term “optionally substituted heteroaryl” means a heteroaryl group described above, which is optionally substituted independently with one, two, or three substituents, preferably one or two substituents selected from the group consisting of halogen, hydroxy, trifluoromethyl, C₁₋₆ alkyl, halo C₁₋₆ alkyl, C₁₋₆ alkoxy, amino, nitro, aminocarbonyl, carboxyl, C₁₋₆ alkoxycarbonyl and C₁₋₆ alkylcarbonyl, preferably selected from the group consisting of halogen, hydroxy, trifluoromethyl, C₁₋₆ alkyl, halo C₁₋₆ alkyl, C₁₋₆ alkoxy, amino, nitro, aminocarbonyl and C₁₋₆ alkylcarbonyl, more preferably selected from the group consisting of halogen, C₁₋₆ alkyl and aminocarbonyl.

Preferred radicals for the chemical groups whose definitions are given above are those specifically exemplified in Examples.

Compounds of formula (I) can form pharmaceutically acceptable acid addition salts. Examples of such pharmaceutically acceptable salts are salts of compounds of formula (I) with physiologically compatible mineral acids, such as hydrochloric acid, sulphuric acid, sulphurous acid or phosphoric acid; or with organic acids, such as methanesulphonic acid, p-toluenesulphonic acid, acetic acid, lactic acid, trifluoroacetic acid, citric acid, fumaric acid, maleic acid, tartaric acid, succinic acid or salicylic acid. The term “pharmaceutically acceptable salts” refers to such salts. Compounds of formula (I) in which a COOH group is present can further form salts with bases. Examples of such salts are alkaline, earth-alkaline and ammonium salts such as e.g. Na—, K—, Ca— and trimethylammonium salts. The term “pharmaceutically acceptable salts” also refers to such salts. Acid addition salts as described above are preferred.

“Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N,O-dimethylhydroxylamino.

“Optional” or “optionally” means that the subsequently described event or circumstance may but need not occur, and that the description includes instances where the event or circumstance occurs and instances in which it does not. For example, “aryl group optionally substituted with an alkyl group” means that the alkyl may but need not be present, and the description includes situations where the aryl group is substituted with an alkyl group and situations where the aryl group is not substituted with the alkyl group.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

“Protecting group” refers to a grouping of atoms that when attached to a reactive group in a molecule masks, reduces or prevents that reactivity. Examples of protecting groups can be found in T. W. Green and P. G. Futs, Protective Groups in Organic Chemistry, (Wiley, 2^{nd} ed. 1991) and Harrison and Harrison et al., Compendium of Synthetic Organic Methods, Vols. 1-8 (John Wiley and Sons, 1971-1996). Representative amino protecting groups include formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC). Representative hydroxy protecting groups include those where the hydroxy group is either acylated or alkylated such as benzyl, and trityl ethers as well as alkyl ethers, tetrahydropyranyl ethers, trialkylsilyl ethers and allyl ethers.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Prodrugs” means any compound which releases an active parent drug according to Formula (I) in vivo when such a prodrug is administered to a mammalian subject. Prodrugs of a compound of Formula (I) are prepared by modifying functional groups present in the compound of Formula (I) in such a way that the modifications may be cleaved in vivo to release the parent compound. Prodrugs include compounds of Formula (I) wherein a hydroxy, an amino or an amidino group in a compound of Formula (I) is bonded to any group that may be cleaved in vivo to regenerate the free parent group, respectively. Examples of prodrugs include, but are not limited to esters, carbonates, carbamates, amidoximes and derivatives thereof.

Compounds that have the same molecular Formula but differ in the nature or sequence of bonding of their atoms or the arrangement of their atoms in space are termed “isomers.” Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

While the broadest definition of this invention is described before, certain compounds of Formula (I) are preferred.

i) A preferred compound of the invention is a compound of Formula (I) wherein Ar is aryl or heteroaryl, which is optionally substituted by one or two substituents independently selected from the group consisting of halogen, especially fluoro or chloro, C₁₋₆ alkyl, especially methyl, optionally substituted aryl-C₁₋₆ alkyl, especially benzyl, fluoro C₁₋₆ alkyl, especially trifluoromethyl, hydroxy, C₁₋₆ alkoxycarbonyl, especially methoxycarbonyl, carboxy, nitro, cyano, hydroxy C₁₋₆ alkyl-aminocarbonyl, optionally substituted heterocyclyl-carbonyl, especially morpholinylcarbonyl, C₁₋₆ alkoxy C₁₋₆ alkylaminocarbonyl, C₁₋₆ alkoxy and amino, in which C₁₋₆ alkoxy may optionally be substituted by one or two substituents independently selected from the group consisting of hydroxy, carboxy, C₁₋₆ alkoxycarbonyl, optionally substituted heteroaryl, carbamoyl and mono C₁₋₆ alkyl substituted amino-carbonyl, and amino may optionally be substituted by one or two substituents independently selected from the group consisting of optionally substituted aryl-sulfonyl, especially phenylsulfonyl, optionally substituted heteroaryl-C₁₋₆ alkyl, especially pyridyl C₁₋₆ alkyl, C₁₋₆ alkyl, carbamoyl C₁₋₆ alkyl and C₁₋₆ alkylsulfonyl. More preferred substituents are halogen, especially fluoro or chloro, C₁₋₆ alkyl, especially methyl, C₁₋₆ alkoxy, especially methoxy, hydroxy C₁₋₆ alkoxy, especially 2-hydroxyethoxy, optionally substituted heteroaryl-C₁₋₆ alkoxy, especially pyridyl C₁₋₆ alkyl, carbamoyl C₁₋₆ alkoxy, especially carbamoylmethoxy, mono C₁₋₆ alkyl substituted aminocarbonyl C₁₋₆ alkoxy, especially N-methylcarbamoylmethoxy, C₁₋₆ alkoxycarbonyl, especially methoxycarbonyl, nitro, or C₁₋₆ alkoxycarbonyl C₁₋₆ alkoxy.

When Ar is aryl, a preferred aryl is phenyl.

When Ar is heteroaryl, a preferred heteroaryl is a monocyclic radical of five or six ring atoms having one or two ring heteroatoms selected from N and O, more preferably furyl, pyridyl, pyridazinyl, pyrimidinyl, pyrazinyl, pyrazolyl, isoxazolyl, oxazolyl, oxadiazolyl, imidazolyl, pyrrolyl or tetrazolyl, especially pyridyl, oxazolyl, isoxazolyl, imidazolyl or pyrazolyl.

ii) Another preferred compound of the invention is a compound of Formula (I) wherein X is X-1 and n is 1.

a) Among the compounds mentioned under ii), a preferred compound of the invention is a compound of Formula (I) wherein X is X-1, n is 1 and Y is Y-1. In this case, R² is preferably amino optionally substituted by one or two substituents selected independently from the group consisting of C₁₋₆ alkyl, optionally substituted aryl and optionally substituted heterocyclyl-C₁₋₆ alkyl, more preferably R² is amino, di C₁₋₆ alkylamino or amino substituted by one substituent selected from the group consisting of C₁₋₆ alkyl, optionally substituted aryl and optionally substituted heterocyclyl-C₁₋₆ alkyl, especially amino or mono C₁₋₆ alkylamino (C₁₋₆ alkyl-NH—).

A preferred mono C₁₋₆ alkylamino is methylamino, and a preferred di C₁₋₆ alkylamino is dimethylamino. A preferred optionally substituted aryl-amino is optionally substituted phenyl-amino, more preferred is halogen substituted phenyl-amino. A preferred halogen in the halogen substituted phenyl-amino is fluoro or chloro, especially fluoro. A preferred heterocyclyl group in the optionally substituted heterocyclyl-C₁₋₆ alkylamino is pyrrolidinyl, pyrrolinyl, imidazolidinyl, imidazolinyl, pyrazolidinyl, pyrazolinyl, piperidyl, piperazinyl, morpholinyl, pyranyl, tetrahydropyranyl, 4,5-dihydro-oxazolyl or 4,5-dihydro-thiazolyl, a more preferred heterocyclyl group is piperidinyl, morpholinyl, pyrrolidinyl, tetrahydrofuranyl or tetrahydropyranyl, especially morpholinyl.

When X is X-1, n is 1 and Y is Y-1, Ar is preferably one of those mentioned under i).

Particularly preferred compounds in this group are:
N-(4-Carbamimidoyl-2-carbamoylmethoxybenzyl)-3-fluoro-benzamide hydrochloride;
N-(4-Carbamimidoyl-2-carbamoylmethoxybenzyl)-3-methoxy-benzamide hydrochloride;
4-Amino-N-(4-Carbamimidoyl-2-carbamoylmethoxybenzyl)-3-methyl-benzamide acetic acid salt;
5-Methyl-isoxazole-3-carboxylic acid 4-carbamimidoyl-2-carbamoylmethoxy-benzylamide acetic acid salt;
3-Methyl-isoxazole-5-carboxylic acid 4-carbamimidoyl-2-carbamoylmethoxy-benzylamide acetic acid salt;
N-(4-Carbamimidoyl-2-carbamoylmethoxybenzyl)-3-methyl-benzamide hydrochloride;
2-Benzyl-5-methyl-2H-pyrazole-3-carboxylic acid 4-carbamimidoyl-2-carbamoylmethoxy-benzylamide acetic acid salt;
N-(4-Carbamimidoyl-2-carbamoylmethoxybenzyl)-5-methyl-nicotinamide hydrochloride;
N-(4-Carbamimidoyl-2-carbamoylmethoxybenzyl)-5-methyl-nicotinamide ammoniumchloride;
N-(4-Carbamimidoyl-2-carbamoylmethoxybenzyl)-2-methyl-isonicotinamide acetic acid salt;
2-Benzenesulfonylamino-N-(4-carbamirnidoyl-2-methylcarbamoylmethoxy-benzyl)-5-methyl-benzamide hydrochloride;
N-(4-Carbamimidoyl-2-methylcarbamoylmethoxy-benzyl)-2,5-dichloro-benzamide hydrochloride;
N-(4-Carbamimidoyl-2-methylcarbamoylmethoxy-benzyl)-3-chloro-4-fluoro-benzamide hydrochloride;
N-(4-Carbamimidoyl-2-methylcarbamoylmethoxy-benzyl)-3,5-dichloro-4-fluoro-benzamide hydrochloride;
N-(4-Carbamimidoyl-2-methylcarbamoylmethoxy-benzyl)-3,5-dichloro-benzamide hydrochloride;
N-(4-Carbamimidoyl-2-methylcarbamoylmethoxy-benzyl)-5-chloro-nicotinamide hydrochloride;
N-(4-Carbamimidoyl-2-methylcarbamoylmethoxy-benzyl)-3-trifluoromethyl-benzamide hydrochloride;
N-(4-Carbamimidoyl-2-methylcarbamoylmethoxy-benzyl)-3-chloro-5-methoxy-benzamide hydrochloride;
N-(4-Carbamimidoyl-2-methylcarbamoylmethoxy-benzyl)-5-chloro-2-hydroxy-benzamide hydrochloride;
2,5-Dimethyl-2H-pyrazole-3-carboxylic acid 4-carbamimidoyl-2-methylcarbamoylmethoxy-benzylamide hydrochloride;
1,5-Dimethyl-1H-pyrazole-3-carboxylic acid 4-carbamimidoyl-2-methylcarbamoylmethoxy-benzylamide hydrochloride;
2-Methyl-oxazole-4-carboxylic acid 4-carbamimidoyl-2-methylcarbamoylmethoxy-benzylamide hydrochloride;
N-(4-Carbamimidoyl-2-carbamoylmethoxy-benzyl)-4-fluoro-3-methyl-benzamide acetic acid salt;
N-(4-Carbamimidoyl-2-methylcarbamoylmethoxy-benzyl)-4-fluoro-3-methyl-benzamide hydrochloride;
[1-Amino-1-{4-[(4-fluoro-3-methyl-benzoylamino)-methyl]-3-methylcarbamoylmethoxy-phenyl}-meth-(Z)-ylidene]-carbamic acid ethyl ester;
4-Fluoro-N-[4-(N-hydroxycarbamimidoyl)-2-methylcarbamoylmethoxy-benzyl]-3-methyl-benzamide;
(RS)—N-(4-Carbamimidoyl-2-carbamoylmethoxy-benzyl)-3-chloro-benzamide hydrochloride;
N-(4-Carbamimidoyl-2-methylcarbamoylmethoxy-benzyl)-3-chloro-benzamide acetic acid salt;
N-{4-Carbamimidoyl-2-[(4-fluoro-phenylcarbamoyl)-methoxy]-benzyl}-3-chloro-benzamide acetic acid salt;
N-{4-Carbamimidoyl-2-[(2-morpholin-4-yl-ethylcarbamoyl)-methoxy]-benzyl}-3-chloro-benzamide hydrochloride;
N-(4-Carbamimidoyl-2-methylcarbamoylmethoxy-benzyl)-5-chloro-isophthalamic acid methyl ester hydrochloride;
N-(4-Carbamimidoyl-2-methylcarbamoylmethoxy-benzyl)-5-chloro-isophthalamic acid;
N-(4-Carbamimidoyl-2-methylcarbamoylmethoxy-benzyl)-5-chloro-N′-(2-methoxy-ethyl)-isophthalamide hydrochloride;
N-(4-Carbamimidoyl-2-methylcarbamoylmethoxy-benzyl)-3-chloro-5-(morpholine-4-carbonyl)-benzamide hydrochloride;
N-(4-Carbamimidoyl-2-methylcarbamoylmethoxy-benzyl)-5-chloro-N′-(2-hydroxy-ethyl)-isophthalamide hydrochloride;
N-(4-Carbamimidoyl-2-methylcarbamoylmethoxy-benzyl)-5-chloro-2-methylamino-benzamide hydrochloride;
N-(4-Carbamimidoyl-2-methylcarbamoylmethoxy-benzyl)-5-chloro-2-(2-pyridin-4-yl-ethylamino)-benzamide hydrochloride;
3-Amino-N-(4-carbamimidoyl-2-methylcarbamoylmethoxy-benzyl)-benzamide hydrochloride;
N-(4-Carbamimidoyl-2-methylcarbamoylmethoxy-benzyl)-3-hydroxy-5-methyl-benzamide hydrochloride;
[3-(4-Carbamimidoyl-2-methylcarbamoylmethoxy-benzylcarbamoyl)-5-methyl-phenoxy]-acetic acid;
N-(4-Carbamimidoyl-2-methylcarbamoylmethoxy-benzyl)-3-chloro-5-hydroxy-benzamide hydrochloride;
(4-Carbamimidoyl-2-carbamoylmethoxy-benzyl)-3-chloro-5-nitro-benzamide acetic acid salt;
(4-Carbamimidoyl-2-methylcarbamoylmethoxy-benzyl)-3-chloro-5-nitro-benzamide acetic acid salt;
N-(4-Carbamimidoyl-2-methylcarbamoylmethoxy-benzyl)-3-chloro-5-fluoro-benzamide hydrochloride;
N-(4-Carbamimidoyl-2-methylcarbamoylmethoxy-benzyl)-3-chloro-5-fluoro-benzamide hydrochloride;
N-(4-Carbamimidoyl-2-methylcarbamoylmethoxy-benzyl)-3-chloro-2 fluoro-5-methoxy-benzamide hydrochloride;
N-(4-Carbamimidoyl-2-methylcarbamoylmethoxy-benzyl)-3-chloro-2,4-difluoro-benzamide hydrochloride;
N-(4-Carbamimidoyl-2-carbamoylmethoxy-benzyl)-3-carbamoylmethoxy-5-chloro-benzamide hydrochloride;
N-(4-Carbamimidoyl-2-methylcarbamoylmethoxy-benzyl)-3-chloro-5-methylcarbamoylmethoxy-benzamide hydrochloride;
N-(4-Carbamimidoyl-2-methylcarbamoylmethoxy-benzyl)-3-(2-hydroxy-ethoxy)-5-methyl-benzamide hydrochloride;
N-(4-Carbamimidoyl-2-methylcarbamoylmethoxy-benzyl)-3-carbamoylmethoxy-5-methyl-benzamide acetic acid salt;
N-(4-Carbamimidoyl-2-methylcarbamoylmethoxy-benzyl)-3-chloro-5-(pyridin-4-ylmethoxy)-benzamide hydrochloride;
N-(4-Carbamimidoyl-2-carbamoylmethoxy-benzyl)-3-chloro-5-(pyridin-4-ylmethoxy)-benzamide hydrochloride;
N-(4-Carbamimidoyl-2-carbamoylmethoxy-benzyl)-3-chloro-5-(pyridin-3-ylmethoxy)-benzamide hydrochloride;
N-(4-Carbamimidoyl-2-carbamoylmethoxy-benzyl)-3-chloro-5-(pyridin-2-ylmethoxy)-benzamide hydrochloride;
N-(4-Carbamimidoyl-2-methylcarbamoylmethoxy-benzyl)-3-chloro-5-(1-methyl-1H-imidazol-2-ylmethoxy)-benzamide hydrochloride;
3-Amino-N-(4-carbamimidoyl-2-carbamoylmethoxy-benzyl)-5-chloro-4-fluoro-benzamide hydrochloride;
3-Acetylamino-N-(4-carbamimidoyl-2-methylcarbamoylmethoxy-benzyl)-5-chloro-4-fluoro-benzamide hydrochloride;
N-(4-Carbamimidoyl-2-carbamoylmethoxy-benzyl)-3-chloro-5-isobutyrylamino-benzamide;
N-(4-Carbamimidoyl-2-methylcarbamoylmethoxy-benzyl)-3-chloro-5-isobutyrylamino-benzamide;
{5-Carbamimidoyl-2-[(3-chloro-5-isobutyrylamino-benzoylamino)-methyl]-phenoxy}-acetic acid ethyl ester;
3-Acetylamino-N-(4-carbamimidoyl-2-methylcarbamoylmethoxy-benzyl)-5-chloro-benzamide;
N-(4-Carbamimidoyl-2-methylcarbamoylmethoxy-benzyl)-3-chloro-5-methanesulfonylamino-benzamide;
N-(4-Carbamimidoyl-2-carbamoylmethoxy-benzyl)-3-chloro-5-methanesulfonylamino-benzamide;
N-(4-Carbamimidoyl-2-carbamoylmethoxy-benzyl)-3-(carbamoylmethyl-methanesulfonyl-amino)-5-chloro-benzamide.

b) Among the compounds mentioned under ii), another preferred compound of the invention is a compound of Formula (I) wherein X is X-1, n is 1 and Y is Y-2. In this case, R² is preferably hydroxy or C₁₋₆ alkoxy, especially methoxy. R³ is preferably hydrogen. When X is X-1, n is 1 and Y is Y-2, Ar is preferably one of those mentioned under i), and especially C₁₋₆ alkylphenyl.

Particularly preferred compounds in this group are:
4-{5-Carbamimidoyl-2-[(3-methylbenzoylamino)-methyl]-phenoxymethyl}-benzoic acid methyl ester hydrochloride;
4-{5-Carbamimidoyl-2-[(3-methylbenzoylamino)-methyl]-phenoxymethyl}-benzoic acid.

c) Among the compounds mentioned under ii), another preferred compound of the invention is a compound of Formula (I) wherein X is X-1, n is 1 and Y is absent. In this case, R² is preferably heteroaryl optionally substituted by one or two substituents selected from the group consisting of C₁₋₆ alkyl, carboxy and C₁₋₆ alkoxycarbonyl. A preferred heteroaryl group for R² is a monocyclic radical of five or six ring atoms having one or two ring heteroatoms selected from N and O, more preferably furyl, pyridyl, pyridazinyl, pyrimidinyl, pyrazinyl, pyrazolyl, isoxazolyl, oxazolyl, oxadiazolyl, imidazolyl, pyrrolyl or tetrazolyl, especially pyridyl, pyrazolyl, oxazolyl or isoxazolyl.

When X is X-1, n is 1 and Y is absent, Ar is preferably one of those mentioned under i), and especially phenyl optionally substituted by one or two substituents selected from the group consisting of C₁₋₆ alkyl and amino.

Particularly preferred compounds in this group are:
4-Amino-N-[4-carbamimidoyl-2-(pyridin-2-ylmethoxy)-benzyl]-3-methyl-benzamide acetic acid salt;
N-[4-Carbamimidoyl-2-(pyridin-2-ylmethoxy)-benzyl]-3-methyl-benzamide hydrochloride;
5-{5-Carbamimidoyl-2-[(3-methyl-benzoylamino)-methyl]-phenoxymethyl}-2-methyl-2H-pyrazole-3-carboxylic acid ethyl ester hydrochloride;
5-{5-Carbamimidoyl-2-[(3-methyl-benzoylamino)-methyl]-phenoxymethyl}-2-methyl-2H-pyrazole-3-carboxylic acid;
N-[4-Carbamimidoyl-2-(pyridin-2-ylmethoxy)-benzyl]-3-chloro-5-(pyridin-2-ylmethoxy)-benzamide hydrochloride.

iii) Another preferred compound of the invention is a compound of Formula (I) wherein X is X-1 and n is 2.

A preferred compound in this group is a compound of Formula (I) wherein Y is absent. In this case, R² is preferably C₁₋₆ alkylcarbonylamino, especially acetylamino, C₁₋₆ alkylsulfanylamino, C₁₋₆ alkylsulfinylamino, C₁₋₆ alkylsulfonylamino, heterocyclyl or optionally substituted aryl-carbonylamino, and more preferably C₁₋₆ alkylcarbonylamino, especially acetylamino, C₁₋₆ alkylsulfonylamino, heterocyclyl, especially 1,3-dioxo-isoindolynyl or optionally substituted aryl-carbonylamino, further more preferably optionally substituted aryl-carbonylamino or heterocydyl, especially 1,3-dioxo-isoindolynyl. A preferred optionally substituted aryl-carbonylamino is optionally substituted phenyl-carbonylamino, more preferred is halogen substituted phenyl-carbonylamino. A preferred halogen in the halogen substituted phenyl-carbonylamino is fluoro or chloro, especially fluoro.

When X is X-1, n is 2 and Y is absent, Ar is preferably one of those mentioned under i), and especially phenyl optionally substituted by one or two substituents selected from the group consisting of C₁₋₆ alkyl and halogen such as fluoro, chloro.

Particularly preferred compounds in this group are:
N-[2-(2-Acetylamino-ethoxy)-4-carbamimidoyl-benzyl]-4-fluoro-3-methyl-benzamide hydrochloride;
N-[4-Carbamimidoyl-2-(2-methanesulfonylaminoethoxy)-benzyl]-4-fluoro-3-methyl-benzamide hydrochloride;
N-{4-Carbamimidoyl-2-[2-(2-fluorobenzoylamino)-ethoxy]-benzyl}-4-fluoro-3-methyl-benzamid hydrochloride;
N-[2-(2-{[(3-Chlorobenzoyl)amino]methyl}-5-carbamimidoylphenoxy)ethyl]-2-fluorobenzamide;
N-{4-Carbamimidoyl-2-[2-(1,3-dioxo-1,3-dihydro-isoindol-2-yl)-ethoxy]-benzyl}-4-fluoro-3-methyl-benzamide hydrochloride.

iv) Another preferred compound of the invention is a compound of Formula (I) wherein X is X-2 and n is 1.

a) Among the compounds mentioned under iv), a preferred compound of the invention is a compound of Formula (I) wherein X is X-2, n is 1 and Y is Y-1. In this case, R² is preferably hydroxy, C₁₋₆ alkoxy, especially methoxy or ethoxy, optionally substituted heterocydyl or amino optionally substituted by one or two substituents independently selected from the group consisting of C₁₋₆ alkyl, hydroxy C₁₋₆ alkyl, optionally substituted heterocydyl, optionally substituted heteroaryl and optionally substituted aryl. More preferably R2 is hydroxy, C₁₋₆ alkoxy, especially methoxy or ethoxy, amino, mono C₁₋₆ alkylamino, especially methylamino, di C₁₋₆ alkylamino, especially dimethylamino, optionally substituted heterocyclyl, optionally substituted heteroaryl-amino, hydroxy C₁₋₆ alkyl-amino or optionally substituted aryl-amino. A preferred heterocyclyl group in the optionally substituted heterocyclyl is pyrrolidinyl, pyrrolinyl, imidazolidinyl, imidazolinyl, pyrazolidinyl, pyrazolinyl, piperidyl, piperazinyl, morpholinyl, pyranyl, tetrahydropyranyl, 4,5-dihydro-oxazolyl or 4,5-dihydro-thiazolyl, a more preferred heterocyclyl group is piperidinyl, morpholinyl, pyrrolidinyl, tetrahydrofuranyl or tetrahydropyranyl, especially morpholinyl. Non-substituted heterocyclyl is preferred. A preferred optionally substituted aryl-amino is optionally substituted phenyl-amino, especially phenylamino. A preferred heteroaryl group in the optionally substituted heteroaryl-amino is a monocyclic radical of five or six ring atoms having one or two ring heteroatoms selected from N and O, more preferably furyl, pyridyl, pyridazinyl, pyrimidinyl, pyrazinyl, pyrazolyl, isoxazolyl, oxazolyl, oxadiazolyl, imidazolyl, pyrrolyl or tetrazolyl, especially pyridyl or isoxazolyl. N-hydroxy C₁₋₆ alkyl-N-optionally substituted aryl-amino and N-hydroxy C₁₋₆ alkyl-N-optionally substituted heteroaryl-amino are also preferred as R². R¹ is preferably hydrogen.

When X is X-2, n is 1 and Y is Y-1, Ar is preferably one of those mentioned under i), and especially phenyl optionally substituted by one or two substituents selected from the group consisting of C₁₋₆ alkyl and halogen such as fluoro, chloro.

Particularly preferred compounds in this group are:
{5-Carbamimidoyl-2-[(3-methyl-benzoylamino)-methyl]-phenylamino}acetic acid ethyl ester hydrochloride;
{5-Carbamimidoyl-2-[(3-methyl-benzoylamino)-methyl]-phenylamino}acetic acid;
N-[4-Carbamimidoyl-2-(methylcarbamoylmethyl-amino)-benzyl]-3-methyl-benzamide hydrochloride;
N-[4-Carbamimidoyl-2-(dimethylcarbamoylmethyl-amino)-benzyl]-3-methyl-benzamide hydrochloride;
N-[4-Carbamimidoyl-2-(2-morpholin-4-yl-2-oxo-ethylamino)-benzyl]-3-methyl-benzamide hydrochloride;
N-[4-Carbamimidoyl-2-(phenylcarbamoylmethyl-amino)-benzyl]-3-methyl-benzamide hydrochloride;
{5-Carbamimidoyl-2-[(4-fluoro-3-methyl-benzoylamino)-methyl]-phenylamino}acetic acid ethyl ester hydrochloride;
{5-Carbamimidoyl-2-[(4-fluoro-3-methyl-benzoylamino)-methyl]-phenylamino}acetic acid;
N-{4-Carbamimidoyl-2-[(pyridin-2-ylcarbamoylmethyl)-amino]-benzyl}-3-chloro-benzamide hydrochloride;
N-{4-Carbamimidoyl-2-[(pyridin-3-ylcarbamoylmethyl)-amino]-benzyl}-3-chloro-benzamide hydrochloride;
N-{4-Carbamimidoyl-2-[(isoxazol-3-ylcarbamoylmethyl)-amino]-benzyl}-3-chloro-benzamide hydrochloride;
N-[4-Carbamimidoyl-2-({[(2-hydroxy-ethyl)-pyridin-2-yl-carbamoyl]-methyl}-amino)-benzyl]-3-chloro-benzamide hydrochloride;
N-[4-Carbamimidoyl-2-({[(2-hydroxy-ethyl)-phenyl-carbamoyl]-methy-}-amino)-benzyl]-3-chloro-benzamide hydrochloride;
N-(4-Carbamimidoyl-2-{[(1-methyl-piperidin-4-ylcarbamoyl)-methyl]-amino}-benzyl)-3-chloro-benzamide acetic acid salt;
{5-Carbamimidoyl-2-[(3-chloro-5-methoxy-benzoylamino)-methyl]-phenylamino}-acetic acid;
{5-Carbamimidoyl-2-[(3-chloro-5-methoxy-benzoylamino)-methyl]-phenylamino}-acetic acid ethyl ester hydrochloride.

b) Among the compounds mentioned under iv), another preferred compound of the invention is a compound of Formula (I) wherein X is X-2, n is 1 and Y is Y-2. In this case, R² is preferably hydroxy, C₁₋₆ alkoxy, especially methoxy, amino, mono C₁₋₆ alkylamino, di C₁₋₆ alkylamino, C₁₋₆ alkoxy C₁₋₆ alkyl-amino, especially ethoxymethyl-amino, or optionally substituted heterocyclyl, more preferably hydroxy, C₁₋₆ alkoxy, especially methoxy, amino, C₁₋₆ alkoxy C₁₋₆ alkyl-amino, especially ethoxymethyl-amino, or optionally substituted heterocyclyl.

A preferred heterocyclyl group in the optionally substituted heterocyclyl is pyrrolidinyl, pyrrolinyl, imidazolidinyl, imidazolinyl, pyrazolidinyl, pyrazolinyl, piperidyl, piperazinyl, morpholinyl, pyranyl, tetrahydropyranyl, 4,5-dihydro-oxazolyl or 4,5-dihydro-thiazolyl, a more preferred heterocyclyl group is piperidinyl, morpholinyl, pyrrolidinyl, tetrahydrofuranyl or tetrahydropyranyl, especially morpholinyl. Non-substituted heterocyclyl is preferred. R¹ is preferably hydrogen. R³ is preferably hydrogen or halogen, such as fluoro, chloro. When X is X-2, n is 1 and Y is Y-2, Ar is preferably one of those mentioned under i), and especially phenyl optionally substituted by one or two substituents selected from the group consisting of C₁₋₆ alkyl and halogen such as fluoro, chloro.

Particularly preferred compounds in this group are:
4-({5-Carbamimidoyl-2-[(3-methyl-benzoylamino)-methyl]-phenylamino}-methyl)-3-fluoro-benzoic acid methyl ester hydrochloride;
4-({5-Carbamimidoyl-2-[(3-methyl-benzoylamino)-methyl]-phenylamino}-methyl)-3-fluoro-benzoic acid;
N-{2-{[3-(Aminocarbonyl)benzyl]amino}-4-[amino(imino)methyl]-benzyl}-3-chlorobenzamide hydrochloride;
3-({5-Carbamimidoyl-2-[(3-chloro-benzoylamino)-methyl]-phenylamino}-methyl)-benzoic acid methyl ester hydrochloride;
3-({5-Carbamimidoyl-2-[(3-chloro-benzoylamino)-methyl]-phenylamino}-methyl)-benzoic acid;
N-{4-[Amino(hydroxyimino)methyl]-2-[(3-{[(2-methoxyethyl)amino]carbonyl}benzyl)amino]benzyl}-3-chlorobenzamide;
N-{4-[Amino(imino)methyl]-2-[(3-{[(2-methoxyethyl)amino]carbonyl}benzyl)amino]benzy}-3-chlorobenzamide acetic acid salt;
3-Chloro-N-{4-(N-hydroxycarbamimidoyl)-2-[3-(morpholine-4-carbonyl)-benzylamino]-benzyl}-benzamide;
N-{4-[Amino(imino)methyl]-2-{[3-(4-morpholinylcarbonyl)benzyl]amino}benxyl}-3-chlorobenzamide acetic acid salt;
N-{2-{[4-(Aminocarbonyl)benzyl]amino}-4-[amino(hydroxyimino)methyl]benzyl}-3-chlorobenzamide;
N-{2-{[4-(Aminocarbonyl)benzyl]amino}-4-[amino(imino)methyl]benzyl}-3-chlorobenzamide acetic acid salt.

c) Among the compounds mentioned under iv), another preferred compound of the invention is a compound of Formula (I) wherein X is X-2, n is 1 and Y is absent. In this case, R² is preferably hydrogen, optionally substituted heteroaryl or optionally substituted aryl, more preferably optionally substituted phenyl. Especially phenyl is preferred. R¹ is preferably hydrogen or optionally substituted aryl C₁₋₆ alkyl, especially benzyl.

When X is X-2, n is 1 and Y is absent, Ar is preferably one of those mentioned under i), and especially phenyl optionally substituted by C₁₋₆ alkyl.

Particularly preferred compounds in this group are:
N-(2-Benzylamino-4-carbamimidoyl-benzyl)-3-methyl-benzamide hydrochloride;
N-(4-Carbamimidoyl-2-dibenzylamino-benzyl)-3-methyl-benzamide hydrochloride;
6-({5-Carbamimidoyl-2-[(3-chloro-benzoylamino)-methyl]-phenylamino}-methyl)-nicotinamide hydrochloride;
N-[4-Carbamimidoyl-2-(2-fluoro-benzylamino)-benzyl]-3-chloro-5-methanesulfonylamino-benzamide.

v) Another preferred compound of the invention is a compound of Formula (I) wherein X is X-2 and n is 2.

A preferred compound in this group is a compound of Formula (I) wherein Y is absent. In this case, R² is preferably hydroxy or C₁₋₆ alkoxy, more preferably hydroxy. R¹ is preferably hydrogen or hydroxy C₁₋₆ alkyl.

When X is X-2, n is 2 and Y is absent, Ar is preferably one of those mentioned under i), and especially phenyl optionally substituted by one or two substituents selected from the group consisting of halogen, C₁₋₆ alkyl and C₁₋₆ alkoxy.

Particularly preferred compounds in this group are:
N-[4-Carbamimidoyl-2-(2-hydroxy-ethylamino)-benzyl]-3-methyl-benzamide hydrochloride;
N-[4-Carbamimidoyl-2-(2-hydroxy-ethylamino)-benzyl]-3-chloro-benzamide hydrochloride;
N-[4-Carbamimidoyl-2-(2-hydroxy-ethylamino)-benzyl]-3-chloro-5-methoxy-benzamide hydrochloride;
N-{2-[Bis-(2-hydroxy-ethyl)-amino]-4-carbamimidoyl-benzyl}-3-chloro-5-methoxy-benzamide hydrochloride;
N-[4-Carbamimidoyl-2-(2-hydroxy-ethylamino)-benzyl]-3-chloro-5-hydroxy-benzamide hydrochloride;
N-4-Carbamimidoyl-2-ethylamino-benzyl)-3-chloro-5-methanesulfonylamino-benzamide.

vi) Another preferred compound of the invention is a compound of Formula (I) wherein X is X-2 and n is 0.

a) Among the compounds mentioned under vi), a preferred compound of the invention is a compound of Formula (I) wherein X is X-2, n is 0 and Y is Y-1. In this case, R² is preferably optionally substituted aryl-C₁₋₆ alkyl, optionally substituted heteroaryl-C₁₋₆ alkyl or optionally substituted heterocyclyl-C₁₋₆ alky, more preferably optionally substituted aryl-C₁₋₆ alkyl. Non substituted phenyl C₁₋₆ alkyl, especially benzyl is further more preferred. R¹ is preferably hydrogen.

When X is X-2, n is 0 and Y is Y-1, Ar is preferably one of those mentioned under i), and especially phenyl optionally substituted by one or two C₁₋₆ alkyl groups.

Particularly preferred compounds in this group are:
N-(4-Carbamimidoyl-2-phenylacetylamino-benzyl)-3-methyl-benzamide hydrochloride.

c) Among the compounds mentioned under vi), another preferred compound of the invention is a compound of Formula (I) wherein X is X-2, n is 0 and Y is absent. In this case, R² is preferably hydrogen or C₁₋₆ alkyl, especially hydrogen. R¹ is preferably hydrogen.

When X is X-2, n is 0 and Y is absent, Ar is preferably one of those mentioned under i), and especially phenyl optionally substituted by one or two C₁₋₆ alkyl groups.

Particularly preferred compounds in this group are:
N-(2-Amino-4-carbamimidoyl-benzyl)-3-methyl-benzamide hydrochloride.

vii) Another preferred compound of the invention is a compound of Formula (I) wherein X is X-3.

In this case, Ar is preferably one of those mentioned under i), and especially phenyl optionally substituted by one or two C₁₋₆ alkyl groups.

Particularly preferred compounds in this group are:
N-(4-Carbamimidoyl-2-nitro-benzyl)-3-methyl-benzamide hydrochloride.

viii) Another preferred compound of the invention is a compound of Formula (I) wherein
Ar is aryl or heteroaryl, which is optionally substituted by one, two or three substituents independently selected from the group consisting of halogen, C₁₋₆ alkyl, C₂₋₆ alkenyl, C₂₋₆ alkynyl, C₃₋₇ cycloalkyl, C₃₋₇ cycloalkyl C₁₋₆ alkyl, optionally substituted aryl-C₁₋₆ alkyl, optionally substituted heteroaryl-C₁₋₆ alkyl, optionally substituted heterocyclyl-C₁₋₆ alkyl, fluoro C₁₋₆ alkyl, hydroxy, C₁₋₆ alkoxycarbonyl, carboxy, nitro, cyano, hydroxy C₁₋₆ alkyl-aminocarbonyl, optionally substituted heterocyclyl-carbonyl, C₁₋₆ alkoxy C₁₋₆ alkyl-aminocarbonyl, C₁₋₆ alkoxy and amino, in which C₁₋₆ alkoxy may optionally be substituted by one or two substituents independently selected from the group consisting of hydroxy, optionally substituted heteroaryl, optionally substituted aryl, optionally substituted heterocyclyl, carbamoyl, mono C₁₋₆ alkyl substituted aminocarbonyl, carboxy and C₁₋₆ alkoxycarbonyl, and amino may optionally be substituted by one or two substituents independently selected from the group consisting of optionally substituted aryl-sulfanyl, optionally substituted aryl-sulfinyl, optionally substituted aryl-sulfonyl, optionally substituted heteroaryl-C₁₋₆ alkyl, C₁₋₆ alkyl, carbamoyl C₁₋₆ alkyl, C₁₋₆ alkylcarbamoyl, C₁₋₆ alkylsulfanyl, C₁₋₆ alkylsulfinyl and C₁₋₆ alkylsulfonyl;
X is X-1: —O—(CH₂)ₙ—Y—R², X-2: —N(R¹)—(CH₂)ₙ—Y—R² or X-3: —NO₂;
Y is Y-1: —C(═O)—, Y-2: 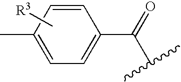
   or absent;
R¹ is hydrogen, C₁₋₆ alkyl, C₃₋₇ cycloalkyl, C₃₋₇ cydoalkyl C₁₋₆ alkyl, optionally substituted aryl-C₁₋₆ alkyl or hydroxy C₁₋₆ alkyl, C₁₋₆ alkoxy C₁₋₆ alkyl;
R² is hydrogen, C₁₋₆ alkyl, C₃₋₇ cycloalkyl, C₃₋₇ cycloalkyl C₁₋₆ alkyl, C₂₋₆ alkenyl, C₂₋₆ alkynl, C₁₋₆ alkoxy, hydroxy, optionally substituted aryl, optionally substituted heterocydyl, optionally substituted aryl-C₁₋₆ alkyl, optionally substituted heteroaryl-C₁₋₆ alkyl, optionally substituted heterocyclyl-C₁₋₆ alkyl, nitro, cyano, heteroaryl optionally substituted by one or two substituents independently selected from the group consisting of C₁₋₆ alkyl, carboxy, carbamoyl and C₁₋₆ alkoxycarbonyl or amino optionally substituted by one or two substituents independently selected from the group consisting of C₁₋₆ alkyl, C₁₋₆ alkylcarbonyl, C₁₋₆ alkylsulfanyl, C₁₋₆ alkylsulfinyl, C₁₋₆ alkylsulfonyl, optionally substituted aryl-carbonyl, optionally substituted aryl, optionally substituted heterocyclyl-C₁₋₆ alkyl, C₁₋₆ alkoxy C₁₋₆ alkyl, optionally substituted heteroaryl, optionally substituted heterocyclyl and hydroxy C₁₋₆ alkyl;
R³ is hydrogen, halogen or C₁₋₆ alkyl;
n is an integer from 0 to 2;
provided that X is not C₁₋₆ alkoxy;
and prodrugs and pharmaceutically acceptable salts thereof;
wherein
the term “aryl” means a phenyl or a naphthyl group;
the term “optionally substituted aryl” means an aryl group, which is optionally substituted by one to five substituents independently selected from the group consisting of halogen, hydroxy, trifluoromethyl, C₁₋₆ alkyl, halo C₁₋₆ alkyl, C₁₋₆ alkoxy, amino, nitro, aminocarbonyl and C₁₋₆ alkylcarbonyl;
the term “heterocyclyl” means non-aromatic monocyclic radicals of three to eight ring atoms in which one or two ring atoms are heteroatoms selected from NR^{x} {wherein R^{x} is hydrogen or C₁₋₆ alkyl}, O, or S(O)ₙ (where n is an integer from 0 to 2), the remaining ring atoms being C, and the non-aromatic monocydic ring may optionally be fused to a C₃₋₇ cycloalkyl, aryl or heteroaryl ring, with the understanding that the attachment point of the heterocyclyl radical is on said non-aromatic monocyclic ring, and one or two carbon atoms of said non-aromatic monocyclic ring may optionally be replaced with a carbonyl group;
the term “optionally substituted heterocyclyl” means a heterocyclyl group, which is optionally substituted independently with one, two, or three substituents selected from the group consisting of halogen, hydroxy, trifluoromethyl, C₁₋₆ alkyl, halo C₁₋₆ alkyl, C₁₋₆ alkoxy, amino, nitro, aminocarbonyl and C₁₋₆ alkylcarbonyl;
the term “heteroaryl” means a monocyclic or bicyclic radical of 5 to 12 ring atoms having at least one aromatic ring containing one, two, or three ring heteroatoms selected from N, O, and S, the remaining ring atoms being C, with the understanding that the attachment point of the heteroaryl radical will be on an aromatic ring;
the term “optionally substituted heteroaryl” means a heteroaryl group, which is optionally substituted independently with one, two, or three substituents selected from the group consisting of halogen, hydroxy, trifluoromethyl, C₁₋₆ alkyl, halo C₁₋₆ alkyl, C₁₋₆ alkoxy, amino, nitro, aminocarbonyl and C₁₋₆ alkylcarbonyl.

ix) Another preferred compound of the invention is a compound of Formula (I) wherein aryl or heteroaryl as Ar has at least one halogen substituent, preferably at meta position when Ar is phenyl. A preferred halogen is chlorine or fluorine.

x) Another preferred compound of the invention is a compound of Formula (I) wherein X is X-1.

xi) Another preferred compound of the invention is a compound of Formula (I) wherein X is X-2.

xii) Another preferred compound of the invention is a compound of Formula (I) wherein X is X-2, n is 0, Y is Y-2.

xiii) Another preferred compound of the invention is a compound of Formula (I) wherein Ar is aryl, preferably phenyl, substituted, preferably at meta position, by amino substituted by a substituent selected from the group consisting of optionally substituted aryl-sulfonyl, optionally substituted heteroaryl-sulfonyl, optionally substituted heterocyclyl-sulfonyl, optionally substituted aryl-C₁₋₆ alkylsulfonyl, optionally substituted heteroaryl-C₁₋₆ alkylsulfonyl and optionally substituted heterocyclyl-C₁₋₆ alkylsulfonyl, preferably optionally substituted aryl-C₁₋₆ alkylsulfonyl, and another substituent which is mono- or di-C₁₋₆ alkyl substituted aminocarbonyl-C₁₋₆ alkyl. Fluorophenylmethylsulfonyl or phenylmethylsulfonyl is more preferred as optionally substituted aryl-C₁₋₆ alkylsulfonyl. Carbamoylmethyl or methylcarbamoylmethyl is preferred as mono- or di-C₁₋₆ alkyl substituted aminocarbonyl-C₁₋₆ alkyl. Preferably, Ar has another substituent, preferably at meta position when aryl is a phenyl, which is halogen, especially chlorine.

a) Among the compounds mentioned under xiii), a preferred compound of the invention is a compound of Formula (I) wherein X is X-1, n is 1 and Y is Y-1. In this case, R² is preferably amino.

Particularly preferred compounds in this group are:
N-(4-Carbamimidoyl-2-carbamoylmethoxy-benzyl)-3-chloro-5-(methylcarbamoylmethyl-phenylmethanesulfonyl-amino)-benzamide;
N-(4-Carbamimidoyl-2-carbamoylmethoxy-benzyl)-3-[carbamoylmethyl-(4-fluoro-phenylmethanesulfonyl)-amino]-5-chloro-benzamide;
N-(4-carbamimidoyl-2-carbamoylmethoxy-benzyl)-3-[carbamoylmethyl-(3-fluoro-phenylmethanesulfonyl)-amino]-5-chloro-benzamide;
and N-(4-carbamimidoyl-2-carbamoylmethoxy-benzyl)-3-[carbamoylmethyl-(2-fluoro-phenylmethanesulfonyl)-amino]-5-chloro-benzamide.

xvi) Another preferred compound of the invention is a prodrug of the compound of Formula (I), which is 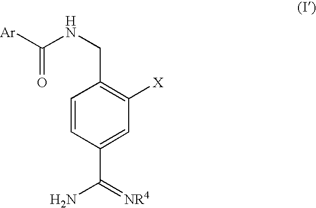
wherein R⁴ is hydroxy, OR⁵, —C(═O)OR⁵ or —C(═O)R⁵, and R⁵ is C₁₋₆ alkyl, C₃₋₇ cycloal or phenyl which is optionally substituted by one to five, preferable one or two substituents selected from the group consisting of halogen, C₁₋₆ alkyl and C₁₋₆ alkoxy. R⁴ is preferably hydroxy or —C(═O)OR⁵.

The compounds of the present invention can be prepared in a number of ways known to one skilled in the art. Preferred methods include, but are not limited to, the general synthetic procedures described below. In some cases in the structures set out in the general procedures and in the Examples below, the second or third valences for nitrogen are not represented. In those cases, as is apparent from the accompanying text, the valences are occupied by hydrogen. Similarly, on those occassions where an oxygen atom is missing a second valence, the valence is occupied by hydrogen (e.g., in acetic acid), as is dear from the accompanying text. In addition, where “Cl” is presented in isolation, it is meant to represent HCl, as is evident from the accompanying text.

The starting materials and reagents used in preparing these compounds are either available from commercial suppliers such as Aldrich Chemical Co., (Milwaukee, Wis., USA), Bachem (Torrance, Calif., USA), Enika-Chemie, or Sigma (St. Louis, Mo., USA), Maybridge (Dist: Ryan Scientific, P.O. Box 6496, Columbia, S.C. 92960), Bionet Research Ltd., (Cornwall PL32 9QZ, UK), Menai Organics Ltd., (Gwynedd, N. Wales, UK), Butt Park Ltd., (Dist. Interchim, Montlucon Cedex, France) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 1992), and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography. Such materials may be characterized using conventional means, including physical constants and spectral data.

The compounds of Formula (I) are prepared, for example, by converting the compounds of Formula (II) to the compounds of Formula (I). 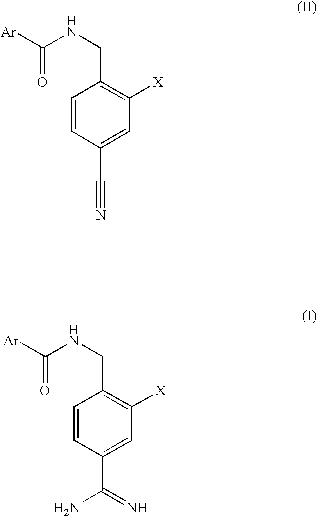
Ar and X have the significances given above. If desired, a reactive group present in an obtained compound of Formula (I) is modified and, if desired, a compound of Formula (I) obtained is converted into a physiologically compatible salt or a salt of a compound of Formula (I) is converted into the free acid or base.

The conversion of the nitrile group in a compound of Formula (II) into a carbamimidoyl group —C(NH)NH₂ can be carried out according to methods known per se. For example, the conversion of the nitrile group into a carbamimidoyl group can be carried out by treating a compound of formula (II) in a solvent, such as ethanol or methanol, or a solvent mixture, such as chloroform and methanol or chloroform and ethanol, with a dry stream of hydrogen chloride, conveniently at a temperature below 10° C. The solution containing the iminoether can be evaporated and the residue can be treated with gaseous ammonia or an ammonium salt in methanol or ethanol. In doing so, other reactive groups present in the compound of formula (I) and sensitive towards treatment with hydrogen chloride or gaseous ammonia or ammonium chloride can be modified. For example, in the case of treatment with hydrogen chloride, a benzyloxy group can be converted into the hydroxy group. In the case of treatment with gaseous ammonia in methanol or ethanol, a C₁₋₆-alkoxy-carbonyl group can be converted into a carbamoyl group.

The conversion of the nitrile group in a compound of Formula (II) into a carbamimidoyl group —C(NH)NH₂ can also be carried out via a two step procedure. For example, the conversion of the nitrile group into a N-hydroxy-carbamimidoyl group can be performed by dissolving a compound of formula (II) in a solvent, such as DMF, ethanol or methanol, treating the solution with hydroxylamine or a salt of hydroxylamine with an inorganic acid, such as hydroxylamine hydrochloride, and thereafter with a base, such as diisopropylethylamine or triethylamine, sodium hydride or sodium methanolate, conveniently at a temperature up to 80° C. The compound obtained can be converted into a compound of Formula (I) by hydrogenation in a solvent, such as ethanol, methanol, ethyl acetate, dioxane, THF or glacial acetic acid, or a solvent mixture, such as ethanol and glacial acetic acid, with hydrogen and a catalyst, such as palladium, platinum or nickel. In doing so, other reactive groups present in the compound of Formula (I) and sensitive towards the reducing agent can be modified.

Modifications of functional groups present in a compound of Formula (I) include especially the esterification of a carboxy group, the saponification of an ester group and the cleavage of an ether group, such as an arylalkyl ether group, e.g. the benzyl ether group. All of these reactions can be carried out according to methods known per se.

Prodrugs of the compounds of Formula (I) can be prepared, for example, by reacting a compound of Formula (I)
with a chloroformic acid C₁₋₆ alkyl ester or with a chloroformic acid aryl ester in a solvent, such as dichloromethane, dioxane or DMF, or a solvent mixture, such as dichloromethane and water or ethyl acetate and water, in the presence of an organic base, such as pyridine or triethylamine, or an inorganic base, such as sodium hydroxide, sodium carbonate or potassium hydrogen carbonate or
with an aryl carboxylic acid chloride in a solvent, such as dichloromethane, dioxane or DMF, or a solvent mixture, such as dichloromethane and water or ethyl acetate and water, in the presence of an organic base, such as pyridine or triethylamine, or an inorganic base, such as sodium hydroxide, sodium carbonate or potassium hydrogen carbonate or
with hydroxylamine or a salt of hydroxylamine with an inorganic acid, such as hydroxylamine hydrochloride, in a solvent such as DMF, DMA, ethanol or methanol, in the presence of an organic base, such as pyridine, diisopropylethylamine or triethylamine, or an inorganic base, such as sodium hydroxide, sodium hydride, sodium methanolate, sodium carbonate or potassium hydrogen carbonate.

A compound of Formula (II) wherein X has the significance of a hydroxy group and/or wherein Ar is aryl or heteroaryl substituted by one or two hydroxy groups can be reacted:
with an alkylating agent such as an appropriately substituted alkyl bromide, alkyl iodide or alkyl mesylate in the presence of a base such as potassium carbonate or cesium carbonate in a solvent such as DMF, acetonitrile or acetone, or
by a Mitsunobu reaction with an appropriately substituted alcohol in the presence of DEAD, DIAD or di-tert.-butyl-azodicarboxylate, and triphenylphosphine in a solvent such as THF or dioxane.

Furthermore, a compound of Formula (II) wherein X has the significance of an amino group and/or wherein Ar is aryl or heteroaryl substituted by one or two amino groups can be reacted:
with an alkylating agent such as an appropriately substituted alkyl bromide, alkyl iodide or alkyl mesylate in the presence of an organic base such as triethyl amine or diisopropyl ethyl amine in a solvent such as DMF or DMA, or
with an appropriately substituted aldehyde and a reducing agent like sodium cyanoborohydride in the presence of an acidic catalyst like ZnCl₂ in a solvent like methanol or ethanol, or
with an acyl or a sulfonyl chloride or a chloroformic acid ester in the presence of an organic base such as triethylamine or diisopropylethylamine in a solvent such as DMF, THF or acetonitrile.

Further modifications of functional groups present in a compound of Formula (II) include especially the esterification of a carboxy group, the saponification of an ester group and the cleavage of an ether group, such as an arylalkyl ether group, e.g. the benzyl ether group, the reduction of a nitro group, the acylation of an amino group and the removal of protecting groups. All of these reactions can be carried out according to methods known per se.

Compounds of Formula (II) can be prepared according to general methods known per se, e.g. by coupling of an appropriately substituted 4-aminomethyl benzonitrile (III) and an acid of formula (IV) in the presence of a coupling reagent such as BOP or EDCI/HOBt and an organic base such as triethylamine or diisopropylethylamine in a solvent such as THF.

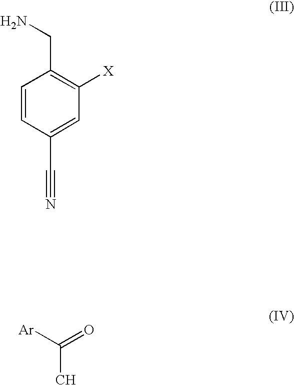

Compounds of Formula (III) and (IV) are known per se, and can be prepared according to general methods known per se, e.g. as described hereinafter and/or as described in the Examples or in analogy to those methods described in the Examples.

The compounds of Formula (I) are active compounds and inhibit the formation of coagulation factors Xa, IXa and thrombin induced by factor VIIa and tissue factor or are derivatives which are converted under physiological conditions to such active compounds. These compounds consequently influence both platelet aggregation which is induced by these factors and plasmatic blood coagulation. They therefore inhibit the formation of thrombin and can be used for the treatment and/or prevention of diseases, such as arterial and venous thrombosis, deep vein thrombosis, pulmonary embolism, unstable angina pectoris, cardiac infarction, stroke due to atrial fibrillation, inflammation and arteriosclerosis. Furthermore, these compounds have an effect on tumour cells and prevent metastases. They can therefore also be used as antitumour agents. Prevention and/or treatment of thrombosis, particularly arterial or deep vein thrombosis, is the preferred indication.

The inhibition of the amidolytic activity of factor VIIa/tissue factor complex by the compounds in accordance with the invention can be demonstrated with the aid of a chromogenic peptide substrate as described hereinafter.

The compounds of Formula (I) and/or their pharmaceutically acceptable salts can be used as medicaments, e.g. in the form of pharmaceutical preparations for enteral, parenteral or topical administration. They can be administered, for example, perorally, e.g. in the form of tablets, coated tablets, dragees, hard and soft gelatine capsules, solutions, emulsions or suspensions, rectally, e.g. in the form of suppositories, parenterally, e.g. in the form of injection solutions or suspensions or infusion solutions, or topically, e.g. in the form of ointments, creams or oils. Oral administration is preferred.

The production of the pharmaceutical preparations can be effected in a manner which will be familiar to any person skilled in the art by bringing the described compounds of formula I and/or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragees and hard gelatine capsules. Suitable carrier materials for soft gelatine capsules are, for example, vegetable oils, waxes, fats and semi-solid and liquid polyols (depending on the nature of the active ingredient no carriers might, however, be required in the case of soft gelatine capsules). Suitable carrier materials for the production of solutions and syrups are, for example, water, polyols, sucrose, invert sugar. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of the compounds of formula I can vary within wide limits depending on the disease to be controlled, the age and the individual condition of the patient and the mode of administration, and will, of course, be fitted to the individual requirements in each particular case. For adult patients a daily dosage of about 1 to 1000 mg, especially about 1 to 100 mg, comes into consideration. Depending on severity of the disease and the precise pharmacokinetic profile the compound could be administered with one or several daily dosage units, e.g. in 1 to 3 dosage units.

The pharmaceutical preparations conveniently contain about 1-500 mg, preferably 1-100 mg, of a compound of formula I.

The following Examples serve to illustrate the present invention in more detail. They are, however, not intended to limit its scope in any manner.

### EXAMPLES

Abbreviations

BOP=(benzotriazol-1-yloxy)-tris-(dimethylamino)-phosphonium-hexafluorophosphat, CAS=Chemical Abstract Services, DEAD=diethyl azodicarboxylate, DMF=dimethyl formamide, EDCI=1-[3-(dimethylamino)propyl]-3-ethylcarbodiimide hydrochloride, EtOH=ethanol, HOBT=1-hydroxybenzotriazole, MS=mass spectroscopy, MeOH=methanol, r.t.=room temperature, THF=tetrahydrofuran

### General Procedures

### General Procedure A: Coupling of an Aryl Carboxylic Acid with a Primary Amine using BOP as a Coupling Reagent

To a stirred solution of the amine (1 eq) in THF is added the acid (1.2 eq), N-ethyl-diisopropylamine (1.2 eq) and BOP-reagent (1.2 eq). The mixture is then stirred at r.t. under an argon atmosphere for 3-24 h. The mixture is diluted with EtOAc, washed with water, sat. Na₂CO₃ solution and water; dried (MgSO₄), filtered and concentrated. The crude product can be purified by chromatography (silicagel) or by crystallization.

### General Procedure B: Reduction of an Aromatic Nitro Group

To a stirred solution of the nitro compound in THF and ethanol is added palladium/C. After 2 to 24 h stirring at r.t. under hydrogen atmosphere the mixture is filtered and the filtrate is concentrated. The crude product can be purified by flash chromatography (silicagel) or by crystallization.

### General Procedure C: Conversion of an Aromatic Nitrile into an Amidine (Pinner Reaction)

Dry HCl gas is passed over a cooled (−10° C.), stirred solution of the starting material in CHCl₃/EtOH (or MeOH) 5:1 for 15 min. The flask is stoppered and left at 4° C. overnight. If conversion is not complete, the reaction mixture is allowed to warm to r.t. The mixture is concentrated (rotavapor and high vacuum) at r.t. The residue is dissolved in EtOH and treated with a 2.0 M NH₃ solution in EtOH. The resulting mixture is stirred at r.t. for sensitive compounds or 60° C. for 2-18 h. The mixture is then concentrated (rotavapor) and purified by chromatography (silicagel).

### Preparation of Building Blocks (BB)

### BB1: 4-Aminomethyl-3-hydroxy-benzonitrile hydrochloride

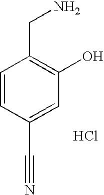

To a solution of 4-formyl-3-hydroxy-benzonitrile (CAS 84102-89-6) (6.90 g) in dry ethanol (165 ml) was added sodium acetate (4.23 g) and hydroxylamine hydrochloride (3.58 g). The mixture was stirred at r.t. for 1 h. The solvent was evaporated and the product was purified by flash chromatography (cyclohexane/EtOAc 8:2=>1:1) to give 3-hydroxy-4-(hydroxyimino-methyl)-benzonitrile (4.70 g). Light yellow solid. MS 162.0 ([M]⁺)

A solution of 3-hydroxy-4-(hydroxyimino-methyl)-benzonitrile (1.79 g) in acetic acid (16.6 ml) was stirred at 65° C. Zinc powder (6.59 g) was added portionwise during 30 min. After stirring for a further 1.5 h, the reaction mixture was filtered and the filtrate was concentrated to dryness. 1 N HCl (55.3 ml) was added and the solvent was evaporated. The same procedure was repeated with with water (2×), EtOH (2×) and toluene (2×). The resulting colorless solid was dissolved in diethyl ether, filtered and the filtrate was concentrated to give 4-aminomethyl-3-hydroxy-benzonitrile hydrochloride (colorless solid, 2.5 g). MS 149.2 ([M+H]⁺)

### BB2: 2-(2-Aminomethyl-5-cyano-phenoxy)-acetamide hydrochloride

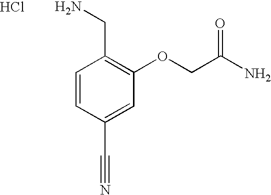

To a solution of 4-aminomethyl-3-hydroxy-benzonitrile hydrochloride (BB1, 2.0 g) and triethylamine (2.19 g) in dichloromethane (20 ml) was added di-tert.-butyldicarbonate (2.41 g). The mixture was stirred at r.t. for 3.5 h. The mixture was washed with water (3×), dried, filtered and concentrated. The crude product was dissolved in DMF (15.5 ml). Cesium carbonate (4.00 g) and iodoacetamide (2.27 g) were added and the mixture was stirred at r.t. for 3 days. Water was added and the mixture was extracted with EtOAc. The org. phase was washed with water, dried, filtered and concentrated. The crude product was dissolved in MeOH and then concentrated to obtain a thick suspension. The solid was filtered off and washed with a small amount of MeOH. This procedure was repeated with the mother liquor to give (2-carbamoylmethoxy-4-cyano-benzyl)-carbamic acid tert-butyl ester (a total of 1.88 g) as a colorless solid. MS 304.2 ([M−H]⁻)

The BOC protecting group of (2-carbamoylmethoxy-4-cyano-benzyl)-carbamic acid tert-butyl ester was removed using HCl in dioxane to give 2-(2-aminomethyl-5-cyano-phenoxy)-acetamide hydrochloride as an off-white powder. MS 206.1 ([M+H]⁺)

### BB3: 2-(2-Aminomethyl-5-cyano-phenoxy)-N-methyl-acetamide hydrochloride

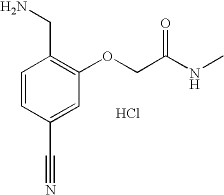

To a solution of 4-aminomethyl-3-hydroxy-benzonitrile hydrochloride (BB1, 3.5 g) and triethylamine (3.76 g) in dichloromethane (40 ml) was added di-tert.-butyldicarbonate (4.055 g). The mixture was stirred at r.t. for 24 h. The mixture was washed with water (3×), dried, filtered and concentrated. The crude product was dissolved in DMA (40 ml). Cesium carbonate (7.52 g) and 2-chloro-N-methylacetamide (2.28 g) were added and the mixture was stirred at r.t. for 24 h. Water was added and the mixture was extracted with EtOAc. The org. phase was washed with water and brine, dried, filtered and concentrated. The crude product was treated with EtOAc and stirred for 10 min. The solid was filtered off. The mother liquor was concentrated and the residue was treated with diethyl ether. The solid was filtered off. The combined solids were dried to give a total of 3.17 g of (4-cyano-2-methylcarbamoylmethoxy-benzyl)-carbamic acid tert-butyl ester as a colorless solid. MS 320.4 ([M+H]⁺)

The BOC protecting group of (4-cyano-2-methylcarbamoylmethoxy-benzyl)-carbamic acid tert-butyl ester was removed using HCl in dioxane to give 2-(2-aminomethyl-5-cyano-phenoxy)-N-methyl-acetamide hydrochloride as a colorless solid. MS 220.4 ([M+H]⁺)

### BB4: 4-Aminomethyl-3-nitro-benzonitrile

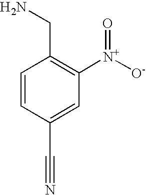

To a mechanically stirred solution of 4-bromomethyl-3-nitro-benzonitrile (21.7 g, CAS 223 512-70-7) in chloroform (250 ml) under argon atmosphere was added hexamethylenetetramine (7.1 g). A white precipitate appeared a few minutes after the addition. After 3 hrs heating to reflux (oil bath 80° C.) he mixture was cooled to r.t. The solid was collected by filtration, washed with chloroform and dried under high vacuum) to give 1-(4-cyano-2-nitro-benzyl)-3,5,7-triaza-1-azonia-tricyclodecane hydrobromide (13.8 g). Off-white powder.

To a mechanically stirred suspension of 1-(4-cyano-2-nitro-benzyl)-3,5,7-triaza-1-azonia-tricyclodecane hydrobromide (13.8 g) in ethanol (150 ml) under argon atmosphere, was added concentrated aqueous HCl (20 ml). After 6 hours stirring at reflux the mixture was concentrated, diluted with NaOH 1N until pH>12. The product was extracted with EtOAc. The combined organic phases were washed twice with water and with brine. Then the solution was dried over MgSO₄), filtered and concentrated to give 4-aminomethyl-3-nitro-benzonitrile (5.8 g) as yellow solid.

### Example 1

1.1 3-Fluorobenzoic acid was coupled with 4-aminomethyl-3-hydroxy-benzonitrile hydrochloride (BB1) according to general procedure A to give N-(4-cyano-2-hydroxy-benzyl)-3-fluoro-benzamide. Off-white solid. MS 269.2 ([M−H]⁻)

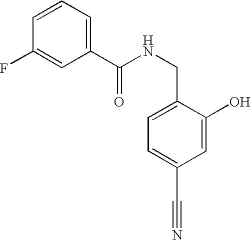

1.2 To a solution of N-(4-cyano-2-hydroxy-benzyl)-3-fluoro-benzamide (200 mg) in acetone (2 ml) were added cesium carbonate (291 mg) and iodoacetamide (168 mg). The reaction mixture was stirred at r.t. overnight. The solvent was evaporated. The residue was washed with water and dried to give N-(2-carbamoylmethoxy-4-cyano-benzyl)-3-fluoro-benzamide (229 mg) as a colorless solid. MS 328.1 ([M+H]⁺)

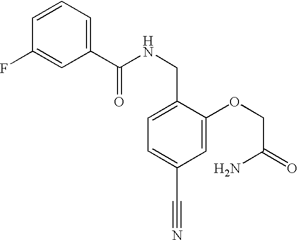

1.3 N-(2-Carbamoylmethoxy-4-cyano-benzyl)-3-fluoro-benzamide was converted to N-(4-carbamimidoyl-2-carbamoylmethoxy-benzyl)-3-fluoro-benzamide hydrochloride according to general procedure C. Colorless solid. MS 345.2 ([M+H]⁺)

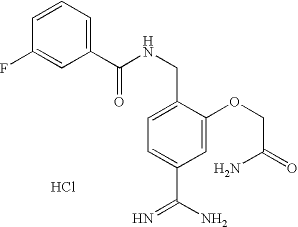

### Example 2

2.1 3-Methoxybenzoic acid was coupled with 4-aminomethyl-3-hydroxy-benzonitrile hydrochloride (BB1) according to general procedure A to give N-(4-cyano-2-hydroxy-benzyl)-3-methoxy-benzamide. Off-white solid. MS 281.3 ([M−H]⁻)

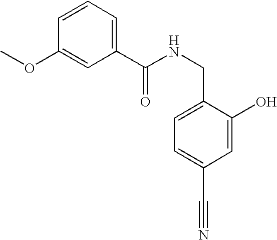

2.2 In analogy to example 1.2, N-(4-cyano-2-hydroxy-benzyl)-3-methoxy-benzamide was alkylated with iodoacetamide to give N-(2-carbamoylmethoxy-4-cyano-benzyl)-3-methoxy-benzamide as a colorless solid. MS 340.1 ([M+H]⁺)

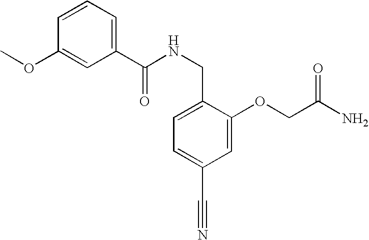

2.3 N-(2-Carbamoylmethoxy-4-cyano-benzyl)-3-methoxy-benzamide was converted to N-(4-carbamimidoyl-2-carbamoylmethoxy-benzyl)-3-methoxy-benzamide hydrochloride according to general procedure C. Light yellow solid. MS 357.2 ([M+H]⁺)

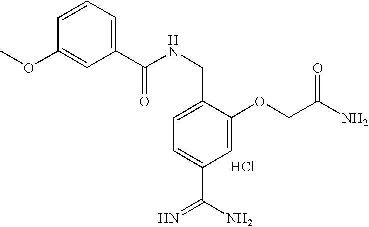

### Example 3

3.1 4-tert-Butoxycarbonylamino-3-methyl-benzoic acid (CAS 180976-94-7) was coupled with 4-aminomethyl-3-hydroxy-benzonitrile hydrochloride (BB1) according to general procedure A to give [4-(4-cyano-2-hydroxy-benzylcarbamoyl)-2-methyl-phenyl]-carbamic acid tert-butyl ester. Off-white solid. MS 380.3 ([M−H]⁻)

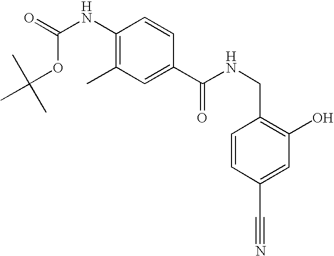

3.2 To a solution of [4-(4-cyano-2-hydroxy-benzylcarbamoyl)-2-methyl-phenyl]-carbamic acid tert-butyl ester (150 mg) in N,N-dimethylacetamide (10 ml) were added cesium carbonate (309 mg) and 2-(bromomethyl)-pyridine hydrobromide (122 mg). The mixture was stirred at r.t. overnight. Water and ethyl acetate were added and the mixture was extracted with ethyl acetate. The org. phase was washed with water (3×), dried, filtered and concentrated. The product was purified by chromatography (SiO₂, CH₂Cl₂=>CH₂Cl₂/MeOH 4:1) to give {4-[4-cyano-2-(pyridin-2-ylmethoxy)-benzylcarbamoyl]-2-methyl-phenyl}-carbamic acid tert-butyl ester (156 mg) as a light yellow solid. MS 473.3 ([M+H]⁺)

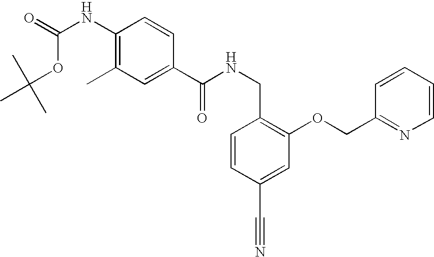

3.3 {4-[4-Cyano-2-(pyridin-2-ylmethoxy)-benzylcarbamoyl]-2-methyl-phenyl}-carbamic acid tert-butyl ester was converted to 4-amino-N-[4-carbamimidoyl-2-(pyridin-2-ylmethoxy)-benzyl]-3-methyl-benzamide acetic acid salt according to general procedure C. Off-white solid. MS 390.2 ([M+H]⁺)

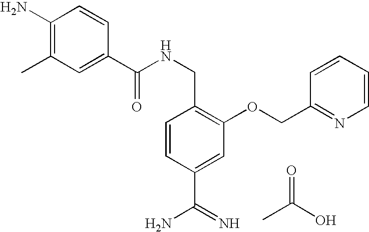

### Example 4

4.1 In analogy to example 1.2, [4-(4-cyano-2-hydroxy-benzylcarbamoyl)-2-methyl-phenyl]-carbamic acid tert-butyl ester (example 3.1) was alkylated with iodoacetamide to give [4-(2-carbamoylmethoxy-4-cyano-benzylcarbamoyl)-2-methyl-phenyl]-carbamic acid tert-butyl ester as a colorless solid. MS 439.4 ([M+H]⁺)

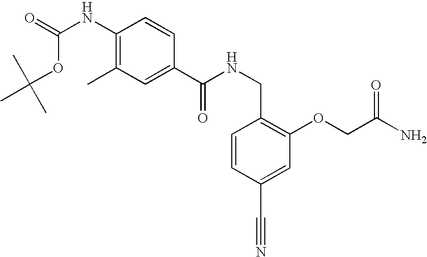

4.2 [4-(2-Carbamoylmethoxy-4-cyano-benzylcarbamoyl)-2-methyl-phenyl]-carbamic acid tert-butyl ester was converted to 4-amino-N-(4-carbamimidoyl-2-carbamoylmethoxy-benzyl)-3-methyl-benzamide acetic acid salt according to general procedure C. Off-white solid. MS 356.2 ([M+H]⁺)

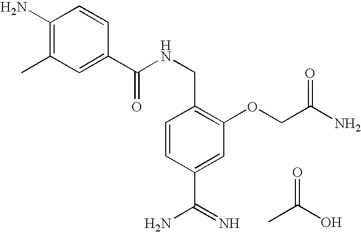

### Example 5

5.1 5-Methyl-3-isoxazolecarboxylic acid (CAS 3405-77-4) was coupled with 2-(2-aminomethyl-5-cyano-phenoxy)-acetamide hydrochloride (BB2) according to general procedure A to give 5-methyl-isoxazole-3-carboxylic acid 2-carbamoylmethoxy-4-cyano-benzylamide. Off-white solid. MS 315.0 ([M+H]⁺)

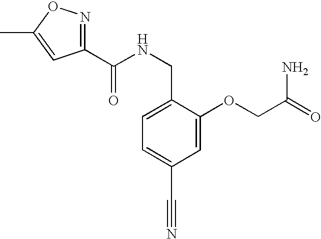

5.2 5-Methyl-isoxazole-3-carboxylic acid 2-carbamoylmethoxy-4-cyano-benzylamide was converted to 5-methyl-isoxazole-3-carboxylic acid 4-carbamimidoyl-2-carbamoylmethoxy-benzylamide acetic acid salt according to general procedure C. Colorless solid. MS 332.2 ([M+H]⁺)

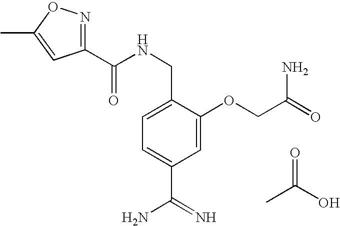

### Example 6

6.1 3-Methyl-5-isoxazolecarboxylic acid (CAS 4857-42-5) was coupled with 2-(2 aminomethyl-5-cyano-phenoxy)-acetamide hydrochloride (BB2) according to general procedure A. The product of this reaction was not obtained pure and was directly converted to 3-methyl-isoxazole-5-carboxylic acid 4-carbamimidoyl-2-carbamoylmethoxy-benzylamide acetic acid salt according to general procedure C. Light brown solid. MS 332.3 ([M+H]⁺)

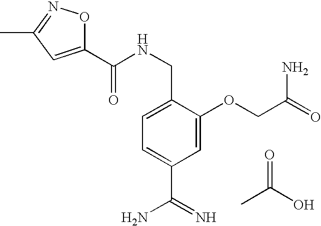

### Example 7

7.1 3-Methylbenzoic acid was coupled with 4-aminomethyl-3-hydroxy-benzonitrile hydrochloride (BB1) according to general procedure A to give N-(4-cyano-2-hydroxy-benzyl)-3-methyl-benzamide. Colorless oil. MS 265.0 ([M−H]⁻)

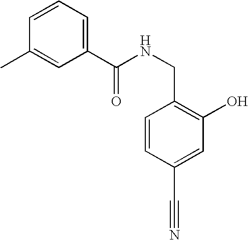

7.2 In analogy to example 1.2, N-(4-cyano-2-hydroxy-benzyl)-3-methyl-benzamide was alkylated with iodoacetamide to give N-(2-carbamoylmethoxy-4-cyano-benzyl)-3-methyl-benzamide as a light yellow solid. MS 324.3 ([M+H]⁺)

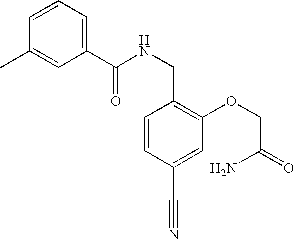

7.3 N-(2-Carbamoylmethoxy-4-cyano-benzyl)-3-methyl-benzamide was converted to N-(4-carbamimidoyl-2-carbamoylmethoxy-benzyl)-3-methyl-benzamide hydrochloride according to general procedure C. Colorless solid. MS 341.3 ([M+H]⁺)

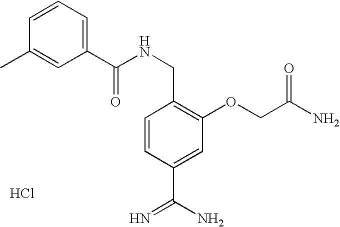

### Example 8

8.1 In analogy to example 1.2, N-(4-cyano-2-hydroxy-benzyl)-3-methyl-benzamide (example 7.1) was alkylated with methyl-4-bromomethyl benzoate in acetone with potassium carbonate as a base to give 4-{5-cyano-2-[(3-methyl-benzoylamino)-methyl]-phenoxymethyl}-benzoic acid methyl ester as a colorless solid. MS 415.4 ([M+H]⁺)

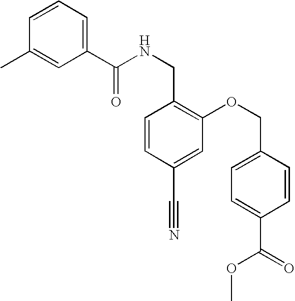

8.2 4-{5-Cyano-2-[(3-methyl-benzoylamino)-methyl]-phenoxymethyl}-benzoic acid methyl ester was converted to 4-{5-carbamimidoyl-2-[(3-methyl-benzoylamino)-methyl]-phenoxymethyl}-benzoic acid methyl ester hydrochloride according to general procedure C. Colorless foam. MS 432.4 ([M+H]⁺)

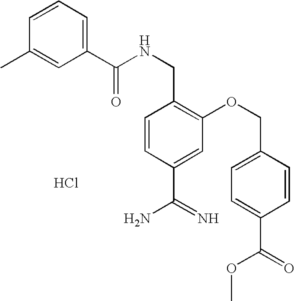

8.3 To a suspension of 4-{5-carbamimidoyl-2-[(3-methyl-benzoylamino)-methyl]-phenoxymethyl}-benzoic acid methyl ester hydrochloride (78 mg) in THF (0.6 ml) was added 1 M NaOH (0.33 ml). The mixture was stirred for 30 min at 0° C. and for 1.5 h at r.t. A further 0.17 ml 1 M NaOH was added and stirred for 5 h at r.t. The mixture was neutralized using 1 M HCl. The THF was removed under reduced pressure at r.t. The solid was collected by filtration, washed with water and dried to give 4-{5-carbamimidoyl-2-[(3-methyl-benzoylamino)-methyl]-phenoxymethyl}-benzoic acid (65 mg) as a colorless solid. MS 418.2 ([M+H]⁺)

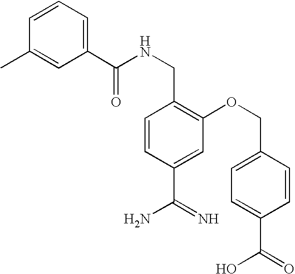

### Example 9

9.1 In analogy to example 1.2, N-(4-cyano-2-hydroxy-benzyl)-3-methyl-benzamide (example 7.1) was alkylated with 2-(bromomethyl)-pyridine hydrobromide in acetone with potassium carbonate as a base to give N-[4-cyano-2-(pyridin-2-ylmethoxy)-benzyl]-3-methyl-benzamide as a colorless foam. MS 358.0 ([M+H]⁺)

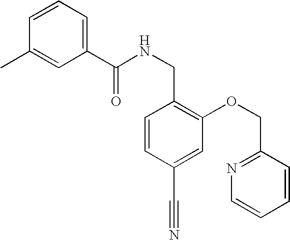

9.2 N-[4-Cyano-2-(pyridin-2-ylmethoxy)-benzyl]-3-methyl-benzamide was converted to N-[4-carbamimidoyl-2-(pyridin-2-ylmethoxy)-benzyl]-3-methyl-benzamide hydrochloride according to general procedure C. Colorless solid. MS 375.4 ([M+H]⁺)

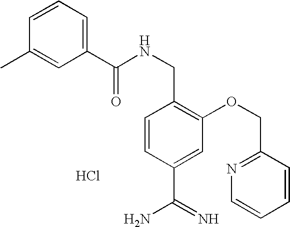

### Example 10

10.1 In analogy to example 1.2, N-(4-cyano-2-hydroxy-benzyl)-3-methyl-benzamide (example 7.1) was alkylated with 5-bromomethyl-2-methyl-2H-pyrazole-3-carboxylic acid ethyl ester (CAS 199480-29-0) in acetone with potassium carbonate as a base to give 5-{5-cyano-2-[(3-methyl-benzoylamino)-methyl]-phenoxymethyl}-2-methyl-2H-pyrazole-3-carboxylic acid ethyl ester as a colorless foam. MS 433.5 ([M+H]⁺)

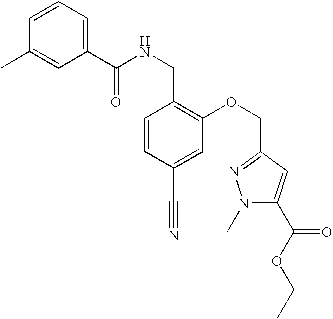

10.2 5-{5-Cyano-2-[(3-methyl-benzoylamino)-methyl]-phenoxymethyl}-2-methyl-2H-pyrazole-3-carboxylic acid ethyl ester was converted to 5-{5-carbamrnimidoyl-2-[(3-methyl-benzoylamino)-methyl]-phenoxymethyl}-2-methyl-2H-pyrazole-3-carboxylic acid ethyl ester hydrochloride according to general procedure C. Colorless solid. MS 450.3 ([M+H]⁺)

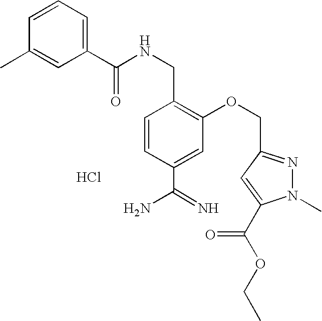

10.3 In analogy to example 8.3, 5-{5-carbamimidoyl-2-[(3-methyl-benzoylamino)-methyl]-phenoxymethyl}-2-methyl-2H-pyrazole-3-carboxylic acid ethyl ester hydrochloride was hydrolyzed to give 5-{5-carbamimidoyl-2-[(3-methyl-benzoylamino)-methyl]-phenoxymethyl}-2-methyl-2H-pyrazole-3-carboxylic acid as a colorless solid. MS 422.2 ([M+H]⁺)

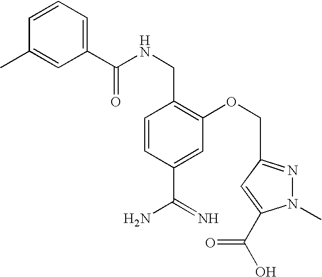

### Example 11

11.1 Benzyl-3-methyl-5-pyrazolecarboxylic acid (CAS 1141-70-4) was coupled with 4-aminomethyl-3-hydroxy-benzonitrile hydrochloride (BB1) according to general procedure A to give 2-benzyl-5-methyl-2H-pyrazole-3-carboxylic acid 4-cyano-2-hydroxy-benzylamide. Colorless solid. MS 345.3 ([M−H]⁻)

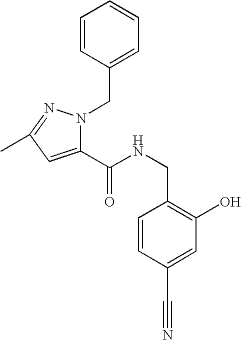

11.2 In analogy to example 1.2, 2-benzyl-5-methyl-2H-pyrazole-3-carboxylic acid 4-cyano-2-hydroxy-benzylamide was alkylated with iodoacetamide to give 2-benzyl-5-methyl-2H-pyrazole-3-carboxylic acid 2-carbamoylmethoxy-4-cyano-benzylamide as a colorless solid. MS 404.4 ([M+H]⁺)

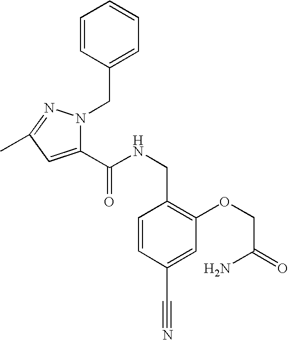

11.3 2-Benzyl-5-methyl-2H-pyrazole-3-carboxylic acid 2-carbamoylmethoxy-4-cyano-benzylamide was converted to 2-benzyl-5-methyl-2H-pyrazole-3-carboxylic acid 4-carbamimidoyl-2-carbamoylmethoxy-benzylamide acetic acid salt according to general procedure C. Colorless solid. MS 421.2 ([M+H]⁺)

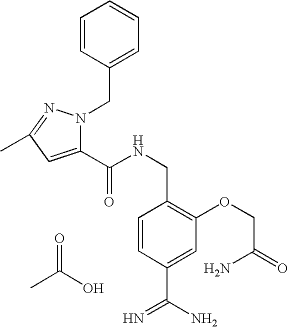

### Example 12

12.1 5-Methylnicotinic acid (CAS 3222-49-9) was coupled with 4-aminomethyl-3-hydroxy-benzonitrile hydrochloride (BB1) according to general procedure A to give N-(4-cyano-2-hydroxy-benzyl)-5-methyl-nicotinamide. Colorless solid. MS 266.3 ([M−H]⁻)

12.2 In analogy to example 1.2, N-(4-cyano-2-hydroxy-benzyl)-5-methyl-nicotinamide was alkylated with iodoacetamide to give N-(2-carbamoylmethoxy-4-cyano-benzyl)-5-methyl-nicotinamide as a colorless solid. MS 325.3 ([M+H]⁺)

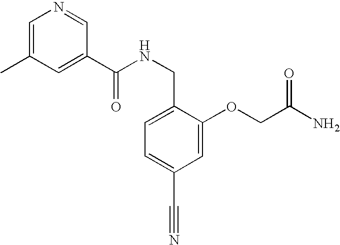

12.3 N-(2-Carbamoylmethoxy-4-cyano-benzyl)-5-methyl-nicotinamide was converted to N-(4-carbamimidoyl-2-carbamoylmethoxy-benzyl)-5-methyl-nicotinamide; compound with hydrochloride and ammoniumchloride according to general procedure C. Colorless solid. MS 342.0 ([M+H]⁺)

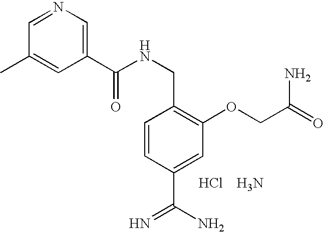

### Example 13

13.1 2-Methylisonicotinic acid (CAS 4021-11-8) was coupled with 4-aminomethyl-3-hydroxy-benzonitrile hydrochloride (BB1) according to general procedure A to give N-(4-cyano-2-hydroxy-benzyl)-2-methyl-isonicotinamide. Off-white solid. MS 266.3 ([M−H]⁻)

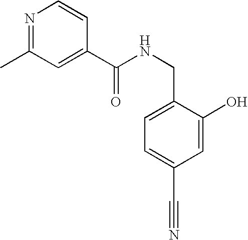

13.2 In analogy to example 1.2, N-(4-cyano-2-hydroxy-benzyl)-2-methyl-isonicotonamide was alkylated with iodoacetamide to give N-(2-carbamoylmethoxy-4-cyano-benzyl)-2-methyl-isonicotinamide as a yellow solid. MS 325.0 ([M+H]⁺)

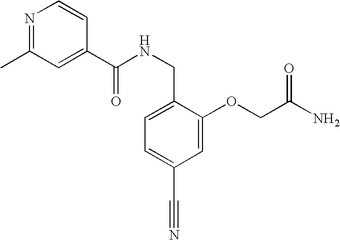

13.3 N-(2-Carbamoylmethoxy-4-cyano-benzyl)-2-methyl-isonicotinamide was converted to N-(4-carbamirnidoyl-2-carbamoylmethoxy-benzyl)-2-methyl-isonicotinamide acetic acid salt according to general procedure C. Colorless solid. MS 342.2 ([M+H]⁺)

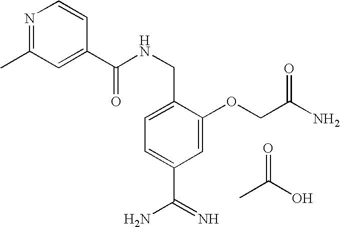

### Example 14

14.1 A mixture of 2-benzenesulfonylamino-5-methyl-benzoic acid (CAS 138964-56-4, 1.0 g) and thionyl chloride (4.23 g) was stirred under an argon atmosphere at 60° C. for 3.5 h. The solvent was evaporated. Toluene was added and again evaporated. This procedure was repeated once. The crude acid chloride was dried under high vacuum and used without further purification.

To a suspension of 2-(2-aminomethyl-5-cyano-phenoxy)-N-methyl-acetamide hydrochloride (BB3, 150 mg) in CH₂Cl₂ (6 ml) were added triethylamine (148 mg) and the crude acid chloride (182 mg). The mixture was stirred at r.t. for 24 h. The mixture was washed with 1 M HCl and with saturated NaHCO₃ solution. The aqueous phase was extracted with CH₂Cl₂. The combined organic phase was dried, filtered and concentrated. The crude product was purified by chromatography (SiO₂, cyclohexane/EtOAc 7:3=>2:8) to give 2-benzenesulfonylamino-N-(4-cyano-2-methylcarbamoylmethoxy-benzyl)-5-methyl-benzamide as a colorless foam. MS 493.1 ([M+H]⁺)

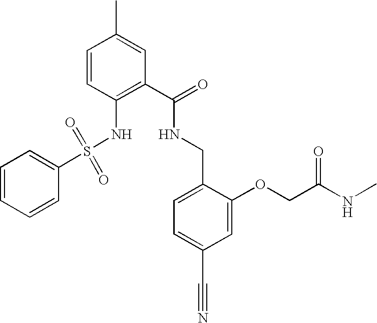

14.2 2-Benzenesulfonylamino-N-(4-cyano-2-methylcarbamoylmethoxy-benzyl)-5-methyl-benzamide was converted to 2-benzenesulfonylamino-N-(4-carbamimidoyl-2-methylcarbamoylmethoxy-benzyl)-5-methyl-benzamide hydrochloride according to general procedure C. Colorless foam. MS 510.4 ([M+H]⁺)

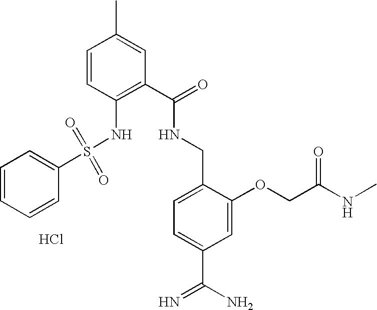

### Example 15

15.1 2,5-Dichlorobenzoic acid was coupled with 2-(2-aminomethyl-5-cyano-phenoxy)-N-methyl-acetamide hydrochloride (BB3) according to general procedure A to give 2,5-dichloro-N-(4-cyano-2-methylcarbamoylmethoxy-benzyl)-benzamide. Light yellow solid. MS 392.1 ([M+H]⁺)

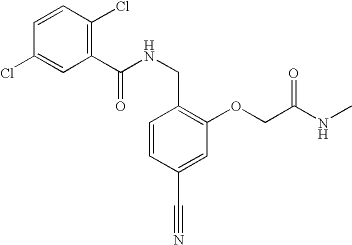

15.2 2,5-Dichloro-N-(4-cyano-2-methylcarbamoylmethoxy-benzyl)-benzamide was converted to N-(4-carbamimidoyl-2-methylcarbamoylmethoxy-benzyl)-2,5-dichloro-benzamide hydrochloride according to general procedure C. Colorless foam. MS 409.2 ([M+H]⁺)

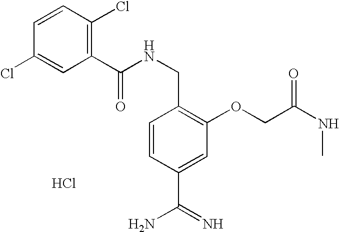

### Example 16

16.1 3-Chloro-4-fluorobenzoic acid was coupled with 2-(2-aminomethyl-5-cyano-phenoxy)-N-methyl-acetamide hydrochloride (BB3) according to general procedure A to give 3-chloro-N-(4-cyano-2-methylcarbamoylmethoxy-benzyl)-4-fluoro-benzamide. Light yellow solid. MS 376.2 ([M+H]⁺)

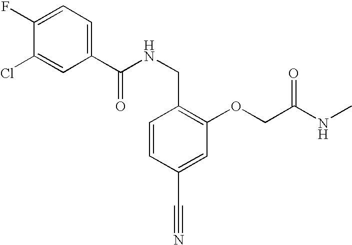

16.2 3-Chloro-N-(4-cyano-2-methylcarbamoylmethoxy-benzyl)-4-fluoro-benzamide was converted to N-(4-carbamimidoyl-2-methylcarbamoylmethoxy-benzyl)-3-chloro-4-fluoro-benzanide hydrochloride according to general procedure C. Colorless solid. MS 393.2 ([M+H]⁺)

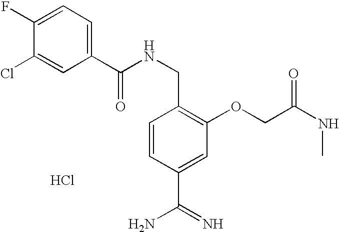

### Example 17

17.1 3,5-Dichloro-4-fluorobenzoic acid (CAS 98191-30-1) was coupled with 2-(2-aminomethyl-5-cyano-phenoxy)-N-methyl-acetamide hydrochloride (BB3) according to general procedure A to give 3,5-dichloro-N-(4-cyano-2-methylcarbamoylmethoxy-benzyl)-4-fluoro-benzamide. Light yellow solid. MS 410.1 ([M+H]⁺)

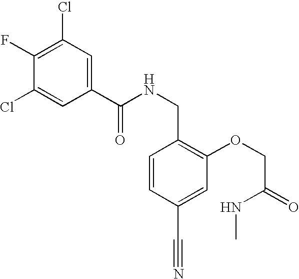

17.2 3,5-Dichloro-N-(4-cyano-2-methylcarbamoylmethoxy-benzyl)-4-fluoro-benzamide was converted to N-(4-carbamimidoyl-2-methylcarbamoylmethoxy-benzyl)-3,5-dichloro-4-fluoro-benzamide hydrochloride according to general procedure C. Colorless solid. MS 427.3 ([M+H]⁺)

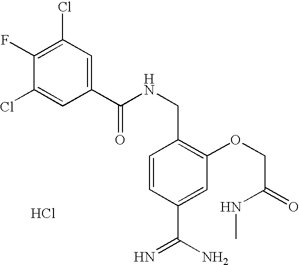

### Example 18

18.1 3,5-Dichlorobenzoic acid was coupled with 2-(2-aminomethyl-5-cyano-phenoxy)-N-methyl-acetamide hydrochloride (BB3) according to general procedure A to give 3,5-dichloro-N-(4-cyano-2-methylcarbamoylmethoxy-benzyl)-benzamide. Light yellow solid. MS 392.1 ([M+H]⁺)

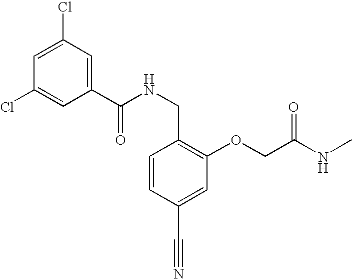

18.2 3,5-Dichloro-N-(4-cyano-2-methylcarbamoylmethoxy-benzyl)-benzamide was converted to N-(4-carbamimidoyl-2-methylcarbamoylmethoxy-benzyl)-3,5-dichloro-benzamide hydrochloride according to general procedure C. Colorless solid. MS 409.2 ([M+H]⁺)

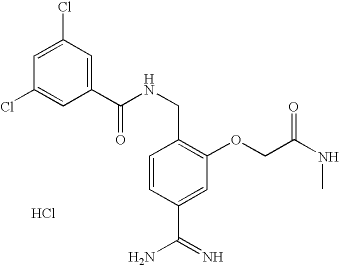

### Example 19

19.1 5-Chloronicotinic acid (CAS 22620-27-5) was coupled with 2-(2-aminomethyl-5-cyano-phenoxy)-N-methyl-acetamide hydrochloride (BB3) according to general procedure A to give 5-chloro-N-(4-cyano-2-methylcarbamoylmethoxy-benzyl)-nicotinamide. Colorless solid. MS 359.3 ([M+H]⁺)

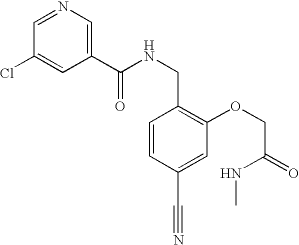

19.2 5-Chloro-N-(4-cyano-2-methylcarbamoylmethoxy-benzyl)-nicotinamide was converted to N-(4-carbamimidoyl-2-methylcarbamoylmethoxy-benzyl)-5-chloro-nicotinamide hydrochloride according to general procedure C. Colorless foam. MS 376.3 ([M+H]⁺)

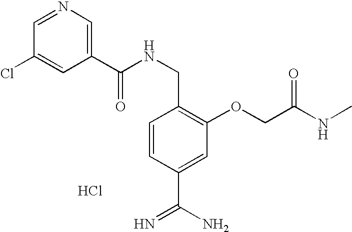

### Example 20

20.1 3-Methylbenzoic acid was coupled with 4-aminomethyl-3-nitro-benzonitrile (BB4) according to general procedure A to give N-(4-cyano-2-nitro-benzyl)-3-methyl-benzamide. Light yellow solid.

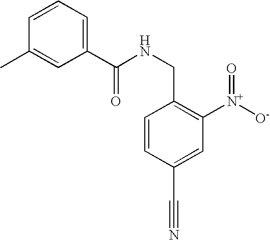

20.2 N-(4-Cyano-2-nitro-benzyl)-3-methyl-benzamide was converted to N-(4-carbamimidoyl-2-nitro-benzyl)-3-methyl-benzamide hydrohloride according to general procedure C. Light yellow solid. MS 312.9 ([M+H]⁺)

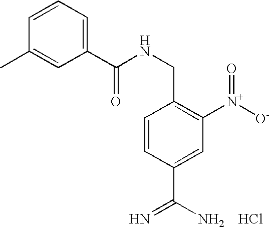

### Example 21

21.1 The nitro group of N-(4-cyano-2-nitro-benzyl)-3-methyl-benzamide (example 20.1) was reduced according to general procedure B to give N-(2-amino-4-cyano-benzyl)-3-methyl-benzamide. Light yellow solid. MS 266.2 ([M+H]⁺)

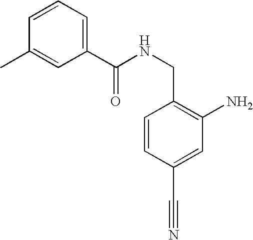

21.2 N-(2-Amino-4-cyano-benzyl)-3-methyl-benzamide was converted to N-(2-amino-4-carbamimidoyl-benzyl)-3-methyl-benzamide hydrochloride according to general procedure C. Off-white solid. MS 283.1 ([M+H]⁺)

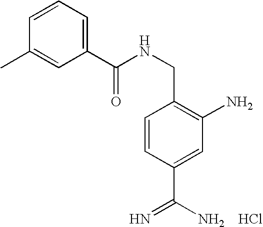

### Example 22

22.1 To a solution of N-(2-Amino-4-cyano-benzyl)-3-methyl-benzamide (example 21.1, 150 mg) in N,N-dimethylacetamide (0.6 ml) were added benzyl bromide (148 mg), N-ethyl-diisopropyl amine (110 mg) and tetrabutylammonium iodide (10 mg). The mixture was stirred for 67 h at 50° C. and for 1 h at 100° C. After cooling to r.t., the mixture was diluted with water and extracted with EtOAc. The organic phase was washed with water, dried, filtered and concentrated under reduced pressure. The products were purified by chromatography (SiO₂, cyclohexane/EtOAc 1:0=>1:1) to give N-(2-benzylamino-4-cyano-benzyl)-3-methyl-benzamide (65 mg, MS 356.2 ([M+H]⁺)) and N-(4-cyano-2-dibenzylamino-benzyl)-3-methyl-benzamide (148 mg, MS 446.1 ([M+H]⁺)) as colorless solids.

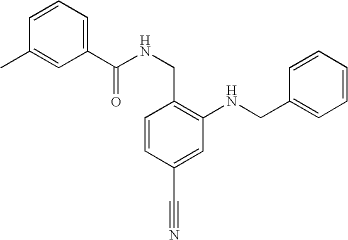

22.2 N-(2-Benzylamino-4-cyano-benzyl)-3-methyl-benzamide was converted to N-(2-benzylamino-4-carbamimidoyl-benzyl)-3-methyl-benzamide hydrochloride according to general procedure C. Off-white solid. MS 373.3 ([M+H]⁺)

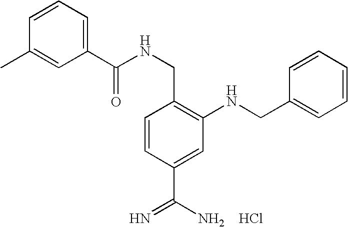

### Example 23

23.1 N-(4-Cyano-2-dibenzylamino-benzyl)-3-methyl-benzamide (example 22.1) was converted to N-(4-carbamimidoyl-2-dibenzylamino-benzyl)-3-methyl-benzamide hydrochloride according to general procedure C. Colorless solid. MS 463.4 ([M+H]⁺)

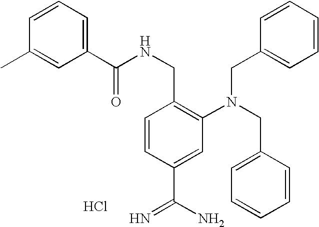

### Example 24

24.1 In analogy to example 22.1, N-(2-Amino-4-cyano-benzyl)-3-methyl-benzamide (example 21.1) was alkylated with ethyliodoacetate. The product of this reaction was converted to {5-carbamimidoyl-2-[(3-methyl-benzoylamino)-methyl]-phenylamino}-acetic acid ethyl ester hydrochloride according to general procedure C. Colorless solid. MS 369.2 ([M+H]⁺)

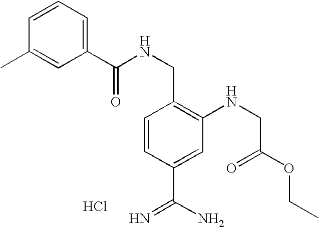

24.2 In analogy to example 8.3, {5-carbamimidoyl-2-[(3-methyl-benzoylamino)-methyl]-phenylamino}-acetic acid ethyl ester hydrochloride was hydrolyzed to give {5-carbamimidoyl-2-[(3-methyl-benzoylamino)-methyl]-phenylamino}-acetic acid. Colorless solid. MS 341.3 ([M+H]⁺)

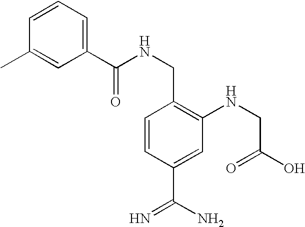

### Example 25

25.1 In analogy to example 22.1, N-(2-Amino-4-cyano-benzyl)-3-methyl-benzamide (example 21.1) was alkylated with 4-bromomethyl-3-fluoro-benzoic acid methyl ester to give 4-({5-cyano-2-[(3-methyl-benzoylamino)-methyl]-phenylamino}-methyl)-3-fluoro-benzoic acid methyl ester as an off-white solid. MS 432.4 ([M+H]⁺)

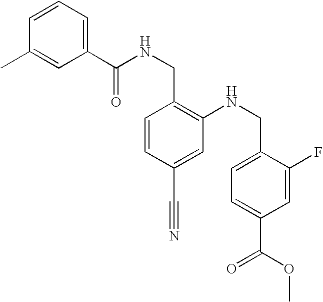

25.2 4-({5-Cyano-2-[(3-methyl-benzoylamino)-methyl]-phenylamino}-methyl)-3-fluoro-benzoic acid methyl ester was converted to 4-({5-carbamimidoyl-2-[(3-methyl-benzoylamino)-methyl]-phenylamino}-methyl)-3-fluoro-benzoic acid methyl ester hydrochloride according to general procedure C. Off-white solid. MS 449.2 ([M+H]⁺)

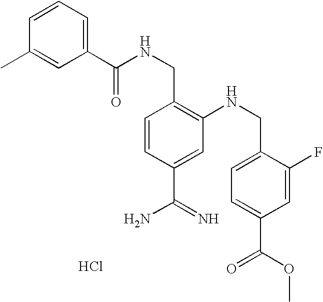

25.3 In analogy to example 8.3, 4-({5-carbarmimidoyl-2-[(3-methyl-benzoylamino)-methyl]-phenylmino}-methyl)-3-fluoro-benzoic acid methyl ester hydrochloride was hydrolyzed to give 4-({5-carbamimidoyl-2-[(3-methyl-benzoylamino)-methyl]-phenylamino}-methyl)-3fluoro-benzoic acid. Colorless solid. MS 435.2 ([M+H]⁺)

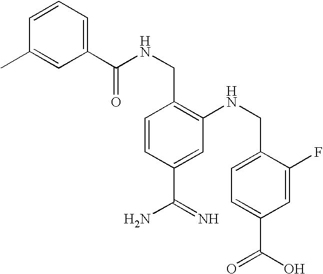

### Example 26

26.1 In analogy to example 22.1, N-(2-Amino-4-cyano-benzyl)-3-methyl-benzamide (example 21.1) was alkylated with 2-bromoethanol to give N-[4-cyano-2-(2-hydroxy-ethylamino)-benzyl]-3-methyl-benzamide as a yellow oil. MS 310.3 ([M+H]⁺)

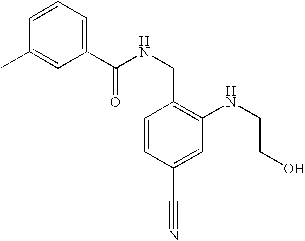

26.2 N-[4-Cyano-2-(2-hydroxy-ethylamino)-benzyl]-3-methyl-benzamide was converted to N-[4-carbamimidoyl-2-(2-hydroxy-ethylamino)-benzyl]-3-methyl-benzamide hydrochloride according to general procedure C. Off-white foam. MS 327.2 ([M+H]⁺)

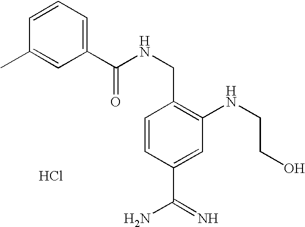

### Example 27

27.1 To a suspension of N-(2-Amino-4-cyano-benzyl)-3-methyl-benzamide (example 21.1, 60 mg) in CH₂Cl₂ (2.2 ml) were added pyridine (90 mg), dioxane (0.5 ml) and phenylacetyl chloride (43 mg). The mixture was stirred at r.t. for 5.5 h and then washed with 1 M HCl and with brine. The acqueous phase was extracted with CH₂Cl₂. The combined org. phase was dried, filtered and concentrated. The product was purified by chromatography (SiO2, cyclohexane/EtOAc 4:1=>1:4) to give N-(4-cyano-2-phenylacetylamino-benzyl)-3-methyl-benzamide (77 mg) as a colorless solid. MS 384.3 ([M+H]⁺)

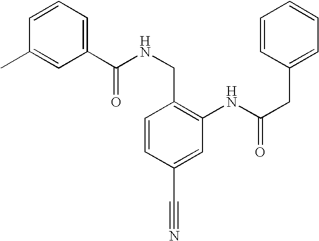

27.2 N-(4-Cyano-2-phenylacetylamino-benzyl)-3-methyl-benzamide was converted to N-(4-carbamimidoyl-2-phenylacetylamino-benzyl)-3-methyl-benzamide hydrochloride according to general procedure C. Colorless solid. MS 401.5 ([M+H]⁺)

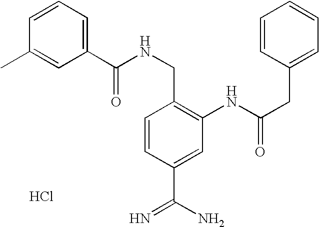

### Example 28

28.1 In analogy to example 22.1, N-(2-Amino-4-cyano-benzyl)-3-methyl-benzamide (example 21.1) was alkylated with 2-chloro-N-methylacetamide to give N-[4-cyano-2-(methylcarbamoylmethyl-amino)-benzyl]-3-methyl-benzamide as an off-white solid. MS 337.3 ([M+H]⁺)

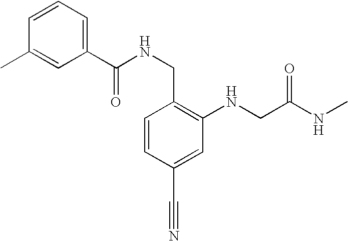

28.2 N-[4-cyano-2-(methylcarbamoylmethyl-amino)-benzyl]-3-methyl-benzamide was converted to N-[4-carbamimidoyl-2-(methylcarbamoylmethyl-amino)-benzyl]-3-methyl-benzamide hydrochloride according to general procedure C. Off-white solid. MS 354.2 ([M+H]⁺

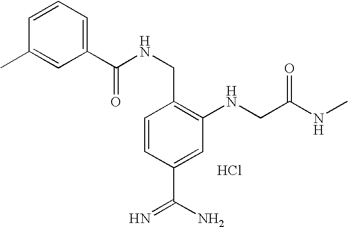

### Example 29

29.1 In analogy to example 22.1, N-(2-Amino-4-cyano-benzyl)-3-methyl-benzamide (example 21.1) was alkylated with 2-chloro-N,N-dimethylacetamide to give N-[4-cyano-2-(dimethylcarbamoylmethyl-amino)-benzyl]-3-methyl-benzamide as a light yellow solid. MS 351.2 ([M+H]⁺)

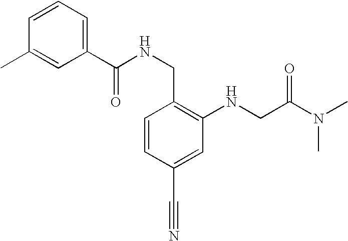

29.2 N-[4-Cyano-2-(dimethylcarbamoylmethyl-amino)-benzyl]-3-methyl-benzamide was converted to N-[4-carbamimidoyl-2-(dimethylcarbamoylmethyl-amino)-benzyl]-3-methyl-benzamide hydrochloride according to general procedure C. Off-white solid. MS 368.3 ([M+H]⁺)

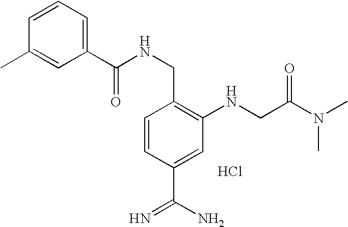

### Example 30

30.1 In analogy to example 22.1, N-(2-Amino-4-cyano-benzyl)-3-methyl-benzamide (example 21.1) was alkylated with 4-(2-chloroacetyl)morpholine to give N-[4-cyano-2-(2-morpholin-4-yl-2-oxo-ethylamino)-benzyl]-3-methyl-benzamide as a light yellow solid.

30.2 N-[4-Cyano-2-(2-morpholin-4-yl-2-oxo-ethylamino)-benzyl]-3-methyl-benzamide was converted to N-[4-carbamimidoyl-2-(2-morpholin-4-yl-2-oxo-ethylamino)-benzyl]-3-methyl-benzamide hydrochloride according to general procedure C. Light yellow solid. MS 410.4 ([M+H]⁺)

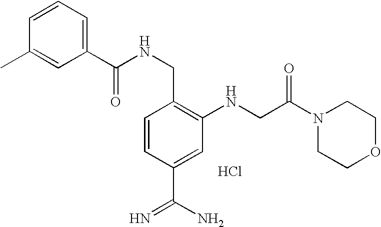

### Example 31

31.1 In analogy to example 22.1, N-(2-Amino-4-cyano-benzyl)-3-methyl-benzamide (example 21.1) was alkylated with N-chloroacetylaniline to give N-[4-cyano-2-(phenylcarbamoylmethyl-amino)-benzyl]-3-methyl-benzamide as an off-white solid. MS 399.3 ([M+H]⁺)

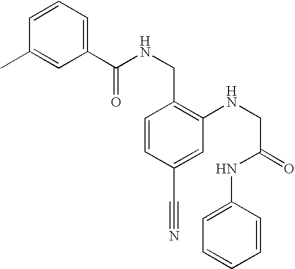

31.2 N-[4-Cyano-2-(phenylcarbamoylmethyl-amino)-benzyl]-3-methyl-benzamide was converted to N-[4-carbamimidoyl-2-(phenylcarbamoylmethyl-amino)-benzyl]-3-methyl-benzamide hydrochloride according to general procedure C. Off-white solid. MS 416.3 ([M+H]⁺)

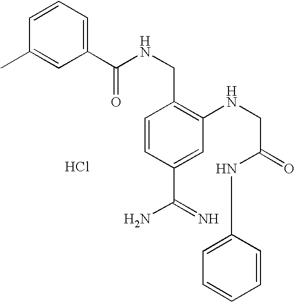

### Example 32

32.1 3-(Trifluoromethyl)-benzoic acid was coupled with 2-(2-aminomethyl-5-cyano-phenoxy)-N-methyl-acetamide hydrochloride (BB3) according to general procedure A to give N-(4-cyano-2-methylcarbamoylmethoxy-benzyl)-3-trifluoromethyl-benzamide. Colorless solid.

MS 392.2 ([M+H]⁺)

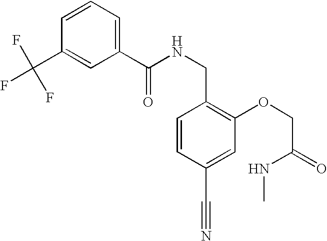

32.2 N-(4-Cyano-2-methylcarbamoylmethoxy-benzyl)-3-trifluoromethyl-benzamide was converted to N-(4-carbamimidoyl-2-methylcarbamoylmethoxy-benzyl)-3-trifluoromethyl-benzamide hydrochloride according to general procedure C. Colorless foam. MS 409.3 ([M+H]⁺)

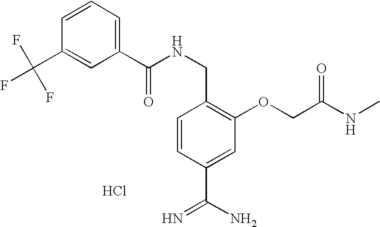

### Example 33

33.1 3-Chloro-5-methoxy-benzoic acid (CAS 82477-67-6) was coupled with 2-(2-aminomethyl-5-cyano-phenoxy)-N-methyl-acetamide hydrochloride (BB3) according to general procedure A to give 3-chloro-N-(4-cyano-2-methylcarbamoylmethoxy-benzyl)-5-methoxy-benzamide. Colorless solid. MS 388.5 ([M+H]⁺)

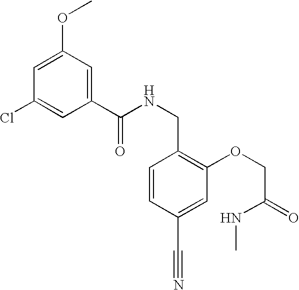

33.2 3-Chloro-N-(4-cyano-2-methylcarbamoylmethoxy-benzyl)-5-methoxy-benzamide was converted to N-(4-carbamimidoyl-2-methylcarbamoylmethoxy-benzyl)-3-chloro-5-methoxy-benzamide hydrochloride according to general procedure C. Colorless solid. MS 405.3 ([M+H]⁺)

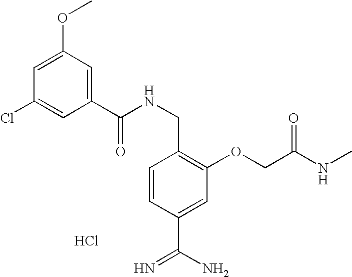

### Example 34

34.1 5-Chlorosalicylic acid was coupled with 2-(2-aminomethyl-5-cyano-phenoxy)-N-methyl-acetamide hydrochloride (BB3) according to general procedure A to give 5-chloro-N-(4-cyano-2-methylcarbamoylmethoxy-benzyl)2-hydroxy-benzamide. Off-white solid.

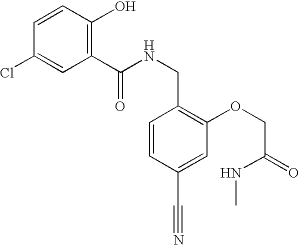

34.2 5-Chloro-N-(4-cyano-2-methylcarbamoylmethoxy-benzyl)-2-hydroxy-benzamide was converted to N-(4-carbamimidoyl-2-methylcarbamoylmethoxy-benzyl)-5-chloro-2-hydroxy-benzamide hydrochloride according to general procedure C. Colorless foam. MS 391.2 ([M+H]⁺)

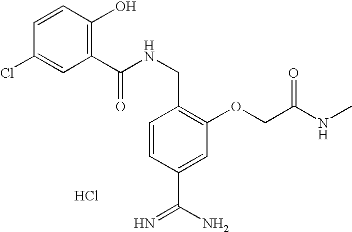

### Example 35

35.1 2,5-Dimethyl-2H-pyrazole-3-carboxylic acid (CAS 5744-56-9) was coupled with 2-(2-aminomethyl-5-cyano-phenoxy)-N-methyl-acetamide hydrochloride (BB3) according to general procedure A to give 2,5-dimethyl-2H-pyrazole-3-carboxylic acid 4-cyano-2-methylcarbamoylmethoxy-benzylamide. Colorless solid. MS 342.2 ([M+H]⁺)

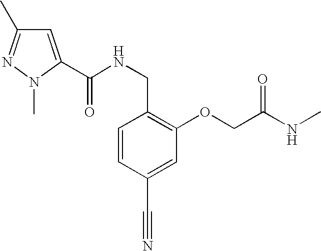

35.2 2,5-Dimethyl-2H-pyrazole-3-carboxylic acid 4-cyano-2-methylcarbamoylmethoxy-benzylamide was converted to 2,5-dimethyl-2H-pyrazole-3-carboxylic acid 4-carbamimidoyl-2-methylcarbamoylmethoxy-benzylamide hydrochloride according to general procedure C. Colorless solid. MS 359.0 ([M+H]⁺)

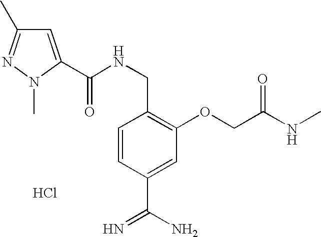

### Example 36

36.1 1,5-Dimethyl-1H-pyrazole-3-carboxylic acid (CAS 5744-59-2) was coupled with 2-(2-aminomethyl-5-cyano-phenoxy)-N-methyl-acetamide hydrochloride (BB3) according to general procedure A to give 1,5-dimethyl-1H-pyrazole-3-carboxylic acid 4-cyano-2-methylcarbamoylmethoxy-benzylamide. Colorless solid. MS 342.1 ([M+H]⁺)

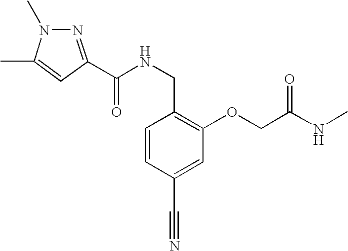

36.2 1,5-Dimethyl-1H-pyrazole-3-carboxylic acid 4-cyano-2-methylcarbamoylmethoxy-benzylamide was converted to 1,5-dimethyl-1H-pyrazole-3-carboxylic acid 4-carbamimidoyl-2-methylcarbamoylmethoxy-benzylamide hydrochloride according to general procedure C. Colorless solid. MS 359.3 ([M+H]⁺)

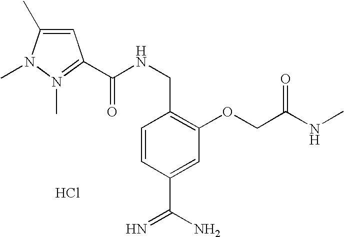

### Example 37

37.1 2-Methyloxazole-4-carboxylic acid (CAS 23012-17-1) was coupled with 2-(2-aminomethyl-5-cyano-phenoxy)-N-methyl-acetamide hydrochloride (BB3) according to general procedure A to give 2-methyl-oxazole-4-carboxylic acid 4-cyano-2-methylcarbamoylmethoxy-benzylamide. Colorless solid. MS 329.2 ([M+H]⁺)

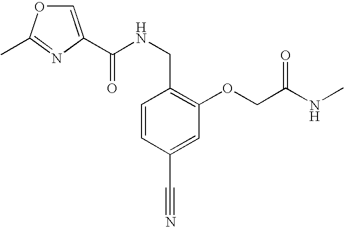

37.2 2-Methyl-oxazole-4-carboxylic acid 4-cyano-2-methylcarbamoylmethoxy-benzylamide was converted to 2-methyl-oxazole-4-carboxylic acid 4-carbamimidoyl-2-methylcarbamoylmethoxy-benzylamide hydrochloride according to general procedure C. Colorless solid. MS 345.9 ([M+H]⁺)

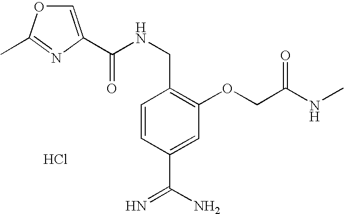

### Example 38

38.1 4-Fluoro-3-methylbenzoic acid was coupled with 4-aminomethyl-3-hydroxy-benzonitrile hydrochloride (BB1) according to general procedure A to give N-(4-cyano-2-hydroxy-benzyl)-4-fluoro-3-methyl-benzamide. Light yellow oil. MS 283.1 ([M−H]⁻)

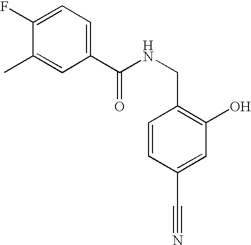

38.2 In analogy to example 1.2, N-(4-cyano-2-hydroxy-benzyl)-4-fluoro-3-methyl-benzamide was alkylated with iodoacetamide to give N-(2-carbamoylmethoxy-4-cyano-benzyl)-4-fluoro-3-methyl-benzamide as a colorless solid. MS 342.0 ([M+H]⁺)

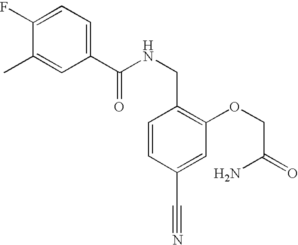

38.3 N-(2-Carbamoylmethoxy-4-cyano-benzyl)-4-fluoro-3-methyl-benzamide was converted to N-(4-carbamimidoyl-2-carbamoylmethoxy-benzyl)-4-fluoro-3-methyl-benzamide acetic acid salt according to general procedure C. Colorless solid. MS 359.3 ([M+H]⁺)

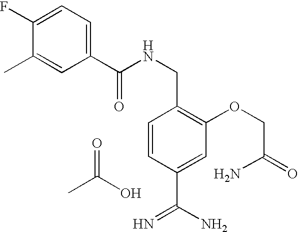

### Example 39

39.1 In analogy to example 1.2, N-(4-cyano-2-hydroxy-benzyl)-4-fluoro-3-methyl-benzamide (example 38.1) was alkylated with 2-chloro-N-methylacetamide to give N-(4-cyano-2-methylcarbamoylmethoxy-benzyl)-4-fluoro-3-methyl-benzamide as a colorless solid. MS 356.2 ([M+H]⁺)

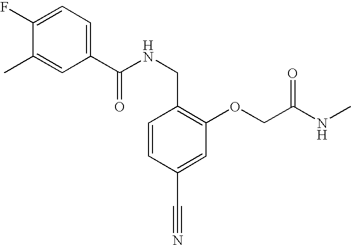

39.2 N-(4-Cyano-2-methylcarbamoylmethoxy-benzyl)-4-fluoro-3-methyl-benzamide was converted to N-(4-carbamimidoyl-2-methylcarbamoylmethoxy-benzyl)-4-fluoro-3-methyl-benzamide hydrochloride according to general procedure C. Colorless solid. MS 373.3 ([M+H]⁺)

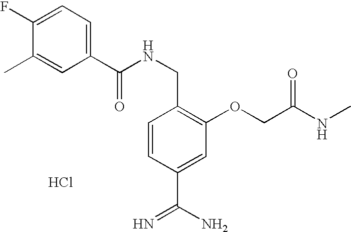

39.3 To a solution of N-(4-carbamimidoyl-2-methylcarbamoylmethoxy-benzyl)-4-fluoro-3-methyl-benzamide hydrochloride (149 mg) in N,N-dimethylacetamide (1.4 ml) were added ethylchloroformate (40 mg) and triethylamine (111 mg) at 0° C. The mixture was stirred at 0° C. for 2 h. Water was added and the mixture was extracted with ethyl acetate. The org. phase was washed with water. The product precipitated in the organic phase and was filtered off. An additional batch of product was obtained by concentration of the filtrate and suspension of the residue in CH₂Cl₂. The solid was filtered off. Both batches of product were combined and dried to give a total of 115 mg of [1-amino-1-{4-[(4-fluoro-3-methyl-benzoylamino)-methyl]-3-methylcarbamoylmethoxy-phenyl}-meth-(Z)-ylidene]-carbamic acid ethyl ester as a colorless solid. MS 445.2 ([M+H]⁺)

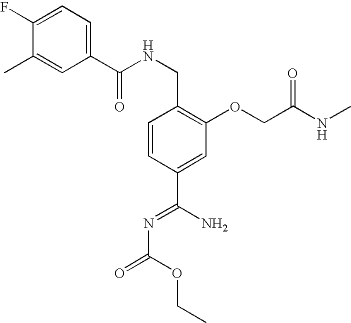

39.4 To a suspension of N-(4-cyano-2-methylcarbamoylmethoxy-benzyl)-4-fluoro-3-methyl-benzamide (example 39.1, 100 mg) in dry ethanol (2.6 ml) were added hydroxylamine hydrochloride (78 mg) and triethylamine (228 mg). The mixture was stirred at r.t. overnight. The solvent was evaporated. Water was added and the mixture was extracted with CH₂Cl₂. The product precipitated in the organic phase and was filtered off to give 99 mg of 4-fluoro-N-[4-(N-hydroxycarbamimidoyl)-2-methylcarbamoylmethoxy-benzyl]-3-methyl-benzamide as a colorless solid. MS 389.2 ([M+H]⁺)

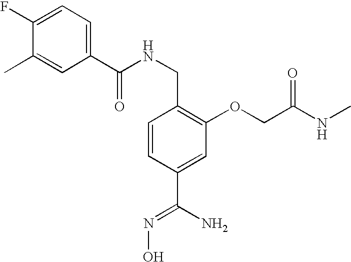

### Example 40

40.1 To a solution of N-(4-cyano-2-hydroxy-benzyl)-4-fluoro-3-methyl-benzamide (example 38.1, 1 g) in THF (30 ml) were added triphenylphosphine (1.384 g) and BOC-glycinol (0.868 g). The mixture was cooled to 0° C. and diethylazodicarboxylate (0.988 g) was added dropwise. The ice bath was removed and the mixture was stirred at r.t. for 4 days. The solvent was evaporated and the product was purified by chromatography (SiO₂, CH₂Cl₂=>CH₂Cl₂/MeOH 9:1) to give 1.432 g of (2-{5-cyano-2-[(4-fluoro-3-methyl-benzoylamino)-methyl]-phenoxy}-ethyl)-carbamic acid tert-butyl ester as a colorless foam. MS 428.5 ([M+H]⁺)

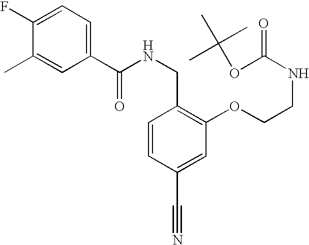

40.2 The BOC protecting group in (2-{5-cyano-2-[(4-fluoro-3-methyl-benzoylamino)-methyl]-phenoxy}-ethyl)-carbamic acid tert-butyl ester was removed using standard conditions (CF₃COOH in CH₂Cl₂) to give N-[2-(2-amino-ethoxy)-4-cyano-benzyl]-4-fluoro-3-methyl-benzamide as a colorless foam. MS 328.2 ([M+H]⁺)

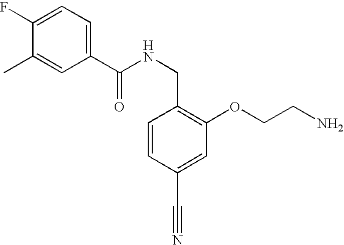

40.3 To a suspension of N-[2-(2-amino-ethoxy)-4-cyano-benzyl]-4-fluoro-3-methyl-benzamide (100 mg) in CH₂Cl₂ (3 ml) were added pyridine (121 mg), dioxane (0.7 ml) and acetyl chloride (29 mg). The mixture was stirred for 4.5 h at r.t. The mixture was diluted with CH₂Cl₂ and was washed with 1 M HCl and brine. The acqueous phase was extracted with CH₂Cl₂. The combined org. phase was dried, filtered and concentrated to give N-[2-(2-acetylamino-ethoxy)-4-cyano-benzyl]-4-fluoro-3-methyl-benzamide (121 mg) as a colorless solid. MS 370.1 ([M+H]⁺)

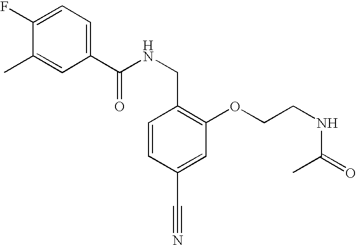

This product was used in the next step without further purification.

40.4 N-[2-(2-Acetylamino-ethoxy)-4-cyano-benzyl]-4-fluoro-3-methyl-benzamide was converted to N-[2-(2-acetylamino-ethoxy)-4-carbamimidoyl-benzyl]-4-fluoro-3-methyl-benzamide hydrochloride according to general procedure C. Colorless solid. MS 387.4 ([M+H]⁺)

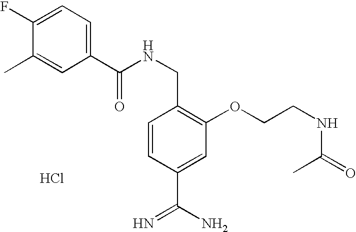

### Example 41

41.1 In analogy to example 40.3, N-[2-(2-amino-ethoxy)-4-cyano-benzyl]-4-fluoro-3-methyl-benzamide (example 40.2) was reacted with methanesulfonyl chloride and pyridine in CH₂Cl₂/dioxane to give N-[4-cyano-2-(2-methanesulfonylamino-ethoxy)-benzyl]-4-fluoro-3-methyl-benzamide as a colorless foam. MS 406.2 ([M+H]⁺)

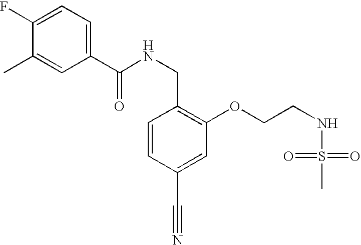

41.2 N-[4-Cyano-2-(2-methanesulfonylamino-ethoxy)-benzyl]-4-fluoro-3-methyl-benzamide was converted to N-[4-carbamimidoyl-2-(2-methanesulfonylamino-ethoxy)-benzyl]-4-fluoro-3-methyl-benzamide hydrochloride according to general procedure C. Colorless foam. MS 423.0 ([M+H]⁺)

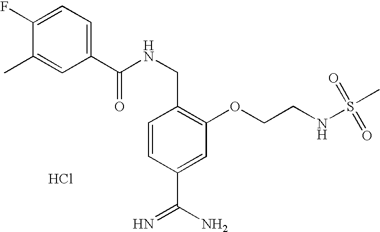

### Example 42

42.1 In analogy to example 40.3, N-[2-(2-amino-ethoxy)-4-cyano-benzyl]-4-fluoro-3-methyl-benzamide (example 40.2) was reacted with 2-fluorobenzoyl chloride and pyridine in CH₂Cl₂/dioxane to give N-{4-cyano-2-[2-(2-fluoro-benzoylamino)-ethoxy]-benzyl}-4-fluoro-3-methyl-benzamide as a colorless foam. MS 450.1 ([M+H]⁺)

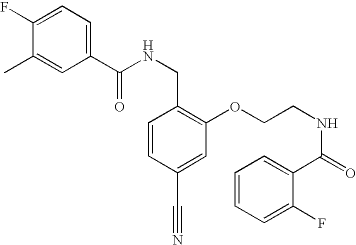

42.2 N-{4-Cyano-2-[2-(2-fluoro-benzoylamino)-ethoxy]-benzyl}-4-fluoro-3-methyl-benzamide was converted to N-{4-carbamimidoyl-2-[2-(2-fluoro-benzoylamino)-ethoxy]-benzyl}-4-fluoro-3-methyl-benzamide hydrochloride according to general procedure C. Colorless foam. MS 467.4 ([M+H]⁺)

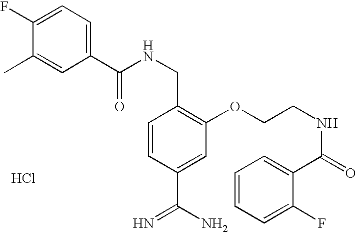

### Example 43

43.1 4-Fluoro-3-methylbenzoic acid was coupled with 4-aminomethyl-3-nitro-benzonitrile (BB4) according to general procedure A to give N-(4-cyano-2-nitro-benzyl)-4-fluoro-3-methyl-benzamide. Light yellow solid.

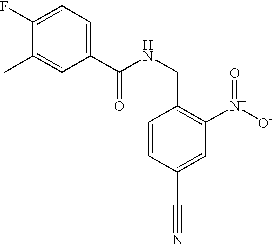

43.2 The nitro group of N-(4-cyano-2-nitro-benzyl)-4-fluoro-3-methyl-benzamide was reduced according to general procedure B to give N-(2-amino-4-cyano-benzyl)-4-fluoro-3-methyl-benzamide. Light yellow solid. MS 284.1 ([M+H]⁺)

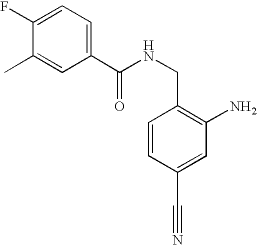

43.3 In analogy to example 22.1, N-(2-amino-4-cyano-benzyl)-4-fluoro-3-methyl-benzamide was alkylated with ethyliodoacetate to give {5-cyano-2-[(4-fluoro-3-methyl-benzoylamino)-methyl]-phenylamino}-acetic acid ethyl ester as an orange, amorphous solid. MS 370.1 ([M+H]⁺)

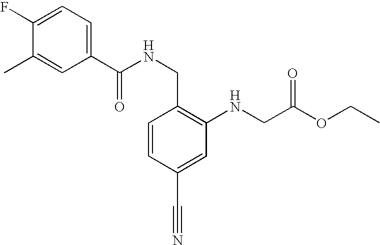

43.4 {5-Cyano-2-[(4-fluoro-3-methyl-benzoylamino)-methyl]-phenylamino}-acetic acid ethyl ester was converted to {5-carbamimidoyl-2-[(4-fluoro-3-methyl-benzoylamino)-methyl]-phenylamino}-acetic acid ethyl ester hydrochloride according to general procedure C. Off-white solid. MS 387.4 ([M+H]⁺)

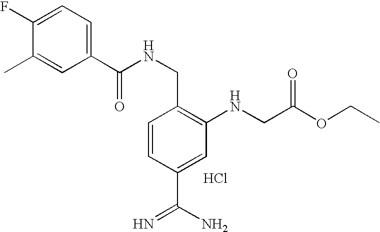

43.5 In analogy to example 8.3, {5-carbamimidoyl-2-[(4-fluoro-3-methyl-benzoylamino)-methyl]-phenylamino}-acetic acid ethyl ester hydrochloride was hydrolyzed to give {5-carbamimidoyl-2-[(4-fluoro-3-methyl-benzoylamino)-methyl]-phenylamino}-acetic acid. Colorless solid. MS 359.3 ([M+H]⁺)

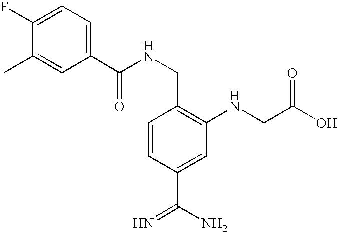

### Example 44

44.1 3-Chlorobenzoic acid was coupled with 4-aminomethyl-3-hydroxy-benzonitrile hydrochloride (BB1) according to general procedure A to give 3-chloro-N-(4-cyano-2-hydroxy-benzyl)-benzamide. Colorless solid.

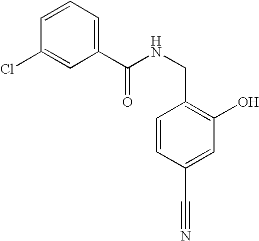

44.2 In analogy to example 1.2, 3-chloro-N-(4-cyano-2-hydroxy-benzyl)-benzamide was alkylated with iodoacetamide to give N-(2-carbamoylmethoxy-4-cyano-benzyl)-3-chloro-benzamide as a colorless solid. MS 344.1 ([M+H]⁺)

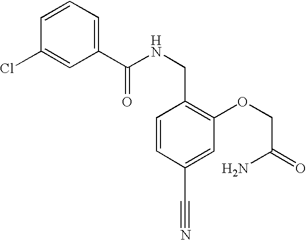

44.3 N-(2-Carbamoylmethoxy-4-cyano-benzyl)-3-chloro-benzamide was converted to (RS)—N-(4-carbamimidoyl-2-carbamoylmethoxy-benzyl)-3-chloro-benzamide hydrochloride according to general procedure C. Colorless solid. MS 361.3 ([M+H]⁺)

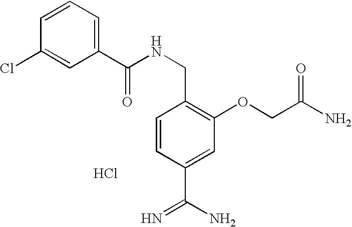

### Example 45

45.1 In analogy to example 1.2, 3-chloro-N-(4-cyano-2-hydroxy-benzyl)-benzamide (example 44.1) was alkylated with 2-chloro-N-methylacetamide to give 3-chloro-N-(4-cyano-2-methylcarbamoylmethoxy-benzyl)-benzamide as a colorless solid. MS 356.2 ([M−H]⁻)

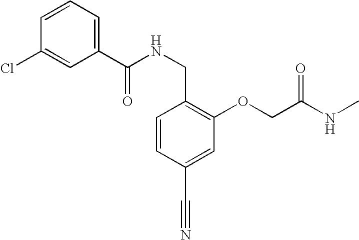

45.2 3-Chloro-N-(4-cyano-2-methylcarbamoylmethoxy-benzyl)-benzamide was converted to N-(4-carbamimidoyl-2-methylcarbamoylmethoxy-benzyl)-3-chloro-benzamide acetic acid salt according to general procedure C. Colorless solid. MS 375.3 ([M+H]⁺)

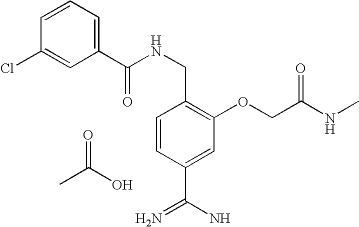

### Example 46

46.1 In analogy to example 1.2, 3-chloro-N-(4-cyano-2-hydroxy-benzyl)-benzamide (example 44.1) was alkylated with N-(chloroacetyl)-4-fluoroaniline to give 3-chloro-N-{4-cyano-2-[(4-fluoro-phenylcarbamoyl)-methoxy]-benzyl}-benzamide as a colorless solid. MS 436.5 ([M−H]⁻)

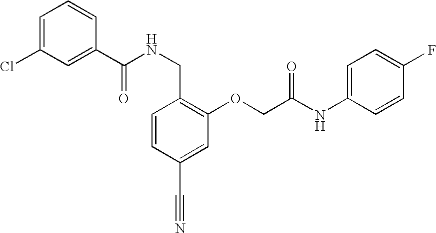

46.2 3-Chloro-N-{4-cyano-2-[(4-fluoro-phenylcarbamoyl)-methoxy]-benzyl}-benzamide was converted to N-{4-carbamimidoyl-2-[(4-fluoro-phenylcarbamoyl)-methoxy]-benzyl}-3-chloro-benzamide acetic acid salt according to general procedure C. Colorless solid. MS 455.4 ([M+H]⁺)

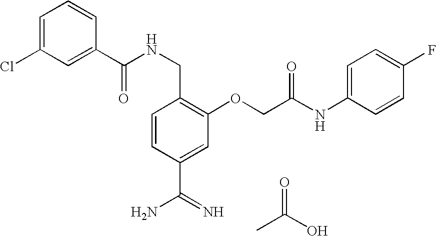

### Example 47

47.1 In analogy to example 1.2, 3-chloro-N-(4-cyano-2-hydroxy-benzyl)-benzamide (example 44.1) was alkylated with 2-chloro-N-(2-morpholin-4-yl-ethyl)-acetamide (CAS 112361-76-9) to give 3-chloro-N-{4-cyano-2-[(2-morpholin-4-yl-ethylcarbamoyl)-methoxy]-benzyl}-benzamide as a colorless foam. MS 457.4 ([M+H]⁺)

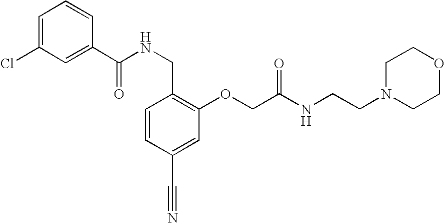

47.2 3-Chloro-N-{4-cyano-2-[(2-morpholin-4-yl-ethylcarbamoyl)-methoxy]-benzyl}-benzamide was converted to N-{4-carbamimidoyl-2-[(2-morpholin-4-yl-ethylcarbamoyl)-methoxy]-benzyl}-3-chloro-benzamide hydrochloride according to general procedure C. Colorless solid. MS 474.2 ([M+H]⁺)

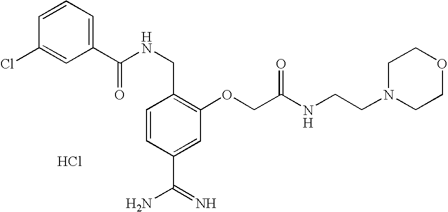

### Example 48

48.1 To a solution of 2-fluoro-N-(2-hydroxy-ethyl)-benzamide (CAS 111904-31-5, 1.685 g) in THF (50 ml) were added N-ethyldiisopropyl amine (1.455 g) and methanesulfochloride (1.277 g) at 0° C. The mixture was stirred for 2 h at 0° C. and for 2.5 h at r.t. The mixture was poured into an ice cold solution of KHSO₄ and extracted with ethyl acetate. The org. phase was washed with water, dried, filtered and concentrated to give methanesulfonic acid 2-(2-fluoro-benzoylamino)-ethyl ester (2.087 g) as a colorless solid. MS 261.9 ([M+H]⁺)

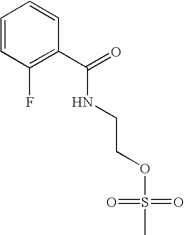

48.2 In analogy to example 1.2, 3-chloro-N-(4-cyano-2-hydroxy-benzyl)-benzamide (example 44.1) was alkylated with methanesulfonic acid 2-(2-fluoro-benzoylamino)-ethyl ester to give N-[2-(2-{[(3-chlorobenzoyl)amino]methyl}-5-cyanophenoxy)ethyl]-2-fluorobenzamide as a colorless foam. MS 452.2 ([M+H]⁺)

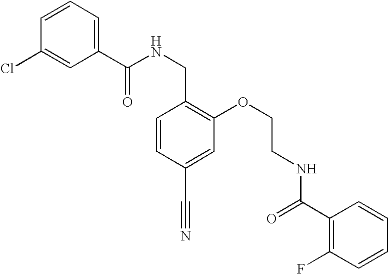

48.3 N-[2-(2-{[(3-Chlorobenzoyl)amino]methyl}-5-cyanophenoxy)ethyl]-2-fluorobenzamide was converted to N-[2-(2-{[(3-chlorobenzoyl)amino]methyl}-5-carbamimidoylphenoxy)ethyl]-2-fluorobenzamide according to general procedure C. Colorless solid. MS 469.2 ([M+H]⁺)

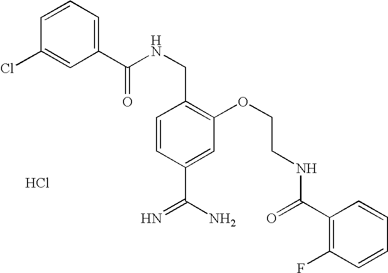

### Example 49

49.1 3-Chlorobenzoic acid was coupled with 4-aminomethyl-3-nitro-benzonitrile (BB4) according to general procedure A to give 3-chloro-N-(4-cyano-2-nitro-benzyl)-benzamide. Light yellow solid.

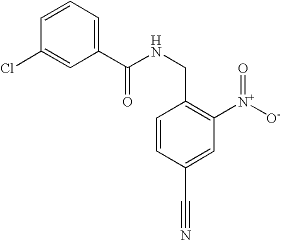

49.2 To a suspension of 3-chloro-N-(4-cyano-2-nitro-benzyl)-benzamide (7.11 g) in acetic acid (20 ml) was slowly added zinc powder (11.8 g). The mixture was stirred for 2.5 h ar r.t. The solid was filtered off and washed with acetic acid, ethanol and tetrahydrofuran. The filtrate was combined and concentrated. The residue was dissolved in dichloromethane containing methanol and washed with 1 M NaOH solution. The acqueous phase was extracted with dichloromethane. The combined organic phase was dried, filtered and concentrated to give N-(2-amino-4-cyano-benzyl)-3-chloro-benzamide (5.41 g) as a light yellow solid.

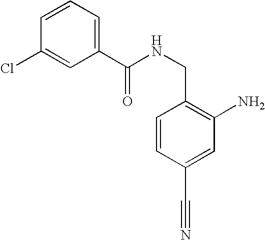

49.3 In analogy to example 22.1, N-(2-amino-4-cyano-benzyl)-3-chloro-benzamide was alkylated with 2-bromoethanol to give 3-chloro-N-[4-cyano-2-(2-hydroxy-ethylamino)-benzyl]-benzamide as a colorless solid. MS 328.2 ([M−H]⁻)

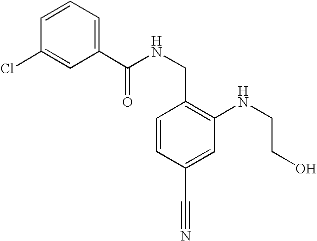

49.4 3-Chloro-N-[4-cyano-2-(2-hydroxy-ethylamino)-benzyl]-benzamide was converted to N-[4-carbamimidoyl-2-(2-hydroxy-ethylamino)-benzyl]-3-chloro-benzamide hydrochloride according to general procedure C. Light yellow foam. MS 346.8 ([M+H]⁺)

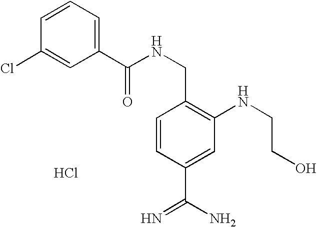

### Example 50

50.1 In analogy to example 22.1, N-(2-amino-4-cyano-benzyl)-3-chloro-benzamide (example 49.2) was alkylated with 3-(chloromethyl)-benzamide to give N-(2-{[3-(aminocarbonyl)benzyl]amino}-4-cyanobenzyl)-3-chlorobenzamide as a colorless solid. MS 419.0 ([M+H]⁺)

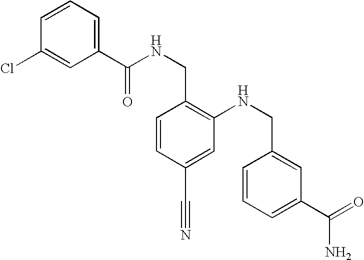

50.2 N-(2-{[3-(Aminocarbonyl)benzyl]amino}-4-cyanobenzyl)-3-chlorobenzamide was converted to N-{2-{[3-(aminocarbonyl)benzyl]amino}-4-[amino(imino)methyl]benzyl}-3-chlorobenzamide hydrochloride according to general procedure C. Light yellow solid. MS 436.1 ([M+H]⁺)

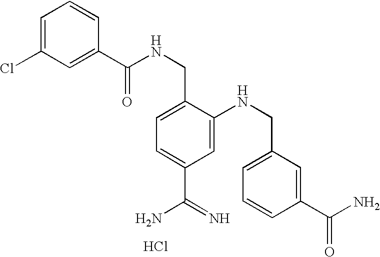

### Example 51

50.1 In analogy to example 22.1, N-(2-amino-4-cyano-benzyl)-3-chloro-benzamide (example 49.2) was alkylated with methyl 3-(bromomethyl)benzoate. The product of this reaction could not be obtained pure and was directly converted according to general procedure C to give 3-({5-carbamimidoyl-2-[(3-chloro-benzoylamino)-methyl]-phenylamino}-methyl)-benzoic acid methyl ester hydrochloride as a yellow solid. MS 451 ([M+H]⁺)

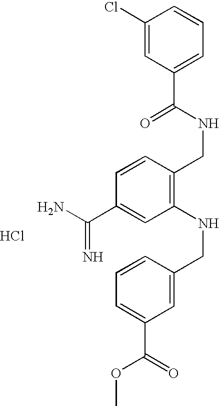

51.2 In analogy to example 8.3, 3-({5-carbamimidoyl-2-[(3-chloro-benzoylamino)-methyl]-phenylamino}-methyl)-benzoic acid methyl ester hydrochloride was hydrolyzed to give 3-({5-carbamimidoyl-2-[(3-chloro-benzoylamino)-methyl]-phenylamino}-methyl)-benzoic acid as a light yellow solid. MS 437.4 ([M+H]⁺)

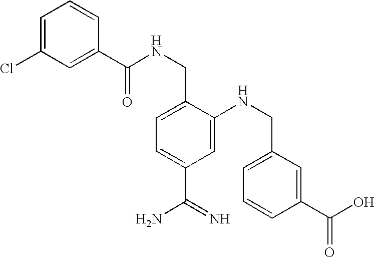

### Example 52

52.1 In analogy to example 22.1, N-(2-amino-4-cyano-benzyl)-3-chloro-benzamide (example 49.2) was alkylated with methyl 3-(bromomethyl)benzoate. The product of this reaction could not be obtained pure and was directly hydrolyzed in analogy to example 8.3 to give 3-({2-[(3-chloro-benzoylamino)-methyl]-5-cyano-phenylamino}-methyl)-benzoic acid as a light yellow solid. MS 418 ([M−H]⁻)

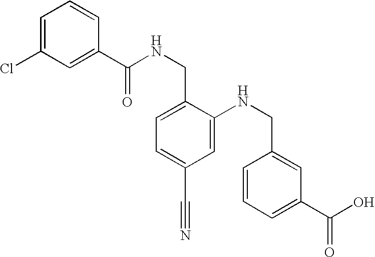

52.2 3-({2-[(3-Chloro-benzoylamino)-methyl]-5-cyano-phenylamino}-methyl)-benzoic acid was coupled with 2-methoxyethylamine according to general procedure A to give 3-chloro-N-{4-cyano-2-[(3-{[(2-methoxyethyl)amino]carbonyl}benzyl)amino]benzyl}benzamide. Light yellow solid. MS 477.4 ([M+H]⁺)

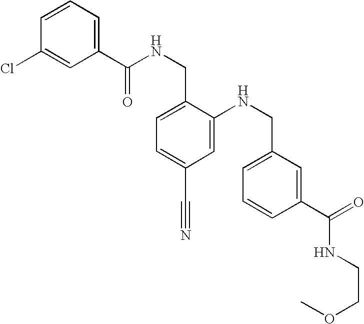

52.3 In analogy to example 39.4, 3-chloro-N-{4-cyano-2-[(3-{[(2-methoxyethyl)amino]carbonyl}benzyl)amino]benzyl}benzamide was treated with hydroxylamine hydrochloride and triethylamine to give N-{4-[amino(hydroxyimino)methyl]-2-[(3-{[(2-methoxyethyl)amino]carbonyl}benzyl)amino]benzyl}-3-chlorobenzamide. Colorless solid. MS 510.5 ([M+H]⁺)

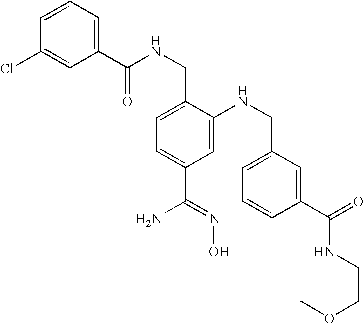

52.4 A solution of N-{4-[amino(hydroxyimino)methyl]-2-[(3-{[(2-methoxyethyl)amino]carbonyl}benzyl)amino]benzyl}-3-chlorobenzamide in ethanol/tetrahydrofuran and acetic acid was hydrogenated at normal pressure for 14 h using Raney nickel as a catalyst. The catalyst was filtered off and the filtrate was concentrated. The product was purified by chromatography (SiO₂, ethyl acetate/acetone/water/methanol 6:2:1:1) to give N-{4-[amino(imino)methyl]-2-[(3-{[(2-methoxyethyl)amino]carbonyl}benzyl)amino]benzyl}-3-chlorobenzamide acetic acid salt as a yellow solid. MS 494.5 ([M+H]⁺)

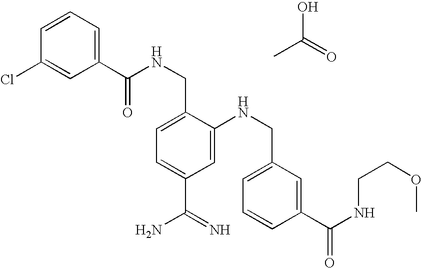

### Example 53

53.1 3-({2-[(3-Chloro-benzoylamino)-methyl]-5-cyano-phenylamino}-methyl)-benzoic acid (example 52.1) was coupled with morpholine according to general procedure A to give 3-chloro-N-{4-cyano-2-[3-(morpholine-4-carbonyl)-benzylamino]-benzyl}-benzamide. Light yellow solid. MS 487.4 ([M−H]⁻)

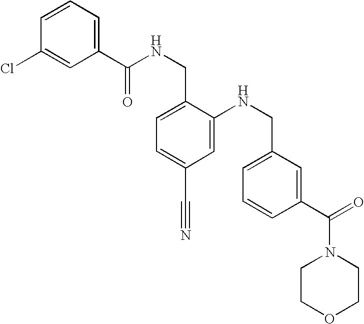

53.2 In analogy to example 39.4, 3-chloro-N-{4-cyano-2-[3-(morpholine-4-carbonyl)-benzylamino]-benzyl}-benzamide was treated with hydroxylamine hydrochloride and triethylamine to give 3-chloro-N-{4-(N-hydroxycarbamimidoyl)-2-[3-(morpholine-4-carbonyl)-benzylamino]-benzyl}-benzamide. Colorless solid. MS 522.5 ([M+H]⁺)

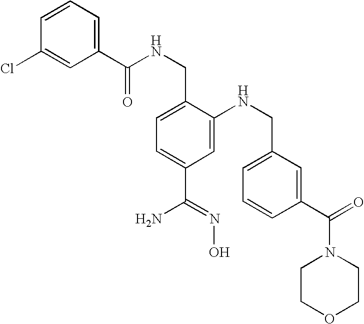

53.3 In analogy to example 52.4, 3-chloro-N-{4-(N-hydroxycarbamimidoyl)-2-[3-(morpholine-4-carbonyl)-benzylamino]-benzyl}-benzamide was hydrogenated to give N-(4-[amino(imino)methyl]-2-{[3-(4-morpholinylcarbonyl)benzyl]amino}benzyl)-3-chlorobenzamide acetic acid salt as an orange solid. MS 506.4 ([M+H]⁺)

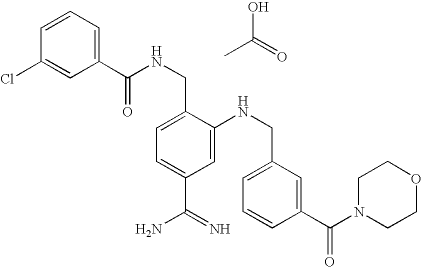

### Example 54

54.1 In analogy to example 22.1, N-(2-amino-4-cyano-benzyl)-3-chloro-benzamide (example 49.2) was alkylated with 4-(bromomethyl)-benzamide to give N-(2-{[4-(aminocarbonyl)benzyl]amino}-4-cyanobenzyl)-3-chlorobenzamide as a light yellow solid. MS 419.3 ([M+H]⁺)

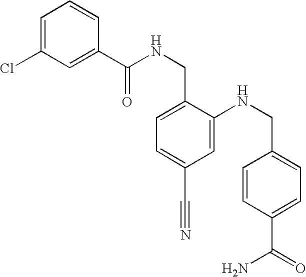

54.2 In analogy to example 39.4, N-(2-{[4-(aminocarbonyl)benzyl]amino}-4-cyanobenzyl)-3-chlorobenzamide was treated with hydroxylamine hydrochloride and triethylamine to give N-{2-{[4-(aminocarbonyl)benzyl]amino}-4-[amino(hydroxyimino)-methyl]benzyl}-3-chlorobenzamide. Colorless solid. MS 452.5 ([M+H]⁺)

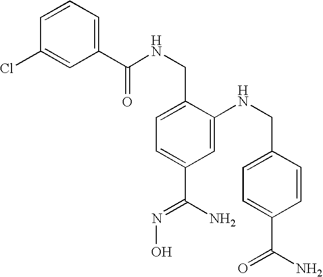

54.3 In analogy to example 52.4, N-{2-{[4-(aminocarbonyl)benzyl]amino}-4-[amino(hydroxyimino)methyl]benzyl}-3-chlorobenzamide was hydrogenated to give N-{2-{[4-(aminocarbonyl)benzyl]amino}-4-[amino(imino) methyl]benzyl}-3-chlorobenzamide acetic acid salt as a light yellow solid. MS 436.1 ([M+H]⁺)

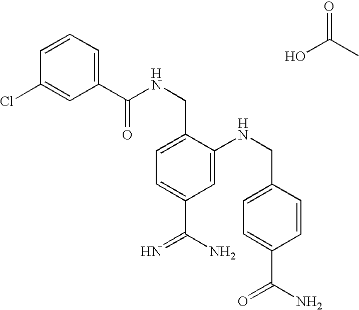

### Example 55

55.1 5-Chloro-isophthalic acid monomethyl ester (CAS 153203-57-7) was coupled with 2-(2-aminomethyl-5-cyano-phenoxy)-N-methyl-acetamide hydrochloride (BB3) according to general procedure A to give 5-chloro-N-(4-cyano-2-methylcarbamoylmethoxy-benzyl)-isophthalamic acid methyl ester. Off-white solid. MS 416.2 ([M+H]⁺)

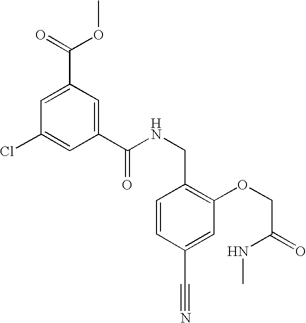

55.2 5-Chloro-N-(4-cyano-2-methylcarbamoylmethoxy-benzyl)-isophthalamic acid methyl ester was converted to N-(4-carbamimidoyl-2-methylcarbamoylmethoxy-benzyl)-5-chloro-isophthalamic acid methyl ester hydrochloride according to general procedure C. Colorless solid. MS 433.0 ([M+H]⁺)

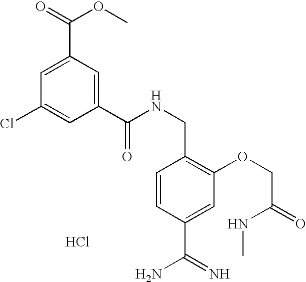

55.3 In analogy to example 8.3, N-(4-carbamimidoyl-2-methylcarbamoylmethoxy-benzyl)-5-chloro-isophthalamic acid methyl ester hydrochloride was hydrolyzed to give N-(4-carbamimidoyl-2-methylcarbamoylmethoxy-benzyl)-5-chloro-isophthalamic acid as a colorless solid. MS 417.3 ([M−H]⁻)

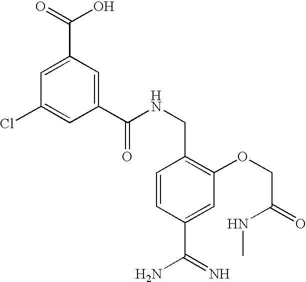

### Example 56

56.1 In analogy to example 8.3, 5-chloro-N-(4-cyano-2-methylcarbamoylmethoxy-benzyl)-isophthalamic acid methyl ester (example 55.1) was hydrolyzed to give 5-chloro-N-(4-cyano-2-methylcarbamoylmethoxy-benzyl)-isophthalamic acid as an off-white solid. MS 400.1 ([M−H]⁻)

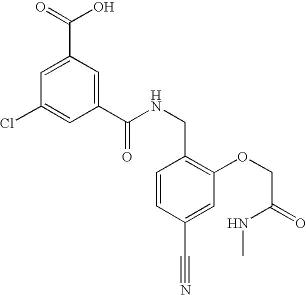

56.2 5-Chloro-N-(4-cyano-2-methylcarbamoylmethoxy-benzyl)-isophthalamic acid was coupled with 2-methoxyethylamine according to general procedure A to give 5-chloro-N-(4-cyano-2-methylcarbamoylmethoxy-benzyl)-N′-(2-methoxy-ethyl)-isophthalamide. Colorless solid. MS 459.4 ([M+H]⁺)

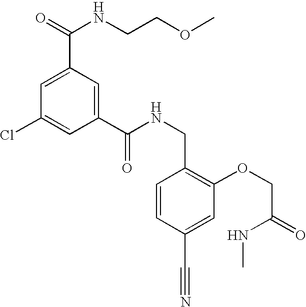

56.3 5-Chloro-N-(4-cyano-2-methylcarbamoylmethoxy-benzyl)-N -(2-methoxy-ethyl)-isophthalamide was converted to N-(4-carbamimidoyl-2-methylcarbamoylmethoxy-benzyl)-5-chloro-N′-(2-methoxy-ethyl)-isophthalamide hydrochloride according to general procedure C. Colorless foam. MS 476.3 ([M+H]⁺)

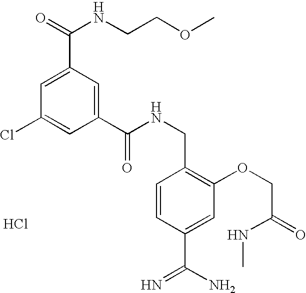

### Example 57

57.1 5-Chloro-N-(4-cyano-2-methylcarbamoylmethoxy-benzyl)-isophthalamic acid (example 56.1) was coupled with morpholine according to general procedure A to give 3-chloro-N-(4-cyano-2-methylcarbamoylmethoxy-benzyl)-5-(morpholine-4-carbonyl)-benzamide. Colorless solid. MS 471.3 ([M+H]⁺)

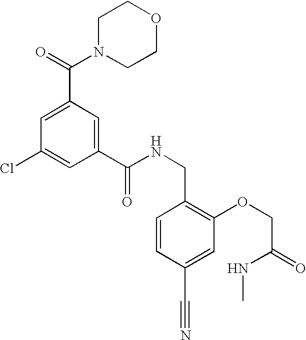

57.2 3-Chloro-N-(4-cyano-2-methylcarbamoylmethoxy-benzyl)-5-(morpholine-4-carbonyl)-benzamide was converted to N-(4-carbamimidoyl-2-methylcarbamoylmethoxy-benzyl)-3-chloro-5-(morpholine-4-carbonyl)-benzamide hydrochloride according to general procedure C. Colorless foam. MS 488.4 ([M+H]⁺)

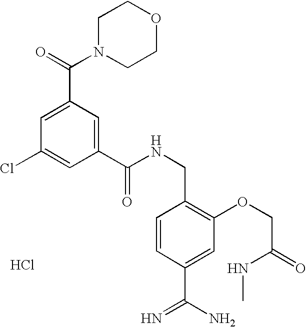

### Example 58

58.1 5-Chloro-N-(4-cyano-2-methylcarbamoylmethoxy-benzyl)-isophthalamic acid (example 56.1) was coupled with ethanolamine according to general procedure A to give 5-chloro-N-(4-cyano-2-methylcarbamoylmethoxy-benzyl)-N′-(2-hydroxy-ethyl)-isophthalamide. Light yellow solid. MS 445.2 ([M+H]⁺)

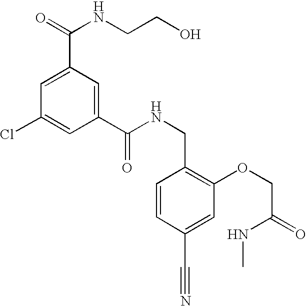

58.2 5-Chloro-N-(4-cyano-2-methylcarbamoylmethoxy-benzyl)-N′-(2-hydroxy-ethyl)-isophthalamide was converted to N-(4-carbamimidoyl-2-methylcarbamoylmethoxy-benzyl)-5-chloro-N′-(2-hydroxy-ethyl)-isophthalamide hydrochloride according to general procedure C. Colorless solid. MS 462.3 ([M+H]⁺)

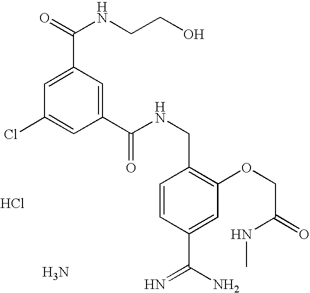

### Example 59

59.1 5-Chloro-2-(methylamino)benzoic acid (CAS 33280-14-7) was coupled with 2-(2-aminomethyl-5-cyano-phenoxy)-N-methyl-acetamide hydrochloride (BB3) according to general procedure A to give 5-chloro-N-(4-cyano-2-methylcarbamoylmethoxy-benzyl)-2-methylamino-benzamide. Off-white solid. MS 387.0 ([M+H]⁺)

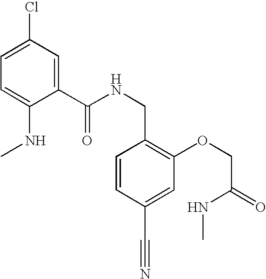

5-Chloro-N-(4-cyano-2-methylcarbamoylmethoxy-benzyl)-2-methylamino-benzamide was converted to N-(4-carbamimidoyl-2-methylcarbamoylmethoxy-benzyl)-5-chloro-2-methylamino-benzamide hydrochloride according to general procedure C. Off-white solid. MS 404.4 ([M+H]⁺)

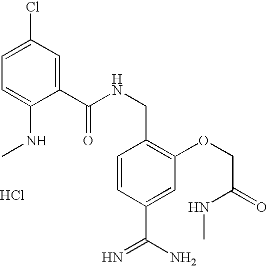

### Example 60

60.1 5-Chloro-N-(2-(4-pyridyl)ethyl)anthranilic acid was coupled with 2-(2-aminomethyl-5-cyano-phenoxy)-N-methyl-acetamide hydrochloride (BB3) according to general procedure A to give 5-chloro-N-(4-cyano-2-methylcarbamoylmethoxy-benzyl)-2-(2-pyridin-4-yl-ethylamino)-benzamide. Colorless solid. MS 478.2 ([M+H]⁺)

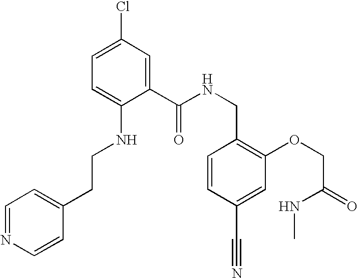

60.2 5-Chloro-N-(4-cyano-2-methylcarbamoylmethoxy-benzyl)-2-(2-pyridin-4-yl-ethylamino)-benzamide was converted to N-(4-carbamimidoyl-2-methylcarbamoylmethoxy-benzyl)-5-chloro-2-(2-pyridin-4-yl-ethylamino)-benzamide hydrochloride according to general procedure C. Light yellow solid. MS 495.5 ([M+H]⁺)

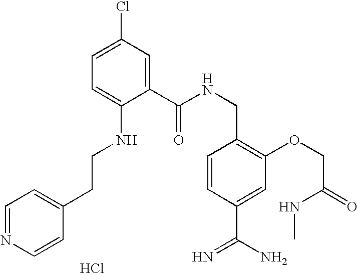

### Example 61

61.1 3-(Boc-Amino)benzoic acid was coupled with 2-(2-aminomethyl-5-cyano-phenoxy)-N-methyl-acetamide hydrochloride (BB3) according to general procedure A to give [3-(4-cyano-2-methylcarbamoylmethoxy-benzylcarbamoyl)-phenyl]-carbamic acid tert-butyl ester. Colorless foam. MS 439.4 ([M+H]⁺)

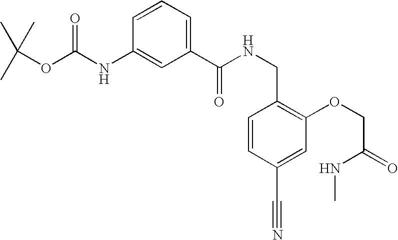

61.2 [3-(4-Cyano-2-methylcarbamoylmethoxy-benzylcarbamoyl)-phenyl]-carbamic acid tert-butyl ester was converted to 3-amino-N-(4-carbamimidoyl-2-methyl-carbamoylmethoxy-benzyl)-benzamide hydrochloride according to general procedure C. Colorless solid. MS 356.2 ([M+H]⁺)

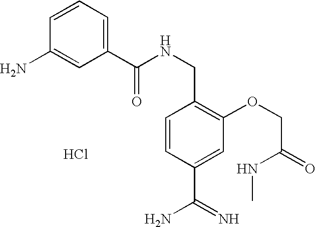

### Example 62

62.1 3-Hydroxy-5-methyl-benzoic acid (CAS 585-81-9) was coupled with 2-(2-aminomethyl-5-cyano-phenoxy)-N-methyl-acetamide hydrochloride (BB3) according to general procedure A to give N-(4-cyano-2-methylcarbamoylmethoxy-benzyl)-3-hydroxy-5-methyl-benzamide. Colorless solid. MS 354.2 ([M+H]⁺)

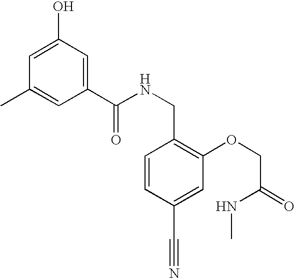

62.2 N-(4-Cyano-2-methylcarbamoylmethoxy-benzyl)-3-hydroxy-5-methyl-benzamide was converted to N-(4-carbamimidoyl-2-methylcarbamoylmethoxy-benzyl)-3-hydroxy-5-methyl-benzamide hydrochloride according to general procedure C. Colorless solid. MS 371.2 ([M+H]⁺)

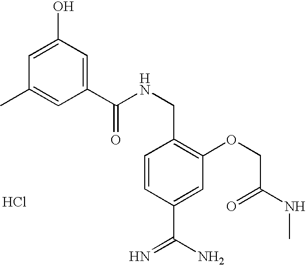

### Example 63

63.1 In analogy to example 3.2, N-(4-cyano-2-methylcarbamoylmethoxy-benzyl)-3-hydroxy-5-methyl-benzamide (example 62.1) was alkylated with ethylchloroacetate and cesium carbonate in dimethylacetamide to give [3-(4-cyano-2-methylcarbamoylmethoxy-benzylcarbamoyl)-5-methyl-phenoxy]-acetic acid ethyl ester. Colorless solid. MS 440.3 ([M+H]⁺)

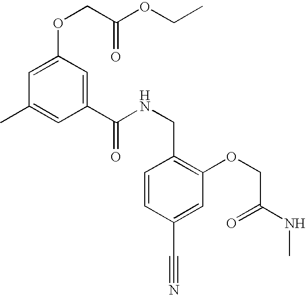

63.2 [3-(4-Cyano-2-methylcarbamoylmethoxy-benzylcarbamoyl)-5-methyl-phenoxy]-acetic acid ethyl ester was converted to [3-(4-carbamimidoyl-2-methylcarbamoylmethoxy-benzylcarbamoyl)-5-methyl-phenoxy]-acetic acid ethyl ester hydrochloride according to general procedure C. Colorless solid. MS 457.5 ([M+H]⁺)

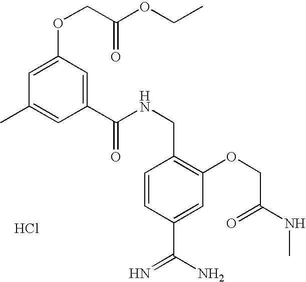

63.3 In analogy to example 8.3, [3-(4-carbamimidoyl-2-methylcarbamoylmethoxy-benzylcarbamoyl-5-methyl-phenoxy]-acetic acid ethyl ester hydrochloride was hydrolyzed to give [3-(4-carbamimidoyl-2-methylcarbamoylmethoxy-benzylcarbamoyl)-5-methyl-phenoxy]-acetic acid as a colorless solid. MS 429.4 ([M+H]⁺)

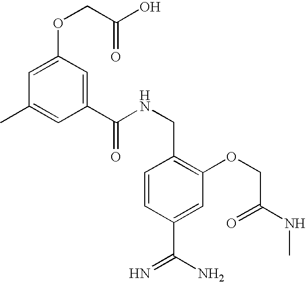

### Example 64

64.1 3-Chloro-5-methoxy-benzoic acid (CAS 82477-67-6) was coupled with 4-aminomethyl-3-nitro-benzonitrile (BB4) according to general procedure A to give 3-chloro-N-(4-cyano-2-nitro-benzyl)-5-methoxy-benzamide. Light yellow solid.

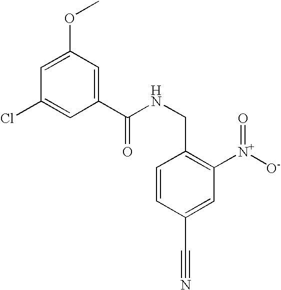

64.2 The nitro group of 3-chloro-N-(4-cyano-2-nitro-benzyl)-5-methoxy-benzamide was reduced according to general procedure B to give N-(2-amino-4-cyano-benzyl)-3-chloro-5-methoxy-benzamide. Light yellow solid.

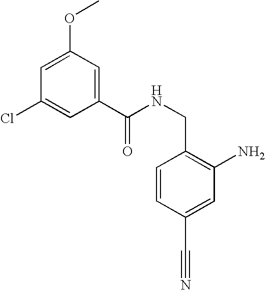

64.3 In analogy to example 22.1, N-(2-amino-4-cyano-benzyl)-3-chloro-5-methoxy-benzamide was alkylated with bromoethanol to give 3-chloro-N-[4-cyano-2-(2-hydroxy-ethylamino)-benzyl]-5-methoxy-benzamide as a light yellow solid.

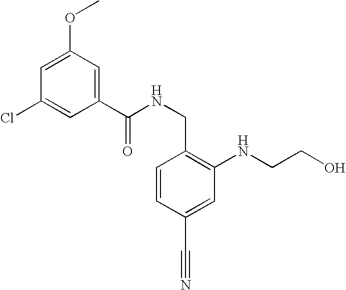

64.4 3-Chloro-N-[4-cyano-2-(2-hydroxy-ethylamino)-benzyl]-5-methoxy-benzamide was converted to N-[4-carbamimidoyl-2-(2-hydroxy-ethylamino)-benzyl]-3-chloro-5-methoxy-benzamide hydrochloride according to general procedure C. Off-white solid. MS 377.3 ([M+H]⁺)

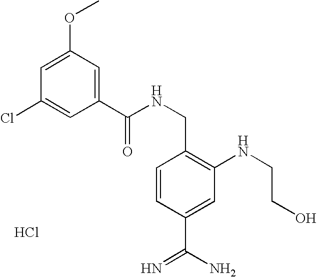

### Example 65

65.1 As a side-product of example 64.3, there was obtained N-{2-[bis-(2-hydroxy-ethyl)-amino]-4-cyano-benzyl}-3-chloro-5-methoxy-benzamide. Light yellow foam. MS 404.4 ([M+H]⁺)

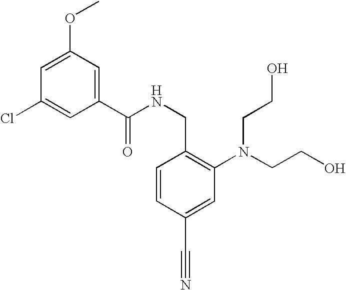

65.2 N-{2-[Bis-(2-hydroxy-ethyl)-amino]-4-cyano-benzyl}-3-chloro-5-methoxy-benzamide was converted to N-{2-[bis-(2-hydroxy-ethyl)-amino]-4-carbamimidoyl-benzyl}-3-chloro-5-methoxy-benzamide hydrochloride according to general procedure C. Light yellow solid. MS 421.1 ([M+H]⁺)

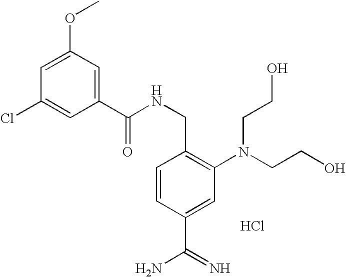

### Example 66

66.1 3-Chloro-5-hydroxy-benzoic acid (CAS 53984-36-4) was coupled with 2-(2-aminomethyl-5-cyano-phenoxy)-N-methyl-acetamide hydrochloride (BB3) according to general procedure A to give 3-chloro-N-(4-cyano-2-methylcarbamoylmethoxy-benzyl)-5-hydroxy-benzamide. Light yellow solid.

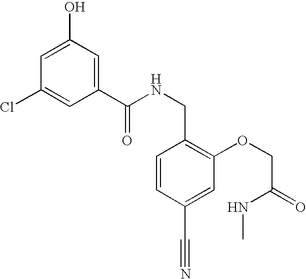

66.2 3-Chloro-N-(4-cyano-2-methylcarbamoylmethoxy-benzyl)-5-hydroxy-benzamide was converted to N-(4-carbamimidoyl-2-methylcarbamoylmethoxy-benzyl)-3-chloro-5-hydroxy-benzamide hydrochloride according to general procedure C. Colorless solid. MS 391.2 ([M+H]⁺)

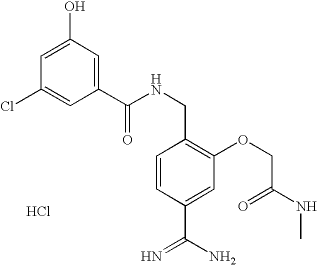

### Example 67

67.1 3-Chloro-5-nitro-benzoic acid (CAS 34662-36-7) was coupled with 4-aminomethyl-3-hydroxy-benzonitrile hydrochloride (BB1) according to general procedure A to give 3-chloro-N-(4-cyano-2-hydroxy-benzyl)-5-nitro-benzamide. Light brown solid.

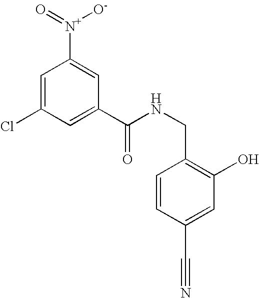

67.2 In analogy to example 3.2, 3-chloro-N-(4-cyano-2-hydroxy-benzyl)-5-nitro-benzamide was alkylated with iodoacetamide and cesium carbonate in acetonitrile. The product of this reaction was converted to (4-carbamimidoyl-2-carbamoylmethoxy-benzyl)-3-chloro-5-nitro-benzamide acetic acid salt according to general procedure C. Light brown solid. MS 406.4 ([M+H]⁺)

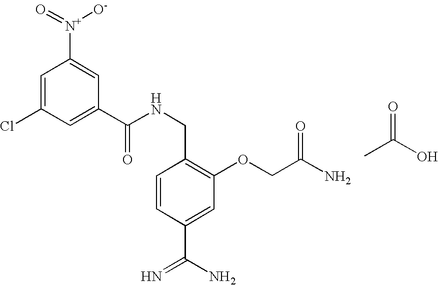

### Example 68

68.1 In analogy to example 3.2, 3-chloro-N-(4-cyano-2-hydroxy-benzyl)-5-nitro-benzamide (example 67.1) was alkylated with 2-chloro-N-methylacetamide and cesium carbonate in acetonitrile. The product of this reaction was converted to (4-carbamimidoyl-2-methylcarbamoylmethoxy-benzyl)-3-chloro-5-nitro-benzamide acetic acid salt according to general procedure C. Off-white solid. MS 420.3 ([M+H]⁺)

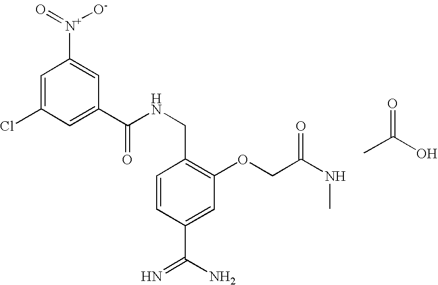

### Example 69

69.1 3-Chloro-5-fluoro-benzoic acid was coupled with 2-(2-aminomethyl-5-cyano-phenoxy)-N-methyl-acetamide hydrochloride (BB3) according to general procedure A to give 3-chloro-N-(4-cyano-2-methylcarbamoylmethoxy-benzyl)-5-fluoro-benzamide. Colorless solid. MS 376.3 ([M+H]⁺)

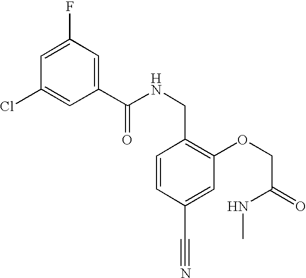

69.2 3-Chloro-N-(4-cyano-2-methylcarbamoylmethoxy-benzyl)-5-fluoro-benzamide was converted to N-(4-carbamimidoyl-2-methylcarbamoylmethoxy-benzyl)-3-chloro-5-fluoro-benzamide hydrochloride according to general procedure C. Colorless solid. MS 393.2 ([M+H]⁺)

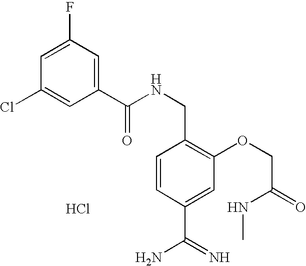

### Example 70

70.1 3-Chloro-2-fluoro-benzoic acid was coupled with 2-(2-aminomethyl-5-cyano-phenoxy)-N-methyl-acetamide hydrochloride (BB3) according to general procedure A to give 3-chloro-N-(4-cyano-2-methylcarbamoylmethoxy-benzyl)-2-fluoro-benzamide. Colorless solid. MS 376.3 ([M+H]⁺)

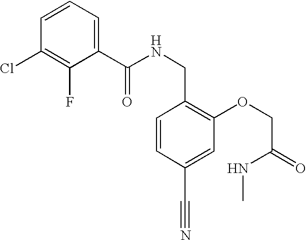

70.2 3-Chloro-N-(4-cyano-2-methylcarbamoylmethoxy-benzyl)-2-fluoro-benzamide was converted to N-(4-carbamimidoyl-2-methylcarbamoylmethoxy-benzyl)-3-chloro-2-fluoro-benzamide hydrochloride according to general procedure C. Colorless solid. MS 393.2 ([M+H]⁺)

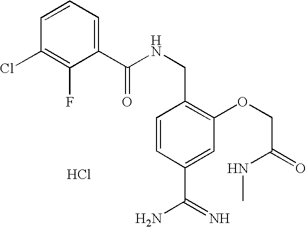

### Example 71

71.1 In analogy to example 22.1, N-(2-amino-4-cyano-benzyl)-3-chloro-benzamide (example 49.2) was alkylated with 6-bromomethyl-nicotinamide to give 6-({2-[(3-chloro-benzoylamino)-methyl]-5-cyano-phenylamino}-methyl)-nicotinamide as a light yellow solid. MS 420.2 ([M+H]⁺)

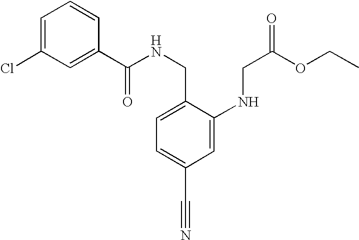

71.2 6-({2-[(3-Chloro-benzoylamino)-methyl]-5-cyano-phenylamino}-methyl)-nicotinamide was converted to 6-({5-carbamimidoyl-2-[(3-chloro-benzoylamino)-methyl]-phenylamino}-methyl)-nicotinamide hydrochloride which contained 5 equivalents of ammoniumchloride according to general procedure C. Light yellow solid. MS 437.3 ([M+H]⁺)

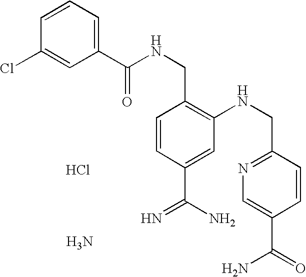

### Example 72

72.1 In analogy to example 22.1, N-(2-amino-4-cyano-benzyl)-3-chloro-benzamide (example 49.2) was alkylated with ethyl bromoacetate to give {2-[(3-chloro-benzoylamino)-methyl]-5-cyano-phenylamino}-acetic acid ethyl ester as a light yellow solid. MS 372.1 ([M+H]⁺)

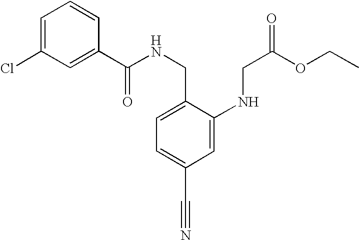

72.2 In analogy to example 8.3, {2-[(3-chloro-benzoylamino)-methyl]-5-cyano-phenylamino}-acetic acid ethyl ester was hydrolyzed to give {2-[(3-chloro-benzoylamino)-methyl]-5-cyano-phenylamino}-acetic acid as a colorless solid. MS 342.1 ([M−H]⁻)

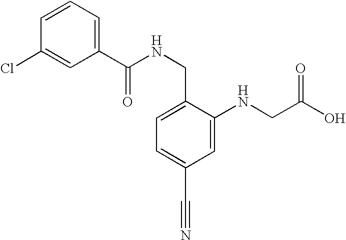

72.3 {2-[(3-Chloro-benzoylamino)-methyl]-5-cyano-phenylamino}-acetic acid was coupled to 2-aminopyridine according to general procedure A to give 3-chloro-N-{4-cyano-2-[(pyridin-2-ylcarbamoylmethyl)-amino]-benzyl}-benzamide as a colorless solid. MS 420.3 ([M+H]⁺)

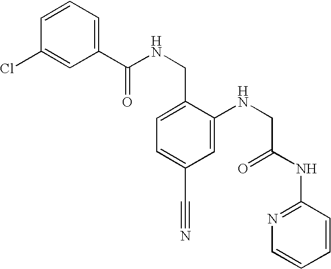

72.4 3-Chloro-N-{4-cyano-2-[(pyridin-2-ylcarbamoylmethyl)-amino]-benzyl}benzamide was converted to N-{4-carbamimidoyl-2-[(pyridin-2-ylcarbamoylmethyl)-amino]-benzyl}-3-chloro-benzamide hydrochloride according to general procedure C. Light brown solid. MS 437.3 ([M+H]⁺)

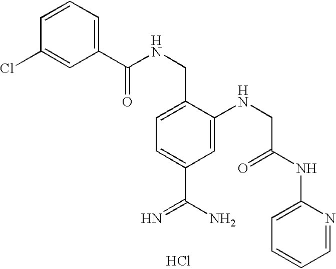

### Example 73

73.1 {2-[(3-Chloro-benzoylamino)-methyl]-5-cyano-phenylamino}-acetic acid (example 72.2) was coupled to 3-aminopyridine according to general procedure A to give 3-chloro-N-{4-cyano-2-[(pyridin-3-ylcarbamoylmethyl)-amino]-benzyl}-benzamide as a colorless solid. MS 420.3 ([M+H]⁺)

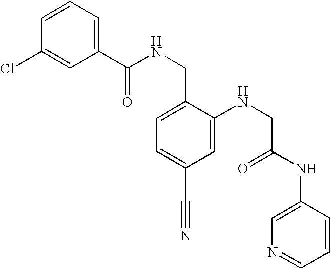

73.2 3-Chloro-N-{4-cyano-2-[(pyridin-3-ylcarbamoylmethyl)-amino]-benzyl}-benzamide was converted to N-{4-carbamimidoyl-2-[(pyridin-3-ylcarbamoylmethyl)-amino]-benzyl}-3-chloro-benzamide hydrochloride according to general procedure C. Light yellow solid. MS 437.3 ([M+H]⁺)

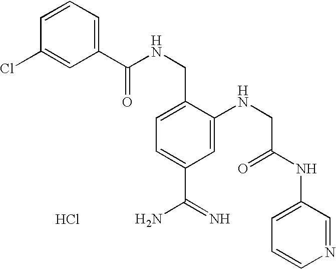

### Example 74

74.1 {2-[(3-Chloro-benzoylamino)-methyl]-5-cyano-phenylamino}-acetic acid (example 72.2) was coupled to 3-aminoisoxazole according to general procedure A to give 3-chloro-N-{4-cyano-2-[(isoxazol-3-ylcarbamoylmethyl)-amino]-benzyl}-benzamide as a yellow foam. MS 410.3 ([M+H]⁺)

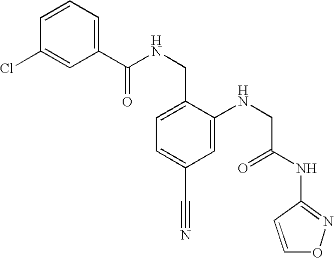

74.2 3-Chloro-N-{4-cyano-2-[(isoxazol-3-ylcarbamoylmethyl)-amino]-benzyl}-benzamide was converted to N-{4-carbamimidoyl-2-[(isoxazol-3-ylcarbamoylmethyl)-amino]-benzyl}-3-chloro-benzamide hydrochloride according to general procedure C. Colorless solid. MS 427.5 ([M+H]⁺)

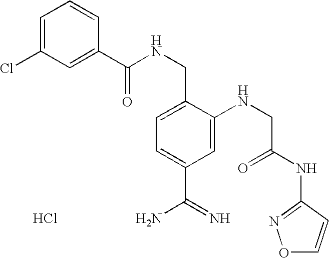

### Example 75

75.1 {2-[(3-Chloro-benzoylamino)-methyl]-5-cyano-phenylamino}-acetic acid (example 72.2) was coupled to 2-(pyridin-2-ylamino)-ethanol hydrochloride (CAS 117043-32-0) according to general procedure A to give 3-chloro-N-[4-cyano-2-({[(2-hydroxy-ethyl)-pyridin-2-yl-carbamoyl]-methyl}-amino)-benzyl]-benzamide as a light brown foam. MS 464.4 ([M+H]⁺)

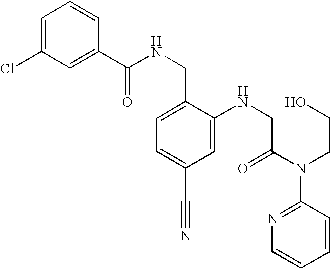

75.2 3-Chloro-N-[4-cyano-2-({[(2-hydroxy-ethyl)-pyridin-2-yl-carbamoyl]-methyl}-amino)-benzyl]-benzamide was converted to N-[4-carbamimidoyl-2-({[(2-hydroxy-ethyl)-pyridin-2-yl-carbamoyl]-methyl}-amino)-benzyl]-3-chloro-benzamide hydrochloride according to general procedure C. Colorless solid. MS 481.4 ([M+H]⁺)

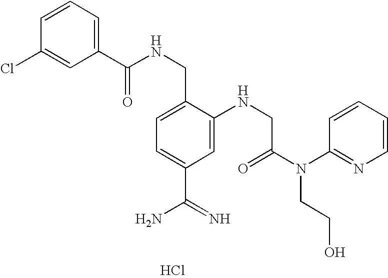

### Example 76

76.1 {2-[(3-Chloro-benzoylamino)-methyl]-5-cyano-phenylamino}-acetic acid (example 72.2) was coupled to N-(2-hydroxyethyl)-aniline according to general procedure A to give 3-chloro-N-[4-cyano-2-({[(2-hydroxy-ethyl)-phenyl-carbamoyl]-methyl}-amino)-benzyl]-benzamide as a light brown solid. MS 463.4 ([M+H]⁺)

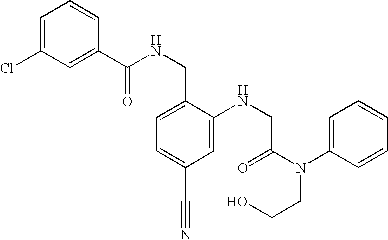

76.2 3-Chloro-N-[4-cyano-2-({[(2-hydroxy-ethyl)-phenyl-carbamoyl]-methyl}-amino)-benzyl]-benzamide was converted to N-[4-carbamimidoyl-2-({[(2-hydroxy-ethyl)-phenyl-carbamoyl]-methyl}-amino)-benzyl]-3-chloro-benzamide hydrochloride according to general procedure C. Colorless solid. MS 480.5 ([M+H]⁺)

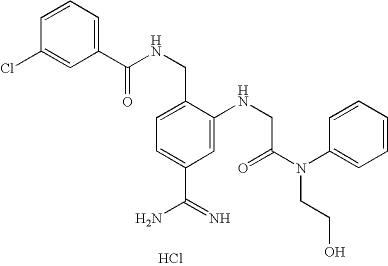

### Example 77

77.1 {2-[(3-Chloro-benzoylamino)-methyl]-5-cyano-phenylamino}-acetic acid (example 72.2) was coupled to 4-amino-N-methylmorpholine according to general procedure A to give 3-chloro-N-(4-cyano-2-{[(1-methyl-piperidin-4-ylcarbamoyl)-methyl]-amino}-benzyl)-benzamide as a yellow foam. MS 440.4 ([M+H]⁺)

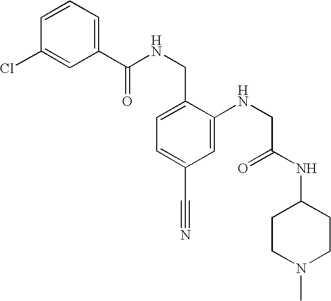

77.2 3-Chloro-N-(4-cyano-2-{[(1-methyl-piperidin-4-ylcarbamoyl)-methyl]-amino}-benzyl)-benzamide was converted to N-(4-carbamimidoyl-2-{[(1-methyl-piperidin-4-ylcarbamoyl)-methyl]-amino}-benzyl)-3-chloro-benzamide acetic acid salt according to general procedure C. Light yellow solid. MS 457.2 ([M+H]⁺)

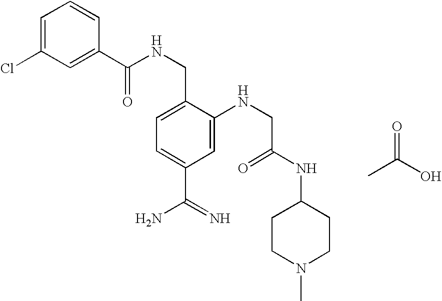

### Example 78

78.1 3-Chloro-2-fluoro-5-methoxy-benzoic acid was coupled with 2-(2-aminomethyl-5-cyano-phenoxy)-N-methyl-acetamide hydrochloride (BB3) according to general procedure A to give 3-chloro-N-(4-cyano-2-methylcarbamoylmethoxy-benzyl)-2-fluoro-5-methoxy-benzamide. Colorless solid. MS 406.3 ([M+H]⁺)

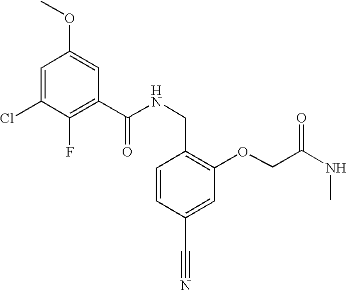

78.2 3-Chloro-N-(4-cyano-2-methylcarbamoylmethoxy-benzyl)-2-fluoro-5-methoxy-benzamide was converted to N-(4-carbamimidoyl-2-methylcarbamoylmethoxy-benzyl)-3-chloro-2-fluoro-5-methoxy-benzamide hydrochloride according to general procedure C. Colorless solid. MS 423.3 ([M+H]⁺)

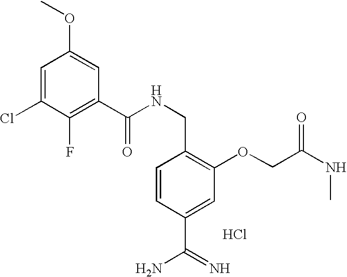

### Example 79

79.1 3-Chloro-2,4-difluorobenzoic acid was coupled with 2-(2-aminomethyl-5-cyano-phenoxy)-N-methyl-acetamide hydrochloride (BB3) according to general procedure A to give 3-chloro-N-(4-cyano-2-methylcarbamoylmethoxy-benzyl)-2,4-difluoro-benzamide. Colorless solid. MS 394.0 ([M+H]⁺)

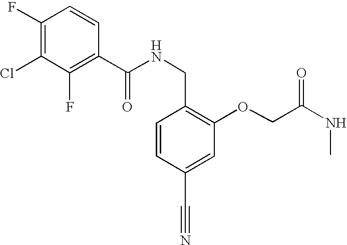

79.2 3-Chloro-N-(4-cyano-2-methylcarbamoylmethoxy-benzyl)-2,4-difluoro-benzamide was converted to N-(4-carbamimidoyl-2-methylcarbamoylmethoxy-benzyl)-3-chloro-2,4-difluoro-benzamide hydrochloride according to general procedure C. Colorless solid. MS 411.0 ([M+H]⁺)

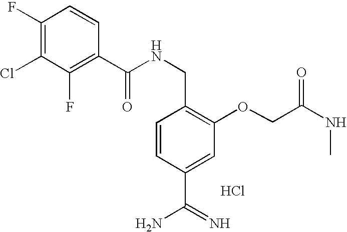

### Example 80

80.1 3-Chloro-5-hydroxy-benzoic acid was coupled with 4-aminomethyl-3-hydroxy-benzonitrile hydrochloride (BB1) according to general procedure A to give 3-chloro-N-(4-cyano-2-hydroxy-benzyl)-5-hydroxy-benzamide. Colorless solid. MS 303.0 ([M+H]⁺)

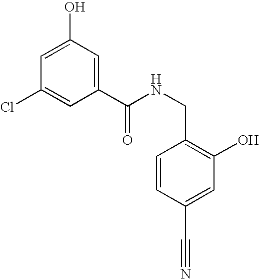

80.2 In analogy to example 3.2, 3-chloro-N-(4-cyano-2-hydroxy-benzyl)-5-hydroxy-benzamide was alkylated with iodoacetamide to give 3-carbamoylmethoxy-N-(2-carbamoylmethoxy-4-cyano-benzyl)-5-chloro-benzamide as a colorless solid. MS 417.4 ([M+H]⁺)

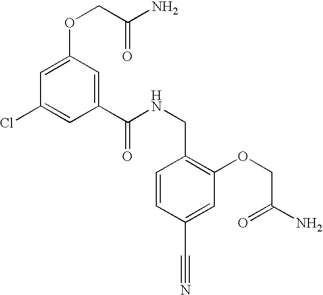

80.3 3-Carbamoylmethoxy-N-(2-carbamoylmethoxy-4-cyano-benzyl)-5-chloro-benzamide was converted to N-(4-carbamimidoyl-2-carbamoylmethoxy-benzyl)-3-carbamoylmethoxy-5-chloro-benzamide hydrochloride according to general procedure C. Colorless solid. MS 434.3 ([M+H]⁺)

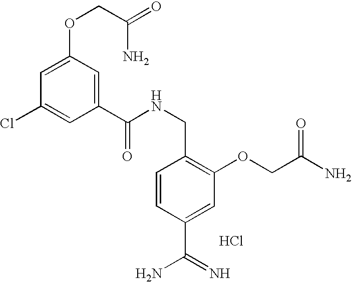

### Example 81

81.1 In analogy to example 3.2, 3-chloro-N-(4-cyano-2-hydroxy-benzyl)-5-hydroxy-benzamide (example 80.1) was alkylated with 2-chloro-N-methylacetamide to give 3-chloro-N-(4-cyano-2-methylcarbamoylmethoxy-benzyl)-5-methylcarbamoylmethoxy-benzamide as a colorless solid. MS 445.4 ([M+H]⁺)

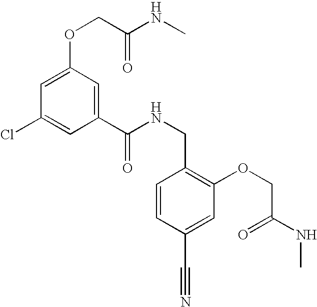

81.2 3-Chloro-N-(4-cyano-2-methylcarbamoylmethoxy-benzyl)-5-methylcarbamoylmethoxy-benzamide was converted to N-(4-carbamimidoyl-2-methylcarbamoylmethoxy-benzyl)-3-chloro-5-methylcarbamoylmethoxy-benzamide hydrochloride according to general procedure C. Colorless solid. MS 462.4 ([M+H]⁺)

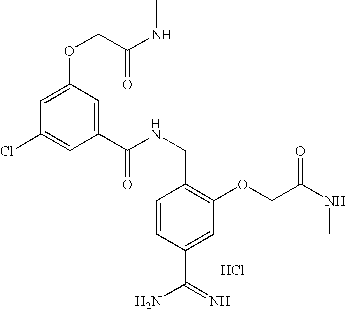

### Example 82

82.1 In analogy to example 40.1, 3-chloro-N-(4-cyano-2-hydroxy-benzyl)-5-hydroxy-benzamide (example 80.1) was reacted with 2-(hydroxymethyl)pyridine, triphenylphosphine and diethylazodicarboxylate to give 3-chloro-N-[4-cyano-2-(pyridin-2-ylmethoxy)-benzyl]-5-(pyridin-2-ylmethoxy)-benzamide as a colorless solid. MS 483.3 ([M+H]⁺)

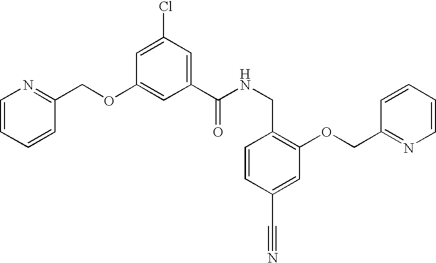

82.2 3-Chloro-N-[4-cyano-2-(pyridin-2-ylmethoxy)-benzyl]-5-(pyridin-2-ylmethoxy)-benzamide was converted to N-[4-carbamimidoyl-2-(pyridin-2-ylmethoxy)-benzyl]-3-chloro-5-(pyridin-2-ylmethoxy)-benzamide hydrochloride according to general procedure C. Colorless solid. MS 502.3 ([M+H]⁺)

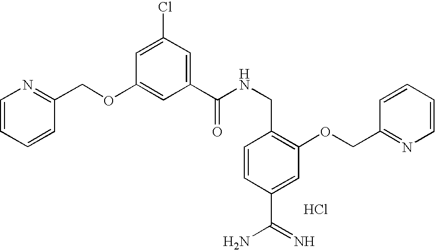

### Example 83

83.1 In analogy to example 3.2, N-(4-cyano-2-methylcarbamoylmethoxy-benzyl)-3-hydroxy-5-methyl-benzamide (example 62.1) was alkylated with bromoethanol to give N-(4-cyano-2-methylcarbamoylmethoxy-benzyl)-3-(2-hydroxy-ethoxy)-5-methyl-benzamide as a colorless foam. MS 398.5 ([M+H]⁺)

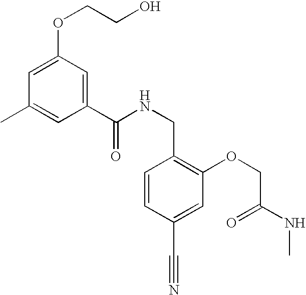

83.2 N-(4-Cyano-2-methylcarbamoylmethoxy-benzyl)-3-(2-hydroxy-ethoxy)-5-methyl-benzamide was converted to N-(4-carbamimidoyl-2-methylcarbamoylmethoxy-benzyl)-3-(2-hydroxy-ethoxy)-5-methyl-benzamide hydrochloride according to general procedure C. Colorless solid. MS 415.4 ([M+H]⁺)

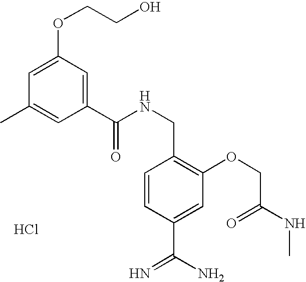

### Example 84

84.1 In analogy to example 3.2, N-(4-cyano-2-methylcarbamoylmethoxy-benzyl)-3-hydroxy-5-methyl-benzamide (example 62.1) was alkylated with iodoacetamide to give 3-carbamoylmethoxy-N-(4-cyano-2-methylcarbamoylmethoxy-benzyl)-5-methyl-benzamide as a colorless solid. MS 411.2 ([M+H]⁺)

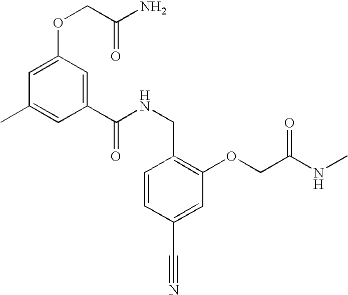

84.2 3-Carbamoylmethoxy-N-(4-cyano-2-methylcarbamoylmethoxy-benzyl)-5-methyl-benzamide was converted to N-(4-carbamimidoyl-2-methylcarbamoylmethoxy-benzyl)-3-carbamoylmethoxy-5-methyl-benzamide acetic acid salt according to general procedure C. Off-white solid. MS 428.5 ([M+H]⁺)

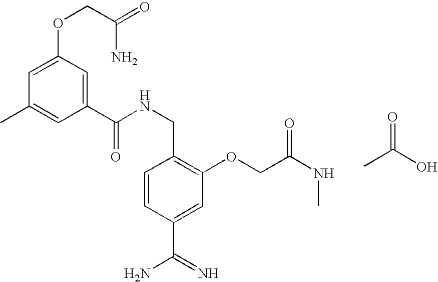

### Example 85

85.1 To a solution of N-[2-(2-Amino-ethoxy)-4-cyano-benzyl]-4-fluoro-3-methyl-benzamide (200 mg, example 40.2) in dioxane (3 ml) were added phthalic acid anhydride (181 mg), triethylamine (56 mg) and 4-dimethylamino pyridine (8 mg). The mixture was stirred at r.t. for 4 days and at 110° C. for 7 days. 4-Dimethylamino pyridine (16 mg), triethylamine (112 mg) and dioxane (6 ml) were added and the mixture was stirred for 13 days at 110° C. The solvent was evaporated. The residue was dissolved in EtOAc and washed with 10% aq. KHSO₄ solution, sat. aq. NaHCO₃ solution and with brine. The combined aqueous phases were extracted with EtOAc. The combined org. phases were dried (MgSO₄), filtered and concentrated to give N-{4-cyano-2-[2-(1,3-dioxo-1,3-dihydro-isoindol-2-yl)-ethoxy]-benzyl}-4-fluoro-3-methyl-benzamide (170 mg) as a colorless solid. MS 458.4 ([M+H]⁺)

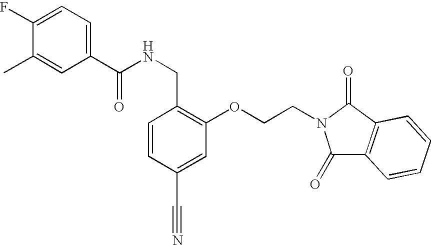

85.2 Dry HCl gas was passed over a cooled (−10° C.), stirred solution of N-{4-cyano-2-[2-(1,3-dioxo-1,3-dihydro-isoindol-2-yl)-ethoxy]-benzyl}-4-fluoro-3-methyl-benzamide (170 mg) in CHCl₃ (4.2 ml) and MeOH (4.2 ml) for 20 min. The flask was stoppered and left at 4° C. overnight. The mixture was concentrated (rotavapor and high vacuum) at r.t. The residue was dissolved in CHCl₃ and rapidly washed with a 5% aq. NaHCO₃ solution. The org. phase was dried immediately, filtered and concentrated. The residue was dissolved in MeOH (1.8 ml). A solution of 28 mg ammonium chloride in 0.28 ml water was added and the mixture was stirred at 65° C. for 3 h. A solution of 28 mg ammonium chloride in 0.28 ml water was added and the mixture was stirred at 65° C. for 4.5 h. 60 mg ammonium chloride and 1 ml MeOH were added and the mixture was stirred at r.t. for 60 h. The solvent was evaporated and the product was purified by chromatography (SiO₂, CH₂Cl₂/MeOH 1:0=>4:1) to give N-{4-carbamimidoyl-2-[2-(1,3-dioxo-1,3-dihydro-isoindol-2-yl)-ethoxy]-benzyl}-4-fluoro-3-methyl-benzamide hydrochloride (26 mg) as a colorless solid. MS 475.2 ([M+H]⁺)

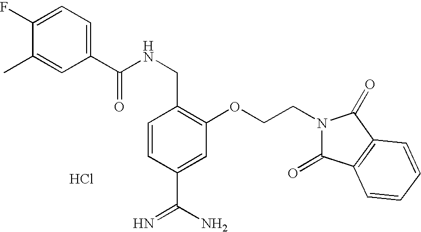

### Example 86

86.1 A solution of 3-chloro-N-[4-cyano-2-(2-hydroxy-ethylamino)-benzyl]-5-methoxy-benzamide (100 mg, example 64.3) in CH₂Cl₂ (5 ml) was cooled to −78° C. A 1 M solution of boron tribromide in CH₂Cl₂ (1.4 ml) was added dropwise. The cooling bath was removed. After reaching r.t., ice was added and the mixture was extracted with EtOAc. The org. phase was washed with water, dried, filtered and concentrated. The product was purified by chromatography (SiO₂, CH₂Cl₂/MeOH 98:2=>9:2) to give 3-chloro-N-[4-cyano-2-(2-hydroxy-ethylamino)-benzyl]-5-hydroxy-benzamide (19 mg) as a grey foam. MS 346.3 ([M+H]⁺)

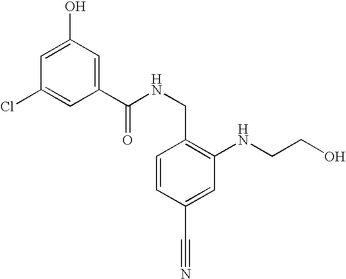

86.2 3-Chloro-N-[4-cyano-2-(2-hydroxy-ethylamino)-benzyl]-5-hydroxy-benzamide was converted to N-[4-carbamimidoyl-2-(2-hydroxy-ethylamino)-benzyl]-3-chloro-5-hydroxy-benzamide hydrochloride according to general procedure C. Brown solid. MS 363.4 ([M+H]⁺)

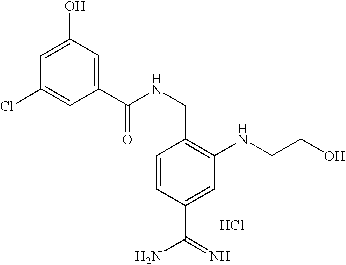

### Example 87

87.1 In analogy to example 22.1, N-(2-amino-4-cyano-benzyl)-3-chloro-5-methoxy-benzamide (example 64.2) was alkylated with ethyl bromoacetate to give {2-[(3-chloro-5-methoxy-benzoylamino)-methyl]-5-cyano-phenylamino}-acetic acid ethyl ester as a colorless solid.

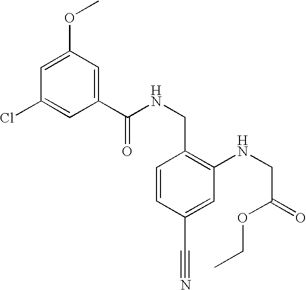

87.2 {2-[(3-Chloro-5-methoxy-benzoylamino)-methyl]-5-cyano-phenylamino}-acetic acid ethyl ester was converted to {5-carbamimidoyl-2-[(3-chloro-5-methoxy-benzoylamino)-methyl]-phenylamino}-acetic acid ethyl ester hydrochloride according to general procedure C. Colorless solid. MS 419.2 ([M+H]⁺)

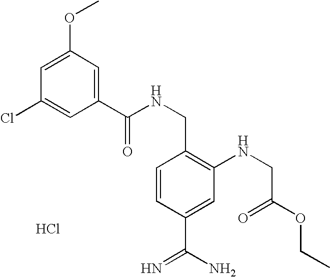

87.3 In analogy to example 8.3, {5-carbamimidoyl-2-[(3-chloro-5-methoxy-benzoylamino)-methyl]-phenylamino}-acetic acid ethyl ester hydrochloride was hydrolyzed to give {5-carbamimidoyl-2-[(3-chloro-5-methoxy-benzoylamino)-methyl]-phenylamino}-acetic acid as a colorless solid. MS 391.1 ([M+H]⁺)

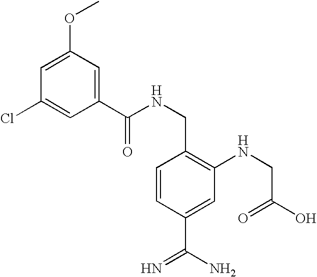

### Example 88

88.1 In analogy to example 40.1, 3-chloro-N-(4-cyano-2-methylcarbamoylmethoxy-benzyl)-5-hydroxy-benzamide (example 66.1) was reacted with 4-hydroxymethyl-pyridine, triphenylphosphine and diethylazodicarboxylate to give 3-chloro-N-(4-cyano-2-methylcarbamoylmethoxy-benzyl)-5-(pyridin-4-ylmethoxy)-benzamide as a colorless solid. MS 463.1 ([M−H]⁻)

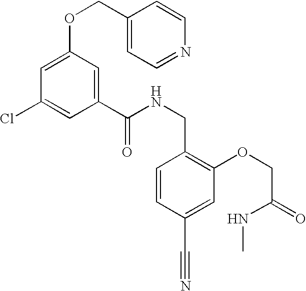

88.2 3-Chloro-N-(4-cyano-2-methylcarbamoylmethoxy-benzyl)-5-(pyridin-4-ylmethoxy)-benzamide was converted to N-(4-carbamimidoyl-2-methylcarbamoylmethoxy-benzyl)-3-chloro-5-(pyridin-4-ylmethoxy)-benzamide hydrochloride according to general procedure C. Colorless solid. MS 482.5 ([M+H]⁺)

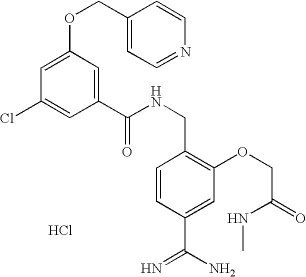

### Example 89

89.1 In analogy to example 40.1, 3-chloro-5-hydroxy-benzoic acid methyl ester (CAS 98406-04-3) was reacted with 4-hydroxymethyl-pyridine, triphenylphosphine and diethylazodicarboxylate. The product of this reaction was hydrolysed in analogy to example 8.3 to give 3-chloro-5-(pyridin-4-ylmethoxy)-benzoic acid as a colorless solid. MS 262.1 ([M−H]⁻)

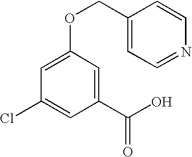

89.2 3-Chloro-5-(pyridin-4-ylmethoxy)-benzoic acid was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-(pyridin-4-ylmethoxy)-benzamide. Light brown solid. MS 451.3 ([M+H]⁺)

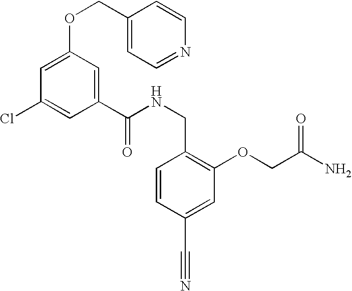

89.3 N-(2-Carbamoylmethoxy-4-cyano-benzyl)-3-chloro-5-(pyridin-4-ylmethoxy)-benzamide was converted to N-(4-carbamimidoyl-2-carbamoylmethoxy-benzyl)-3-chloro-5-(pyridin-4-ylmethoxy)-benzamide hydrochloride according to general procedure C. Colorless solid. MS 468.5 ([M+H]⁺)

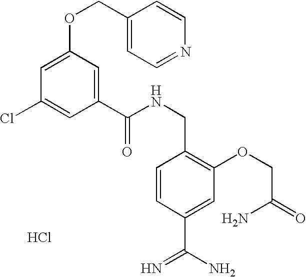

### Example 90

90.1 In analogy to example 40.1, 3-chloro-5-hydroxy-benzoic acid methyl ester (CAS 98406-04-3) was reacted with 3-hydroxymethyl-pyridine, triphenylphosphine and diethylazodicarboxylate. The product of this reaction was hydrolysed in analogy to example 8.3 to give 3-chloro-5-(pyridin-3-ylmethoxy)-benzoic acid as a colorless solid. MS 262.1 ([M−H]⁻)

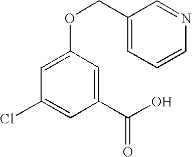

90.2 3-Chloro-5-(pyridin-3-ylmethoxy)-benzoic acid was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-(pyridin-3-ylmethoxy)-benzamide. Light yellow solid. MS 451.3 ([M+H]⁺)

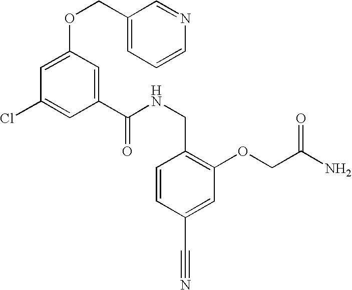

90.3 N-(2-Carbamoylmethoxy-4-cyano-benzyl)-3-chloro-5-(pyridin-3-ylmethoxy)-benzamide was converted to N-(4-carbamimidoyl-2-carbamoylmethoxy-benzyl)-3-chloro-5-(pyridin-3-ylmethoxy)-benzamide hydrochloride according to general procedure C. Colorless solid. MS 468.4 ([M+H]⁺)

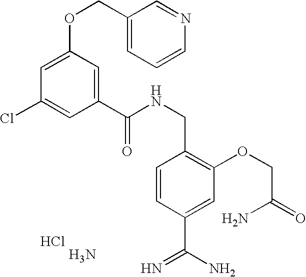

### Example 91

91.1 In analogy to example 40.1, 3-chloro-5-hydroxy-benzoic acid methyl ester (CAS 98406-04-3) was reacted with 2-hydroxymethyl-pyridine, triphenylphosphine and diethylazodicarboxylate. The product of this reaction was hydrolysed in analogy to example 8.3. The product of this reaction was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-(pyridin-2-ylmethoxy)-benzamide. Light yellow solid. MS 451.3 ([M+H]⁺)

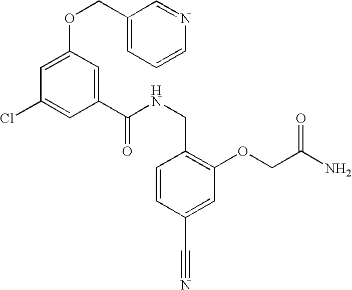

91.2 N-(2-Carbamoylmethoxy-4-cyano-benzyl)-3-chloro-5-(pyridin-2-ylmethoxy)-benzamide was converted to N-(4-carbamimidoyl-2-carbamoylmethoxy-benzyl)-3-chloro-5-(pyridin-2-ylmethoxy)-benzamide hydrochloride according to general procedure C. Colorless solid. MS 468.1 ([M+H]⁺)

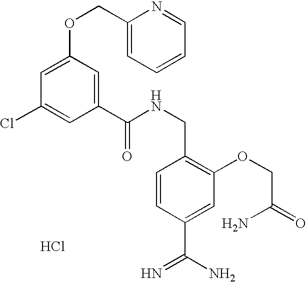

### Example 92

92.1 In analogy to example 3.2, 3-chloro-N-(4-cyano-2-methylcarbamoylmethoxy-benzyl)-5-hydroxy-benzamide (example 66.1) was alkylated with 2-(chloromethyl)-1-methyl-1H-imidazole hydrochloride to give 3-chloro-N-(4-cyano-2-methylcarbamoylmethoxy-benzyl)-5-(1-methyl-1H-imidazol-2-ylmethoxy)-benzamide as a colorless solid. MS 468.4 ([M+H]⁺)

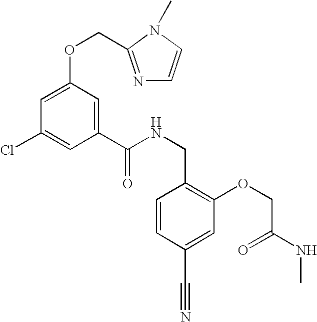

92.2 3-Chloro-N-(4-cyano-2-methylcarbamoylmethoxy-benzyl)-5-(1-methyl-1H-imidazol-2-ylmethoxy)-benzamide was converted to N-(4-carbamimidoyl-2-methylcarbamoyl-methoxy-benzyl)-3-chloro-5-(1-methyl-1H-imidazol-2-ylmethoxy)-benzamide hydrochloride according to general procedure C. Colorless solid. MS 485.5 ([M+H]⁺)

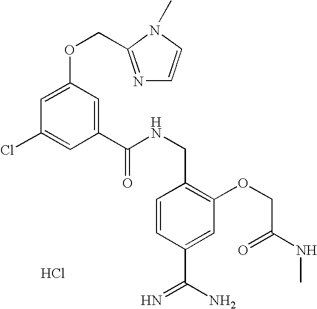

### Example 93

93.1 3-Chloro-4-fluoro-5-nitro-benzoic acid (CAS 132992-43-9) was coupled with 4-aminomethyl-3-hydroxy-benzonitrile hydrochloride (BB1) according to general procedure A to give 3-chloro-N-(4-cyano-2-hydroxy-benzyl)-4-fluoro-5-nitro-benzamide. Yellow solid. MS 348.1 ([M−H]⁻)

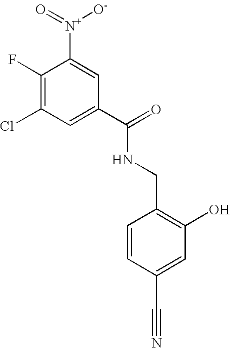

93.2 In analogy to example 49.2, 3-chloro-N-(4-cyano-2-hydroxy-benzyl)-4-fluoro-5-nitro-benzamide was reduced with zinc in acetic acid to give 3-amino-5-chloro-N-(4-cyano-2-hydroxy-benzyl)-4-fluoro-benzamide as a light brown solid. MS 320.1 ([M+H]⁺)

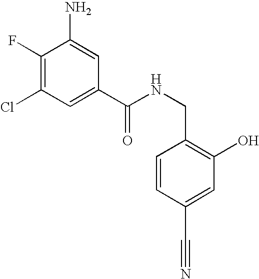

93.3 In analogy to example 3.2, 3-amino-5-chloro-N-(4-cyano-2-hydroxy-benzyl)-4-fluoro-benzamide was alkylated with iodoacetamide to give 3-amino-N-(2-carbamoylmethoxy-4-cyano-benzyl)-5-chloro-4-fluoro-benzamide as a light grey solid. MS 375.3 ([M+H]⁺)

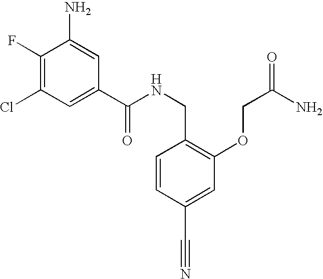

93.4 3-Amino-N-(2-carbamoylmethoxy-4-cyano-benzyl)-5-chloro-4-fluoro-benzamide was converted to 3-amino-N-(4-carbamimidoyl-2-carbamoylmethoxy-benzyl)-5-chloro-4-fluoro-benzamide hydrochloride according to general procedure C. Yellow solid. MS 394.1 ([M+H]⁺)

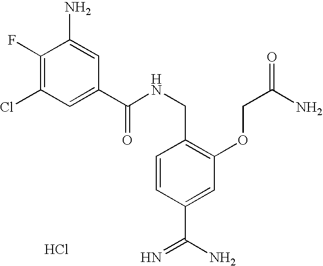

### Example 94

94.1 3-Chloro-4-fluoro-5-nitro-benzoic acid (CAS 132992-43-9) was coupled with 2-(2-aminomethyl-5-cyano-phenoxy)-N-methyl-acetamide hydrochloride (BB3) according to general procedure A to give 3-chloro-N-(4-cyano-2-methylcarbamoylmethoxy-benzyl)-4-fluoro-5-nitro-benzamide. Light yellow solid. MS 421.1 ([M+H]⁺)

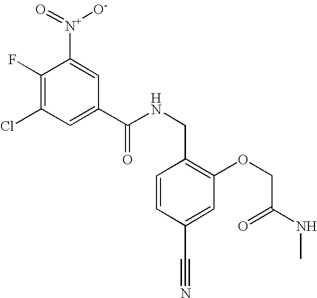

94.2 In analogy to example 49.2, 3-chloro-N-(4-cyano-2-methylcarbamoylmethoxy-benzyl)-4-fluoro-5-nitro-benzamide was reduced with zinc in acetic acid to give 3-amino-5-chloro-N-(4-cyano-2-methylcarbamoylmethoxy-benzyl)-4-fluoro-benzamide as a light grey solid. MS 391.0 ([M+H]⁺)

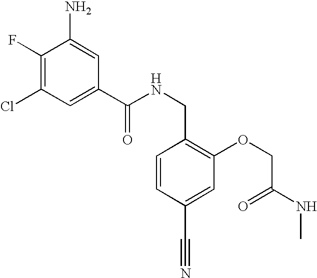

94.3 To a suspension of 3-amino-5-chloro-N-(4-cyano-2-methylcarbamoylmethoxy-benzyl)-4-fluoro-benzamide (460 mg) in THF (3 ml) were added acetic anhydride (61 mg), triethylamine (61 mg) and 4-dimethylaminopyridine (7 mg). After stirring at r.t. for 18 h and at 55° C. for 2 h no reaction ocurred. Triethylamine (122 mg) and acetyl chloride (43 microliter) were added and stirred for 60 h at r.t. Acetyl chloride (43 microliter) was added and the mixture was heated to 50° C. for 8 h. More acetyl chloride (43 microliter) and triethylamine (122 mg) were added twice while stirring at 50° C. was continued for 7 days. After completion of the reaction, the solvents were evaporated. The residue was treated with EtOAc and the product was filtered off to give 3-acetylamino-5-chloro-N-(4-cyano-2-methylcarbamoylmethoxy-benzyl)-4-fluoro-benzamide (114 mg) as a light grey solid. MS 431.4 ([M+H]⁺)

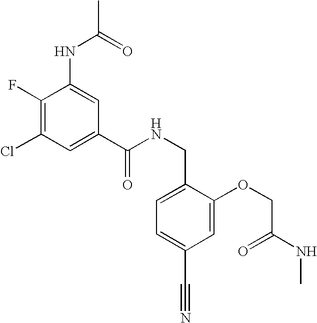

94.4 3-Acetylamino-5-chloro-N-(4-cyano-2-methylcarbamoylmethoxy-benzyl)-4-fluoro-benzamide was converted to 3-acetylamino-N-(4-carbamimidoyl-2-methyl-carbamoylmethoxy-benzyl)-5-chloro-4-fluoro-benzamide hydrochloride according to general procedure C. Colorless solid. MS 450.4 ([M+H]⁺)

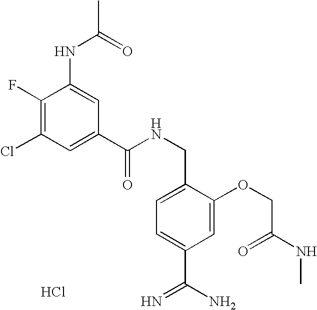

### Example 95

95.1 3-Amino-5-chloro-benzoic acid methyl ester (CAS 21961-31-9, 0.400 g) was dissolved in 10.5 ml of chloroform and treated at 0° C. with 0.344 g of isobutyryl chloride (1.5 eq.) and 0.90 ml of triethylamine. After stirring for 2 h at ambient temperature, the reaction mixture was poured onto crashed ice/HCl-solution, extracted twice with AcOEt, washed with water, dried over sodium sulfate, and evaporated i. V. Flash chromatography (SiO₂, hexane/AcOEt=7/3) yielded finally 0.546 g of pure 3-chloro-5-isobutyrylamino-benzoic acid methyl ester as white waxy solid. MS 256.0 ([M+H]⁺).

It was dissolved in 12.8 ml of THF/EtOH=1/1, treated with 6.4 ml (3 eq.) of 1N NaOH and kept at ambient temperature for 2 h. The reaction mixture was then poured onto crashed ice/AcOEt/HCl dil., the aqueous phase extracted again with AcOEt, the combined organic layers were washed with water, dried over sodium sulfate, and evaporated to dryness to produce 0.529 g of 3-chloro-5-isobutyrylamino-benzoic acid as white solid. MS 240.1 ([M−H]⁻).

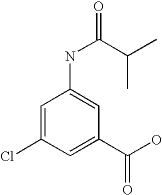

95.2 3-Chloro-5-isobutyrylamino-benzoic acid (0.358 g) was coupled with 4-aminomethyl-3-hydroxy-benzonitrile hydrochloride according to general procedure A to yield after flash chromatography (SiO₂, hexane/AcOEt=7/3) 0.555 g of 3-chloro-(4-cyano-2-hydroxy-benzyl)-5-isobutyrylamino-benzamide as off-white foam. MS 372.2 ([M+H]⁺).

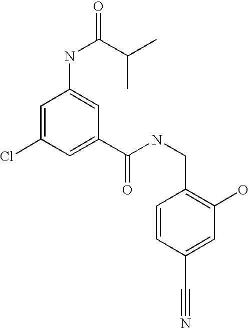

95.3 To a solution of 3-chloro-(4-cyano-2-hydroxy-benzyl)-5-isobutyrylamino-benzamide (0.100 g) in acetonitrile (2.3 ml) were added successively cesium carbonate (0.096 g) and iodoacetamide (0.052 g) and the reaction mixture was stirred at r.t. overnight. Pouring onto crashed ice/NH₄Cl-solution, twofold extraction with AcOEt, washing with brine, drying over sodium sulfate, and evaporation of the solvents, followed by crystallisation from AcOEt, afforded 0.072 g of N-(2-carbamoylmethoxy-4-cyano-benzyl)-3-chloro-5-isobutyrylamino-benzamide. MS 429.4 ([M+H]⁺).

This intermediate was subjected to the Pinner reaction as described in general procedure C to yield after flash chromatography (SiO₂, AcOEt/acetone/AcOH/water=6/2/1/1) and crystallisation from AcOEt 0.070 g of N-(4-carbamimidoyl-2-carbamoylmethoxy-benzyl)-3-chloro-5-isobutyrylamino-benzamide; compound with acetic acid, as white solid. MS 446.3 ([M+H]⁺).

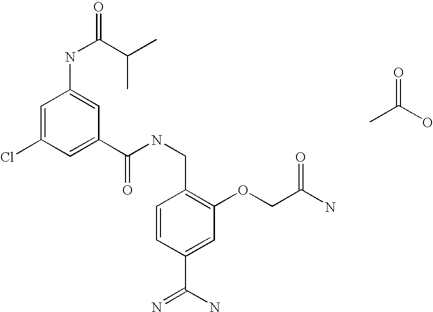

### Example 96

N-(4-Carbamimidoyl-2-methylcarbamoylmethoxy-benzyl)-3-chloro-5-isobutyrylamino-benzamide; compound with acetic acid, was prepared in analogy to example 95, but using in step 3a] 2-chloro-N-methylacetamide/potassium iodide as electrophile instead of iodoacetamide, as white solid. MS [M+H]⁺=460.5

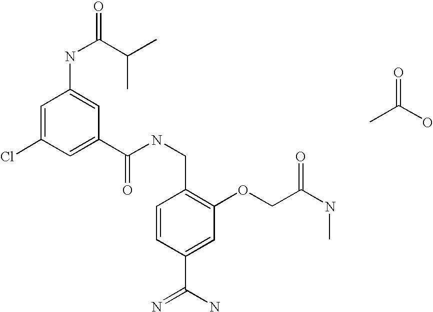

### Example 97

{5-Carbamimidoyl-2-[(3-chloro-5-isobutyrylamino-benzoylamino)-methyl]-phenoxy}-acetic acid ethyl ester; compound with acetic acid, was prepared in analogy to example 95, but using in step 3a] ethyl bromoacetate as electrophile instead of iodoacetamide, as colorless foam. MS [M+H]⁺=475.4.

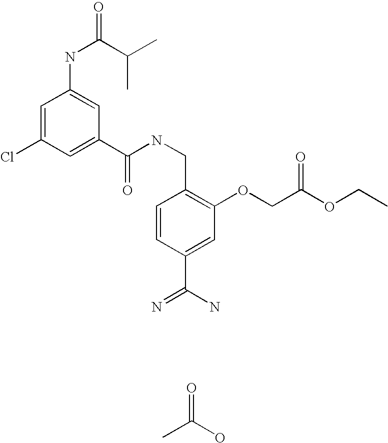

### Example 98

3-Acetylamino-N-(4-carbamimidoyl-2-methylcarbamoylmethoxy-benzyl)-5-chloro-benzamide; compound with HCl, was prepared in analogy to example 96, but using in step 1] acetyl chloride instead of isobutyryl chloride, as light yellow solid. MS [M+H]⁺=432.4.

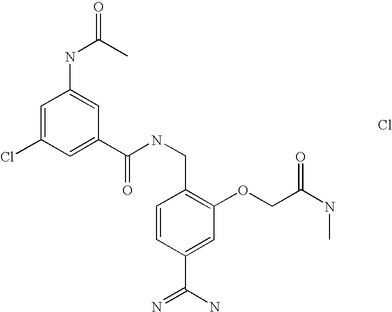

### Example 99

99.1 3-Amino-5-chloro-benzoic acid methyl ester (CAS 21961-31-9, 0.525 g) was dissolved in 5 ml of chloroform and treated at 0° C. with 0.44 ml of methanesulfonyl chloride (2 eq.) and 0.46 ml of pyridine. After stirring for 1 h at ambient temperature, the reaction mixture was poured onto crashed ice/NH₄Cl-solution, extracted twice with AcOEt, washed with water and brine, dried over magnesium sulfate, and evaporated to dryness to give 0.730 g of 3-chloro-5-methanesulfonylamino-benzoic acid methyl ester as off-white crystals.

0.725 g of this ester was dissolved in 2 ml of THF/EtOH=1/1, treated with 11 ml (4 eq.) of 1N NaOH and kept at ambient temperature for 2 h. The reaction mixture was then poured onto crashed ice/AcOEt/HCl dil., the aqueous phase extracted again with AcOEt; the combined organic layers were washed with water and brine, dried over magnesium sulfate, and evaporated to dryness to leave 0.634 g of 3-chloro-5-methanesulfonylamino-benzoic acid as white crystals. MS [M−H]−=247.9.

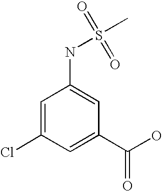

99.2 3-Chloro-5-methanesulfonylamino-benzoic acid was coupled with 4-aminomethyl-3-hydroxy-benzonitrile hydrochloride according to general procedure A to 3-chloro-N-(4-cyano-2-hydroxy-benzyl)-5-methanesulfonylamino-benzamide as light yellow foam. MS [M−H]−=378.1.

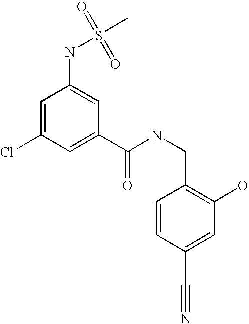

99.3 3-Chloro-N-(4-cyano-2-hydroxy-benzyl)-5-methanesulfonylamino-benzamide (0.212 g) in acetonitrile (3 ml) was treated successively with cesium carbonate (0.209 g), 2-chloro-N-methylacetamide (0.065 g) and potassium iodide (0.100 g), and the reaction mixture was stirred at 40° C. overnight. Pouring onto crashed ice/NH₄Cl-solution, twofold extraction with AcOEt, washing with water and brine, drying over magnesium sulfate, and evaporation of the solvents, followed by crystallisation from AcOEt, afforded 0.070 g of 3-chloro-N-(4-cyano-2-methylcarbamoylmethoxy-benzyl)-5-methanesulfonylamino-benzamide as brownish crystals.

This intermediate was subjected to the Pinner reaction as described in general procedure C to yield after flash chromatography (SiO₂, AcOEt/acetone/AcOH/water=6/2/1/1) and crystallisation from AcOEt 0.055 g of N-(4-carbamimidoyl-2-methylcarbamoylmethoxy-benzyl)-3-chloro-5-methanesulfonylamino-benzamide; compound with acetic acid, as off-white crystals. MS [M−H]−=466.2.

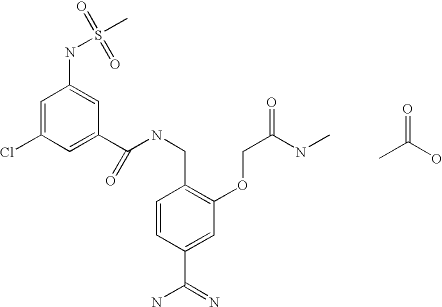

### Example 100

3-Chloro-N-(4-cyano-2-hydroxy-benzyl)-5-methanesulfonylamino-benzamide (0.189 g) in acetonitrile (4 ml) was treated successively with cesium carbonate (0.405 g) and iodoacetamide (0.101 g), and the reaction mixture was stirred at ambient temperature overnight. Pouring onto crashed ice/NH₄Cl-solution, twofold extraction with AcOEt, washing with water and brine, drying over magnesium sulfate, and evaporation of the solvents, followed by crystallisation from AcOEt, yielded 0.039 g of N-(2-carbamoylmethoxy-4-cyano-benzyl)-3-chloro-5-methanesulfonylamino-benzamide as white crystals. MS [M+H]⁺=437.3. The dialkylated product was in this experiment not isolated.

This nitrile was subjected to the Pinner reaction as described in general procedure C to yield after flash chromatography (SiO₂, AcOEt/acetone/AcOH/water=6/2/1/1) N-(4-carbamimidoyl-2-carbamoylmethoxy-benzyl)-3-chloro-5-methanesulfonylamino-benzamide; compound with acetic acid, as off-white solid. MS [M+H]+=454.0.

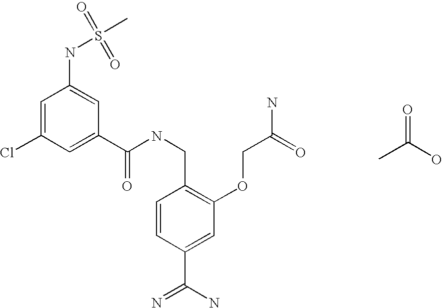

### Example 101

3-Chloro-N-(4-cyano-2-hydroxy-benzyl)-5-methanesulfonylamino-benzamide (0.158 g) in acetonitrile (3 ml) was treated successively with cesium carbonate (0.156 g) and iodoacetamide (0.082 g), and the reaction mixture was stirred at ambient temperature overnight. Pouring onto crashed ice/NH₄Cl-solution, twofold extraction with AcOEt, washing with water and brine, drying over magnesium sulfate, and evaporation of the solvents, followed by flash chromatography (SiO₂, hexane/AcOEt=3/7) yielded in the more polare fractions 0.045 g of N-(2-carbamoylmethoxy-4-cyano-benzyl)-3-(carbamoylmethyl-methanesulfonyl-amino)-5-chloro-benzamide as white crystals. MS [M+H]+=494.4.

This nitrile was subjected to the Pinner reaction as described in general procedure C to yield after direct crystallisation from acetonitrile 0.045 g of N-(4-carbamimidoyl-2-carbamoylmethoxy-benzyl)-3-(carbamoylmethyl-methanesulfonyl-amino)-5-chloro-benzamide; compound with HCl, as white powder. MS [M+H]+=511.4.

### Example 102

102.1 3-Chloro-5-methanesulfonylamino-benzoic acid was coupled with 4-aminomethyl-3-nitro-benzonitrile hydrochloride according to general procedure A to afford, after flash chromatography (SiO₂, hexane/AcOEt=4/6), 3-chloro-N-(4-cyano-2-nitro-benzyl)-5-methanesulfonylamino-benzamide as light yellow crystals. MS [M−H]−=407.2

0.740 g thereof was dissolved in 18 ml of ethanol and hydrogenated over 0.370 g of Pd on charcoal (10%) at atmospheric pressure and ambient temperature. After 15 h, the reaction mixture was filtered over a pad of Celite, rinsed generously with EtOH, and evaporated to dryness. Flash chromatography (SiO₂, hexane/AcOEt=45/55) produced 0.475 g of N-(2-amino-4-cyano-benzyl)-3-chloro-5-methanesulfonylamino-benzamide as off-white foam. MS [M+H]+=379.3.

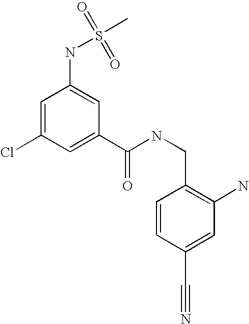

102.2 N-(2-amino-4-cyano-benzyl)-3-chloro-5-methanesulfonylamino-benzamide (0.140 g) and acetaldehyde (0.10 ml, 5 eq.) were dissolved in 4 ml of MeOH. One added a solution of ZnCl₂ (0.201 g, 4 eq.) and NaCNBH₃ (0.070 g, 3. eq.) in 2 ml of MeOH and stirred for 4 h at 55° C. Pouring onto crashed ice/NH₄Cl-solution, twofold extraction with AcOEt, washing with brine, drying over magnesium sulfate, and evaporation of the solvents, followed by flash chromatography (SiO₂, hexane/AcOEt=1/1) gave 0.098 g of 3-chloro-N-(4-cyano-2-ethylamino-benzyl)-5-methanesulfonylamino-benzamide as white crystals. MS [M+OAc]−=465.1.

This nitrile was subjected to the Pinner reaction as described in general procedure C to yield after direct crystallisation from acetonitrile 0.092 g of N-(4-carbamimidoyl-2-ethylamino-benzyl)-3-chloro-5-methanesulfonylamino-benzamide; compound with HCl, as light yellow crystals. MS [M−H]−=422.1.

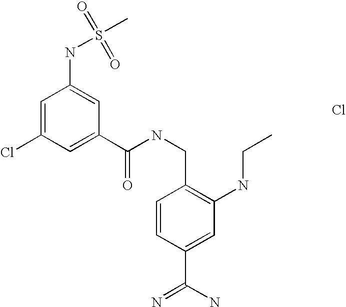

### Example 103

N-[4-Carbamimidoyl-2-(2-fluoro-benzylamino)-benzyl]-3-chloro-5-methanesulfonylamino-benzamide; compound with acetic acid, was prepared in analogy to example 102, but using in step 2] 2-fluorobenzaldehyde instead of acetaldehyde for the reductive amination, and running at the end a flash column chromatography (SiO₂, AcOEt/acetone/AcOH/water=6/2/1/1), as off-white foam. MS [M−H]−=502.1.

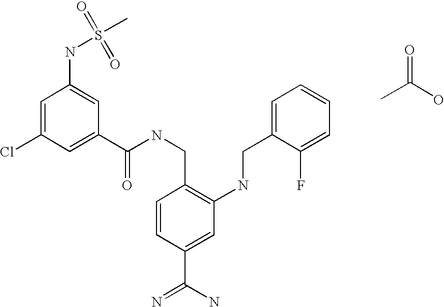

### Example 104

104.1 3-Chloro-4-fluoro-5-nitro-benzoic acid (CAS 132992-43-9) was coupled with 4aminomethyl-3-hydroxy-benzonitrile hydrochloride (BB1) according to general procedure A to give 3-chloro-N-(4-cyano-2-hydroxy-benzyl)-4-fluoro-5-nitro-benzamide. Yellow solid. MS 348.1 ([M−H]⁻)

104.2 In analogy to example 49.2, 3-chloro-N-(4-cyano-2-hydroxy-benzyl)-4-fluoro-5-nitro-benzamide was reduced with zinc powder in acetic acid to give 3-amino-5-chloro-N-(4-cyano-2-hydroxy-benzyl)-4-fluoro-benzamide as a light brown solid. MS 320.1 ([M+H]⁺)

104.3 In analogy to example 1.2, 3-amino-5-chloro-N-(4-cyano-2-hydroxy-benzyl)-4-fluoro-benzamide was alkylated with iodoacetamide to give 3-amino-N-(2-carbamoylmethoxy-4-cyano-benzyl)-5-chloro-4-fluoro-benzamide as a light grey solid. MS 375.3 ([M+H]⁺)

104.4 To a suspension of 3-amino-N-(2-carbamoylmethoxy-4-cyano-benzyl)-5-chloro-4-fluoro-benzamide (200 mg) in dichloromethane (1.5 ml) were added pyridine (47 mg) and methanesulfonyl chloride (73 mg). The mixture was stirred for one week at r.t. and for two weeks at reflux. The light grey solid was filtered off and dried to give N-(2-carbamoylmethoxy-4-cyano-benzyl)-3-chloro-4-fluoro-5-methanesulfonylamino-benzamide (133 mg). MS 455.0 ([M+H]⁺)

104.5 N-(2-Carbamoylmethoxy-4-cyano-benzyl)-3-chloro-4-fluoro-5-methanesulfonylamino-benzamide was converted to N-(4-carbamimidoyl-2-carbamoylmethoxy-benzyl)-3-chloro-4-fluoro-5-methanesulfonylamino-benzamide hydrochloride according to general procedure C. Colorless solid. MS 472.4 ([M+H]⁺)

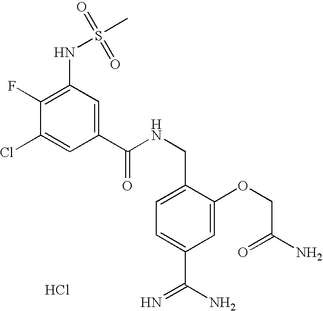

### Example 105

105.1 In analogy to example 104.4, 3-amino-N-(2-carbamoylmethoxy-4-cyano-benzyl)-5-chloro-4-fluoro-benzamide (example 104.3) was reacted with benzenesulfonyl chloride to give 3-benzenesulfonylamino-N-(2-carbamoylmethoxy-4-cyano-benzyl)-5-chloro-4-fluoro-benzamide as a yellow solid. MS 515.0 ([M−H]⁻)

105.2 3-Benzenesulfonylamino-N-(2-carbamoylmethoxy-4-cyano-benzyl)-5-chloro-4-fluoro-benzamide was converted to 3-benzenesulfonylamino-N-(4-carbamimidoyl-2-carbamoylmethoxy-benzyl)-5-chloro-4-fluoro-benzamide hydrochloride according to general procedure C. Colorless solid. MS 534.3 ([M+H]⁺)

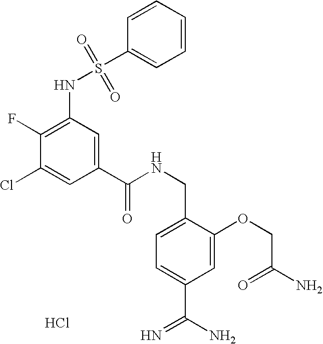

### Example 106

106.1 3-Chloro-5-methoxy-benzoic acid (CAS 82477-67-6) was coupled with 4-aminomethyl-3-hydroxy-benzonitrile hydrochloride (BB1) according to general procedure A to give 3-chloro-N-(4-cyano-2-hydroxy-benzyl)-5-methoxy-benzamide. Off-white solid. MS 315.1 ([M−H]⁻)

106.2 In analogy to example 3.2, 3-chloro-N-(4-cyano-2-hydroxy-benzyl)-5-methoxy-benzamide was alkylated with ethyliodoacetate and cesium carbonate in dimethylacetamide to give {2-[(3-chloro-5-methoxy-benzoylamino)-methyl]-5-cyano-phenoxy}-acetic acid ethyl ester. Off-white solid. MS 403.4 ([M+H]⁺)

106.3 In analogy to example 8.3, {2-[(3-chloro-5-methoxy-benzoylamino)-methyl]-5-cyano-phenoxy}-acetic acid ethyl ester was hydrolyzed to give {2-[(3-chloro-5-methoxy-benzoylamino)-methyl]-5-cyano-phenoxy}-acetic acid. Colorless solid. MS 373.1 ([M−H]⁻)

106.4 {2-[(3-Chloro-5-methoxy-benzoylamino)-methyl]-5-cyano-phenoxy}-acetic acid was reacted with 3-(aminomethyl)-pyridine according to general procedure A to give 3-chloro-N-(4-cyano-2-1{[(pyridin-3-ylmethyl)-carbamoyl]-methoxy}-benzyl)-5-methoxy-benzamide. Colorless solid. MS 465.1 ([M+H]⁺)

106.5 3-Chloro-N-(4-cyano-2-{[(pyridin-3-ylmethyl)-carbamoyl]-methoxy}-benzyl)-5-methoxy-benzamide was converted to N-(4-carbamimidoyl-2-{[(pyridin-3-ylmethyl)-carbamoyl]-methoxy}-benzyl)-3-chloro-5-methoxy-benzamide hydrochloride according to general procedure C. Colorless solid. MS 482.5 ([M+H]⁺)

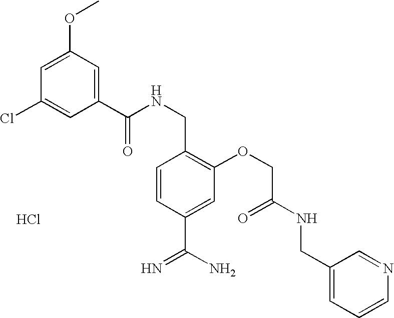

### Example 107

107.1 {2-[(3-Chloro-5-methoxy-benzoylamino)-methyl]-5-cyano-phenoxy}-acetic acid (example 106.3) was reacted with 2-(aminomethyl)-pyridine according to general procedure A to give 3-chloro-N-(4-cyano-2-{[(pyridin-2-ylmethyl)-carbamoyl]-methoxy}-benzyl)-5-methoxy-benzamide. Colorless solid. MS 465.1 ([M+H]⁺)

107.2 3-Chloro-N-(4-cyano-2-{[(pyridin-2-ylmethyl)-carbamoyl]-methoxy}-benzyl)-5-methoxy-benzamide was converted to N-(4-carbamimidoyl-2-{[(pyridin-2-ylmethyl)-carbamoyl]-methoxy}-benzyl)-3-chloro-5-methoxy-benzamide hydrochloride according to general procedure C. Colorless solid. MS 482.5 ([M+H]⁺)

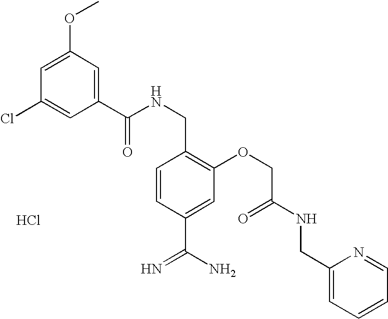

### Example 108

108.1 {2-[(3-Chloro-5-methoxy-benzoylamino)-methyl]-5-cyano-phenoxy}-acetic acid (example 106.3) was reacted with 2-fluorobenzylamine according to general procedure A to give 3-chloro-N-{4-cyano-2-[(2-fluoro-benzylcarbamoyl)-methoxy]-benzyl}-5-methoxy-benzamide. Off-white solid. MS 482.1 ([M+H]⁺)

108.2 3-Chloro-N-{4-cyano-2-[(2-fluoro-benzylcarbamoyl)-methoxy]-benzyl}-5-methoxy-benzamide was converted to N-{4-carbamimidoyl-2-[(2-fluoro-benzylcarbamoyl)-methoxy]-benzyl}-3-chloro-5-methoxy-benzamide hydrochloride according to general procedure C. Off-white solid. MS 499.4 ([M+H]⁺)

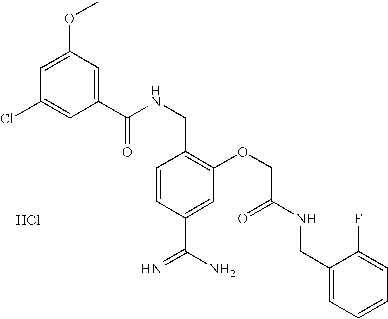

### Example 109

109.1 {2-[(3-Chloro-5-methoxy-benzoylamino)-methyl]-5-cyano-phenoxy}-acetic acid (example 106.3) was reacted with 2-methoxyethylamine according to general procedure A to give 3-chloro-N-{4-cyano-2-[(2-methoxy-ethylcarbamoyl)-methoxy]-benzyl}-5-methoxy-benzamide. Colorless solid. MS 432.3 ([M+H]⁺)

109.2 3-Chloro-N-{4-cyano-2-[(2-methoxy-ethylcarbamoyl)-methoxy]-benzyl}-5-methoxy-benzamide was converted to N-{4-carbamimidoyl-2-[(2-methoxy-ethylcarbamoyl)-methoxy]-benzyl}-3-chloro-5-methoxy-benzamide hydrochloride according to general procedure C. Colorless solid. MS 449.3 ([M+H]⁺)

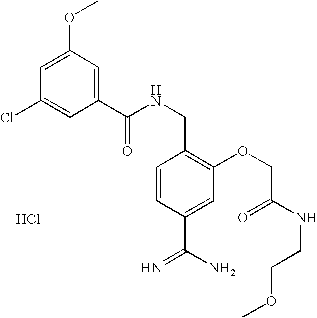

### Example 110

110.1 {2-[(3-Chloro-5-methoxy-benzoylamino)-methyl]-5-cyano-phenoxy}-acetic acid (example 106.3) was reacted with isobutylamine according to general procedure A to give 3-chloro-N-[4-cyano-2-(isobutylcarbamoyl-methoxy)-benzyl]-5-methoxy-benzamide. Off-white solid. MS 430.2 ([M+H]⁺)

110.2 3-Chloro-N-[4-cyano-2-(isobutylcarbamoyl-methoxy)-benzyl]-5-methoxy-benzamide was converted to N-[4-carbamimidoyl-2-(isobutylcarbamoyl-methoxy)-benzyl]-3-chloro-5-methoxy-benzamide hydrochloride according to general procedure C. Light yellow solid. MS 447.3 ([M+H]⁺)

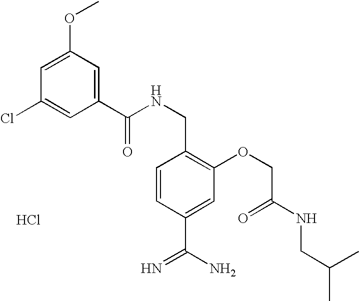

### Example 111

111.1 {2-[(3-Chloro-5-methoxy-benzoylamino)-methyl]-5-cyano-phenoxy}-acetic acid (example 106.3) was reacted with aminomethylcyclopropane according to general procedure A to give 3-chloro-N-{4-cyano-2-[(cyclopropylmethyl-carbamoyl)-methoxy]-benzyl}-5-methoxy-benzamide. Off-white solid. MS 428.1 ([M+H]⁺)

111.2 3-Chloro-N-{4-cyano-2-[(cyclopropylmethyl-carbamoyl)-methoxy]-benzyl}-5-methoxy-benzamide was converted to N-{4-carbamimidoyl-2-[(cydopropylmethyl-carbamoyl)-methoxy]-benzyl}-3-chloro-5-methoxy-benzamide hydrochloride according to general procedure C. Off-white solid. MS 445.2 ([M+H]⁺)

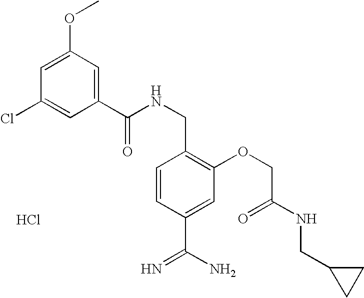

### Example 112

112.1 {2-[(3-Chloro-5-methoxy-benzoylamino)-methyl]-5-cyano-phenoxy}-acetic acid (example 106.3) was reacted with glycine methylester hydrochloride according to general procedure A to give (2-{2-[(3-chloro-5-methoxy-benzoylamino)-methyl]-5-cyano-phenoxy}-acetylamino)-acetic acid methyl ester. Colorless solid. MS 446.2 ([M+H]⁺)

112.1 (2-{2-[(3-Chloro-5-methoxy-benzoylamino)-methyl]-5-cyano-phenoxy}-acetylamino)-acetic acid methyl ester was converted to (2-{5-carbamimidoyl-2-[(3-chloro-5-methoxy-benzoylamino)-methyl]-phenoxy}-acetylamino)-acetic acid ethyl ester hydrochloride according to general procedure C. Light pink solid. MS 477.0 ([M+H]⁺)

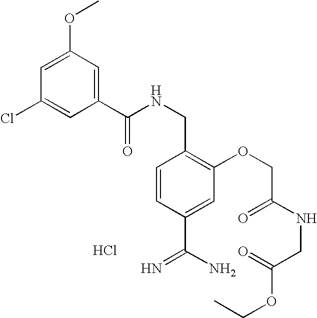

112.3 In analogy to example 8.3, (2-{5-carbamimidoyl-2-[(3-chloro-5-methoxy-benzoylamino)-methyl]-phenoxy}-acetylamino)-acetic acid ethyl ester hydrochloride was hydrolyzed to give (2-{5-carbamimidoyl-2-[(3-chloro-5-methoxy-benzoylamino)-methyl]-phenoxy}-acetylamino)-acetic acid as a colorless solid. MS 449.3 ([M+H]⁺)

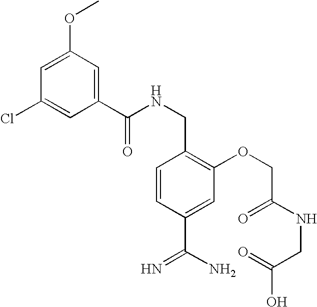

### Example 113

113.1 {2-[(3-Chloro-5-methoxy-benzoylamino)-methyl]-5-cyano-phenoxy}-acetic acid (example 106.3) was reacted with alpha-aminoisobutyric acid methylester hydrochloride according to general procedure A to give 2-(2-{2-[(3-chloro-5-methoxy-benzoylamino)-methyl]-5-cyano-phenoxy}-acetylamino)-2-methyl-propionic acid methyl ester. Colorless solid. MS 474.1 ([M+H]⁺)

113.2 2-(2-{2-[(3-Chloro-5-methoxy-benzoylamino)-methyl]-5-cyano-phenoxy}-acetylamino)-2-methyl-propionic acid methyl ester was converted to 2-(2-{5-carbamimidoyl-2-[(3-chloro-5-methoxy-benzoylamino)-methyl]-phenoxy}-acetylamino)-2-methyl-propionic acid methyl ester hydrochloride according to general procedure C. Colorless solid. MS 491.2 ([M+H]⁺)

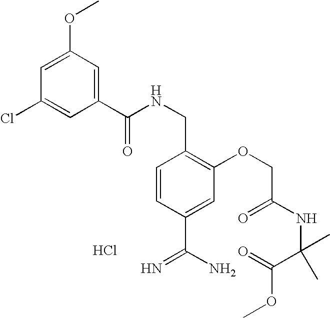

113.3 In analogy to example 8.3, 2-(2-{5-carbamimidoyl-2-[(3-chloro-5-methoxy-benzoylamino)-methyl]-phenoxy}-acetylamino)-2-methyl-propionic acid methyl ester hydrochloride was hydrolyzed to give 2-(2-{5-carbamimidoyl-2-[(3-chloro-5-methoxy-benzoylamino)-methyl]-phenoxy}-acetylamino)-2-methyl-propionic acid as a colorless solid. MS 477.2 ([M+H]⁺)

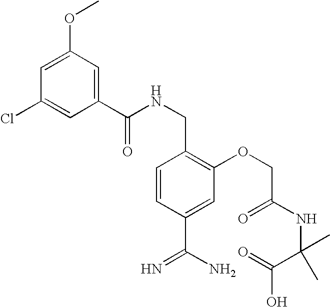

### Example 114

114.1 {2-[(3-Chloro-5-methoxy-benzoylamino)-methyl]-5-cyano-phenoxy}-acetic acid (example 106.3) was reacted with methyl-3-aminobenzoate according to general procedure A to give 3-(2-{2-[(3-chloro-5-methoxy-benzoylamino)-methyl]-5-cyano-phenoxy}-acetylamino)-benzoic acid methyl ester. Off-white solid. MS 509.5 ([M+H]⁺)

114.2 3-(2-{2-[(3-Chloro-5-methoxy-benzoylamino)-methyl]-5-cyano-phenoxy}-acetylamino)-benzoic acid methyl ester was converted to 3-(2-{5-carbamimidoyl-2-[(3-chloro-5-methoxy-benzoylamino)-methyl]-phenoxy}-acetylamino)-benzoic acid methyl ester hydrochloride ammoniumchloride 1:1:1 according to general procedure C. Colorless solid. MS 525.3 ([M+H]⁺)

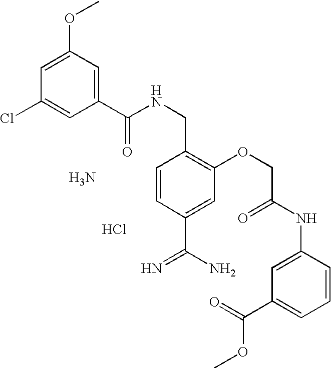

114.3 In analogy to example 8.3, 3-(2-{5-carbamimidoyl-2-[(3-chloro-5-methoxy-benzoylamino)-methyl]-phenoxy}-acetylamino)-benzoic acid methyl ester hydrochloride ammoniumchloride 1:1:1 was hydrolyzed to give 3-(2-{5-carbamimidoyl-2-[(3-chloro-5-methoxy-benzoylamino)-methyl]-phenoxy}-acetylamino)-benzoic acid as a colorless solid. MS 511.4 ([M+H]⁺)

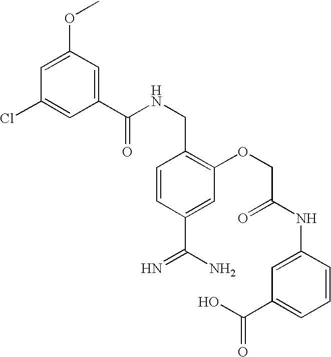

### Example 115

115.1 3-Chloro-2-fluoro-5-(trifluoromethyl) benzoic acid was coupled with 2-(2-aminomethyl-5-cyano-phenoxy)-N-methyl-acetamide hydrochloride (BB3) according to general procedure A to give 3-chloro-N-(4-cyano-2-methylcarbamoylmethoxy-benzyl)-2-fluoro-5-trifluoromethyl-benzamide. Off-white solid. MS 444.1 ([M+H]⁺)

115.2 3-Chloro-N-(4-cyano-2-methylcarbamoylmethoxy-benzyl)-2-fluoro-5-trifluoromethyl-benzamide was converted to N-(4-carbamimidoyl-2-methylcarbamoylmethoxy-benzyl)-3-chloro-2-fluoro-5-trifluoromethyl-benzamide hydrochloride according to general procedure C. Colorless solid. MS 461.3 ([M+H]⁺)

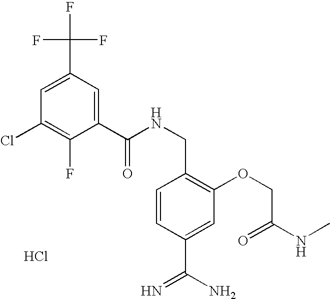

### Example 116

116.1 To a solution of 3-chloro-5-hydroxymethyl-benzoic acid methyl ester (CAS 153203-58-8, 2.89 g) in CH₂Cl₂ was added manganese (IV) oxide (2.78 g). The mixture was stirred at r.t. for 8 h. Mangenese (IV) oxide (1.39 g) was added and the mixture was stirred at 45° C. for 25 h. The solid was filtered off and washed with CH₂Cl₂. The filtrate was concentrated and the product was purified by chromatography (SiO₂, cyclohexane/ethyl acetate 1:0=>4:1) to give 3-chloro-5-formyl-benzoic acid methyl ester (1.38 g) as a colorless solid.

116.2 To a solution of 3-chloro-5-formyl-benzoic acid methyl ester (228 mg) in methanol (3.1 ml) were added 25 % aq. NH₃ solution (1.46 ml) and a solution of glyoxal (8.8 N in water, 0.704 ml). The mixture was stirred at r.t. for 3 h. Water was added and the mixture was extracted with ethyl acetate. The organic phase was washed with water and brine, dried, filtered and concentrated. The product was purified by chromatography (SiO₂, CH₂Cl₂/MeOH 1:0=>9:1) to give 3-chloro-5-(1H-imidazol-2-yl)-benzoic acid methyl ester (113 mg) as a light brown solid. MS 237.1 ([M+H]⁺)

116.3 In analogy to example 8.3, 3-chloro-5-(1H-imidazol-2-yl)-benzoic acid methyl ester was hydrolyzed to give 3-chloro-5-(1H-imidazol-2-yl)-benzoic acid. Off-white solid. MS 221.1 ([M−H]⁻)

116.4 3-Chloro-5-(1H-imidazol-2-yl)-benzoic acid was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-(1H-imidazol-2-yl)-benzamide. Off-white solid. MS 410.2 ([M+H]⁺)

116.5 N-(2-Carbamoylmethoxy-4-cyano-benzyl)-3-chloro-5-( 1H-imidazol-2-yl)-benzamide was converted to N-(4-carbamimidoyl-2-carbamoylmethoxy-benzyl)-3-chloro-5-(1H-imidazol-2-yl)-benzamide hydrochloride according to general procedure C. Light yellow solid. MS 427.3 ([M+H]⁺)

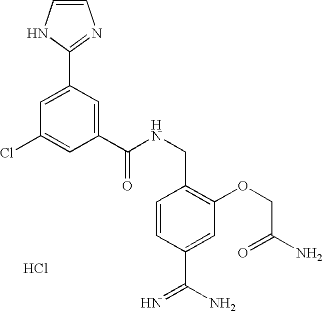

### Example 117

117.1 To a solution of 3-chloro-5-(4,4,5,5-tetramethyl-[1,3,2]dioxaborolan-2-yl)-benzoic acid methyl ester (CAS 408492-29-5, 83 mg) in 1,2-dimethoxyethane (0.8 ml) and isopropanol (0.2 ml) were added cesium fluoride (85 mg) and tetrakis(triphenylphosphine)palladium (9.6 mg). 2-Bromopyridine (44 mg) was added and the mixture was stirred for 1 week at r.t. and for 2 h at 80° C. The mixture was concentrated and the residue was taken up in ethyl acetate and washed with water. The organic phase was dried, filtered and concentrated. The product was purified by chromatography (SiO₂, cyclohexane/ethyl acetate 1:0=>4:1) to give 3-chloro-5-pyridin-2-yl-benzoic acid methyl ester (37 mg) as a colorless solid. MS 247.9 ([M+H]⁺)

117.2 In analogy to example 8.3, 3-chloro-5-pyridin-2-yl-benzoic acid methyl ester was hydrolyzed to give 3-chloro-5-pyridin-2-yl-benzoic acid. Colorless solid. MS 232.1 ([M−H]⁻)

117.3 3-Chloro-5-pyridin-2-yl-benzoic acid was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-pyridin-2-yl-benzamide. Light brown solid. MS 419.3 ([M−H]⁻)

117.4 N-(2-Carbamoylmethoxy-4-cyano-benzyl)-3-chloro-5-pyridin-2-yl-benzamide was converted to N-(4-carbamimidoyl-2-carbamoylmethoxy-benzyl)-3-chloro-5-pyridin-2-yl-benzamide hydrochloride according to general procedure C. Colorless solid. MS 438.4 ([M+H]⁺)

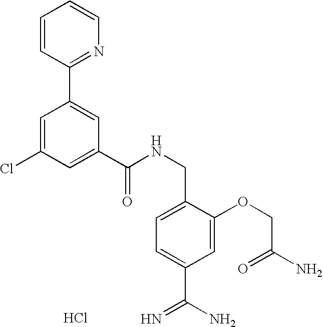

### Example 118

118.1 In analogy to example 40.1, 4-fluorophenol was reacted with 3-chloro-5-hydroxymethyl-benzoic acid methyl ester (CAS 153203-58-8), triphenylphosphine and diethylazodicarboxylate to give 3-chloro-5-(4-fluoro-phenoxymethyl)-benzoic acid methyl ester. Colorless solid.

118.2 In analogy to example 8.3, 3-chloro-5-(4-fluoro-phenoxymethyl)-benzoic acid methyl ester was hydrolyzed to give 3-chloro-5-(4-fluoro-phenoxymethyl)-benzoic acid. Off-white solid. MS 279.0 ([M−H]⁻)

118.3 3-Chloro-5-(4-fluoro-phenoxymethyl)-benzoic acid was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-(4-fluoro-phenoxymethyl)-benzamide. Off-white solid. MS 468.4 ([M+H]⁺)

118.4 N-(2-Carbamoylmethoxy-4-cyano-benzyl)-3-chloro-5-(4-fluoro-phenoxymethyl)-benzamide was converted to N-(4-carbamimidoyl-2-carbamoylmethoxy-benzyl)-3-chloro-5-(4-fluoro-phenoxymethyl)-benzamide hydrochloride according to general procedure C. Light yellow solid. MS 485.1 ([M+H]⁺)

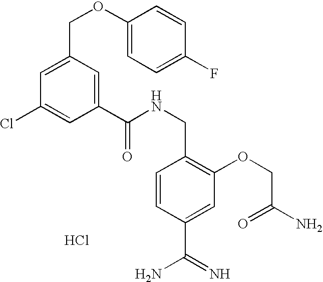

### Example 119

119.1 {2-[(3-Chloro-5-methoxy-benzoylamino)-methyl]-5-cyano-phenoxy}-acetic acid ethyl ester (example 106.2) was converted to {5-carbamimidoyl-2-[(3-chloro-5-methoxy-benzoylamino)-methyl]-phenoxy}-acetic acid methyl ester hydrochloride according to general procedure C using methanol/chloroform as the solvents. Colorless foam. MS 406.4 ([M+H]⁺)

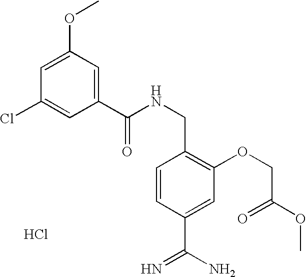

### Example 120

120.1 In analogy to example 39.4, N-{4-carbamimidoyl-2-[(pyridin-3-ylcarbamoylmethyl)-amino]-benzyl}-3-chloro-benzamide hydrochloride (example 73.2) was reacted with hydroxylamine hydrochloride and triethylamine in methanol at r.t. to give 3-chloro-N-{4-(N-hydroxycarbamimidoyl)-2-[(pyridin-3-ylcarbamoylmethyl)-amino]-benzyl}-benzamide as a colorless solid. MS 453.4 ([M+H]⁺).

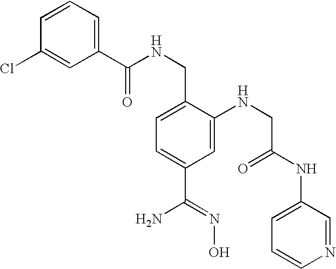

3-Chloro-N-{4-cyano-2-[(pyridin-3-ylcarbamoylmethyl)-amino]-benzyl}-benzamide (example 73.1) was converted to 3-chloro-N-{4-(N-methoxy-carbamimidoyl)-2-[(pyridin-3-ylcarbamoylmethyl)-amino]-benzyl}-benzamide according to general procedure C using methoxyamine hydrochloride and triethylamine instead of ammonia in the second step. Colorless foam. MS 467.4 ([M+H]⁺)

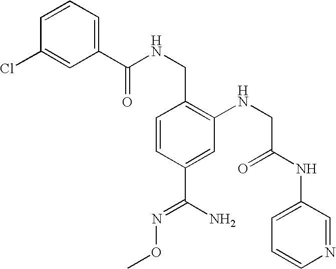

In analogy to example 39.3, N-{4-carbamimidoyl-2-[(pyridin-3-ylcarbamoylmethyl)-amino]-benzyl}-3-chloro-benzamide hydrochloride (example 73.2) was reacted with ethyl chloroformate and triethylamine in dimethylacetamide at 0° C. to give [1-amino-1-{4-[(3-chloro-benzoylamino)-methyl]-3-[(pyridin-3-ylcarbamoylmethyl)-amino]-phenyl}-meth-(Z)-ylidene]-carbamic acid ethyl ester as an off-white solid. MS 509.1 ([M+H]⁺).

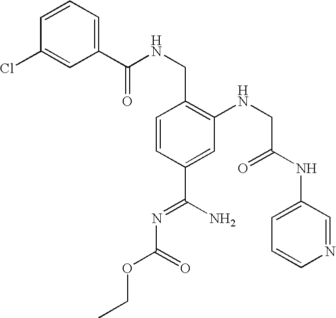

### Example 121

121.1 3-Chloro-5-hydroxy-benzoic acid (CAS 53984-36-4) was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-hydroxy-benzamide. Colorless solid. MS 358.3 ([M−H]⁻).

121.2 In analogy to example 3.2, N-(2-carbamoylmethoxy-4-cyano-benzyl)-3-chloro-5-hydroxy-benzamide was alkylated with 4-fluorobenzylbromide and cesium carbonate in dimethylacetamide to give N-(2-carbamoylmethoxy-4-cyano-benzyl)-3-chloro-5-(4-fluoro-benzyloxy)-benzamide. Colorless solid. MS 468.5 ([M+H]⁺)

121.3 N-(2-Carbamoylmethoxy-4-cyano-benzyl)-3-chloro-5-(4-fluoro-benzyloxy)-benzamide was converted to N-(4-carbamimidoyl-2-carbamoylmethoxy-benzyl)-3-chloro-5-(4-fluoro-benzyloxy)-benzamide hydrochloride according to general procedure C. Colorless solid. MS 485.5 ([M+H]⁺)

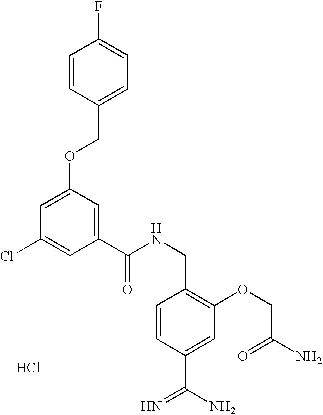

### Example 122

122.1 In analogy to example 40.1, 3-hydroxypyridine was reacted with 3-chloro-5-hydroxymethyl-benzoic acid methyl ester (CAS 153203-58-8), triphenylphosphine and diethylazodicarboxylate. The product of this reaction was hydrolyzed in analogy to example 8.3 to give 3-chloro-5-(pyridin-3-yloxymethyl)-benzoic acid. Light yellow solid. MS 262.1 ([M−H]⁻)

122.2 3-Chloro-5-(pyridin-3-yloxymethyl)-benzoic acid was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-(pyridin-3-yloxymethyl)-benzamide. Off-white solid. MS 451.1 ([M+H]⁺).

122.3 N-(2-Carbamoylmethoxy-4-cyano-benzyl)-3-chloro-5-(pyridin-3-yloxymethyl)-benzamide was converted to N-(4-carbamimidoyl-2-carbamoylmethoxy-benzyl)-3-chloro-5-(pyridin-3-yloxymethyl)-benzamide hydrochloride according to general procedure C. Colorless solid. MS 468.0 ([M+H]⁺)

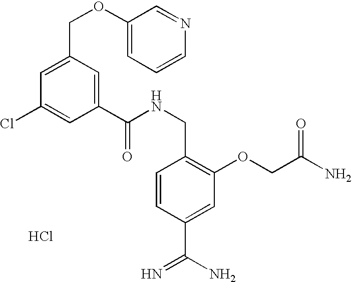

### Example 123

123.1 To a solution of 3-chloro-5-hydroxy-benzoic acid methyl ester (CAS 98406-04-3, 1.65 g) in dimethylacetamide (10 ml) was added NaH (60 % dispersion in mineral oil, 0.42 g) at 0° C. The mixture was stirred for 20 min. 2-Bromoethyl-methyl ether (1.47 g) was added. The ice bath was removed and the mixture was stirred for 10 h at r.t. Water was added and the mixture was extracted with EtOAc. The org. phase was washed with water, dried, filtered and concentrated. The product was purified by chromatography (SiO₂, cyclohexane/EtOAc 3:1=>2:1) to give 3-chloro-5-(2-methoxy-ethoxy)-benzoic acid methyl ester (1.63 g) as a light yellow oil.

123.2 In analogy to example 8.3, 3-chloro-5-(2-methoxy-ethoxy)-benzoic acid methyl ester was hydrolyzed to give 3-chloro-5-(2-methoxy-ethoxy)-benzoic acid. Colorless solid. MS 229.1 ([M−H]⁻)

123.3 3-Chloro-5-(2-methoxy-ethoxy)-benzoic acid was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-(2-methoxy-ethoxy)-benzamide. Off-white solid. MS 418.1 ([M+H]⁺).

123.4 N-(2-Carbamoylmethoxy-4-cyano-benzyl)-3-chloro-5-(2-methoxy-ethoxy)-benzamide was converted to N-(4-carbamimidoyl-2-carbamoylmethoxy-benzyl)-3-chloro-5-(2-methoxy-ethoxy)-benzamide hydrochloride according to general procedure C. Colorless solid. MS 435.3 ([M+H]⁺)

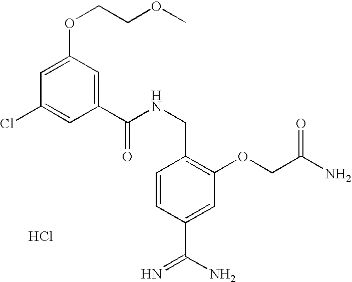

### Example 124

124.1 3-Chloro-5-(2-methoxy-ethoxy)-benzoic acid (example 123.2) was coupled with 4-aminomethyl-3-hydroxy-benzonitrile hydrochloride (BB1) according to general procedure A to give 3-chloro-N-(4-cyano-2-hydroxy-benzyl)-5-(2-methoxy-ethoxy)-benzamide. Colorless foam. MS 361.4 ([M+H]⁺)

124.2 In analogy to example 3.2, 3-chloro-N-(4-cyano-2-hydroxy-benzyl)-5-(2-methoxy-ethoxy)-benzamide was alkylated with N-(chloroacetyl)glycine ethylester and cesium carbonate in dimethylacetamide to give [2-(2-{[3-chloro-5-(2-methoxy-ethoxy)-benzoylamino]-methyl}-5-cyano-phenoxy)-acetylamino]-acetic acid ethyl ester. Colorless solid. MS 504.1 ([M+H]⁺)

124.3 [2-(2-{[3-Chloro-5-(2-methoxy-ethoxy)-benzoylamino]-methyl}-5-cyano-phenoxy)-acetylamino]-acetic acid ethyl ester was converted to [2-(5-carbamimidoyl-2-{[3-chloro-5-(2-methoxy-ethoxy)-benzoylamino]-methyl}-phenoxy)-acetylamino]-acetic acid ethyl ester hydrochloride according to general procedure C. Colorless solid. MS 521.3 ([M+H]⁺)

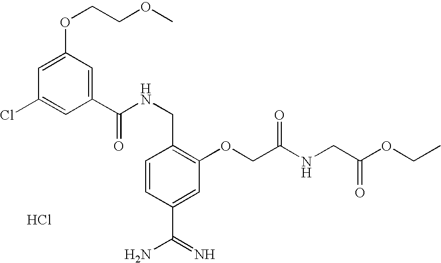

124.4 In analogy to example 8.3, [2-(5-carbamimidoyl-2-{[3-chloro-5-(2-methoxy-ethoxy)-benzoylamino]-methyl}-phenoxy)-acetylamino]-acetic acid ethyl ester hydrochloride was hydrolyzed to give [2-(5-carbamimidoyl-2-{[3-chloro-5-(2-methoxy-ethoxy)-benzoylamino]-methyl}-phenoxy)-acetylamino]-acetic acid as a colorless solid. MS 493.4 ([M+H]⁺)

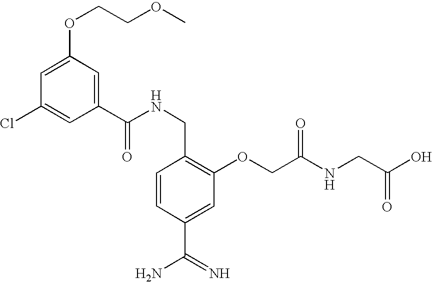

### Example 125

125.1 In analogy to example 3.2, 3-chloro-5-hydroxy-benzoic acid methyl ester (CAS 98406-04-3) was alkylated with 4-chlorobenzyl bromide and cesium carbonate in dimethylacetamide to give 3-chloro-5-(4-chloro-benzyloxy)-benzoic acid methyl ester. Light brown solid.

125.2 In analogy to example 8.3, 3-chloro-5-(4-chloro-benzyloxy)-benzoic acid methyl ester was hydrolyzed to give 3-chloro-5-(4-chloro-benzyloxy)-benzoic acid. Brown solid. MS 295.2 ([M−H]⁻)

125.3 3-Chloro-5-(4-chloro-benzyloxy)-benzoic acid was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-(4-chloro-benzyloxy)-benzamide. Off-white solid. MS 482.3 ([M−H]⁻).

125.4 N-(2-Carbamoylmethoxy-4-cyano-benzyl)-3-chloro-5-(4-chloro-benzyloxy)-benzamide was converted to N-(4-carbamimidoyl-2-carbamoylmethoxy-benzyl)-3-chloro-5-(4-chloro-benzyloxy)-benzamide hydrochloride according to general procedure C. Off-white solid. MS 501.3 ([M+H]⁺)

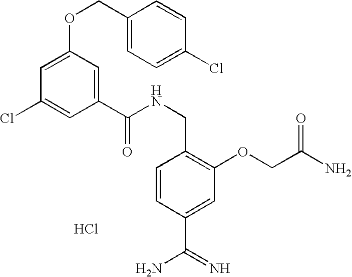

### Example 126

126.1 In analogy to example 3.2, 3-chloro-5-hydroxy-benzoic acid methyl ester (CAS 98406-04-3) was alkylated with 1,3-dichloro-5-(chloromethyl)benzene and cesium carbonate in dimethylacetamide to give 3-chloro-5-(3,5-dichloro-benzyloxy)-benzoic acid methyl ester. Light brown solid

126.2 In analogy to example 8.3, 3-chloro-5-(3,5-dichloro-benzyloxy)-benzoic acid methyl ester was hydrolyzed to give 3-chloro-5-(3,5-dichloro-benzyloxy)-benzoic acid. Off-white solid. MS 329.0 ([M−H]⁻)

126.3 3-Chloro-5-(3,5-dichloro-benzyloxy)-benzoic acid was coupled with 4-aminomethyl-3-hydroxy-benzonitrile hydrochloride (BB1) according to general procedure A to give 3-chloro-N-(4-cyano-2-hydroxy-benzyl)-5-(3,5-dichloro-benzyloxy)-benzamide. Colorless solid. MS 459.3 ([M−H]⁻)

126.4 In analogy to example 1.2, 3-chloro-N-(4-cyano-2-hydroxy-benzyl)-5-(3,5-dichloro-benzyloxy)-benzamide was alkylated with iodoacetamide to give N-(2-carbamoylmethoxy-4-cyano-benzyl)-3-chloro-5-(3,5-dichloro-benzyloxy)-benzamide as a colorless solid.

126.5 N-(2-Carbamoylmethoxy-4-cyano-benzyl)-3-chloro-5-(3,5-dichloro-benzyloxy)-benzamide was converted to N-(4-carbamimidoyl-2-carbamoylmethoxy-benzyl)-3-chloro-5-(3,5-dichloro-benzyloxy)-benzamide hydrochloride according to general procedure C. Colorless solid. MS 537.3 ([M+H]⁺)

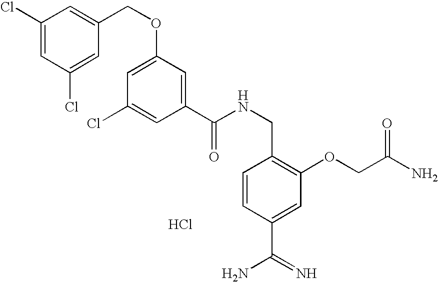

### Example 127

127.1 In analogy to example 3.2, 3-chloro-N-(4-cyano-2-hydroxy-benzyl)-5-(3,5-dichloro-benzyloxy)-benzamide (example 126.3) was alkylated with N-(chloroacetyl)glycine ethylester and cesium carbonate in dimethylacetamide to give [2-(2-{[3-chloro-5-(3,5-dichloro-benzyloxy)-benzoylamino]-methyl}-5-cyano-phenoxy)-acetylamino]-acetic acid ethyl ester as a colorless solid. MS 604.3 ([M+H]⁺)

127.2 [2-(2-{[3-Chloro-5-(3,5-dichloro-benzyloxy)-benzoylamino]-methyl phenoxy)-acetylamino]-acetic acid ethyl ester was converted to [2-(5-carbamimidoyl-2-{[3-chloro-5-(3,5-dichloro-benzyloxy)-benzoylamino]-methyl}-phenoxy)-acetylamino]-acetic acid ethyl ester hydrochloride according to general procedure C. Colorless solid. MS 623.1 ([M+H]⁺)

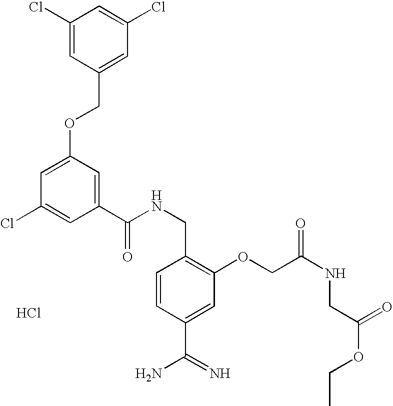

127.3 In analogy to example8.3, [2-(5-carbamimidoyl-2-{[3-chloro-5-(3,5-dichloro-benzyloxy)-benzoylamino]-methyl}-phenoxy)-acetylamino]-acetic acid ethyl ester hydrochloride was hydrolyzed to give [2-(5-carbamimidoyl-2-{[3-chloro-5-(3,5-dichloro-benzyloxy)-benzoylamino]-methyl}-phenoxy)-acetylamino]-acetic acid. Colorless solid. MS 593.2 ([M+H]⁺)

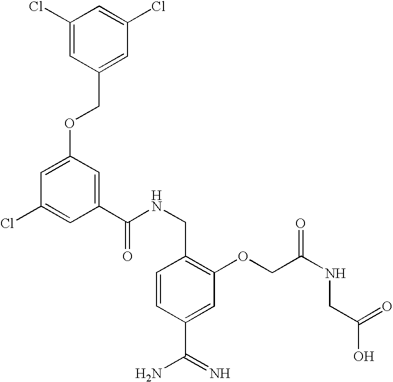

### Example 128

128.1 To a stirred solution of 3-amino-5-chloro-benzoic acid methyl ester (CAS 21961-31-9, 0.55 g) and pyridine (0.478 ml) in CH₂Cl₂ (14.8 ml) was added slowly a solution of chlorobutyrylchloride (0.372 ml) in CH₂Cl₂ (1.5 ml) at 0° C. The cooling bath was removed and the mixture was warmed to r.t. The mixture was diluted with diethyl ether and washed with 1 M HCl and with water. The organic phase was dried (MgSO₄), filtered, concentrated and dried under high vacuum to give 3-chloro-5-(4-chloro-butyrylamino)-benzoic acid methyl ester as a light brown solid. MS 290.0 ([M+H]⁺)

128.2 To a solution of 3-chloro-5-(4-chloro-butyrylamino)-benzoic acid methyl ester (914 mg) in THF (18.4 ml) was added potassium-tert-butylate (354 mg) at 0° C. The mixture was stirred at 0° C. for 1 hour and at r.t. for 5 h. The mixture was concentrated. The residue was dissolved in diethyl ether and washed with water (3×). The organic phase was dried (MgSO₄), filtered and concentrated. The product was purified by column chromatography (SiO₂, cyclohexane=>cyclohexane/ethyl acetate 2:3) to give 3-chloro-5-(2-oxo-pyrrolidin-1-yl)-benzoic acid methyl ester (414 mg) as a colorless solid. MS 254.4 ([M+H]⁺)

128.3 In analogy to example 8.3, 3-chloro-5-(2-oxo-pyrrolidin-1-yl)-benzoic acid methyl ester was hydrolyzed to give 3-chloro-5-(2-oxo-pyrrolidin-1-yl)-benzoic acid. Colorless solid. MS 237.8 ([M−H]⁻)

128.4 3-Chloro-5-(2-oxo-pyrrolidin-1-yl)-benzoic acid was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-(2-oxo-pyrrolidin-1-yl)-benzamide. Colorless solid. MS 427.4 ([M+H]⁺)

128.5 N-(2-Carbamoylmethoxy-4-cyano-benzyl)-3-chloro-5-(2-oxo-pyrrolidin-1-yl)-benzamide was converted to N-(4-carbamimidoyl-2-carbamoylmethoxy-benzyl)-3-chloro-5-(2-oxo-pyrrolidin-1-yl)-benzamide hydrochloride according to general procedure C. Colorless solid. MS 444.5 ([M+H]⁺)

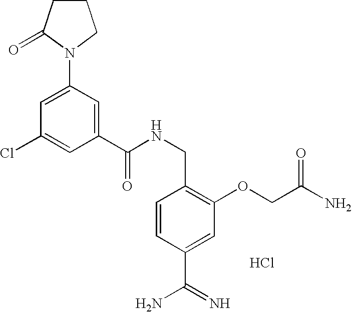

### Example 129

128.1 A stirred solution of 3-amino-5-chloro-benzoic acid methyl ester (CAS 21961-31-9, 1.94 g) in THF (120 ml) was cooled down to −74° C. A 1 M solution of sodium bis(trimethylsilyl)amide in THF (31.4 ml) was slowly (5 min) added. The mixture was stirred at −74° C. for 10 min. 3-Chloropropanesulfonyl chloride (2.83 g) was slowly added. The mixture was stirred for 1 h at −74° C. A 1 M solution of sodium bis(trimethylsilyl)amide in THF (6.3 ml) was added. After 5 min, 3-chloropropanesulfonyl chloride (0.61 ml) was added. The mixture was stirred at −74° C. for 2 h. Another 0.61 ml of 3-chloropropanesulfonyl chloride was added and the mixture was stirred for 1.5 h. The reaction mixture was poured into a mixture of ice water (100 ml), saturated aqueous NH₄Cl solution (100 ml) and ethyl acetate (250 ml). The mixture was extracted with ethyl acetate. The organic phase was washed with brine, dried, filtered and concentrated to give 6 g of a crude product as a brown oil.

The crude product (3.26 g) was dissolved in N,N-dimethylacetamide (30 ml). Potassium-tert-butylate (1.15 g) and potassium iodide (50 mg) were added=>exothermic reaction. The mixture was stirred for 10 min at r.t. and then heated to 80° C. for 4 h and to 40° C. for 14 h. The mixture was poured into ice water and extracted with ethyl acetate. The organic phase was washed with water, dried, filtered and concentrated. The product was purified by column chromatography (SiO₂, CH₂Cl₂=>CH₂Cl₂/MeOH 4:1) to give 3-chloro-5-(1,1-dioxo-isothiazolidin-2-yl)-benzoic acid methyl ester (244 mg) as an off-white oil. MS 307.0 ([M+NH₄]⁺)

129.2 In analogy to example 8.3, 3-chloro-5-(1,1-dioxo-isothiazolidin-2-yl)-benzoic acid methyl ester was hydrolyzed to give 3-chloro-5-(1,1-dioxo-isothiazolidin-2-yl)-benzoic acid. Off-white solid. MS 274.0 ([M−H]⁻)

129.3 3-Chloro-5-(1,1-dioxo-isothiazolidin-2-yl)-benzoic acid was coupled with 2-(2-aminomethyl-5-cyano-phenoxy)-acetamide hydrochloride (BB2) according to general procedure A to give N-(2-carbamoylmethoxy-4-cyano-benzyl)-3-chloro-5-(1,1-dioxo-isothiazolidin-2-yl)-benzamide. White solid. MS 463.0 ([M+H]⁺)

129.4 N-(2-Carbamoylmethoxy-4-cyano-benzyl)-3-chloro-5-(1,1-dioxo-isothiazolidin-2-yl)-benzamide was converted to N-(4-carbamimidoyl-2-carbamoylmethoxy-benzyl)-3-chloro-5-(1,1-dioxo-isothiazolidin-2-yl)-benzamide hydrochloride according to general procedure C. White solid. MS 479.8 ([M+H]⁺)

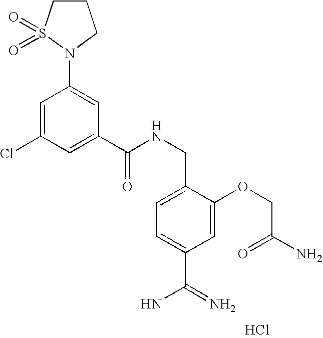

### Example 130

130.1 3-(5-Methyl-benzoimidazol-1-yl)-benzoic acid (CAS 211555-39-4) was coupled with 2-(2-aminomethyl-5-cyano-phenoxy)-acetamide hydrochloride (BB2) according to general procedure A to give N-(2-carbamoylmethoxy-4-cyano-benzyl)-3-(5-methyl-benzoimidazol-1-yl)-benzamide. Off-white solid. MS 440.4 ([M+H]³⁰ )

130.2 N-(2-Carbamoylmethoxy-4-cyano-benzyl)-3-(5-methyl-benzoimidazol-1-yl)-benzamide was converted to N-(4-carbamimidoyl-2-carbamoylmethoxy-benzyl)-3-(5-methyl-benzoimidazol-1-yl)-benzamide hydrochloride according to general procedure C. Colorless foam. MS 457.5 ([M+H]⁺)

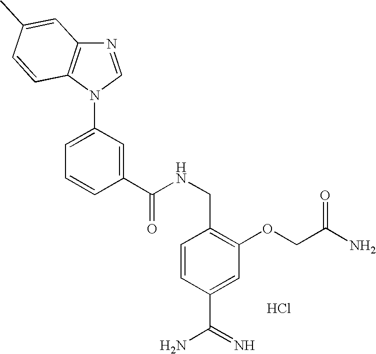

### Example 131

131.1 Benzoic acid was coupled with 2-(2-aminomethyl-5-cyano-phenoxy)-acetamide hydrochloride (BB2) according to general procedure A to give N-(2-carbamoylmethoxy-4-cyano-benzyl)-benzamide. Off-white solid. MS 310.4 ([M+H]⁺)

131.2 N-(2-Carbamoylmethoxy-4-cyano-benzyl)-benzamide was converted to N-(4-carbamimidoyl-2-carbamoylmethoxy-benzyl)-benzamide hydrochloride according to general procedure C. Off-white solid. MS 327.0 ([M+H]⁺)

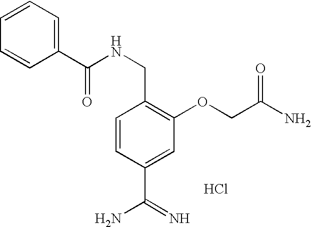

### Example 132

132.1 3,5-Bis(dimethylamino)benzoic acid was coupled with 2-(2-aminomethyl-5-cyano-phenoxy)-acetamide hydrochloride (BB2) according to general procedure A to give N-(2-carbamoylmethoxy-4-cyano-benzyl)-3,5-bis-dimethylamino-benzamide. Yellow foam. MS 396.3 ([M+H]⁺)

132.2 N-(2-Carbamoylmethoxy-4-cyano-benzyl)-3,5-bis-dimethylamino-benzamide was converted to N-(4-carbamimidoyl-2-carbamoylmethoxy-benzyl)-3,5-bis-dimethylamino-benzamide hydrochloride according to general procedure C. Off-white solid. MS 327.0 ([M+H]⁺)

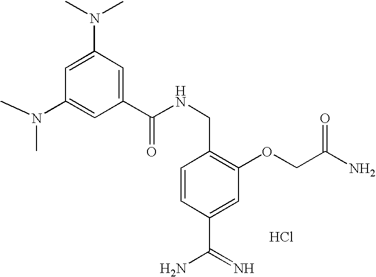

### Example 133

133.1 3-Chloro-5-isobutyrylamino-benzoic acid (Example 95.1, 0.529 g) was coupled with 4-aminomethyl-3-hydroxy-benzonitrile hydrochloride according to general procedure A to yield, after flash chromatography (SiO₂, hexane/AcOEt=7/3), 0.912 g of 3-chloro-N-(4-cyano-2-nitro-benzyl)-5-isobutyrylamino-benzamide, MS 418.2 ([M+NH4]⁺).

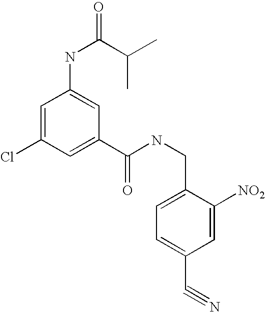

133.2 0.450 g of 3-chloro-N-(4-cyano-2-nitro-benzyl)-5-isobutyrylamino-benzamide was dissolved in 9 ml of ethyl acetate and hydrogenated over 0.180 g of Pd on charcoal (10%) at atmospheric pressure and ambient temperature. After 6 h, the reaction mixture was filtered over a pad of Celite, rinsed generously with ethyl acetate, and evaporated to dryness to yield 0.363 g of N-(2-amino-4-cyano-benzyl)-3-chloro-5-isobutyrylamino-benzamide as yellow foam. MS [M+H]⁺=371.0.

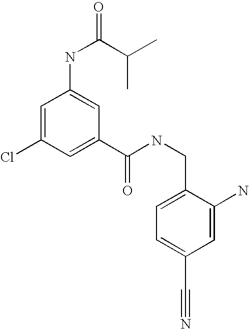

133.3 N-(2-Amino-4-cyano-benzyl)-3-chloro-5-isobutyrylamino-benzamide (0.180 g) and acetaldehyde (0.14 ml, 5 eq.) were dissolved in 8 ml of MeOH. One added a solution of ZnCl₂ (0.265 g, 4 eq.) and NaCNBH₃ (0.092 g, 3. eq.) in 2 ml of MeOH and stirred for 2 h at ambient temperature. Pouring onto crashed ice/NH₄Cl-solution, twofold extraction with AcOEt, washing with water, drying over sodium sulfate, and evaporation of the solvents, followed by flash chromatography (SiO₂, hexane/AcOEt=6/4) afforded 0.157 g of 3-chloro-N-(4-cyano-2-ethylamino-benzyl)-5-isobutyrylamino-benzamide as off-white solid. MS [M+H]⁺=399.4.

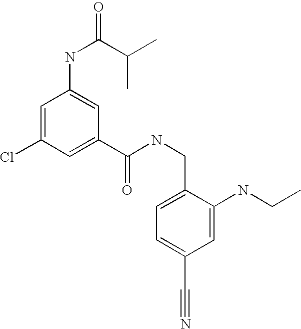

133.4 3-Chloro-N-(4-cyano-2-ethylamino-benzyl)-5-isobutyrylamino-benzamide was subjected to the Pinner reaction as described in general procedure C to yield after flash chromatography (SiO₂, AcOEt/acetone/AcOH/water=6/2/1/1) and crystallization from acetonitrile/diethyl ether 0.097 g of (4-carbamimidoyl-2-ethylamino-benzyl)-3-chloro-5-isobutyrylamino-benzamide; compound with acetic acid, as light yellow solid. MS [M+H]⁺=416.3.

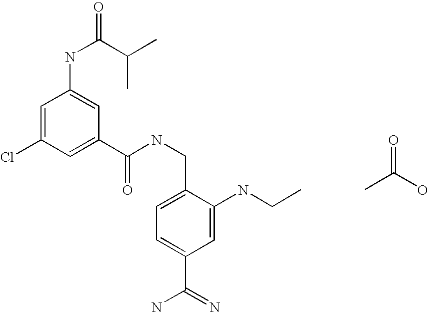

### Example 134

[4-Carbamimidoyl-2-(2-fluoro-benzylamino)-benzyl]-3-chloro-5-isobutyrylamino-benzamide; compound with acetic acid, was prepared in analogy to example 133, but using in step 3] 2-fluorobenzaldehyde instead of acetaldehyde for the reductive amination, as light yellow solid. MS [M+H]⁺=496.1.

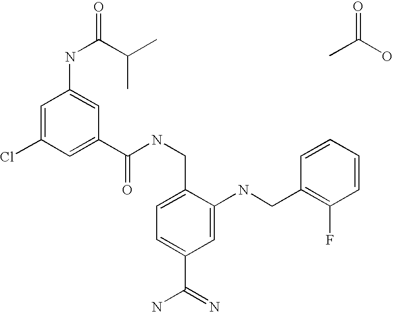

### Example 135

135.1 3-Chloro-5-phenylacetylamino-benzoic acid was prepared in analogy to example 95.1, but using phenyl-acetyl chloride instead of isobutyryl chloride, as white crystals. MS [M−H]⁻=288.1.

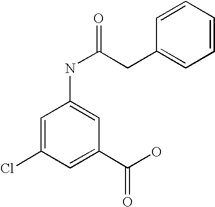

135.2 3-Chloro-5-phenylacetylamino-benzoic acid (0.340 g) was coupled with 4-aminomethyl-3-hydroxy-benzonitrile hydrochloride according to general procedure A to yield, after flash chromatography (SiO₂, hexane/AcOEt=1/1), 0.465 g of 3-chloro-N-(4-cyano-2-hydroxy-benzyl)-5-phenylacetylamino-benzamide as white crystals. MS [M−H]⁻=418.0.

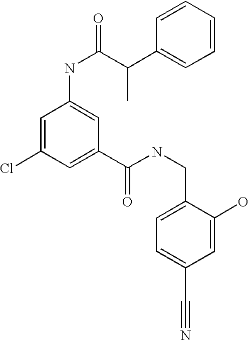

135.3 3-Chloro-N-(4-cyano-2-hydroxy-benzyl)-5-phenylacetylamino-benzamide (0.142 g) in acetonitrile (3 ml) was treated successively with cesium carbonate (0.132 g) and iodoacetamide (0.069 g), and the reaction mixture was stirred at ambient temperature overnight. Pouring onto crashed ice/NH₄Cl-solution, twofold extraction with AcOEt, washing with water and brine, drying over magnesium sulfate, and evaporation of the solvents, followed by crystallisation from AcOEt, yielded 0.117 g of N-(2-carbamoylmethoxy-4-cyano-benzyl)-3-chloro-5-phenylacetylamino-benzamide as white crystals. MS [M+NH₄]⁺=494.5.

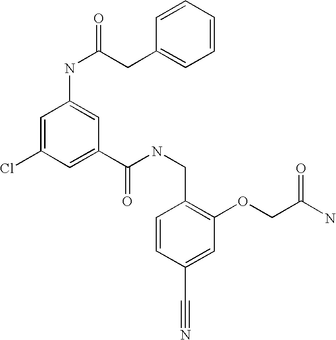

135.4 N-(2-carbamoylmethoxy-4-cyano-benzyl)-3-chloro-5-phenylacetylamino-benzamide was subjected to the Pinner reaction as described in general procedure C to yield after flash chromatography (SiO₂, AcOEt/acetone/AcOH/water=6/2/1/1) and crystallization from acetonitrile 0.049 g of N-(4-carbamimidoyl-2-carbamoylmethoxy-benzyl)-3-chloro-5-phenylacetylamino-benzamide; compound with acetic acid, as off-white crystals. MS [M+H]⁺=494.5.

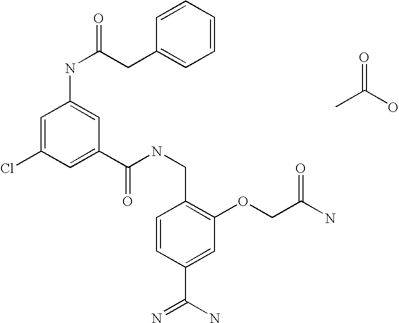

### Example 136

N-(4-Carbamimidoyl-2-methylcarbamoylmethoxy-benzyl)-3-chloro-5-phenylacetylamino-benzamide; compound with acetic acid, was prepared in analogy to example 135, but using in step 3] 2-chloro-N-methylacetamide/KI as electrophile instead of iodoacetamide, as white crystals. MS [M+H]⁺=508.7.

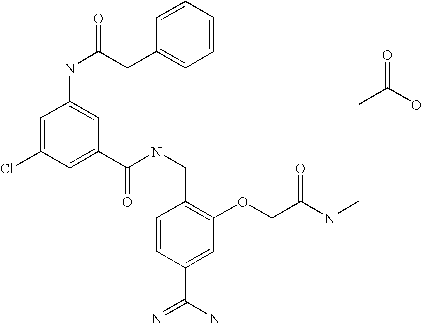

### Example 137

N-{4-Carbamimidoyl-2-[(pyridin-2-ylmethyl)-amino]-benzyl}-3-chloro-5-methanesulfonylamino-benzamide; compound with HCl, was prepared in analogy to example 102, but using in step 2] pyridine-2-carboxaldehyde instead of acetaldehyde for the reductive amination, as white crystals. MS [M−H]−=485.4.

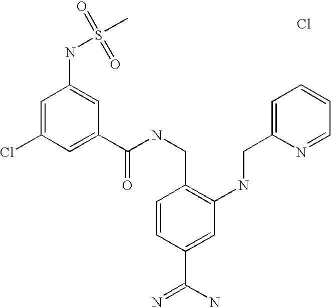

### Example 138

138.1 3-Chloro-N-(4-cyano-2-methylcarbamoylmethoxy-benzyl)-5-(methanesulfonyl-methylcarbamoylmethyl-amino)-benzamide was prepared from 3-chloro-N-(4-cyano-2-hydroxy-benzyl)-5-methanesulfonylamino-benzamide as described in example 101, but using 2-chloro-N-methylacetamide/KI as electrophile instead of iodoacetamide, as off-white crystals. MS [M+H]⁺=522.5.

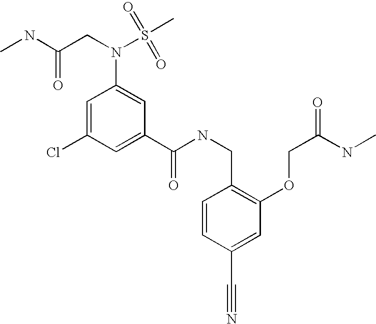

138.2 3-Chloro-N-(4-cyano-2-methylcarbamoylmethoxy-benzyl)-5-(methanesulfonyl-methylcarbamoylmethyl-amino)-benzamide was subjected to the Pinner reaction as described in general procedure C to yield, after flash chromatography (SiO₂, AcOEt/acetone/AcOH/water=6/2/1/1), N-(4-Carbamimidoyl-2-methylcarbamoylmethoxy-benzyl)-3-chloro-5-(methanesulfonyl-methylcarbamoylmethyl-amino)-benzamide; compound with acetic acid, as off-white crystals. MS [M+H]⁺=539.4.

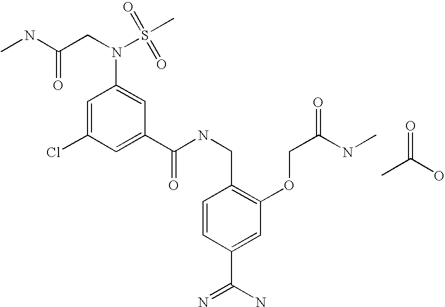

### Example 139

{[3-(4-Carbamimidoyl-2-ethoxycarbonylmethoxy-benzylcarbamoyl)-5-chloro-phenyl]-methanesulfonyl-amino}-acetic acid ethyl ester; compound with acetic acid, was prepared in analogy to example 138, but using as electrophile ethyl bromoacetate instead of 2-chloro-N-methylacetamide/KI, as light yellow viscous oil. MS [M+H]⁺=569.4.

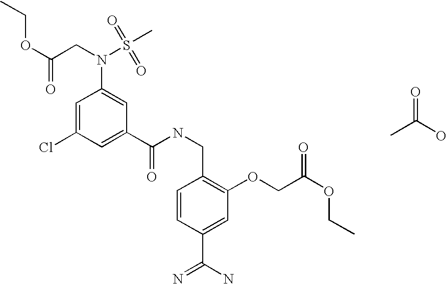

### Example 140

140.1 3-Chloro-5-methanesulfonylamino-benzoic acid methyl ester (0.320 g) in acetonitrile (6 ml) was treated successively with cesium carbonate (0.870 g) and benzyl bromide (0.415 g), and the reaction mixture was stirred at 40° C. for 6 h. Pouring onto crashed ice/NH₄Cl-solution, twofold extraction with AcOEt, washing with water and brine, drying over magnesium sulfate, and evaporation of the solvents, followed by flash chromatography (SiO₂, hexane/AcOEt=7/3), produced 0.371 g of 3-(benzyl-methanesulfonyl-amino)-5-chloro-benzoic acid methyl ester as off-white crystals. MS [M]⁺=353.0.

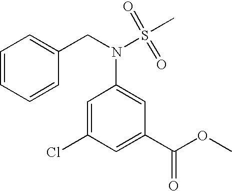

140.2 0.368 g of 3-(benzyl-methanesulfonyl-amino)-5-chloro-benzoic acid methyl ester was dissolved in 8.4 ml of THE/EtOH=1/1, treated with 4.2 ml (4 eq.) of 1N NaOH and kept at ambient temperature for 1.5 h. The reaction mixture was then poured onto crashed ice/AcOEt/HCl dil., the aqueous phase extracted again with AcOEt; the combined organic layers were washed with water, dried over magnesium sulfate, and evaporated to dryness to leave, after crystallisation from hexane/AcOEt 0.346 g of 3-(benzyl-methanesulfonyl-amino)-5-chloro-benzoic acid as off-white crystals. MS [M−H]⁻=338.0.

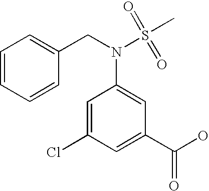

140.3 3-(Benzyl-methanesulfonyl-amino)-5-chloro-benzoic acid (0.326 g) was coupled with 4-aminomethyl-3-hydroxy-benzonitrile hydrochloride according to general procedure A to yield, after flash chromatography (SiO₂, hexane/AcOEt=1/1), 0.502 g of 3-(benzyl-methanesulfonyl-amino)-5-chloro-N-(4-cyano-2-hydroxy-benzyl)-benzamide as white foam. MS [M+NH₄]⁺=487.4.

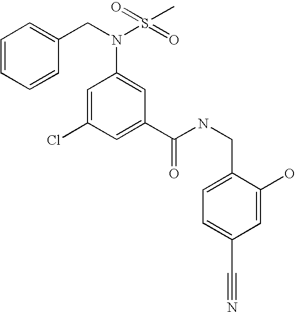

140.3 3-(Benzyl-methanesulfonyl-amino)-5-chloro-N-(4-cyano-2-hydroxy-benzyl)-benzamide (0.155 g) was condensed with iodoacetamide as described in example 95.3 to yield 0.118 g of 3-(benzyl-methanesulfonyl-amino)-N-(2-carbamoylmethoxy-4-cyano-benzyl)-5-chloro-benzamide as white crystals. MS [M+NH₄]⁺=544.4.

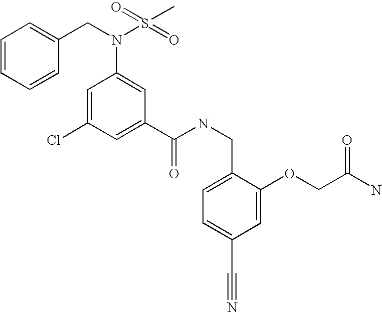

140.5 nzyl-methanesulfonyl-amino)-N-(2-carbamoylmethoxy-4-cyano-benzyl)-5-chloro-benzamide was subjected to the Pinner reaction as described in general procedure C to yield, after flash chromatography (SiO₂, AcOEt/acetone/AcOH/water=11/2/1/1) and crystallization from acetonitrile, 0.089 g of 3-(benzyl-methanesulfonyl-amino)-N-(4-carbamimidoyl-2-carbamoylmethoxy-benzyl)-5-chloro-benzamide; compound with acetic acid, as white solid. MS [M+H]⁺=544.4.

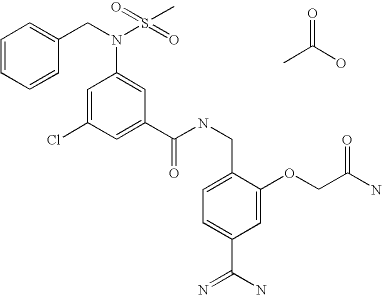

### Example 141

3-(Benzyl-methanesulfonyl-amino)-N-(4-carbamimidoyl-2-methylcarbamoylmethoxy-benzyl)-5-chloro-benzamide; compound with acetic acid, was prepared in analogy to example 140, but using in step 4] 2-chloro-N-methylacetamide/KI as electrophile instead of iodoacetamide, as white crystals. MS [M+H]⁺=558.4

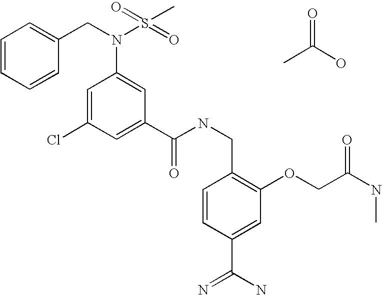

### Example 142

N-(4-Carbamimidoyl-2-carbamoylmethoxy-benzyl)-3-(carbamoylmethyl-ethanesulfonyl-amino)-5-chloro-benzamide; compound with acetic acid, was prepared in analogy to example 101, but using ethanesulfonylchloride instead of methanesulfonylchloride as starting material, as colorless solid. MS [M+H]⁺=525.1.

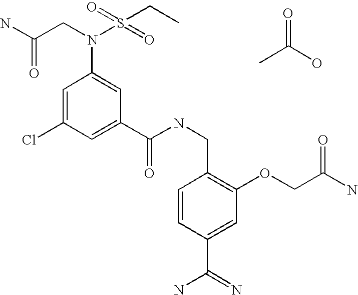

### Example 143

143.1 ro-5-(propane-1-sulfonylamino)-benzoic acid methyl ester was prepared as described in example 99.1, but using propanesulfonylchloride instead of methanesulfonylchloride, as off-white solid. MS [M+NH₄]⁺=309.1.

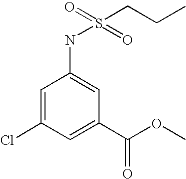

143.2 ro-5-(propane-1-sulfonylamino)-benzoic acid methyl ester (0.210 g) in acetonitrile (3.6 ml) was treated successively with cesium carbonate (0.281 g) and iodoacetamide (0.146 g), and the reaction mixture was stirred at 40° C.for 2 h. Pouring onto crashed ice/NH₄Cl-solution, twofold extraction with AcOEt, washing with water, drying over sodium sulfate, and evaporation of the solvents, followed by flash chromatography (SiO₂, hexane/AcOEt=1/1), afforded 0.233 g of 3-[carbamoylmethyl-(propane-1-sulfonyl)-amino]-5-chloro-benzoic acid methyl ester as colorless solid. MS [M+NH₄]⁺=366.2.

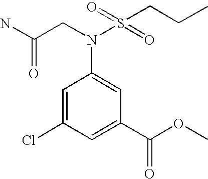

143.3 [Carbamoylmethyl-(propane-1-sulfonyl)-amino]-5-chloro-benzoic acid methyl ester (0.233 g) was dissolved in 5.3 ml of THF/EtOH=1/1, treated with 2.67 ml (4 eq.) of 1N NaOH and kept at ambient temperature for 2 h. The reaction mixture was then poured onto crashed ice/AcOEt/HCl dil., the aqueous phase extracted again with AcOEt; the combined organic layers were washed with water, dried over sodium sulfate, and evaporated to dryness to leave 0.188 g of 3-[carbamoylmethyl-(propane-1-sulfonyl)-amino]-5-chloro-benzoic acid as off-white solid. MS [M]⁺=334.0.

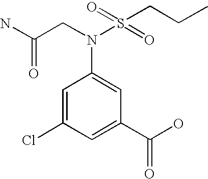

143.4 3-[Carbamoylmethyl-(propane-1-sulfonyl)-amino]-5-chloro-benzoic acid (0.188 g) was coupled with 4-aminomethyl-3-hydroxy-benzonitrile hydrochloride according to general procedure A to yield, after flash chromatography (SiO₂, hexane/AcOEt=3/7), 0.169 g of 3-[carbamoylmethyl-(propane-1-sulfonyl)-amino]-5-chloro-N-(4-cyano-2-hydroxy-benzyl)-benzamide as off-white foam. MS [M+NH₄]⁺=482.6.

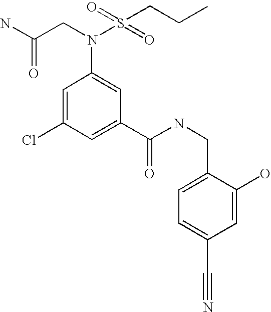

143.5 3-[Carbamoylmethyl-(propane-1-sulfonyl)-amino]-5-chloro-N-(4-cyano-2-hydroxy-benzyl)-benzamide (0.169 g) in acetonitrile (3 ml) was treated successively with cesium carbonate (0.130 g), 2-chloro-N-methylacetamide (0.041 g) and potassium iodide (0.060 g), and the reaction mixture was stirred at 40° C. for three days. Pouring onto crashed ice/NH₄Cl-solution, twofold extraction with large amounts of AcOEt, washing with water, drying over sodium sulfate, and evaporation of the solvents, followed by crystallisation from AcOEt/hexane, afforded 0.159 g of 3-[carbamoylmethyl-(propane-1-sulfonyl)-amino]-5-chloro-N-(4-cyano-2-methylcarbamoylmethoxy-benzyl)-benzamide as light yellow foam. MS [M+H]⁺=536.3.

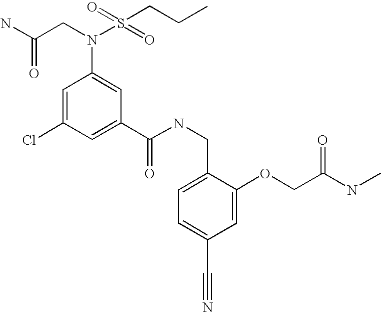

143.6 3-[Carbamoylmethyl-(propane-1-sulfonyl)-amino]-5-chloro-N-(4-cyano-2-methylcarbamoylmethoxy-benzyl)-benzamide was subjected to the Pinner reaction as described in general procedure C to yield, after flash chromatography (SiO₂, AcOEt/acetone/AcOH/water=11/2/1/1) and crystallization from acetonitrile, 0.067 g of N-(4-carbamimidoyl-2-methylcarbamoylmethoxy-benzyl)-3-[carbamoylmethyl-(propane-1-sulfonyl)-amino]-5-chloro-benzamide; compound with acetic acid, as off-white solid. MS [M+H]⁺=553.3.

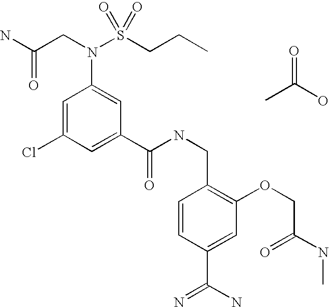

### Example 144

N-(4-Carbamimidoyl-2-methylcarbamoylmethoxy-benzyl)-3-chloro-5-[methylcarbamoylmethyl-(propane-1-sulfonyl)-amino]-benzamide; compound with acetic acid, was prepared in analogy to example 143, but using in step 2 2-chloro-N-methylacetamide/KI as electrophile instead of iodoacetamide, as off-white solid. MS [M+H]⁺=567.3.

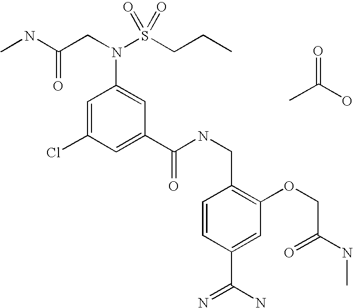

### Example 145

N-(4-Carbamimidoyl-2-carbamoylmethoxy-benzyl)-3-chloro-5-[methylcarbamoylmethyl-(propane-1-sulfonyl)-amino]-benzamide; compound with acetic acid, was prepared in analogy to example 144, but using in the penultimate step iodoacetamide as electrophile instead of 2-chloro-N-methylacetamide/KI, as off-white semisolid. MS [M+H]⁺=553.3.

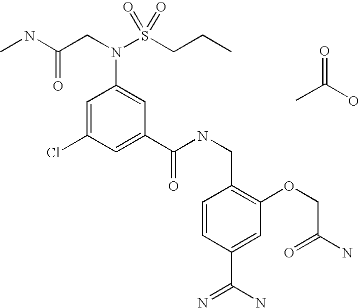

### Example 146

N-(4-Carbamimidoyl-2-carbamoylmethoxy-benzyl)-3-(carbamoylmethyl-phenylmethanesulfonyl-amino)-5-chloro-benzamide; compound with acetic acid, was prepared in analogy to example 145, but starting the whole reaction sequence with phenyl-methanesulfonyl chloride, as off-white solid. MS [M+H]⁺=587.3.

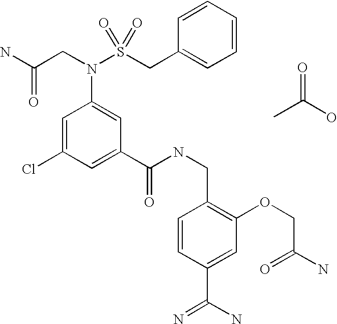

### Example 147

N-(4-Carbamimidoyl-2-carbamoylmethoxy-benzyl)-3-chloro-5-(methylcarbamoylmethyl-phenylmethanesulfonyl-amino)-benzamide; compound with acetic acid, was prepared in analogy to example 146, but using for the first alkylation step 2-chloro-N-methylacetamide/KI as electrophile instead of iodoacetamide, as off-white solid. MS [M+H]⁺=601.3.

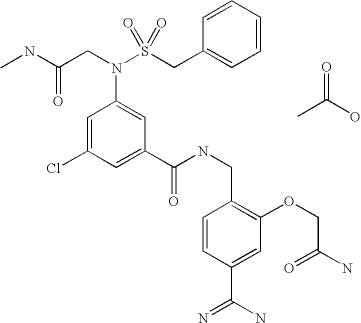

### Example 148

N-(4-Carbamimidoyl-2-methylcarbamoylmethoxy-benzyl)-3-chloro-5-(methylcarbamoylmethyl-phenylmethanesulfonyl-amino)-benzamide; compound with acetic acid, was prepared in analogy to example 146, but using for both alkylation steps 2-chloro-N-methylacetamide/KI as electrophile instead of iodoacetamide, as off-white solid. MS [M+H]⁺=615.4.

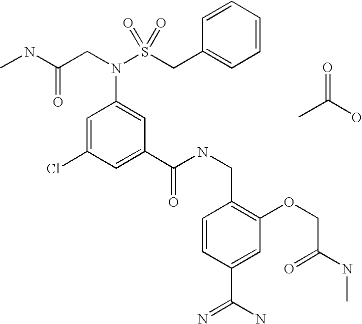

### Example 149

N-(4-Carbamimidoyl-2-carbamoylmethoxy-benzyl)-3-[carbamoylmethyl-(propane-2-sulfonyl)-amino]-5-chloro-benzamide; compound with acetic acid, was prepared in analogy to example 146, but starting the whole reaction sequence with isopropylsulfonyl chloride instead of α-toluenesulphonyl chloride, as off-white solid. MS [M+H]⁺=539.3.

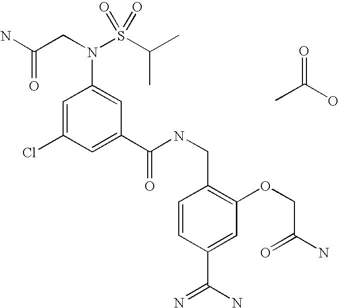

### Example 150

N-(4-Carbamimidoyl-2-methylcarbamoylmethoxy-benzyl)-3-[carbamoylmethyl-(propane-2-sulfonyl)-amino]-5-chloro-benzamide; compound with acetic acid, was prepared in analogy to example 149, but using for the second alkylation step 2-chloro-N-methylacetamide/KI as electrophile instead of iodoacetamide, as off-white solid. MS [M+H]⁺=553.3.

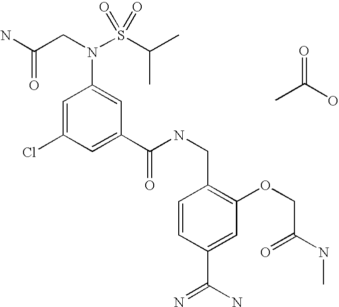

### Example 151

151.1 3-Chloro-5-methanesulfonylamino-benzoic acid methyl ester (0.438 g) in acetonitrile (9 ml) was treated successively with cesium carbonate (2.165 g), KI (0.551 g), and 2-chloromethylpyridine hydrochloride (0.532 g), and the reaction mixture was stirred at 45° C. for 3 h. Pouring onto crashed ice/NH₄Cl-solution, twofold extraction with AcOEt, washing with water and brine, drying over magnesium sulfate, and evaporation of the solvents, followed by flash chromatography (SiO₂, hexane/AcOEt=1/1), yielded 0.496 g of 3-chloro-5-(methanesulfonyl-pyridin-2-ylmethyl-amino)-benzoic acid methyl ester as off-white crystals. MS [M+H]⁺=355.0.

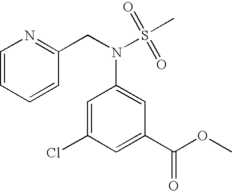

151.2 3-Chloro-5-(methanesulfonyl-pyridin-2-ylmethyl-amino)-benzoic acid methyl ester (0.492 g) was dissolved in 11 ml of THF/EtOH=1/1, treated with 5.54 ml (4 eq.) of 1N NaOH and kept at ambient temperature for 2.5 h. The reaction mixture was then poured onto crashed ice/AcOEt/HCl dil., the aqueous phase extracted again with AcOEt; the combined organic layers were washed with water, dried over magnesium sulfate, and evaporated to dryness to leave 0.482 g of 3-chloro-5-(methanesulfonyl-pyridin-2-ylmethyl-amino)-benzoic acid as off-white foam. MS [M−H]⁻=339.0.

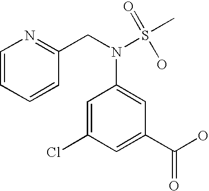

151.3 3-Chloro-5-(methanesulfonyl-pyridin-2-ylmethyl-amino)-benzoic acid (0.478 g) was coupled with 4-aminomethyl-3-hydroxy-benzonitrile hydrochloride according to general procedure A to yield, after direct crystallisation from methanol, 0.585 g of 3-chloro-N-(4-cyano-2-hydroxy-benzyl)-5-(methanesulfonyl-pyridin-2-ylmethyl-amino)-benzamide as white crystals. MS [M+H]⁺=471.0.

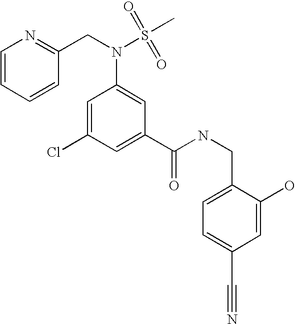

151.4 3-Chloro-N-(4-cyano-2-hydroxy-benzyl)-5-(methanesulfonyl-pyridin-2-ylmethyl-amino)-benzamide (0.133 g) in acetonitrile (3 ml) was treated successively with cesium carbonate (0.110 g) and iodoacetamide (0.057 g), and the reaction mixture was. stirred at 20° C. for two days. Pouring onto crashed ice/NH₄Cl-solution, twofold extraction with AcOEt, washing with water and brine, drying over magnesium sulfate, and evaporation of the solvents, followed by direct crystalisation from AcOEt/hexane, produced 0.147 g of N-(2-carbamoylmethoxy-4-cyano-benzyl)-3-chloro-5-(methanesulfonyl-pyridin-2-ylmethyl-amino)-benzamide as light yellow crystals. MS [M+H]⁺=528.0.

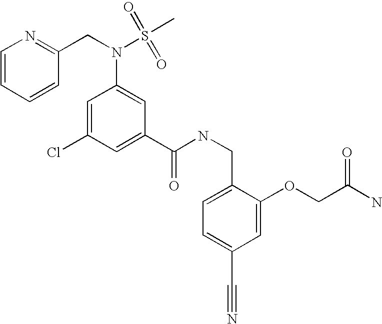

151.5 N-(2-carbamoylmethoxy-4-cyano-benzyl)-3-chloro-5-(methanesulfonyl-pyridin-2-ylmethyl-amino)-benzamide (0.144 g) was subjected to the Pinner reaction as described in general procedure C to yield, after crystallisation from AcOEt/acetone/AcOH/water=11/2/1/1, 0.116 g of N-(4-carbamimidoyl-2-carbamoylmethoxy-benzyl)-3-chloro-5-(methanesulfonyl-pyridin-2-ylmethyl-amino)-benzamide; compound with HCl, as off-white crystals. MS [M+H]⁺=545.2.

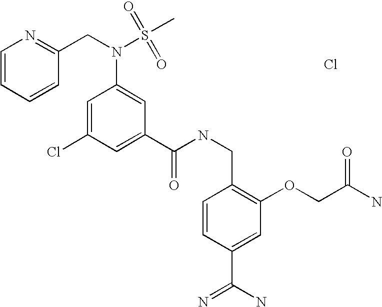

### Example 152

N-(4-Carbamimidoyl-2-carbamoylmethoxy-benzyl)-3-chloro-5-(ethanesulfonyl-pyridin-2-ylmethyl-amino)-benzamide; compound with HCl, was prepared in analogy to example 151, but starting the whole reaction sequence with ethanesulfonyl chloride instead of methanesulphonyl chloride, as white crystals. MS [M+H]⁺=559.3.

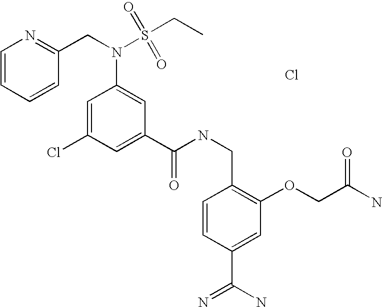

### Example 153

[2-(5-Carbamimidoyl-2-{[3-chloro-5-(ethanesulfonyl-pyridin-2-ylmethyl-amino)-benzoylamino]-methyl}-phenoxy)-acetylamino]-acetic acid ethyl ester; compound with acetic acid, was prepared in analogy to example 152, but alkylating in Step 4 with N-(chloroacetyl)glycine ethyl ester instead of iodoacetamide, as off-white foam. MS [M+H]⁺=645.2.

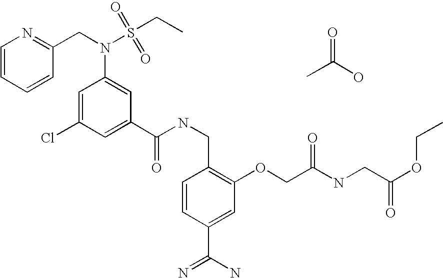

### Example 154

[2-(5-Carbamimidoyl-2-{[3-chloro-5-(methanesulfonyl-pyridin-2-ylmethyl-amino)-benzoylamino]-methyl}-phenoxy)-acetylamino]-acetic acid ethyl ester; compound with acetic acid, was prepared in analogy to example 153, but starting the whole reaction sequence with methanesulfonyl chloride instead of ethanesulfonyl chloride, as off-white foam. MS [M+H]⁺=631.0.

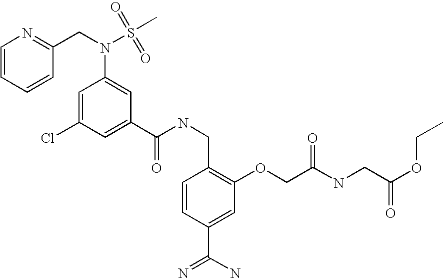

### Example 155

N-(4-Carbamimidoyl-2-methylcarbamoylmethoxy-benzyl)-3-chloro-5-(ethanesulfonyl-pyridin-2-ylmethyl-amino)-benzamide; compound with HCl, was prepared in analogy to example 152, but using for the second alkylation step 2-chloro-N-methylacetamide/KI as electrophile instead of iodoacetamide, as white crystals. MS [M+H]⁺=573.3.

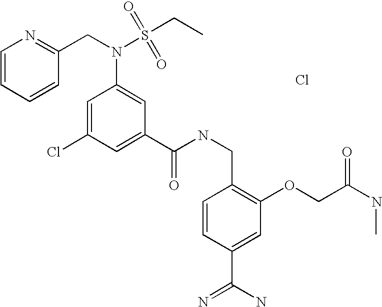

### Example 156

[2-(5-Carbamimidoyl-2-{[3-chloro-5-(ethanesulfonyl-pyridin-2-ylmethyl-amino)-benzoylamino]-methyl}-phenoxy)-acetylamino]-acetic acid ethyl ester; compound with acetic acid (example 153, 0.144 g), was dissolved in 1.4 ml of THF, treated with 0.85 ml (4 eq.) of 1N LiOH and kept at ambient temperature for 2 h, when TLC analysis indicated the absence of starting material. The reaction mixture was then carefully evaporated to dryness and poured directly on a flash column (SiO2). Elution with AcOEt/acetone/AcOH/water=6/2/1/1 delivered 0.147 g of [2-(5-carbamimidoyl-2-{[3-chloro-5-(ethanesulfonyl-pyridin-2-ylmethyl-amino)-benzoylamino]-methyl}-phenoxy)-acetylamino]-acetic acid; compound with acetic acid, as white foam. MS [M−H]⁻=615.3.

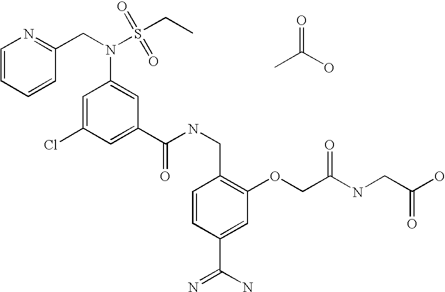

### Example 157

[2-(5-Carbamimidoyl-2-{[3-chloro-5-(methanesulfonyl-pyridin-2-ylmethyl-amino)-benzoylamino]-methyl}-phenoxy)-acetylamino]-acetic acid; compound with acetic acid, was prepared in analogy to example 156, but using as starting material [2-(5-carbamimidoyl-2-{[3-chloro-5-(methanesulfonyl-pyridin-2-ylmethyl-amino)-benzoylamino]-methyl}-phenoxy)-acetylamino]-acetic acid ethyl ester; compound with acetic acid (example 154), instead of [2-(5-carbamimidoyl-2-{[3-chloro-5-(ethanesulfonyl-pyridin-2-ylmethyl-amino)-benzoylamino]-methyl}-phenoxy)-acetylamino]-acetic acid ethyl ester; compound with acetic acid, as off-white crystals. MS [M−H]⁻=601.1.

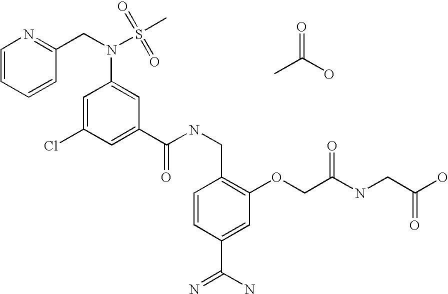

### Example 158

[2-(5-Carbamimidoyl-2-{[3-(carbamoylmethyl-methanesulfonyl-amino)-5-chloro-benzoylamino]-methyl}-phenoxy)-acetylamino]-acetic acid ethyl ester; compound with HCl, was prepared in analogy to example 154, but using in the first alkylation step iodoacetamide instead of 2-chloromethylpyridine hydrochloride, as off-white solid. MS [M+H]⁺=597.1.

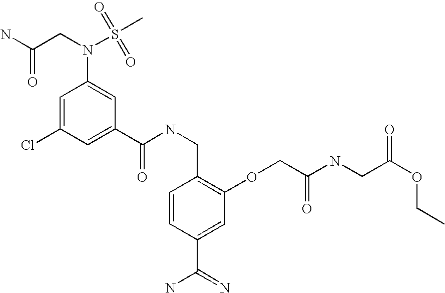

### Example 159

N-(4-Carbamimidoyl-2-carbamoylmethoxy-benzyl)-3-chloro-5-(phenylmethanesulfonyl-pyridin-2-ylmethyl-amino)-benzamide; compound with HCl, was prepared in analogy to example 152, but starting the whole reaction sequence α-toluenesulphonyl chloride instead of ethanesulfonyl chloride, as off-white solid. MS [M+H]⁺=621.2.

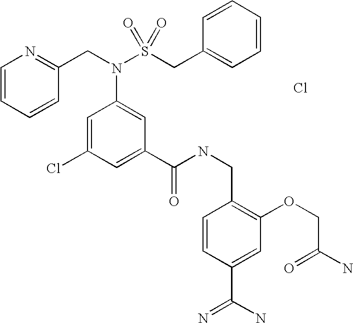

### Example 160

[2-(5-Carbamimidoyl-2-{[3-(carbamoylmethyl-methanesulfonyl-amino)-5-chloro-benzoylamino]-methyl}-phenoxy)-acetylamino]-acetic acid; compound with acetic acid, prepared in analogy to example 156, but using [2-(5-carbamimidoyl-2-{[3-(carbamoylmethyl-methanesulfonyl-amino)-5-chloro-benzoylamino]-methyl}-phenoxy)-acetylamino]-acetic acid ethyl ester; compound with HCl (example 158) as substrate, as off-white solid. MS [M−H]⁻=567.2.

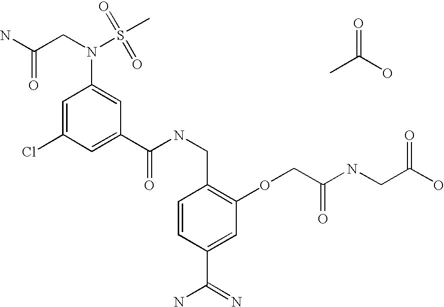

### Example 161

161.1 3-Amino-5-chloro-benzoic acid methyl ester (CAS 21961-31-9, 0.100 g) and acetone (0.40 ml, 10 eq.) were dissolved in 8 ml of MeOH. One added a solution of ZnCl₂ (0.294 g, 4 eq.) and NaCNBH₃ (0.102 g, 3. eq.) in 2 ml of MeOH and stirred for 20 h at 40° C. Pouring onto crashed ice/NH₄Cl-solution, twofold extraction with AcOEt, washing with water, drying over sodium sulfate, and evaporation of the solvents, followed by flash chromatography (SiO₂, hexane/AcOEt=9/1) yielded 0.094 g of 3-chloro-5-isopropylamino-benzoic acid methyl ester as yellow oil. MS [M+H]⁺=227.9.

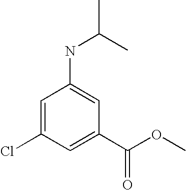

161.2 3-Chloro-5-isopropylamino-benzoic acid methyl ester (0.092 g) in acetonitrile (1 ml) was treated successively with cesium carbonate (0.197 g), potassium iodide (0.067 g), and benzyl bromide (0.124 g), and the mixture was allowed to react at 40° C. for two days. Pouring onto crashed ice/NH₄Cl-solution, twofold extraction with AcOEt, washing with water, drying over sodium sulfate, and evaporation of the solvents, followed by flash chromatography (SiO₂, hexane/AcOEt=9/1), produced 0.111 g of 3-(benzyl-isopropyl-amino)-5-chloro-benzoic acid methyl ester as light yellow oil. MS [M+H]⁺=318.0.

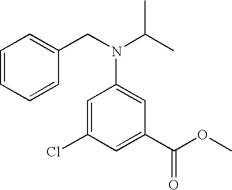

161.3 3-(Benzyl-isopropyl-amino)-5-chloro-benzoic acid methyl ester (0.111 g) was dissolved in 2.8 ml of THF/EtOH=1/1, treated with 1.40 ml (4 eq.) of 1N NaOH and kept at ambient temperature for 2 h. The reaction mixture was then poured onto crashed ice/AcOEt/HCl dil., the aqueous phase extracted again with AcOEt; the combined organic layers were washed with water, dried over sodium sulfate, and evaporated to dryness to leave 0.105 g of 3-(benzyl-isopropyl-amino)-5-chloro-benzoic acid as yellow oil. MS [M+H]⁺=304.0.

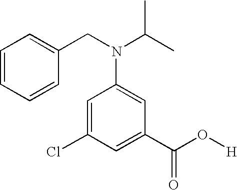

161.4 3-(Benzyl-isopropyl-amino)-5-chloro-benzoic acid (0.105 g) was coupled with 4-aminomethyl-3-hydroxy-benzonitrile hydrochloride according to general procedure A to yield, after flash chromatography (SiO₂, hexane/AcOEt=3/7), 0.132 g of 3-(benzyl-isopropyl-amino)-5-chloro-N-(4-cyano-2-hydroxy-benzyl)-benzamide as light yellow foam. MS [M−H]⁻=432.3.

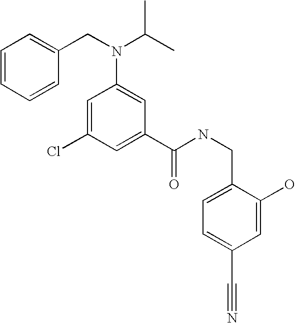

161.5 3-(Benzyl-isopropyl-amino)-5-chloro-N-(4-cyano-2-hydroxy-benzyl)-benzamide (0.130 g) in acetonitrile (2.3 ml) was treated successively with cesium carbonate (0.122 g) and iodoacetamide (0.061 g), and the reaction mixture was stirred at ambient temperature over night. Pouring onto crashed ice/NH₄Cl-solution, twofold extraction with large amounts of AcOEt, washing with water, drying over sodium sulfate, and evaporation of the solvents, followed by direct crystallisation from AcOEt/hexane, generated 0.120 g of 3-(benzyl-isopropyl-amino)-N-(2-carbamoylmethoxy-4-cyano-benzyl)-5-chloro-benzamide as off-white solid. MS [M+H]⁺=491.3.

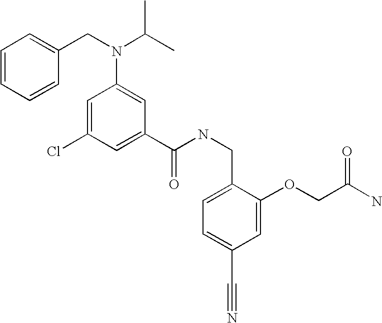

161.6 3-Benzyl-isopropyl-amino)-N-(2-carbamoylmethoxy-4-cyano-benzyl)-5-chloro-benzamide (0.120 g) was subjected to the Pinner reaction as described in general procedure C to yield, after twofold crystallisation from acetonitrile/diethyl ether, 0.138 g of 3-(benzyl-isopropyl-amino)-N-(4-carbamimidoyl-2-carbamoylmethoxy-benzyl)-5-chloro-benzamide; compound with HCl, as off-white solid. MS [M+H]⁺=508.4.

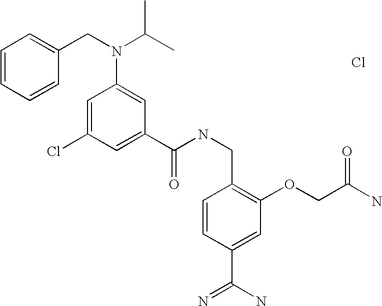

### Example 162

N-(4-Carbamimidoyl-2-methylcarbamoylmethoxy-benzyl)-3-chloro-5-(phenylmethanesulfonyl-pyridin-2-ylmethyl-amino)-benzamide; compound with 6 acetic acid, was prepared in analogy to example 159, but using in the second alkylation step step 2-chloro-N-methylacetamide/KI instead of iodoacetamide, as light brown solid. MS [M+H]⁺=635.2.

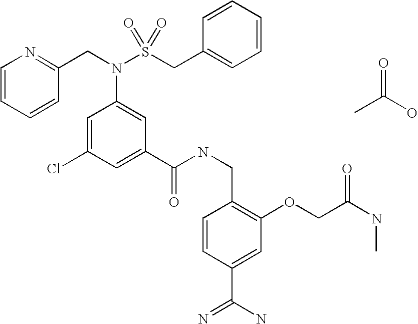

### Example 163

N-(4-Carbamimidoyl-2-carbamoylmethoxy-benzyl)-3-chloro-5-(isopropyl-pyridin-2-ylmethyl-amino)-benzamide; compound with acetic acid, was prepared in analogy to example 161, but using in the second step 2-chloromethyl-pyridine as alkylating agent instead of benzyl bromide, as off-white solid. MS [M+H]⁺=509.4.

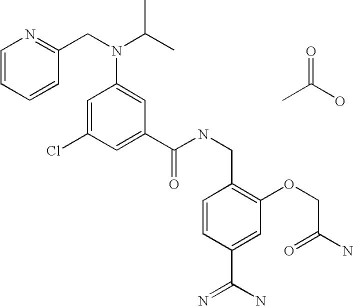

### Example 164

164.1 3-Chloro-5-methanesulfonylamino-benzoic acid methyl ester (0.570 g) in acetonitrile (10 ml) was treated successively with cesium carbonate (0.845 g), and iodoacetamide (0.440 g), and the mixture was allowed to react at ambient temperature for one day. Pouring onto crashed ice/NH₄Cl-solution, twofold extraction with AcOEt, washing with water, drying over sodium sulfate, and evaporation of the solvents, followed by crystallization from AcOEt/hexane produced 0.543 g of 3-(carbamoylmethyl-methanesulfonyl-amino)-5-chloro-benzoic acid methyl ester as off-white crystals. MS [M+H]⁺=321.0.

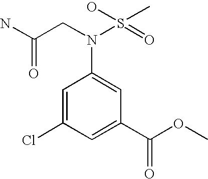

164.2 3-(Carbamoylmethyl-methanesulfonyl-amino)-5-chloro-benzoic acid methyl ester (0.539 g) was dissolved in 13.4 ml of THF/EtOH=1/1, treated with 6.70 ml (4 eq.) of 1N NaOH and kept at ambient temperature for 2 h. The reaction mixture was then poured onto crashed ice/AcOEt/HCl dil., the aqueous phase extracted again with AcOEt; the combined organic layers were washed with water, dried over magnesium sulfate, and evaporated to dryness to leave, after crystallization from AcOEt/hexane 0.436 g of 3-chloro-5-methanesulfonylamino-benzoic acid as white crystals. MS [M−H]⁻=305.0.

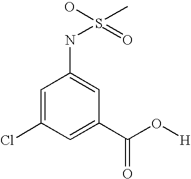

164.3 3-Chloro-5-methanesulfonylamino-benzoic acid (0.434 g) was coupled with 4-aminomethyl-3-hydroxy-benzonitrile hydrochloride according to general procedure A to yield, after flash chromatography (SiO₂, AcOEt), 0.671 g of 3-(carbamoylmethyl-methanesulfonyl-amino)-5-chloro-N-(4-cyano-2-nitro-benzyl)-benzamide, as light yellow crystals. MS [M+OAc]⁺=524.0.

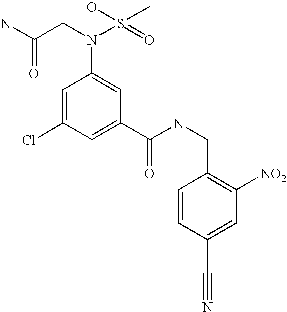

164.4 3-(Carbamoylmethyl-methanesulfonyl-amino)-5-chloro-N-(4-cyano-2-nitro-benzyl)-benzamide(0.670 g) was dissolved in 15 ml of AcOEt and hydrogenated over 0.27 g of Pd on charcoal (10%) at atmospheric pressure and ambient temperature. After 15 h, the reaction mixture was filtered over a pad of Celite and rinsed generously with AcOEt and MeOH. The combined filtrates were evaporated to dryness to leave 0.444 g of N-(2-amino-4-cyano-benzyl)-3-(carbamoylmethyl-methanesulfonyl-amino)-5-chloro-benzamide as off-white crystals. MS [M+H]⁺=436.1.

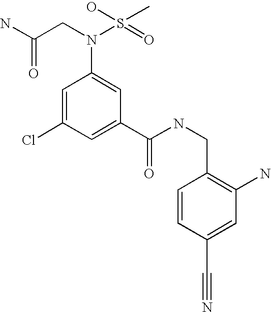

164.5 N-(2-Amino-4-cyano-benzyl)-3-(carbamoylmethyl-methanesulfonyl-amino)-5-chloro-benzamide (0.134 g) and 2-fluorobenzaldehyde (0.114 g, 3 eq.) were dissolved in 3 ml of MeOH. One added a solution of ZnCl₂ (0.168 g, 4 eq.) and NaCNBH₃ (0.058 g, 3. eq.) in 1.5 ml of MeOH and stirred for 21 h at 60° C. Pouring onto crashed ice/NH₄Cl-solution, twofold extraction with AcOEt, washing with brine, drying over magnesium sulfate, and evaporation of the solvents, followed by flash chromatography (SiO₂, hexane/AcOEt=25/75) afforded 0.112 g of 3-(carbamoylmethyl-methanesulfonyl-amino)-5-chloro-N-[4-cyano-2-(2-fluoro-benzylamino)-benzyl]-benzamide as white foam. MS [M+H]+=544.3.

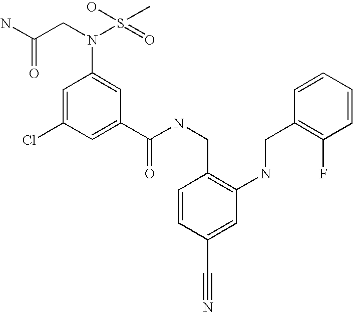

164.6 3-(Carbamoylmethyl-methanesulfonyl-amino)-5-chloro-N-[4-cyano-2-(2-fluoro-benzylamino)-benzyl]-benzamide (0.109 g) was subjected to the Pinner reaction as described in general procedure C to yield after flash chromatography (SiO₂, AcOEt/acetone/AcOH/water=6/2/1/1) and crystallisation from acetonitrile 0.027 g of N-[4-carbamimidoyl-2-(2-fluoro-benzylamino)-benzyl]-3-(carbamoylmethyl-methanesulfonyl-amino)-5-chloro-benzamide; compound with acetic acid, as white crystals. MS [M+H]+=561.4.

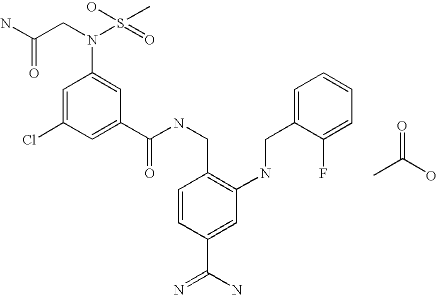

### Example 165

N-(4-Carbamimidoyl-2-ethylamino-benzyl)-3-(carbamoylmethyl-methanesulfonyl-amino)-5-chloro-benzamide; compound with acetic acid, was prepared in analogy to example 164, but using for the reductive amination acetaldehyde instead of 2-fluorobenzaldehyde, as white crystals. MS [M+H]⁺=481.1.

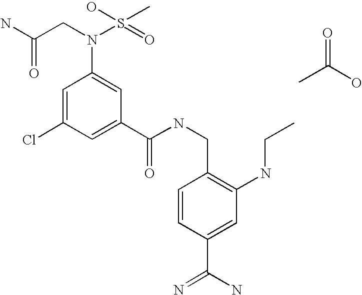

### Example 166

N-(4-Carbamimidoyl-2-ethylamino-benzyl)-3-[carbamoylmethyl-(propane-1-sulfonyl)-amino]-5-chloro-benzamide; compound with acetic acid, was prepared in analogy to example 165, but using for the very first step propanesulfonylchloride instead of methanesulfonylchloride, as light yellow waxy soid. MS [M+H]⁺=509.3.

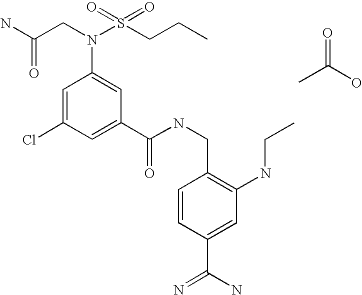

### Example 167

N-(4-Carbamimidoyl-2-ethylamino-benzyl)-3-[carbamoylmethyl-(propane-1-sulfonyl)-amino]-5-chloro-benzamide; compound with acetic acid, was prepared in analogy to example 146, but starting the whole reaction sequence with (4-fluoro-phenyl)-methanesulfonyl chloride instead of phenyl-methanesulfonyl chloride, as off-white solid. MS [M+H]⁺=605.0.

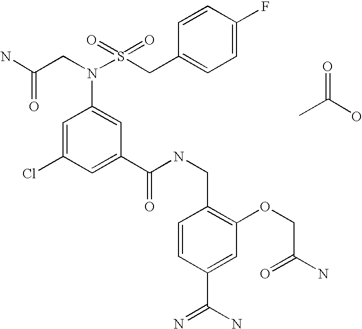

### Example 168

N-(4-Carbamimidoyl-2-carbamoylmethoxy-benzyl)-3-[carbamoylmethyl-(3-fluoro-phenylmethanesulfonyl)-amino]-5-chloro-benzamide; compound with acetic acid, was prepared in analogy to example 146, but starting the whole reaction sequence with (3-fluoro-phenyl)-methanesulfonyl chloride instead of phenyl-methanesulfonyl chloride, as off-white waxy solid. MS [M+H]⁺=605.4.

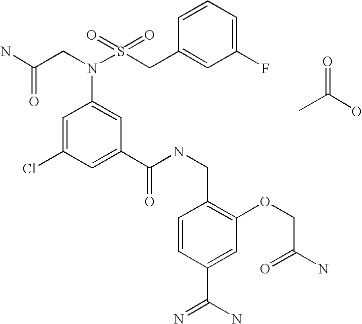

### Example 169

N-(4-Carbamimidoyl-2-carbamoylmethoxy-benzyl)-3-[carbamoylmethyl-(2-fluoro-phenylmethanesulfonyl)-amino]-5-chloro-benzamide; compound with acetic acid, was prepared in analogy to example 146, but starting the whole reaction sequence with (2-fluoro-phenyl)-methanesulfonyl chloride instead of phenyl-methanesulfonyl chloride, as light brown foam. MS [M+H]⁺=605.1.

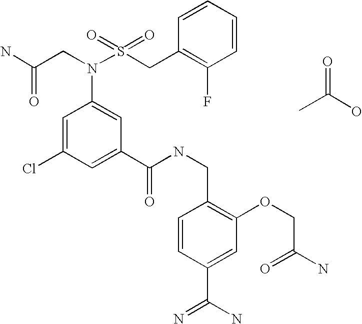

### Example 170

3-(Carbamoylmethyl-phenylmethanesulfonyl-amino)-5-chloro-benzoic acid (0.132 g, intermediate of example 146) was coupled with [1-amino-1-(4-aminomethyl-phenyl)-meth-(Z)-ylidene]-carbamic acid benzyl ester according to general procedure A to give, after flash chromatography (SiO₂, hexane/AcOEt=3/7), [1-amino-1-(4-aminomethyl-phenyl)-meth-(Z)-ylidene]-carbamic acid benzyl ester as white solid (0.076 g).

This intermediate was dissolved in 2.8 ml of EtOH and 0.50 ml of acetic acid and hydrogenated over 0.030 g of Pd on charcoal (10%) at atmospheric pressure and ambient temperature. After 5 h, the reaction mixture was filtered over a pad of Celite and rinsed generously with EtOH. The combined filtrates were evaporated to dryness and the residue crystallized from acetonitrile/diethyl ether to yield 0.038 g of N-(4-carbamimidoyl-benzyl)-3-(carbamoylmethyl-phenylmethanesulfonyl-amino)-5-chloro-benzamide; compound with acetic acid, as off-white solid. [M+H]⁺=514.1.

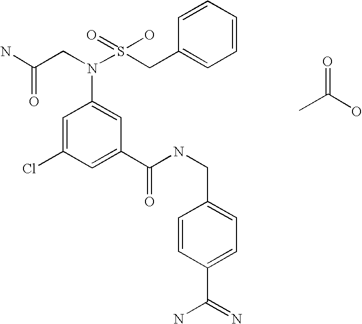

### Example 171

N-(4-Carbamimidoyl-benzyl)-3-[carbamoylmethyl-(3-fluoro-phenylmethanesulfonyl)-amino]-5-chloro-benzamide; compound with acetic acid, was prepared in analogy to example 170, but starting the whole reaction sequence with (3-fluoro-phenyl)-methanesulfonyl chloride instead of phenyl-methanesulfonyl chloride, as yellow solid. MS [M+H]⁺=532.1.

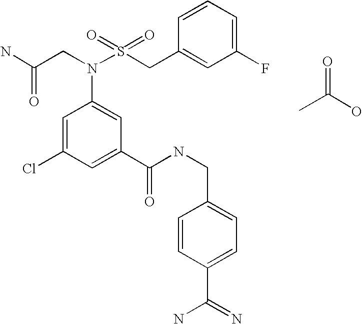

### Example 172

172.1 3-(Benzyl-methanesulfonyl-amino)-5-chloro-N-(4-cyano-2-hydroxy-benzyl)-benzamide (0.160 g, example 140.5) was treated successively with cesium carbonate (0.333 g) and methyl bromoacetate (0.104 g), and the reaction mixture was stirred at ambient temperature for 2 h. Pouring onto crashed ice/NH₄Cl-solution, twofold extraction with AcOEt, washing with water and brine, drying over sodium sulfate, and evaporation of the solvents, followed by flash chromatography (SiO₂, hexane/AcOEt=1/1) afforded 0.161 g of (2-{[3-(benzyl-methanesulfonyl-amino)-5-chloro-benzoylamino]-methyl}-5-cyano-phenoxy)-acetic acid ethyl ester as off-white foam. MS [M+H]⁺=542.3.

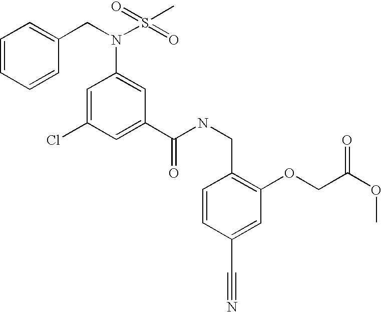

172.2 (2-{[3-(Benzyl-methanesulfonyl-amino)-5-chloro-benzoylamino]-methyl}-5-cyano-phenoxy)-acetic acid methyl ester (0.160 g) was dissolved in 1.5 ml of MeOH, treated with 2-aminomethyl-pyridine (0.128 g), and refluxed over night, whereby most of the solvent had evaporated. Direct flash chromatography (SiO₂, AcOEt) yielded 0.160 g of 3-(benzyl-methanesulfonyl-amino)-5-chloro-N-(4-cyano-2-{[(pyridin-2-ylmethyl)-carbamoyl]-methoxy}-benzyl)-benzamide as light yellow foam. MS [M+H]⁺=618.2.

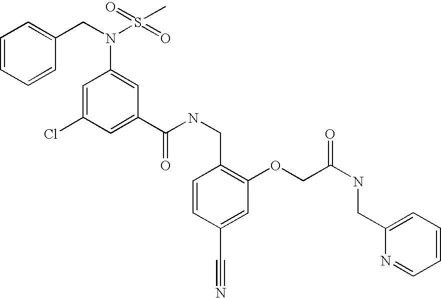

172.3 3-(Benzyl-methanesulfonyl-amino)-5-chloro-N-(4-cyano-2-{[(pyridin-2-ylmethyl)-carbamoyl]-methoxy}-benzyl)-benzamide (0.160 g) was subjected to the Pinner reaction as described in general procedure C to yield, after direct crystallisation from acetonitrile/MeOH, 0.090 g of 3-(enzyl-methanesulfonyl-amino)-N-(4-carbamimidoyl-2-{[(pyridin-2-ylmethyl)-carbamoyl]-methoxy}-benzyl)-5-chloro-benzamide; compound with HCl as light brown crystals. MS [M+H]+=635.1.

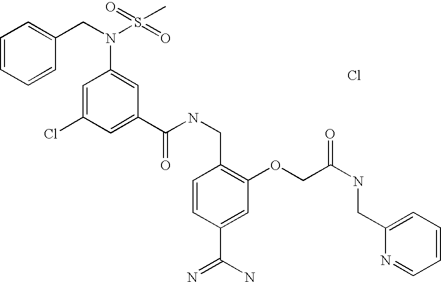

### Example 173

N-(4-Carbamimidoyl-2-{[(pyridin-3-ylmethyl)-carbamoyl]-methoxy}-benzyl)-3-chloro-5-nitro-benzamideacetic acid; compound with acetic acid, was prepared in analogy to example 172, but starting the reaction sequence with 3-chloro-5-nitro-benzoic acid (CAS 34662-36-7) instead of 3-(benzyl-methanesulfonyl-amino)-5-chloro-benzoic acid, as off-white solid. MS [M+H]+=497.

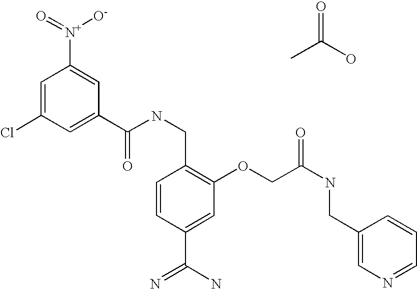

### Formulation Examples

The following are representative pharmaceutical Formulations containing a compound of Formula (I).

### Example A

Film coated tablets containing the following ingredients can be manufactured in a conventional manner:

| | | |
|---|---|---|
| Ingredients | a. | b |

| | | | | |
|---|---|---|---|---|
| Kernel: | | | | |
| Compound of formula (I) | 10.0 | mg | 200.0 | mg |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxyde (yellow) | 0.8 | mg | 1.6 | mg |
| Titan dioxide | 0.8 | mg | 1.6 | mg |

The active ingredient is sieved and mixed with microcrystalline cellulose and the mixture is granulated with a solution of polyvinylpyrrolidon in water. The granulate is mixed with sodium starch glycolate and magesiumstearate and compressed to yield kernels of 120 or 350 mg respectively. The kernels are lacquered with an aqueous solution/suspension of the above mentioned film coat.

### Example B

Capsules containing the following ingredients can be manufactured in a conventional manner:

| Ingredients | Per capsule | |
|---|---|---|
| Compound of formula (I) | 25.0 | mg |
| Lactose | 150.0 | mg |
| Maize starch | 20.0 | mg |
| Talc | 5.0 | mg |

The components are sieved and mixed and filled into capsules of size 2.

### Example C

Injection solutions can have the following composition:

| Compound of formula (I) | 3.0 | mg |
|---|---|---|
| Polyethylene Glycol 400 | 150.0 | mg |
| Acetic Acid | q.s. ad pH 5.0 | |
| Water for injection solutions | ad 1.0 | ml |

The active ingredient is dissolved in a mixture of Polyethylene Glycol 400 and water for injection (part). The pH is adjusted to 5.0 by Acetic Acid. The volume is adjusted to 1.0 ml by addition of the residual amount of water. The solution is filtered, filled into vials using an appropriate overage and sterilized.

### Example D

Soft gelatin capsules containing the following ingredients can be manufactured in a conventional manner:

| (ii) Capsule contents | | |
|---|---|---|
| Compound of formula (I) | 5.0 | mg |
| Yellow wax | 8.0 | mg |
| Hydrogenated Soya bean oil | 8.0 | mg |
| Partially hydrogenated plant oils | 34.0 | mg |
| Soya bean oil | 110.0 | mg |
| Weight of capsule contents | 165.0 | mg |
| (iii) Gelatin capsule | | |
| Gelatin | 75.0 | mg |
| Glycerol 85% | 32.0 | mg |
| Karion 83 | 8.0 | mg |
| | (dry matter) | |
| Titan dioxide | 0.4 | mg |
| Iron oxide yellow | 1.1 | mg |

The active ingredient is dissolved in a warm melting of the other ingredients and the mixture is filled into soft gelatin capsules of appropriate size. The filled soft gelatin capsules are treated according to the usual procedures.

### Example E

Sachets containing the following ingredients can be manufactured in a conventional manner:

| | | |
|---|---|---|
| Compound of formula (I) | 50.0 | mg |
| Lactose, fine powder | 1015.0 | mg |
| Microcristalline cellulose (AVICEL PH 102) | 1400.0 | mg |
| Sodium carboxymethyl cellulose | 14.0 | mg |
| Polyvinylpyrrolidon K 30 | 10.0 | mg |
| Magnesiumstearate | 10.0 | mg |
| Flavoring additives | 1.0 | mg |

The active ingredient is mixed with lactose, microcristalline cellulose and sodium carboxymethyl cellulose and granulated with a mixture of polyvinylpyrrolidon in water. The granulate is mixed with magnesiumstearate and the flavouring additives and filled into sachets.

### Biological Example

Measurements were carried out by an automated robotic assay on microtitre plates at room temperature. To this end, 100 μl of a solution of 26 nM of tissue factor, 9 nM of soluble factor VIIa and 8 mM of calcium chloride were added to 25 μl of a solution of the inhibitor in a buffer [pH 7.5, 100 mM, comprising 0.14M NaCl, 0.1M N-(2-hydroxyethyl)piperazine-N′-(2-ethanesulphonic acid) (HEPES), 0.5 mg/l of fatty-acid-free BSA (bovine serum albumin) and 0.05% NaN₃] in each well of the plate. After an incubation time of 15 minutes the reaction was started by the addition of 50 μl of chromogenic substrate Chromozym-tPA (3.5 mM, MeSO₂-D-Phe-Gly-Arg-paranitroamlide) and the hydrolysis of the substrate was followed spectrophotometrically on a kinetic microtitre plate reader over 10 minutes. Using the plot of the inhibition curves, the Ki values were determined according to the method described in Biochem. J. 55, 1953, 170-171.

The activity of the low molecular weight substances can, moreover, be characterized in the “prothrombin time” (PT) clotting test. The substances are prepared as a 10 mM solution in DMSO or DMSO/0.1M HCl (DHCl) and thereafter made up to the desired dilution in the same solvent. Thereafter, 0.25 ml of human plasma (obtained from whole blood anticoagulated with 1/10 volume of 108 mM Na citrate) was placed in the instrument-specific sample container. In each case 5 μl of each dilution of the substance-dilution series was then mixed with the plasma provided. This plasma/inhibitor mixture was incubated at 37° C. for 2 minutes. Thereafter, there were pipetted to the semi-automatic device (ACL, Automated Coagulation Laboratory (Instrument Laboratory)) 50 μl of plasma/inhibitor mixture in the measurement container. The clotting reaction was initiated by the addition of 0.1 ml of Innovin® (recombinant human tissue factor combined with calcium buffer and synthetic phospholipids( Dade Behring®, Inc.)). The time up to the fibrin cross-linking was determined photooptically from the ACL. The inhibitor concentration which brought about a doubling of the PT clotting time was determined by means of a graph.

The Ki values of the active compounds of the present invention amount to about 0.001 to 50 μM, especially about 0.001 to 1 μM. The PT values amount to about 1 to 100 μM, especially to about 1 to 10 μM.

| Example | Ki [μM] |
|---|---|
| 18.2 | 0.062 |
| 24.1 | 0.081 |
| 42.2 | 0.051 |

What is claimed is:

1. A compound of formula (I) 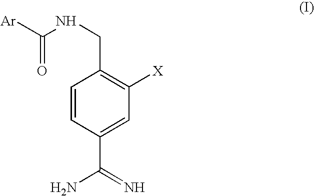
wherein
Ar is selected from the group consisting of aryl and heteroaryl, wherein Ar is optionally substituted by one, two or three substituents independently selected from the group consisting of halogen, C₁₋₆ alkyl, C₂₋₆ alkenyl, C₂₋₆ alkynyl, C₃₋₇ cycloalkyl, C₃₋₇ cycloalkyl C₁₋₆ alkyl, optionally substituted heterocyclyl, optionally substituted heteroaryl, optionally substituted aryl, optionally substituted aryl-C₁₋₆ alkyl, optionally substituted heteroaryl-C₁₋₆ alkyl, optionally substituted heterocyclyl-C₁₋₆ alkyl, optionally substituted aryloxy-C₁₋₆ alkyl, optionally substituted heteroaryloxy-C₁₋₆ alkyl, optionally substituted heterocyclyloxy-C₁₋₆ alkyl, optionally substituted aryl-C₁₋₆ alkoxy, optionally substituted heteroaryl-C₁₋₆ alkoxy, optionally substituted heterocyclyl-C₁₋₆ alkoxy, fluoro C₁₋₆ alkyl, hydroxy, C₁₋₆ alkoxycarbonyl, carboxy, nitro, cyano, hydroxy C₁₋₆ alkyl-aminocarbonyl, optionally substituted heterocyclyl-carbonyl, optionally substituted heteroaryl-carbonyl, optionally substituted aryl-carbonyl, C₁₋₆ alkoxy C₁₋₆ alkyl-aminocarbonyl, C₁₋₆ alkoxy C₁₋₆ alkoxy, C₁₋₆ alkoxy, and amino, wherein C₁₋₆ alkoxy may optionally be substituted by one or two substituents independently selected from the group consisting of hydroxy, optionally substituted heteroaryl, optionally substituted aryl, optionally substituted heterocyclyl, carbamoyl, mono- or di-C₁₋₆ alkyl substituted aminocarbonyl, carboxyl and C₁₋₆ alkoxycarbonyl, and amino may optionally be substituted by one or two substituents independently selected from the group consisting of optionally substituted aryl-sulfanyl, optionally substituted aryl-sulfinyl, optionally substituted aryl-sulfonyl, optionally substituted heteroaryl-sulfanyl, optionally substituted heteroaryl-sulfinyl, optionally substituted heteroaryl-sulfonyl, optionally substituted heterocyclyl-sulfanyl, optionally substituted heterocyclyl-sulfinyl, optionally substituted heterocyclyl-sulfonyl, optionally substituted aryl-C₁₋₆ alkylsulfanyl, optionally substituted aryl-C₁₋₆ alkylsulfinyl, optionally substituted aryl-C₁₋₆ alkylsulfonyl, optionally substituted heteroaryl-C₁₋₆ alkylsulfanyl, optionally substituted heteroaryl-C₁₋₆ alkylsulfinyl, optionally substituted heteroaryl-C₁₋₆ alkylsulfonyl, optionally substituted heterocyclyl-C₁₋₆ alkylsulfanyl, optionally substituted heterocycyl-C₁₋₆ alkylsulfinyl, optionally substituted heterocyclyl-C₁₋₆ alkylsulfonyl, optionally substituted heteroaryl-C₁₋₆ alkyl, optionally substituted aryl-C₁₋₆ alkyl, optionally substituted heterocyclyl-C₁₋₆ alkyl, optionally substituted aryl-C₁₋₆ alkylcarbonyl, optionally substituted heteroaryl-C₁₋₆ alkylcarbonyl, optionally substituted heterocyclyl-C₁₋₆ alkylcarbonyl, mono- or di-C₁₋₆ alkyl substituted aminocarbonyl-C₁₋₆ alkyl, C₁₋₆ alkoxycarbonyl-C₁₋₆ alkyl, C₁₋₆ alkyl, carbamoyl C₁₋₆ alkyl, C₁₋₆ alkylcarbamoyl, alkylcarbonyl, C₁₋₆ alkylsulfanyl, C₁₋₆ alkylsulfinyl and C₁₋₆ alkylsulfonyl;
X is selected from the group consisting of X-I: —O—(CH₂)ₙ—Y—R², X-2:—N(R¹)—(CH₂)ₙ—Y—R², X-3:—NO₂ and X-4: hydrogen;
Y is absent or is selected from the group consisting of Y-1:—C(═O)— and Y-2: 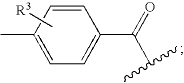
R¹ is selected from the group consisting of hydrogen, C₁₋₆ alkyl, C₃₋₇ cycloalkyl, C₃₋₇ cycloalkyl C₁₋₆ alkyl, optionally substituted aryl-C₁₋₆ alkyl or hydroxy C₁₋₆ alkyl, C₁₋₆ alkoxy C₁₋₆ alkyl;
R² is selected from the group consisting of hydrogen, C₁₋₆ alkyl, C₃₋₇ cycloalkyl, C₃₋₇ cycloalkyl C₁₋₆ alkyl, C₂₋₆ alkenyl, C₂₋₆ alkynyl, C₁₋₆ alkoxy, hydroxy, optionally substituted aryl, optionally substituted heterocyclyl, optionally substituted aryl-C₁₋₆ alkyl, optionally substituted heteroaryl-C₁₋₆ alkyl, optionally substituted heterocyclyl-C₁₋₆ alkyl, nitro, cyano, heteroaryl optionally substituted by one or two substituents independently selected from the group consisting of C₁₋₆ alkyl, carboxy, carbamoyl and C₁₋₆ alkoxycarbonyl and amino optionally substituted by one or two substituents independently selected from the group consisting of C₁₋₆ alkyl, C₃₋₇ cycloalkyl, C₃₋₇ cycloalkyl C₁₋₆ alkyl, C₁₋₆ alkylcarbonyl, C₁₋₆ alkylsulfanyl, C₁₋₆ alkylsulfinyl, C₁₋₆ alkylsulfonyl, optionally substituted aryl-carbonyl, optionally substituted heteroaryl-carbonyl, optionally substituted heterocyclyl-carbonyl, optionally substituted aryl, optionally substituted aryl-C₁₋₆ alkyl, optionally substituted heterocyclyl-C₁₋₆ alkyl, C₁₋₆ alkoxy C₁₋₆ alkyl, C₁₋₆ alkoxycarbonyl-C₁₋₆ alkyl, carboxyl-C₁₋₆ alkyl, optionally substituted heteroaryl, optionally substituted heteroaryl-C₁₋₆ alkyl, optionally substituted heterocyclyl and hydroxy C₁₋₆ alkyl;
R³ is selected from the group consisting of hydrogen, halogen and C₁₋₆ alkyl;
n is an integer from 0 to 2;
provided that X is not C₁₋₆ alkoxy;
and prodrugs and pharmaceutically acceptable salts thereof.

2. A compound according to claim 1 wherein
Ar is selected from the group consisting of aryl and heteroaryl, wherein Ar is optionally substituted by one, two or three substituents independently selected from the group consisting of halogen, C₁₋₆ alkyl, C₂₋₆ alkenyl, C₂₋₆ alkynyl, C₃₋₇ cycloalkyl, C₃₋₇ cycloalkyl C₁₋₆ alkyl, optionally substituted aryl-C₁₋₆ alkyl, optionally substituted heteroaryl-C₁₋₆ alkyl, optionally substituted heterocyclyl-C₁₋₆ alkyl, fluoro C₁₋₆ alkyl, hydroxy, C₁₋₆ alkoxycarbonyl, carboxy, nitro, cyano, hydroxy C₁₋₆ alkyl-aminocarbonyl, optionally substituted heterocyclyl-carbonyl, C₁₋₆ alkoxy C₁₋₆ alkyl-aminocarbonyl, C₁₋₆ alkoxy and amino, wherein C₁₋₆ alkoxy may optionally be substituted by one or two substituents independently selected from the group consisting of hydroxy, optionally substituted heteroaryl, optionally substituted aryl, optionally substituted heterocyclyl, carbamoyl, mono C₁₋₆ alkyl substituted aminocarbonyl, carboxy and C₁₋₆ alkoxycarbonyl, and amino may optionally be substituted by one or two substituents independently selected from the group consisting of optionally substituted aryl-sulfanyl, optionally substituted aryl-sulfinyl, optionally substituted aryl-sulfonyl, optionally substituted heteroaryl-C₁₋₆ alkyl, C₁₋₆ alkyl, carbamoyl C₁₋₆ alkyl, C₁₋₆ alkylcarbamoyl, C₁₋₆ alkylsulfanyl, C₁₋₆ alkylsulfinyl and C₁₋₆ alkylsulfonyl;
X is selected from the group consisting of X-1:—O—(CH₂)ₙ—Y—R², X-2:—N(R¹)—(CH₂)ₙ—Y—R² and X-3:—NO₂;
Y is absent or is selected from the group consisting of Y-1:—C(═O)— and Y-2: 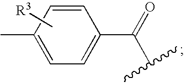
R¹ is selected from the group consisting of hydrogen, C₁₋₆ alkyl, C₃₋₇ cycloalkyl, C₃₋₇ cycloalkyl C₁₋₆ alkyl, optionally substituted aryl-C₁₋₆ alkyl or hydroxy C₁₋₆ alkyl, C₁₋₆ alkoxy C₁₋₆ alkyl;
R² is selected from the group consisting of hydrogen, C₁₋₆ alkyl, C₃₋₇ cycloalkyl, C₃₋₇ cycloalkyl C₁₋₆ alkyl, C₂₋₆ alkenyl, C₂₋₆ alkynyl, C₁₋₆ alkoxy, hydroxy, optionally substituted aryl, optionally substituted heterocyclyl, optionally substituted aryl-C₁₋₆ alkyl, optionally substituted heteroaryl-C₁₋₆ alkyl, optionally substituted heterocyclyl-C₁₋₆ alkyl, nitro, cyano, heteroaryl optionally substituted by one or two substituents independently selected from the group consisting of C₁₋₆ alkyl, carboxy, carbamoyl and C₁₋₆ alkoxycarbonyl, and amino optionally substituted by one or two substituents independently selected from the group consisting of C₁₋₆ alkyl, C₁₋₆ alkylcarbonyl, C₁₋₆ alkylsulfanyl, C₁₋₆ alkylsulfinyl, C₁₋₆ alkylsulfonyl, optionally substituted aryl-carbonyl, optionally substituted aryl, optionally substituted heterocyclyl-C₁₋₆ alkyl, C₁₋₆ alkoxy C₁₋₆ alkyl, optionally substituted heteroaryl, optionally substituted heterocyclyl and hydroxy C₁₋₆ alkyl;
R³ is selected from the group consisting of hydrogen, halogen or C₁₋₆ alkyl;
n is an integer from 0 to 2;
provided that X is not C₁₋₆ alkoxy;
and prodrugs and pharmaceutically acceptable salts thereof.

3. A compound according to claim 1 wherein Ar is optionally substituted by one or two substituents independently selected from the group consisting of halogen, C₁₋₆ alkyl, optionally substituted aryl-C₁₋₆ alkyl, fluoro C₁₋₆ alkyl, hydroxy, C₁₋₆ alkoxycarbonyl, carboxy, nitro, cyano, hydroxy C₁₋₆ alkyl-aminocarbonyl, optionally substituted heterocyclyl-carbonyl, C₁₋₆ alkoxy C₁₋₆ alkyl-aminocarbonyl, C₁₋₆ alkoxy and amino wherein C₁₋₆ alkoxy may optionally be substituted by one or two substituents independently selected from the group consisting of hydroxy, carboxy, C₁₋₆ alkoxycarbonyl, optionally substituted heteroaryl, carbamoyl and mono C₁₋₆ alkyl substituted aminocarbonyl, and amino may optionally be substituted by one or two substituents independently selected from the group consisting of optionally substituted aryl-sulfonyl, optionally substituted heteroaryl-C₁₋₆ alkyl, C₁₋₆ alkyl, carbamoyl C₁₋₆ alkyl and C₁₋₆ alkylsulfonyl

4. A compound according to claim 1 wherein X is X-1 and n is 1.

5. A compound according to claim 4 wherein Y is Y-1.

6. A compound according to claim 5 wherein R² is amino optionally substituted by one or two substituents selected independently from the group consisting of C₁₋₆ alkyl, optionally substituted aryl and optionally substituted heterocyclyl-C₁₋₆ alkyl.

7. A compound according to claim 6 wherein R² is selected from the group consisting of amino and mono C₁₋₆ alkyl substituted amino.

8. A compound according to claim 5 selected from the group consisting of
N-(4-Carbamimidoyl-2-carbamoylmethoxybenzyl)-3-fluoro-benzamide hydrochloride;
N-(4-Carbamimidoyl-2-carbamoylmethoxybenzyl)-3-methoxy-benzamide hydrochloride;
4-Amino-N-(4-Carbamimidoyl-2-carbamoylmethoxybenzyl)-3-methyl-benzamide acetic acid salt;
5-Methyl-isoxazole-3-carboxylic acid 4-carbamimidoyl-2-carbamoylmethoxy-benzylamide acetic acid salt;
3-Methyl-isoxazole-5-carboxylic acid 4-carbamimidoyl-2-carbamoylmethoxy-benzylamide acetic acid salt;
N-(4-Carbamimidoyl-2-carbamoylmethoxybenzyl)-3-methyl-benzamide hydrochloride;
2-Benzyl-5-methyl-2H-pyrazole-3-carboxylic acid 4-carbamimidoyl-2-carbamoylmethoxy-benzylamide acetic acid salt;
N-(4-Carbamimidoyl-2-carbamoylmethoxybenzyl)-5-methyl-nicotinamide hydrochloride;
N-(4-Carbamimidoyl-2-carbamoylmethoxybenzyl)-5-methyl-nicotinamide ammoniumchloride;
N-(4-Carbamimidoyl-2-carbamoylmethoxybenzyl)-2-methyl-isonicotinamide acetic acid salt;
2-Benzenesulfonylamino-N-(4-carbamimidoyl-2-methylcarbamoylmethoxy-benzyl)-5-methyl-benzamide hydrochloride;
N-(4-Carbamimidoyl-2-methylcarbamoylmethoxy-benzyl)-2,5-dichloro-benzamide hydrochloride;
N-(4-Carbamimidoyl-2-methylcarbamoylmethoxy-benzyl)-3-chloro-4-fluoro-benzamide hydrochloride;
N-(4-Carbamimidoyl-2-methylcarbamoylmethoxy-benzyl)-3,5-dichloro-4-fluoro-benzamide hydrochloride;
N-(4-Carbamimidoyl-2-methylcarbamoylmethoxy-benzyl)-3,5-dichloro-benzamide hydrochloride;
N-(4-Carbamimidoyl-2-methylcarbamoylmethoxy-benzyl)-5-chloro-nicotinamide hydrochloride;
N-(4-Carbamimidoyl-2-methylcarbamoylmethoxy-benzyl)-3-trifluoromethyl-benzamide hydrochloride;
N-(4-Carbamimidoyl-2-methylcarbamoylmethoxy-benzyl)-3-chloro-5-methoxy-benzamide hydrochloride;
N-(4-Carbamimidoyl-2-methylcarbamoylmethoxy-benzyl)-5-chloro-2-hydroxy-benzamide hydrochloride;
2,5-Dimethyl-2H-pyrazole-3-carboxylic acid 4-carbamimidoyl-2-methylcarbamoylmethoxy-benzylamide hydrochloride;
1,5-Dimethyl-1H-pyrazole-3-carboxylic acid 4-carbamimidoyl-2-methylcarbamoylmethoxy-benzylamide hydrochloride;
2-Methyl-oxazole-4-carboxylic acid 4-carbamimidoyl-2-methylcarbamoylmethoxy-benzylamide hydrochloride;
N-(4-Carbamimidoyl-2-carbamoylmethoxy-benzyl)-4-fluoro-3-methyl-benzamide acetic acid salt;
N-(4-Carbamimidoyl-2-methylcarbamoylmethoxy-benzyl)-4-fluoro-3-methyl-benzamide hydrochloride;
[1-Amino-1-{4-[(4-fluoro-3-methyl-benzoylamino)-methyl]-3-methylcarbamoylmethoxy-phenyl}-meth-(Z)-ylidene]-carbamic acid ethyl ester;
4-Fluoro-N-[4-(N-hydroxycarbamimidoyl)-2-methylcarbamoylmethoxy-benzyl]-3-methyl-benzamide;
(RS)-N-(4-Carbamimidoyl-2-carbamoylmethoxy-benzyl)-3-chloro-benzamide hydrochloride;
N-(4-Carbamimidoyl-2-methylcarbamoylmethoxy-benzyl)-3-chloro-benzamide acetic acid salt;
N-{4-Carbamimidoyl-2-[(4-fluoro-phenylcarbamoyl)-methoxy]-benzyl}-3-chloro-benzamide acetic acid salt;
N-{4-Carbamimidoyl-2-[(2-morpholin-4-yl-ethylcarbamoyl)-methoxy]-benzyl}-3-chloro-benzamide hydrochloride;
N-(4-Carbamimidoyl-2-methylcarbamoylmethoxy-benzyl)-5-chloro-isophthalamic acid methyl ester hydrochloride;
N-(4-Carbamimidoyl-2-methylcarbamoylmethoxy-benzyl)-5-chloro-isophthalamic acid;
N-(4-Carbamimidoyl-2-methylcarbamoylmethoxy-benzyl)-5-chloro-N′-(2-methoxy-ethyl)-isophthalamide hydrochloride;
N-(4-Carbamimidoyl-2-methylcarbamoylmethoxy-benzyl)-3-chloro-5-(morpholine-4-carbonyl)-benzamide hydrochloride;
N-(4-Carbamimidoyl-2-methylcarbamoylmethoxy-benzyl)-5-chloro-N′-(2-hydroxy-ethyl)-isophthalamide hydrochloride;
N-(4-Carbamimidoyl-2-methylcarbamoylmethoxy-benzyl)-5-chloro-2-methylamino-benzamide hydrochloride;
N-(4-Carbamimidoyl-2-methylcarbamoylmethoxy-benzyl)-5-chloro-2-(2-pyridin-4-yl-ethylamino)-benzamide hydrochloride;
3-Amino-N-(4-carbamimidoyl-2-methylcarbamoylmethoxy-benzyl)-benzamide hydrochloride;
N-(4-Carbamimidoyl-2-methylcarbamoylmethoxy-benzyl)-3-hydroxy-5-methyl-benzamide hydrochloride;
[3-(4-Carbamimidoyl-2-methylcarbamoylmethoxy-benzylcarbamoyl)-5-methyl-phenoxy]-acetic acid;
N-(4-Carbamimidoyl-2-methylcarbamoylmethoxy-benzyl)-3-chloro-5-hydroxy-benzamide hydrochloride;
(4-Carbamimidoyl-2-carbamoylmethoxy-benzyl)-3-chloro-5-nitro-benzamide acetic acid salt;
(4-Carbamimidoyl-2-methylcarbamoylmethoxy-benzyl)-3-chloro-5-nitro-benzamide acetic acid salt;
N-(4-Carbamimidoyl-2-methylcarbamoylmethoxy-benzyl)-3-chloro-5-fluoro-benzamide hydrochloride;
N-(4-Carbamimidoyl-2-methylcarbamoylmethoxy-benzyl)-3-chloro-5-fluoro-benzamide hydrochloride;
N-(4-Carbamimidoyl-2-methylcarbamoylmethoxy-benzyl)-3-chloro-2-fluoro-5-methoxy-benzamide hydrochloride;
N-(4-Carbamimidoyl-2-methylcarbamoylmethoxy-benzyl)-3-chloro-2,4-difluoro-benzamide hydrochloride;
N-(4-Carbamimidoyl-2-carbamoylmethoxy-benzyl)-3-carbamoylmethoxy-5-chloro-benzamide hydrochloride;
N-(4-Carbamimidoyl-2-methylcarbamoylmethoxy-benzyl)-3-chloro-5-methylcarbamoylmethoxy-benzamide hydrochloride;
N-(4-Carbamimidoyl-2-methylcarbamoylmethoxy-benzyl)-3-(2-hydroxy-ethoxy)-5-methyl-benzamide hydrochloride;
N-(4-Carbamimidoyl-2-methylcarbamoylmethoxy-benzyl)-3-carbamoylmethoxy-5-methyl-benzamide acetic acid salt;
N-(4-Carbamimidoyl-2-methylcarbamoylmethoxy-benzyl)-3-chloro-5-(pyridin-4-ylmethoxy)-benzamide hydrochloride;
N-(4-Carbamimidoyl-2-carbamoylmethoxy-benzyl)-3-chloro-5-(pyridin-4-ylmethoxy)-benzamide hydrochloride;
N-(4-Carbamimidoyl-2-carbamoylmethoxy-benzyl)-3-chloro-5-(pyridin-3-ylmethoxy)-benzamide hydrochloride;
N-(4-Carbamimidoyl-2-carbamoylmethoxy-benzyl)-3-chloro-5-(pyridin-2-ylmethoxy)-benzamide hydrochloride;
N-(4-Carbamimidoyl-2-methylcarbamoylmethoxy-benzyl)-3-chloro-5-(1-methyl-1H-imidazol-2-ylmethoxy)-benzamide hydrochloride;
3Amino-N-(4-carbamimidoyl-2-carbamoylmethoxy-benzyl)-5-chloro-4-fluoro-benzamide hydrochloride;
3-Acetylamino-N-(4-carbamimidoyl-2-methylcarbamoylmethoxy-benzyl)-5-chloro-4-fluoro-benzamide hydrochloride;
N-(4-Carbamimidoyl-2-carbamoylmethoxy-benzyl)-3-chloro-5-isobutyrylamino-benzamide;
N-(4-Carbamimidoyl-2-methylcarbamoylmethoxy-benzyl)-3-chloro-5-isobutyrylamino-benzamide;
{5-Carbamimidoyl-2-[(3-chloro-5-isobutyrylamino-benzoylamino)-methyl]-phenoxy}-acetic acid ethyl ester;
3-Acetylamino-N-(4-carbamimidoyl-2-methylcarbamoylmethoxy-benzyl)-5-chloro-benzamide;
N-(4-Carbamimidoyl-2-methylcarbamoylmethoxy-benzyl)-3-chloro-5-methanesulfonylamino-benzamide;
N-(4-Carbamimidoyl-2-carbamoylmethoxy-benzyl)-3-chloro-5-methanesulfonylamino-benzamide; and
N-(4-Carbamimidoyl-2-carbamoylmethoxy-benzyl)-3-(carbamoylmethyl-methanesulfonyl-amino)-5-chloro-benzamide.

9. A compound according to claim 4 wherein Y is absent and R² is heteroaryl optionally substituted by one or two substituents selected from the group consisting of C₁₋₆ alkyl, carboxy and C₁₋₆ alkoxycarbonyl.

10. A compound according to claim 9 selected from the group consisting of 4-Amino-N-[4-carbamimidoyl-2-(pyridin-2-ylmethoxy)-benzyl]-3-methyl-benzamide acetic acid salt;
N-[4-Carbamimidoyl-2-(pyridin-2-ylmethoxy)-benzyl]-3-methyl-benzamide hydrochloride;
5-{5-Carbamimidoyl-2-[(3-methyl-benzoylamino)-methyl]-phenoxymethyl}-2-methyl-2H-pyrazole-3-carboxylic acid ethyl ester hydrochloride;
5-{5-Carbamimidoyl-2-[(3-methyl-benzoylamino)-methyl]-phenoxymethyl}-2-methyl-2H-pyrazole-3-carboxylic acid; and
N-[4-Carbamimidoyl-2-(pyridin-2-ylmethoxy)-benzyl]-3-chloro-5-(pyridin-2-ylmethoxy)-benzamide hydrochloride.

11. A compound according to claim 4 wherein Y is Y-2.

12. A compound according to claim 11 wherein R³ is hydrogen and R² is selected from the group consisting of hydroxy and C₁₋₆ alkoxy.

13. A compound according to claim 12 selected from the group consisting of
4-{5-Carbamimidoyl-2-[(3-methylbenzoylamino)-methyl]-phenoxymethyl}-benzoic acid methyl ester hydrochloride and
4-{5-Carbamimidoyl-2-[(3-methylbenzoylamino)-methyl]-phenoxymethyl}-benzoic acid.

14. A compound according to claim 1 wherein X is X-1 and n is 2.

15. A compound according to claim 14 wherein Y is absent and R² is selected from the group consisting of C₁₋₆ alkylcarbonylamino, C₁₋₆ alkylsulfanylamino, C₁₋₆ alkylsulfinylamino, C₁₋₆ alkylsulfonylamino, heterocyclyl and optionally substituted aryl-carbonylamino.

16. A compound according to claim 15 wherein R² is selected from the group consisting of optionally substituted aryl-carbonylamino and heterocyclyl.

17. A compound according to claim 16 wherein Ar is aryl optionally substituted by a member selected from the group consisting of halogen and C₁₋₆ alkyl.

18. A compound according to claim 17 selected from the group consisting of
N-[2-(2-Acetylamino-ethoxy)-4-Carbamimidoyl-benzyl]-4-fluoro-3-methyl-benzamide hydrochloride;
N-[4-Carbamimidoyl-2-(2-methanesulfonylaminoethoxy)-benzyl]-4-fluoro-3-methyl-benzamide hydrochloride;
N-{4-Carbamimidoyl-2-[2-(2-fluorobenzoylamino)-ethoxy]-benzyl}-4-fluoro-3-methyl-benzamide hydrochloride;
N-[2-(2-{[(3-Chlorobenzoyl)amino]methyl}-5-carbamimidoylphenoxy)ethyl]-2-fluorobenzamide; and
N-{4-Carbamimidoyl-2-[2-(1,3-dioxo-1,3-dihydro-isoindol-2-yl)-ethoxy]-benzyl}-4-fluoro-3-methyl-benzamide hydrochloride.

19. A compound according to claim 1 wherein X is X-2 and n is 1.

20. A compound according to claim 19 wherein Y is Y-1.

21. A compound according to claim 20 wherein R² is selected from the group consisting of hydroxy, C₁₋₆ alkoxy, optionally substituted heterocyclyl and amino optionally substituted by one or two substituents independently selected from the group consisting of C₁₋₆ alkyl, hydroxy C₁₋₆ alkyl, optionally substituted heterocyclyl, optionally substituted heteroaryl and optionally substituted aryl.

22. A compound according to claim 21 wherein R¹ is hydrogen.

23. A compound according to claim 22 selected from the group consisting of
{5-Carbamimidoyl-2-[(3-methyl-benzoylamino)-methyl]-phenylamino}acetic acid ethyl ester hydrochloride;
{5-Carbamimidoyl-2-[(3-methyl-benzoylamino)-methyl]-phenylamino}acetic acid;
N-[4-Carbamimidoyl-2-(methylcarbamoylmethyl-amino)-benzyl]-3-methyl-benzamide hydrochloride;
N-[4-Carbamimidoyl-2-(dimethylcarbamoylmethyl-amino)-benzyl]-3-methyl-benzamide hydrochloride;
N-[4-Carbamimidoyl-2-(2-morpholin-4-yl-2-oxo-ethylamino)-benzyl]-3-methyl-benzamide hydrochloride;
N-[4-Carbamimidoyl-2-(phenylcarbamoylmethyl-amino)-benzyl]-3-methyl-benzamide hydrochloride;
{5-Carbamimidoyl-2-[(4-fluoro-3-methyl-benzoylamino)-methyl]-phenylamino}acetic acid ethyl ester hydrochloride;
{5-Carbamimidoyl-2-[(4-fluoro-3-methyl-benzoylamino)-methyl]-phenylamino}acetic acid;
N-{4-Carbamimidoyl-2-[(pyridin-2-ylcarbamoylmethyl)-amino]-benzyl}-3-chloro-benzamide hydrochloride;
N-(4-Carbamimidoyl-2-[(pyridin-3-ylcarbamoylmethyl)-amino]-benzyl}-3-chloro-benzamide hydrochloride;
N-{4-Carbamimidoyl-2-[(isoxazol-3-ylcarbamoylmethyl)-amino]-benzyl}-3-chloro-benzamide hydrochloride;
N-[4-Carbamimidoyl-2-({[(2-hydroxy-ethyl)-pyridin-2-yl-carbamoyl]-methyl}-amino)-benzyl]-3-chloro-benzamide hydrochloride;
N-[4-Carbamimidoyl-2-({[(2-hydroxy-ethyl)-phenyl-carbamoyl]-methyl}-amino)-benzyl]-3-chloro-benzamide hydrochloride;
N-(4-Carbamimidoyl-2-{[(1-methyl-piperidin-4-ylcarbamoyl)-methyl]-amino}-benzyl)-3-chloro-benzamide acetic acid salt;
{5-Carbamimidoyl-2-[(3-chloro-5-methoxy-benzoylamino)-methyl]-phenylamino}-acetic acid; and
{5-Carbamimidoyl-2-[(3-chloro-5-methoxy-benzoylamino)-methyl]-phenylamino}-acetic acid ethyl ester hydrochloride.

24. A compound according to claim 19 wherein Y is Y-2.

25. A compound according to claim 24 wherein R² is selected from the group consisting of hydroxy, C₁₋₆ alkoxy, amino, mono C₁₋₆ alkylamino, di C₁₋₆ alkylamino, C₁₋₆ alkoxy C₁₋₆ alkyl-amino and optionally substituted heterocyclyl.

26. A compound according to claim 25 wherein R³ is selected from the group consisting of hydrogen or halogen.

27. A compound according to claim 26 wherein R¹ is hydrogen.

28. A compound according to claim 27 selected from the group consisting of
4-({5-Carbamimidoyl-2-[(3-methyl-benzoylamino)-methyl]-phenylamino}-methyl)-3-fluoro-benzoic acid methyl ester hydrochloride;
4-({5-Carbamimidoyl-2-[(3-methyl-benzoylamino)-methyl]-phenylamino}-methyl)-3-fluoro-benzoic acid;
N-{2-{[3-(Aminocarbonyl)benzyl]amino}-4-[amino(imino)methyl]-benzyl}-3-chlorobenzamide hydrochloride;
3-({5-Carbamimidoyl-2-[(3-chloro-benzoylamino)-methyl]-phenylamino}-methyl)-benzoic acid methyl ester hydrochloride;
3-({5-Carbamimidoyl-2-[(3-chloro-benzoylamino)-methyl]-phenylamino}-methyl)-benzoic acid;
N-{4-[Amino(hydroxyimino)methyl]-2-[(3-{[(2-methoxyethyl)amino]carbonyl}benzyl)amino]benzyl}-3-chlorobenzamide;
N-{4-[Amino(imino)methyl]-2-[(3-{[(2-methoxyethyl)amino]carbonyl}benzyl)amino]benzyl}-3-chlorobenzamide acetic acid salt;
3-Chloro-N-{4-(N-hydroxycarbamimidoyl)-2-[3-(morpholine-4-carbonyl)-benzylamino]-benzyl}-benzamide;
N-{4-[Amino(imino)methyl]-2-{[3-(4-morpholinylcarbonyl)benzyl]amino}benzyl}-3-chlorobenzamide acetic acid salt;
N-{2-{[4-(Aminocarbonyl)benzyl]amino}-4-[amino(hydroxyimino)methyl]benzyl}-3-chlorobenzamide; and
N-{2-{[4-(Aminocarbonyl)benzyl]amino}-4-[amino(imino)methyl]benzyl}-3-chlorobenzamide acetic acid salt.

29. A compound according to claim 19 wherein Y is absent and R² is selected from the group consisting of hydrogen, optionally substituted heteroaryl and optionally substituted aryl.

30. A compound according to claim 29 selected from the group consisting-of
N-(2-Benzylamino-4-carbamimidoyl-benzyl)-3-methyl-benzamide hydrochloride;
N-(4-Carbamimidoyl-2-dibenzylamino-benzyl)-3-methyl-benzamide hydrochloride;
6-({5-Carbamimidoyl-2-[(3-chloro-benzoylamino)-methyl]-phenylamino}-methyl)-nicotinamide hydrochloride; and
N-[4-Carbamimidoyl-2-(2-fluoro-benzylamino)-benzyl]-3-chloro-5-methanesulfonylamino-benzamide.

31. A compound according to claim 1 wherein X is X-2, n is 2, Y is absent and R² is selected from the group consisting of hydroxy and C₁₋₆ alkoxy.

32. A compound according to claim 32 selected from the group consisting of
N-[4-Carbamimidoyl-2-(2-hydroxy-ethylamino)-benzyl]-3-methyl-benzamide hydrochloride;
N-[4-Carbamimidoyl-2-(2-hydroxy-ethylamino)-benzyl]-3-chloro-benzamide hydrochloride;
N-[4-Carbamimidoyl-2-(2-hydroxy-ethylamino)-benzyl]-3-chloro-5-methoxy-benzamide hydrochloride;
N-{2-[Bis-(2-hydroxy-ethyl)-amino]-4-carbamimidoyl-benzyl}-3-chloro-5-methoxy-benzamide hydrochloride; and
N-[4-Carbamimidoyl-2-(2-hydroxy-ethylamino)-benzyl]-3-chloro-5-hydroxy-benzamide hydrochloride.

33. A compound according to claim 1 wherein X is X-2, n is 0, and Y is Y-1.

34. A compound according to claim 33 wherein R¹ is hydrogen and R² is selected from the group consisting of optionally substituted aryl-C₁₋₆ alkyl, optionally substituted heteroaryl-C₁₋₆ alkyl and optionally substituted heterocyclyl-C₁₋₆ alkyl.

35. A compound according to claim 34 which is
N-(4-Carbamimidoyl-2-phenylacetylamino-benzyl)-3-methyl-benzamide hydrochloride.

36. A compound according to claim 1 wherein X is X-2, n is 0 and Y is absent.

37. A compound according to claim 36 wherein R¹ is hydrogen and R² is selected from the group consisting of hydrogen and C₁₋₆ alkyl.

38. A compound according to claim 37 which is
N-(2-Amino-4-carbamimidoyl-benzyl)-3-methyl-benzamide hydrochloride.

39. A compound according to claim 1 wherein X is X-3.

40. A compound according to claim 39 which is
N-(4-Carbamimidoyl-2-nitro-benzyl)-3-methyl-benzamide hydrochloride.

41. A compound according to claim 1 wherein X is X-1, n is 1, Y is Y-1 and Ar is phenyl substituted by amino substituted by optionally substituted aryl-C₁₋₆ alkylsulfonyl and a member selected from the group consisting of mono-C₁₋₆ alkyl substituted aminocarbonyl-C₁₋₆ alkyl and di-C₁₋₆ alkyl substituted aminocarbonyl-C₁₋₆ alkyl.

42. A compound according to claim 41 wherein optionally substituted aryl-C₁₋₆ alkylsulfonyl is a member selected from the group consisting of fluorophenylmethylsulfonyl and phenylmethylsulfonyl.

43. A compound according to claim 42 selected from the group consisting of
N-(4-Carbamimidoyl-2-carbamoylmethoxy-benzyl)-3-chloro-5-(methylcarbamoylmethyl-phenylmethanesulfonyl-amino)-benzamide;
N-(4-Carbamimidoyl-2-carbamoylmethoxy-benzyl)-3-[carbamoylmethyl-(4-fluoro-phenylmethanesulfonyl)-amino]-5-chloro-benzamide;
N-(4-carbamimidoyl-2-carbamoylmethoxy-benzyl)-3-[carbamoylmethyl-(3-fluoro-phenylmethanesulfonyl)-amino]-5-chloro-benzamide; and
N-(4-carbamimidoyl-2-carbamoylmethoxy-benzyl)-3-[carbamoylmethyl-(2-fluoro-phenylmethanesulfonyl)-amino]-5-chloro-benzamide.

44. A process for the manufacture of compounds of formula (I) as set forth in claim 1, comprising converting the nitrile group in a compound of Formula (II) 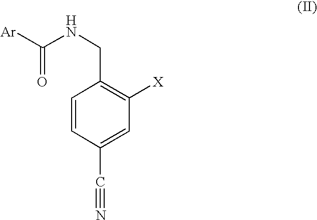
wherein Ar and X are as set forth in claim 1, into a carbamimidoyl group.

45. A composition containing a therapeutically effective amount of a compound according to claim 1 and an excipient.

46. A method for the treatment of diseases associated with the formation of clotting factors Xa, IXa and thrombin induced by factor VIIa and tissue factor comprising the administration of an effective amount of a compound according to claim 1 to a mammal suffering from a disease associated with the formation of clotting factors Xa, IXa and thrombin induced by factor VIIa and tissue factor.

47. The method according to claim 46, wherein the disease is a member selected from the group consisting of arterial and venous thrombosis, deep vein thrombosis, pulmonary embolism, unstable angina pectoris, cardiac infarction, stroke due to atrial fibrillation, inflammation, arteriosclerosis and/or tumour.
